(19) United States
(12) Patent Application Publication
(10) Pub. No.: US 2025/0178987 A1
(43) Pub. Date: Jun. 5, 2025

(54) ORGANIC COMPOUND, ORGANIC LIGHT EMITTING DIODE AND ORGANIC LIGHT EMITTING DEVICE HAVING THEREOF

(71) Applicant: LG Display Co., Ltd., Seoul (KR)

(72) Inventors: Ji-Cheol SHIN, Paju-si (KR); Young-Jun YU, Paju-si (KR); Sang-Beom KIM, Paju-si (KR); Yoo-Jin DOH, Hwaseong-si (KR); Chi-Sik KIM, Hwaseong-si (KR); Young-Kwang KIM, Hwaseong-si (KR); Hae-Yeon KIM, Hwaseong-si (KR)

(21) Appl. No.: 18/968,857

(22) Filed: Dec. 4, 2024

(30) Foreign Application Priority Data
Dec. 5, 2023 (KR) 10-2023-0174650

(51) Int. Cl. C07C 15/28; C07B 59/00; C09K 11/02; C09K 11/06; H10K 50/12; H10K 85/20; H10K 85/60; H10K 101/00

(52) U.S. Cl. CPC C07C 15/28; C07B 59/004; C09K 11/02; C09K 11/06; H10K 50/12; H10K 85/20; H10K 85/615; C07B 2200/05; H10K 2101/27

(57) ABSTRACT

The present disclosure relates to an organic compound, an organic light emitting diode (OLED) and an organic light emitting device having thereof. In particular, an organic compound having the following structure of Chemical Formula 1 is provided. Luminous lifespan of an organic light emitting diode (OLED) and an organic light emitting device containing the OLED (e.g., a display device or a lighting device) can be improved by applying the organic compound in an emissive layer in the OLED.

### CROSS-REFERENCE TO RELATED APPLICATIONS

This application claims priority under 35 U.S.C. § 119(a) to Korean Patent Application No. 10-2023-0174650, filed in the Republic of Korea on Dec. 5, 2023, the entire contents of which are hereby expressly incorporated by reference into the present application.

### BACKGROUND

### Technical Field

The present disclosure relates to organic compounds, and more particularly to, organic compounds with beneficial luminous properties, and organic light emitting diodes (OLEDs) as well as organic light emitting devices (e.g., display devices or lighting devices) comprising the organic compound.

### Description of the Related Art

Flat display devices including an organic light emitting diode (OLED) have been investigated as display devices that can replace a liquid crystal display device (LCD). The electrode configurations in the OLED can implement unidirectional or bidirectional images. Also, the OLED can be formed even on a flexible transparent substrate such as a plastic substrate so that a flexible or a foldable display device can be realized with ease using the OLED. In addition, the OLED can be driven at a lower voltage and the OLED has advantageous high color purity compared to the LCD.

However, there remains a need to develop OLEDs and devices including the OLEDs that have improved luminous efficiency and luminous lifespan. Since fluorescent materials use only singlet excitons in the luminous process, the related art fluorescent material shows low luminous efficiency. Meanwhile, phosphorescent materials can show high luminous efficiency since they use triplet exciton as well as singlet excitons in the luminous process. But such phosphorescent materials comprise metal complexes, which can have a luminous lifespan that is too short for commercial use. As such, there remains a need to develop material with sufficient luminous efficiency and luminous lifespan.

### BRIEF SUMMARY

Accordingly, some embodiments of the present disclosure are directed to organic compounds, organic light emitting diodes and organic light emitting devices that substantially obviate one or more of the problems due to the limitations and disadvantages of the related art.

An aspect of the present disclosure is to provide organic compounds that can be applied into an organic light emitting diodes (OLEDs) with improved driving voltage, luminous efficiency and/or luminous lifespan.

Another aspect of the present disclosure is to provide organic compounds that are proper for OLEDs.

Another aspect of the present disclosure is to provide an OLED and an organic light emitting device (e.g., display device or lighting device) with beneficial driving voltage, luminous efficiency and luminous lifespan.

Additional features and aspects of the present disclosure will be set forth in the description that follows, and in part will be apparent from the description, or can be learned by practice of the disclosed concepts provided herein. Other features and aspects of the disclosed concept can be realized and attained by the structure particularly pointed out in the written description, or derivable therefrom, and the claims hereof as well as the appended drawings.

To achieve these and other aspects of the inventive concepts, as embodied and broadly described, in one aspect, the present disclosure provides an organic compound having the following structure of Chemical Formula 1:

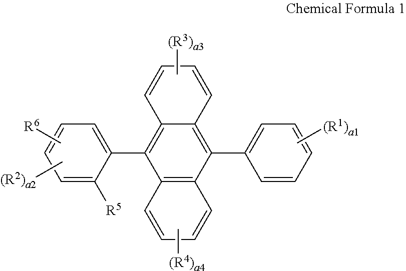

wherein, in Chemical Formula 1,
R¹ is protium, deuterium, tritium, an unsubstituted or substituted C₁-C₁₀ alkyl group or unsubstituted or substituted phenyl, where each R¹ is identical to or different from each other when a1 is 2, 3, 4 or 5, or
   optionally,
two adjacent R¹ when a1 is 2, 3, 4 or 5 are further linked to form a benzene ring unsubstituted or substituted with at least one of deuterium, C₁-C₁₀ alkyl, and a group formed by connecting the two groups mentioned above;
each of R², R³ and R⁴ is independently protium, deuterium, tritium or an unsubstituted or substituted C₁-C₁₀ alkyl group, where each R² is identical to or different from each other when a2 is 2 or 3, each R³ is identical to or different from each other when a3 is 2, 3 or 4, and each R⁴ is identical to or different from each other when a4 is 2, 3 or 4;
each of R⁵ and R⁶ is independently phenyl, biphenyl or naphthyl, wherein each of the phenyl, biphenyl and the naphthyl is independently unsubstituted or substituted with at least one of deuterium, C₁-C₁₀ alkyl, and a group formed by connecting the two groups mentioned above;
a1 is 0, 1, 2, 3, 4 or 5;
a2 is 0, 1, 2 or 3; and
each of a3 and a4 is independently 0, 1, 2, 3 or 4.

As an example, the organic compound can have the following structure of Chemical Formula 2 or Chemical Formula 3:

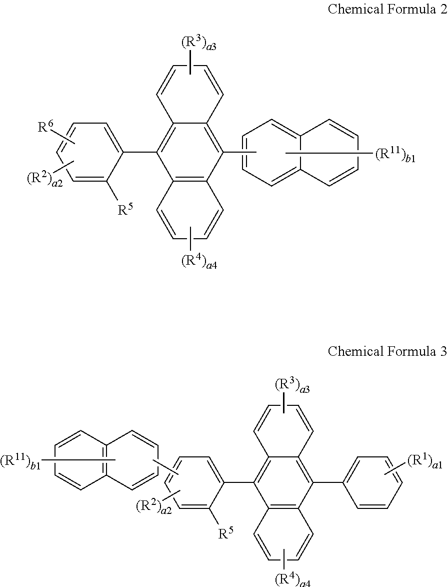

wherein, in Chemical Formulae 2 and 3,
each of R¹, R², R³, R⁴, R⁵, R⁶, a1, a2, a3 and a4 is a same as defined in Chemical Formula 1;
R¹¹ is protium, deuterium or an unsubstituted or deuterium-substituted C₁-C₁₀ alkyl group, where each R¹¹ is identical to or different from each other when b1 is 2, 3, 4, 5, 6 or 7; and
b1 is 0, 1, 2, 3, 4, 5, 6 or 7.

In one embodiment, the organic compound can have the following structure of Chemical Formula 4 or Chemical Formula 5:

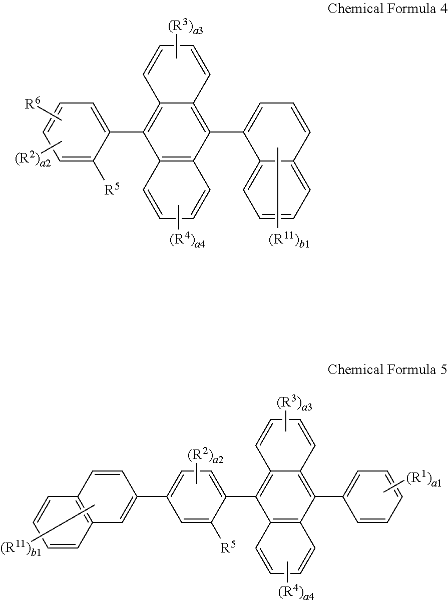

wherein, in Chemical Formulae 4 and 5,
each of R¹, R², R³, R⁴, R⁵, R⁶, a1, a2, a3 and a4 is a same as defined in Chemical Formula 1;
R¹¹ is protium, deuterium or an unsubstituted or deuterium-substituted C₁-C₁₀ alkyl group, where each R¹¹ is identical to or different from each other when b1 is 2, 3, 4, 5, 6 or 7; and
b1 is 0, 1, 2, 3, 4, 5, 6 or 7.

In one embodiment, two adjacent R¹s can be linked to form a benzene ring, the rest three of R¹s can be protium or deuterium, and R⁶ can be naphthyl in Chemical Formula 1.

In another aspect, the present disclosure provides an organic light emitting diode that comprises a first electrode; a second electrode facing the first electrode; and an emissive layer disposed between the first electrode and the second electrode, and the emissive layer can comprises the organic compound.

The emissive layer can comprise one or more emitting material layer and at least one emitting material layer can comprise the organic compound.

The at least one emitting material layer can comprise a host and a dopant, and the host can comprise the organic compound.

The dopant can comprise at least one of blue phosphorescent material, blue fluorescent material and blue delayed fluorescent material.

In one embodiment, the dopant can comprise phosphorescent material of a platinum-containing organometallic compound.

In another embodiment, the dopant can comprise fluorescent material with a polycyclic hetero aromatic ring including boron, and nitrogen, oxygen or sulfur.

In another embodiment, the dopant can comprise a first dopant and a second dopant.

As an example, the first dopant can comprise at least one of blue delayed fluorescent material and blue phosphorescent material and/or the second dopant can comprise blue fluorescent material.

The emissive layer can comprise a single emitting part or multiple emitting parts to form a tandem structure.

In yet another aspect, the present disclosure provides an organic light emitting device, for example, an organic light emitting display device or an organic light emitting illumination device, which comprises a substrate and the organic light emitting diode over the substrate.

In one or more embodiment, the organic compound can exhibit performance adequately utilized in an organic light emitting diode (OLED). The OLED or an organic light emitting device where the organic compound is used as organic electroluminescent material can have beneficial luminous efficiency and luminous lifespan.

It is to be understood that both the foregoing general description and the following detailed description are exemplary and explanatory, and are intended to provide further explanation of the inventive concepts as claimed.

### BRIEF DESCRIPTION OF THE SEVERAL VIEWS OF THE DRAWINGS

The accompanying drawings, which are comprised to provide a further understanding of the disclosure, are incorporated in and constitute a part of this application, illustrate embodiments of the disclosure and together with the description serve to explain principles of the disclosure.

FIG. 1 illustrates a schematic circuit diagram of an organic light emitting display device in accordance with one or more embodiments of the present disclosure.

FIG. 2 illustrates a cross-sectional view of an organic light emitting display device as an example of an organic light emitting device in accordance with an embodiment of the present disclosure.

FIG. 3 illustrates a cross-sectional view of an organic light emitting diode having a single emitting part in accordance with an embodiment of the present disclosure.

FIG. 4 illustrates a cross-sectional view of an organic light emitting diode having a single emitting part in accordance with another embodiment of the present disclosure.

FIG. 5 illustrates a cross-sectional view of an organic light emitting display device in accordance with another embodiment of the present disclosure.

FIG. 6 illustrates a cross-sectional view of an organic light emitting diode having a tandem structure of two emitting parts in accordance with another embodiment of the present disclosure.

FIG. 7 illustrates a cross-sectional view of an organic light emitting diode having a tandem structure of three emitting parts in accordance with another embodiment of the present disclosure.

### DETAILED DESCRIPTION

Reference will now be made in detail to aspects of the disclosure, examples of which are illustrated in the accompanying drawings. Wherever possible, the same reference numbers will be used throughout the drawings to refer to the same or like parts.

All the components of each organic light emitting display device according to all embodiments of the present disclosure are operatively coupled and configured.

### Organic Compound

As used herein, the term “organic electroluminescence material” or “electroluminescence material” indicates material that can be used in an organic light emitting diode, can comprise at least one compound, and, if necessary, can be included in any layer constituting the organic light emitting diode. For example, the organic electroluminescence material can be hole injecting material, hole transporting material, hole auxiliary material, luminescence auxiliary material, electron blocking material, luminescence material such as host material and/or dopant material, electron buffering material, hole blocking material, electron transporting material, electron injecting material, and the like.

As used herein, the term “plural hosts” indicates host material including combinations of two or more compounds that can be included in any emissive layer constituting the organic light emitting diode, and can mean both material before applying to the organic light emitting diode (for example, before deposition) and material after applying to the organic light emitting diode (for example, after deposition). For example, the plural hosts indicate combinations of two or more host materials, and can optionally comprise a conventional material included in the organic electroluminescence material. Two or more compounds included in the plural host materials can be included in one emissive layer or in different emissive layers through the process utilized by conventional means. For example, two or more compounds can be mix-deposited or co-deposited, or individually deposited.

As used herein, the term “hole transporting area” indicates an area where holes are transported between the first electrode and the emitting material layer, and, for example, can comprise at least one of a hole injection layer, a hole transport layer, a hole auxiliary layer, an emitting auxiliary layer and an electron blocking layer. Each of the hole injection layer, the hole transport layer, the hole auxiliary layer, the emitting auxiliary layer and the electron blocking layer can be independently a single layer, or plural layers that two or more layer or three or more layers are stacked.

In one embodiment, the hole transporting area can comprise a first hole transport layer, a second hole transport layer, and optionally a third hole transport layer. Each of the second hole transport layer and the third hole transport layer can be one or more layers of plural hole transport layers, and can comprise one or more of the hole transport layer, the emitting auxiliary layer and the electron blocking layer.

In another embodiment, the first hole transport layer can be disposed between the first electrode and the emitting material layer, and the second hole transport layer can be disposed between the first hole transport layer and the emitting material layer. The second hole transport layer can be any layer acting as the hole transport layer, the emitting auxiliary layer, the hole auxiliary layer and/or the electron blocking layer.

In another embodiment, the hole transporting area can comprise the first hole transport layer, the second hole transport layer and the third hole transport layer. The first hole transport layer can be disposed between the first electrode and the emitting material layer, the second hole transport layer can be disposed between the first hole transport layer and the emitting material layer, and the third hole transport layer can be disposed between the second hole transport layer and the emitting material layer. The third hole transport layer can be any layer acting as the hole transport layer, the emitting auxiliary layer, the hole auxiliary layer and/or the electron blocking layer.

As used herein, the term “unsubstituted” means that hydrogen is directly linked to a carbon atom. “Hydrogen,” as used herein, can refer to protium.

As used herein, “substituted” means that the hydrogen is replaced with a substituent. The substituent can be replaced unlimitedly to hydrogen at any locations. When two or more hydrogens in one group are replaced with the substituents, each substituent can be identical to or different from each other. The maximum numbers of the substituents replaced with a group can be total number of atomic valence that can be substituted to an atom of the group. For example, the substituent can comprise, but is not limited to, an unsubstituted or halogen- and/or deuterium-substituted C₁-C₂₀ alkyl group, an unsubstituted or halogen- and/or deuterium-substituted C₁-C₂₀ alkoxy, halogen, a cyano group, a hydroxyl group, a carboxylic group, a carbonyl group, an amino group, a C₁-C₁₀ alkyl amino group, a C₆-C₃₀ aryl amino group, a C₃-C₃₀ hetero aryl amino group, a nitro group, a hydrazyl group, a sulfonate group, an unsubstituted or halogen- and/or deuterium-substituted C₁-C₁₀ alkyl silyl group, an unsubstituted or halogen- and/or deuterium-substituted C₁-C₁₀ alkoxy silyl group, an unsubstituted or halogen- and/or deuterium-substituted C₃-C₂₀ cycloalkyl silyl group, an unsubstituted or halogen- and/or deuterium-substituted C₆-C₃₀ aryl silyl group, an unsubstituted or halogen- and/or deuterium-substituted C₃-C₃₀ hetero aryl silyl group, an unsubstituted or C₁-C₂₀ alkyl- and/or deuterium-substituted C₆-C₃₀ aryl group, an unsubstituted or C₁-C₂₀ alkyl- and/or deuterium-substituted C₃-C₃₀ hetero aryl group, or any combination of these groups.

As an example, the substituent can comprise, but is not limited to, unsubstituted or substituted methyl, unsubstituted or substituted tert-butyl, unsubstituted or substituted phenyl, unsubstituted or substituted naphthyl, unsubstituted or substituted biphenyl, unsubstituted or substituted diphenyl fluorenyl, unsubstituted or substituted dimethyl fluorenyl, unsubstituted or substituted pyridinyl, unsubstituted or substituted dibenzo-furanyl, unsubstituted or substituted dibenzo-thiophenyl, unsubstituted or substituted carbazolyl, and the like.

As used herein, the term “hetero” in terms such as “a hetero alicyclic ring,” “a hetero aromatic ring,” “a hetero cycloalkyl group,” “a hetero aryl group,” “a hetero aralkyl group,” “a hetero aryloxy group,” “a hetero aryl amino group,” “a hetero aryl silyl group,” “a hetero aryl germanyl group,” “a hetero arylene group,” and the likes means that at least one carbon atom, for example 1 to 5 carbons atoms, constituting an aliphatic chain, an alicyclic group or ring or an aromatic group or ring is substituted with at least one hetero atom selected from the group consisting of N, O, S and P.

As used herein, the term “C₁-C₂₀ alkyl group” indicate a linear or branched alkyl group with 1 to 20 carbon atoms. As an example, the carbon number of the alkyl group can be 1 to 15, for example, 1 to 10. For example, the alkyl group can comprise, but is not limited to, methyl, ethyl, propyl, n-propyl, isopropyl, butyl, n-butyl, isobutyl, tert-butyl, sec-butyl, 1-methyl-butyl, 1-ethyl-butyl, pentyl, n-pentyl, isopentyl, neopentyl, tert-pentyl, hexyl, n-hexyl, 1-methylpentyl, 2-methylpentyl, 4-methyl-2-pentyl, 3,3-dimethylbutyl, 2-ethylbutyl, heptyl, n-heptyl, 1-methylhexyl, octyl, n-octyl, tert-octyl, 1-methylheptyl, 2-ethylhexyl, 2-propylpentyl, n-nonyl, 2,2-dimethylheptyl, 1-ethyl-propyl, 1,1-dimethyl-propyl, isohexyl, 2-methylpentyl, 4-methylhexyl, 5-methylhexyl, and the like. As used herein, the C₁-C₂₀ alkylene group can comprise, but is not limited to, any bivalent linking group corresponding to the above alkyl group.

As used herein, the term “C₃-C₃₀ cycloalkyl group” or “C₃-C₃₀ alicyclic group” indicates a mono- or polycyclic hydrocarbon with 3 to 30 ring carbon atoms. As an example, the cycloalkyl group can have 3 to 20, for example, 3 to 7 ring carbon atoms. For example, the cycloalkyl group can comprise, but is not limited to, cyclopropyl, cyclobutyl, cyclopentyl, cyclohexyl, cycloheptyl, and the like.

As used herein, the term “C₃-C₃₀ cycloakenyl group” indicates a mono- or polycyclic hydrocarbon with 3 to 30 ring carbon atoms and at least one double bond with the ring. As an example, the cycloalkenyl group can have 3 to 20, for example, 3 to 7 ring carbon atoms. For example, the cycloalkenyl group can comprise, but is not limited to, cyclopropenyl, cyclobutenyl and cyclopentenyl.

As used herein, the term “C₃-C₇ hetero cycloalkyl group” indicate a cycloalkyl with 3 to 7, for example, 3 to 5 ring atoms and at least one hetero atom selected from B, N, O, S, Si and P, for example, N, O and S. For example, the hetero cycloalkyl can comprise, but is not limited to, tetrahydrofurnayl, pyrrolidinyl, thiolanyl, tetrahydropyranyl, and the like.

As used herein, the term “C₆-C₃₀ aryl group (or arylene group)” indicates a mono- or polycyclic radical or a corresponding bivalent linker or bridging group derived from aromatic hydrocarbons with 6 to 30 ring carbon atoms, and can comprise a spiro structure. For example, the aryl group or the arylene group can have 6 to 20, for example, 6 to 15 ring carbon atoms.

For example, the C₆-C₃₀ aryl group can comprise, but is not limited to, an unfused or fused aryl group such as phenyl, biphenylyl, terphenylyl, quaterphenyl, naphthyl, binaphthyl, phenylnapthyl, naphtylphenyl, fluorenyl, phenylfluorenyl, dimethylfluorenyl, diphenylfluorenyl, benzo-fluorenyl, diphenyl-benzo-fluorenyl, dibenzo-fluorenyl, phenanthrenyl, benzo-phenanthrenyl, phenylphenanthrenyl, anthryl, benz-anthryl, indenyl, triphenylenyl, pyrenyl, tetracenyl, chrysenyl, benzo-crysenyl, naphthacenyl, fluoranthenyl, bezno-fluoranthenyl, tolyl, xylyl, mesityl, cumenyl, spiro[fluoren-fluoren]yl(spirobifluroneyl), spiro[fluorene-benzofluorene]yl, azulenyl, pentalenyl, indeno-indenyl, heptalenyl, indacenyl, phenalenyl, pentacenyl, indeno-fluorenyl, and the like.

In another embodiment, the C₆-C₃₀ aryl group can comprise, but is not limited to, o-tolyl, m-tolyl, p-tolyl, 2,3-xylyl, 3,4-xylyl, 2,5-xylyl, mesityl, o-cumenyl, m-cumenyl, p-cumenyl, p-t-butylphenyl, p-(2-phenylpropyl)phenyl, 4′-methylbiphenyl, 4″-t-butyl-p-terphenyl-4-yl, o-biphenyl, m-biphenyl, p-biphenyl, o-terphenyl, m-terphenyl-4-yl, m-terphenyl-3-yl, m-terphenyl-2-yl, p-terphenyl-4-yl, p-terphenyl-3-yl, p-terphenyl-2-yl, m-quaterphenyl, 1-naphthyl, 2-naphthyl, 1-fluorenyl, 2-fluorenyl, 3-fluorenyl, 4-fluorenyl, 9-fluorenyl, 9,9-dimethyl-1-fluorenyl, 9,9-dimethyl-2-fluorenyl, 9,9-dimethyl-3-fluorenyl, 9,9-dimethyl-4-fluorenyl, 9,9-diphenyl-1-fluorenyl, 9,9-diphenyl-2-fluorenyl, 9,9-diphenyl-3-fluorenyl, 9,9-diphenyl-4-fluorenyl, 1-anthryl, 2-anthryl, 9-anthryl, 1-phenanthryl, 2-phenanthryl, 3-phenanthryl, 4-phenanthryl, 9-phenanthryl, 1-chrysenyl, 2-chrysenyl, 3-chrysenyl, 4-chrysenyl, 5-chrysenyl, 6-chrysenyl, benzo[c]phenanthryl, benzo[g]chrysenyl, 1-triphenylenyl, 2-triphenylenyl, 3-triphenylenyl, 4-triphenylenyl, 3-fluoranthenyl, 4-fluoranthenyl, 8-fluoranthenyl, 9-fluoranthenyl and benzo-fluoranthenyl. As used herein, the C₆-C₃₀ arylene group can comprise, but is not limited to, any bivalent linking group corresponding to the above aryl group.

As used herein, the term “C₂-C₃₀ hetero aryl group (or hetero arylene group)” indicates a mono- or polycyclic radical or a corresponding bivalent linker or bridging group derived from hetero aromatic hydrocarbons with 2 to 30 ring atoms and at least one hetero atom selected from B, N, O, S, Si, P, Se and Ge. For example, the hetero aryl group can have 5 to 30 ring atoms and have 1 to 4 hetero atoms. The hetero aryl group can have a monocyclic ring or a polycyclic ring fused with at least one 5-membered or 6-membered ring such as a benzene ring. The hetero aryl group or the hetero arylene group can be partially saturated. In addition, the hetero aryl group can comprise a case in which at least one hetero aryl ring is linked with at least one hetero aryl group or aryl group through a single bond.

For example, the C₂-C₃₀ hetero aryl group can comprise, but is not limited to, an unfused or fused hetero aryl group such as furyl, thiophenyl, pyrrolyl, imidazolyl, pyrazolyl, thiazolyl, thiadiazolyl, isothiazolyl, isoxazolyl, oxazolyl, oxadiazolyl, triazinyl, tetrazinyl, triazolyl, tetrazolyl, furazanyl, pyridyl, pyrazinyl, pyridinyl, pyrimidinyl, benzo-furanyl, benzo-thiophenyl, isobenzo-furanyl, dibenzo-furanyl, dibenzo-thiophenyl, benzo-imidazolyl, benzo-thiazolyl, benzo-isothiazolyl, benzo-isoxazolyl, benzo-oxazolyl, imidazopyridyl, imidazopyridinyl, isoindolyl, indolyl, benzo-indolyl, indazolyl, benzo-thiadiazolyl, quinolyl, isoquinolyl, cinnolinyl, quinazolinyl, quinoxalinyl, carbazolyl, aza-carbazolyl, benzo-carbazolyl, dibenzo-carbazolyl, indeno-carbazolyl, indolo-carbazolyl, phenoxazinyl, phenatridinyl, benzo-dioxolyl, indolizinyl, acridinyl, phenazinyl, phenothiazinyl, silafluorenyl, germafluorenyl, and the like.

In another embodiment, the C₂-C₃₀ hetero aryl group can comprise, but is not limited to, 1-pyrrolyl, 2-pyrrolyl, 3-pyrrolyl, 2-pyridinyl, 3-pyridinyl, 4-pyridinyl, 2-pyrimidinyl, 4-pyrimidinyl, 5-pyrimidinyl, 6-pyrimidinyl, 1,2,3-triazin-4-yl, 1,2,4-triazin-3-yl, 1,3,5-triazin-2-yl, 1-imidazolyl, 2-imidazolyl, 1-pyrazolyl, 1-indolizinyl, 2-indolizinyl, 3-indolizinyl, 5-indolizinyl, 6-indolizinyl, 7-indolizinyl, 8-indolizinyl, 2-imidazopyridinyl, 3-imidazopyridinyl, 5-imidazopyridinyl, 6-imidazopyridinyl, 7-imidazopyridinyl, 8-imidazopyridinyl, 1-indolyl, 2-indolyl, 3-indolyl, 4-indolyl, 5-indolyl, 6-indolyl, 7-indolyl, 1-isoindolyl, 2-isoindolyl, 3-isoindolyl, 4-isoindolyl, 5-isoindolyl, 6-isoindolyl, 7-isoindolyl, 2-furyl, 3-furyl, 2-benzo-furanyl, 3-benzo-furanyl, 4-benzo-furanyl, 5-benzo-furanyl, 6-benzo-furanyl, 7-benzo-furanyl, 1-isobenzo-furanyl, 3-isobenzo-furanyl, 4-isobenzo-furanyl, 5-isobenzo-furanyl, 6-isobenzo-furanyl, 7-isobenzo-furanyl, 2-quinolyl, 3-quinolyl, 4-quinolyl, 5-quinolyl, 6-quinolyl, 7-quinolyl, 8-quinolyl, 1-isoquinolyl, 3-isoquinolyl, 4-isoquinolyl, 5-isoquinolyl, 6-isoquinolyl, 7-isoquinolyl, 8-isoquinolyl, 2-quinoxalinyl, 5-quinoxalinyl, 6-quinoxalinyl, 1-carbazoyl, 2-carbazolyl, 3-carbazolyl, 4-carbazolyl, 9-carbazolyl, aza-carbazol-1-yl, aza-carbazol-2-yl, aza-carbazol-3-yl, aza-carbazol-4-yl, aza-carbazol-5-yl, aza-carbazol-6-yl, aza-carbazol-7-yl, aza-carbazol-8-yl, aza-carbazol-9-yl, 1-phenanthridinyl, 2-phenanthridinyl, 3-phenanthridinyl, 4-phenanthridinyl, 6-phenanthridinyl, 7-phenanthridinyl, 8-phenanthridinyl, 9-phenanthridinyl, 10-phenanthridinyl, 1-acridinyl, 2-acridinyl, 3-acridinyl, 4-acridinyl, 9-acridinyl, 2-oxazolyl, 4-oxazolyl, 5-oxazolyl, 2-oxadiazolyl, 5-oxadiazolyl, 3-furazanyl, 2-thienyl, 3-thienyl, 2-methylpyrrol-1-yl, 2-methylpyrrol-3-yl, 2-methylpyrrol-4-yl, 2-methylpyrrol-5-yl, 3-methylpyrrol-1-yl, 3-methylpyrrol-2-yl, 3-methylpyrrol-4-yl, 3-methylpyrrol-5-yl, 2-t-butylpyrrol-4-yl, 3-(2-phenylpropyl)pyrrol-1-yl, 2-methyl-1-indolyl, 4-methyl-1-indolyl, 2-methyl-3-indolyl, 4-methyl-3-indolyl, 2-t-butyl-1-indolyl, 4-t-butyl-1-indolyl, 2-t-butyl-3-indolyl, 4-t-butyl-3-indolyl, 1-dibenzo-furanyl, 2-dibenzo-furanyl, 3-dibenzo-furanyl, 4-dibenzo-furanyl, 1-dibenzo-thiophenyl, 2-dibenzo-thiophenyl, 3-dibenzo-thiophenyl, 4-dibenzo-thiophenyl, 1-silafluorenyl, 2-silafluorenyl, 3-silafluorenyl, 4-silafluorenyl, 1-germafluorenyl, 2-germafluorenyl, 3-germafluorenyl, 4-germafluorenyl, 1-dibenzo-selenophenyl, 2-dibenzo-selenophenyl, 3-dibenzo-selenophenyl, 4-dibenzo-selenophenyl, and the like. As used herein, the C₂-C₃₀ hetero arylene group can comprise, but is not limited to, any bivalent linking group corresponding to the above hetero aryl group.

In addition, the “hetero aryl (or hetero arylene) can be divided into a hetero aryl (or hetero arylene) with electron property and a hetero aryl (or hetero arylene) with hole property. The electron property hetero aryl (hetero arylene) can be a substituent with rich electrons in the nuclear ring. For example, the electron property hetero aryl can comprise, but is not limited to, unsubstituted or substituted pyridinyl, unsubstituted or substituted pyrimidinyl, unsubstituted or substituted triazinyl, unsubstituted or substituted quinazolinyl, unsubstituted or substituted quinoxalinyl, unsubstituted or substituted quinolyl, and the like. The hole property hetero aryl (hetero arylene) can be a substituent with deficient electrons in the nuclear ring. For example, the hole property hetero aryl can comprise, but is not limited to, unsubstituted or substituted carbazolyl, unsubstituted or substituted dibenzo-furanyl, unsubstituted or substituted dibenzo-thiophenyl, and the like.

As used herein, the “fused ring group of C₃-C₃₀ alicyclic ring and C₆-C₃₀ aromatic ring” indicates a functional group of a ring system fused an alicyclic ring with 3 to 30, for example, 3 to 25 or 3 to 18 ring carbon atoms with an aromatic ring with 6 to 30, for example 6 to 25 or 6 to 18 ring carbon atoms. For example, the fused ring group can comprise, but is not limited to, a fused ring with at least one benzene ring and at least one cyclohexane ring, or with at least one naphthalene ring and at least one cyclopentane ring. At least one carbon atom in the fused ring group of C₃-C₃₀ alicyclic ring and C₆-C₃₀ aromatic ring can be substituted with at least one hetero atom selected from B, N, O, S, Si and P, for example, N, O and S.

As used herein, the term “halogen” indicates F, Cl, Br and/or I.

The term ‘ortho- (o-),” “meta- (m-)” and “para- (p-)” indicates a substitution position of all substituents. The ortho-position refers to a position of the substituent is immediately adjacent, for example, in the case of benzene, it refers to the 1st and 2nd positions, and the meta-position refers to a next substitution position after the immediately adjacent substitution position, for example, in the case of benzene, it refers to the 1st and 3rd positions, and the para-position refers to the substitution position following the meta position, for example, in the case of benzene, it refers to the 1st and 4th positions.

As used herein, the term “ring formed by linking adjacent substituents” indicates an unsubstituted or substituted 2-30 membered monocyclic or polycyclic alicyclic ring, aromatic ring or combination ring thereof formed by linking or fusion of two or more adjacent substituents. The ring formed by linking adjacent substituents can be an unsubstituted or substituted 3-26 membered monocyclic or polycyclic alicyclic ring, aromatic ring or combination ring thereof. The ring formed by linking adjacent substituents can comprise at least one hetero atom selected from B, N, O, S, Si and P, for example, N, O and S. In another embodiment, the ring can have 5 to 20 membered ring atoms, or 5 to 15 membered ring atoms.

As used herein, if a substituent is not indicated in the Chemical Formula or chemical structure, it may meat that all positions that can be substituents are hydrogen or deuterium. That is, in case of deuterium, it is an isotype of hydrogen, and some hydrogen atoms may the isotype deuterium. In this case, the contents of deuterium, or deuteration rate, may be 0% to 100%. In case where substituents are not indicated in the Chemical Formula or chemical structure, the contents of the deuterium is 0%, the contents of hydrogen 100%, and all substituents are hydrogen. Unless deuterium is explicitly excluded, hydrogen and deuterium can be mixed in compounds.

As used herein, “combinations thereof” indicates a combination of one or more elements or components from the corresponding list to form a known or chemically stable arrangement that can be recognized by a person skill in the art from corresponding list. For example, alkyl and deuterium can be combined to form a partially or fully deuterated alkyl group; halogen and alkyl can be combined to form a halo alkyl substituent; halogen, alkyl and aryl can be combined to form a halogenated aralkyl group. In one embodiment, exemplary combinations of substituents include up to 50 atoms excluding hydrogen and deuterium, or up to 40 atoms excluding hydrogen and deuterium, or up to 30 atoms excluding hydrogen and deuterium. In another embodiment, combinations of substituents can be those containing up to 20 atoms excluding hydrogen and deuterium.

As used herein, when there are plural substituents represented by the same symbol in the Chemical formula, each substituent represented by the same symbol can be identical to or different from each other.

An organic compound can be explained in detail. In one embodiment, the organic compound can have the following structure of Chemical Formula 1:

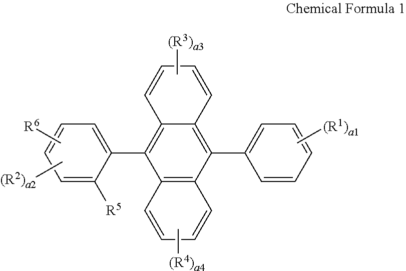

wherein, in Chemical Formula 1,
R¹ is protium, deuterium, tritium, an unsubstituted or substituted C₁-C₁₀ alkyl group or unsubstituted or substituted phenyl, where each R¹ is identical to or different from each other when a1 is 2, 3, 4 or 5, or
optionally,
two adjacent R¹ when a1 is 2, 3, 4 or 5 are further linked to form a benzene ring unsubstituted or substituted with at least one of deuterium, C₁-C₁₀ alkyl, and a group formed by connecting the two groups mentioned above;
each of R², R³ and R⁴ is independently protium, deuterium, tritium or an unsubstituted or substituted C₁-C₁₀ alkyl group, where each R² is identical to or different from each other when a2 is 2 or 3, each R³ is identical to or different from each other when a3 is 2, 3 or 4, and each R⁴ is identical to or different from each other when a4 is 2, 3 or 4;
each of R⁵ and R⁶ is independently phenyl, biphenyl or naphthyl, wherein each of the phenyl, biphenyl and the naphthyl is independently unsubstituted or substituted with at least one of deuterium, C₁-C₁₀ alkyl, and a group formed by connecting the two groups mentioned above;
a1 is 0, 1, 2, 3, 4 or 5;
a2 is 0, 1, 2 or 3; and
each of a3 and a4 is independently 0, 1, 2, 3 or 4.

As one example, R⁵ in Chemical Formula 1 can be phenyl. In one embodiment, R⁵ in Chemical Formula 1 can be phenyl substituted with at least one deuterium. For example, R⁵ in Chemical Formula 1 can be phenyl where all hydrogens are substituted with deuterium.

Each of R² to R⁴ in Chemical Formula 1 can be independently protium or deuterium. For example, each of R² to R⁴ can be protium or at least one of R² to R⁴ can be deuterium. As an example, a2 in Chemical Formula 1 can be 3, and each of a3 and a4 in Chemical Formula 1 can be independently 4.

In another embodiment, at least one naphthyl can be substituted directly or indirectly to a central anthracene ring in the organic compound of Chemical Formula 1. As an example, two adjacent R¹ in Chemical Formula 1 can be linked to form a benzene ring, or R⁶ in Chemical Formula 1 can be naphthyl. An organic compound with such a conformation can have the following structure of Chemical Formula 2 or Chemical Formula 3:

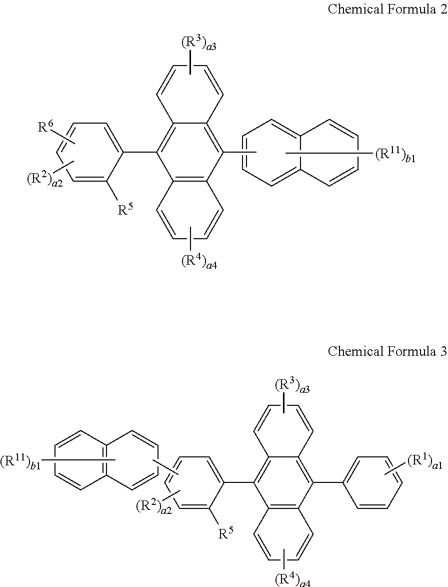

wherein, in Chemical Formulae 2 and 3,
each of R¹, R², R³, R⁴, R⁵, R⁶, a1, a2, a3 and a4 is a same as defined in Chemical Formula 1;
R¹¹ is protium, deuterium or an unsubstituted or deuterium-substituted C₁-C₁₀ alkyl group, where each R¹¹ is identical to or different from each other when b1 is 2, 3, 4, 5, 6 or 7; and
b1 is 0, 1, 2, 3, 4, 5, 6 or 7.

As an example, R¹¹ in Chemical Formula 2 and/or Chemical Formula 3 can be protium or deuterium. At least a part of hydrogen of R¹¹ can be deuterium.

In another embodiment, two adjacent R¹ in Chemical Formula 1 can be fused to the benzene ring to form 1-naphtyl, or R⁶ in Chemical Formula 1 can be 2-naphthyl. An organic compound with such a conformation can have the following structure of Chemical Formula 4 or Chemical Formula 5:

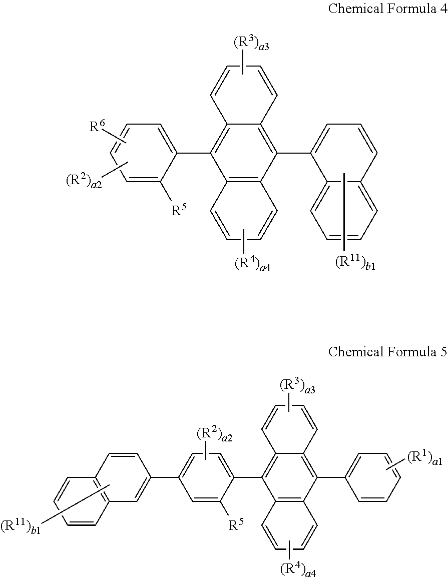

wherein, in Chemical Formulae 4 and 5,
each of R¹, R², R³, R⁴, R⁵, R⁶, a1, a2, a3 and a4 is a same as defined in Chemical Formula 1;
R¹¹ is protium, deuterium or an unsubstituted or deuterium-substituted C₁-C₁₀ alkyl group, where each R¹¹ is identical to or different from each other when b1 is 2, 3, 4, 5, 6 or 7; and
b1 is 0, 1, 2, 3, 4, 5, 6 or 7.

In another embodiment, two adjacent R¹s in Chemical Formula 1 can be linked to form a benzene ring, and thereby linked to the anthracene ring as 1-napthyl form, the rest three of R¹s in Chemical Formula 1 can be protium or deuterium, and R⁶ in Chemical Formula 1 can be naphthyl (e.g., 2-naphthyl).

An organic compound having the structure of Chemical Formulae 1 to 5 can be at least one of or selected from, but is not limited to, the following compounds of Chemical Formula 6:

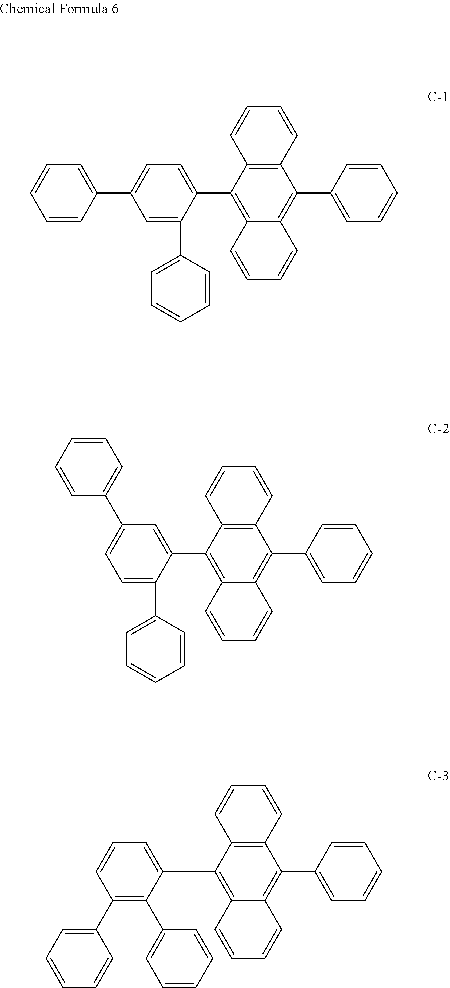

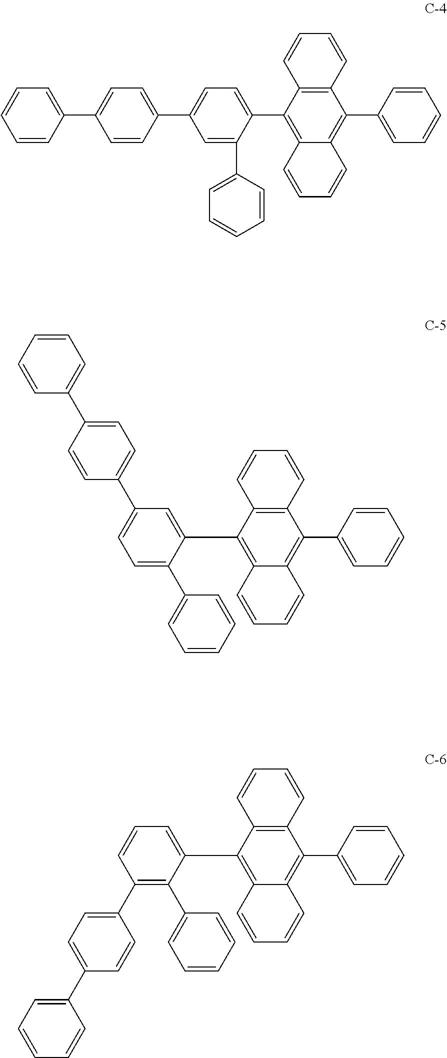

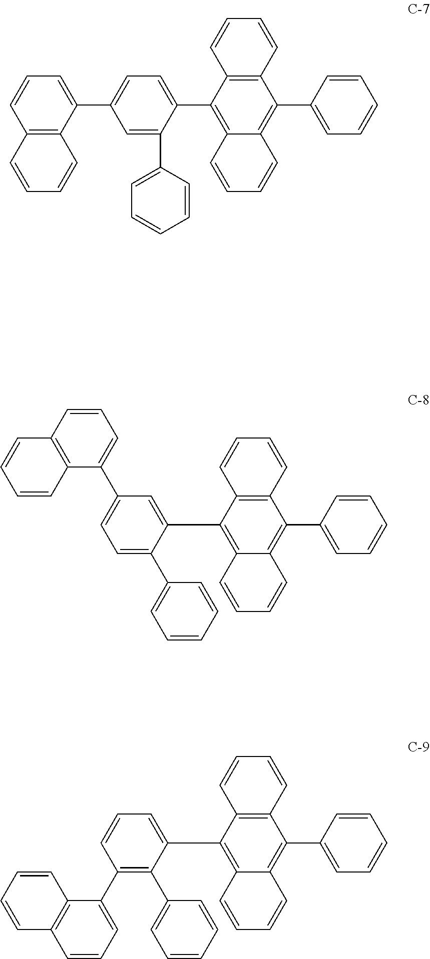

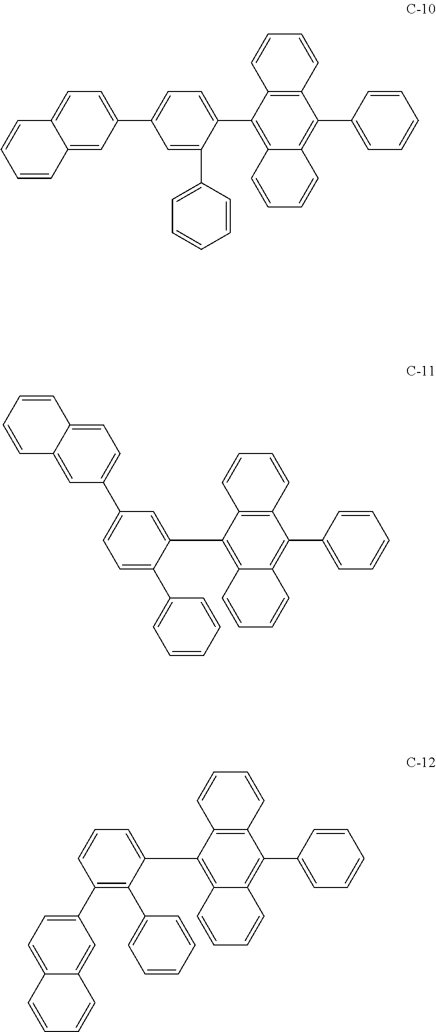

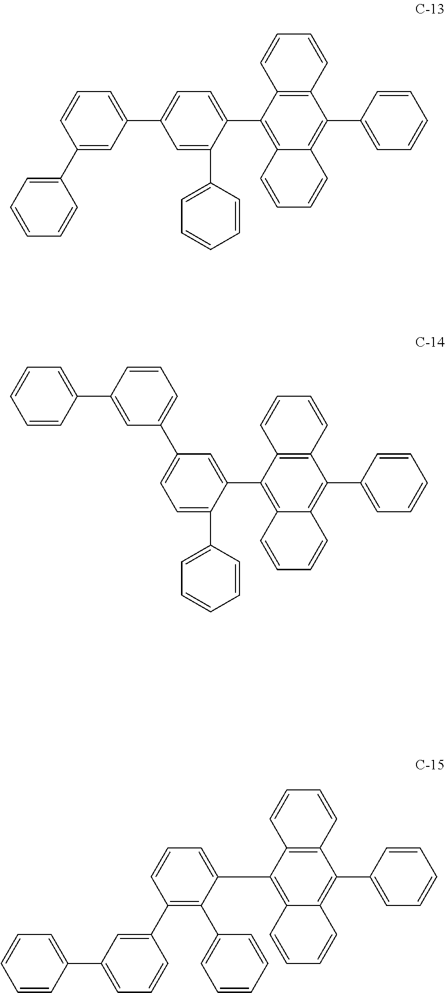

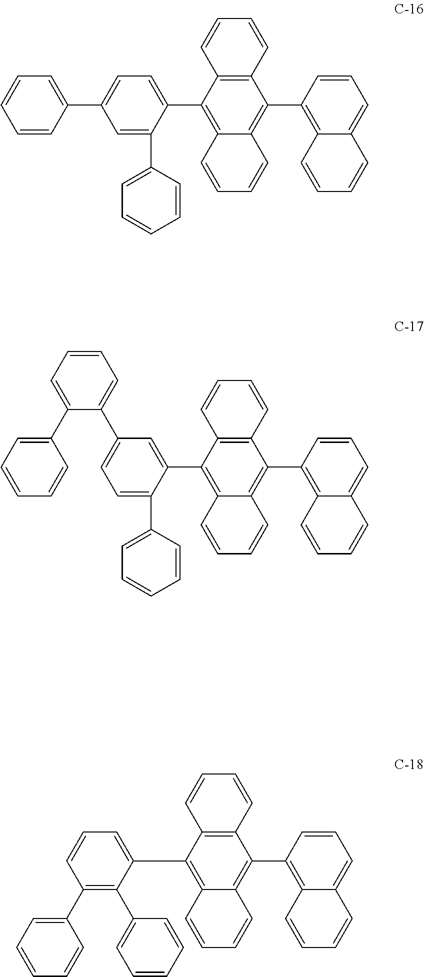

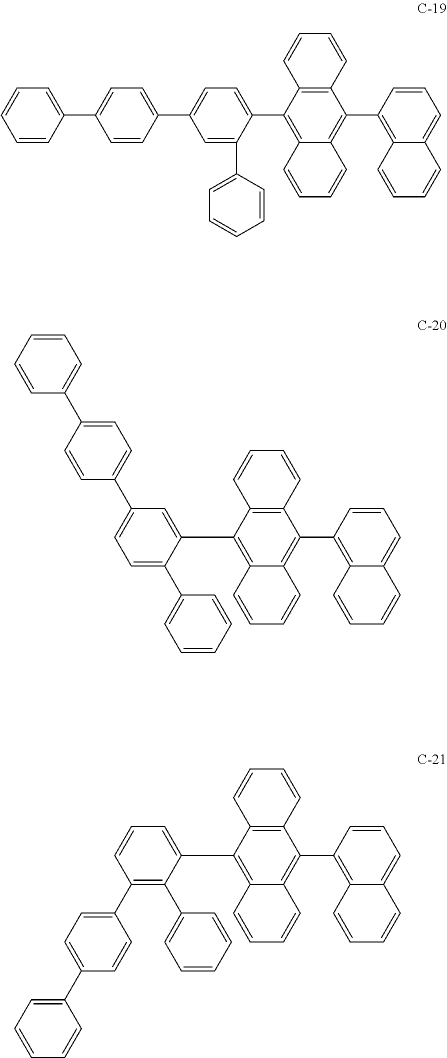

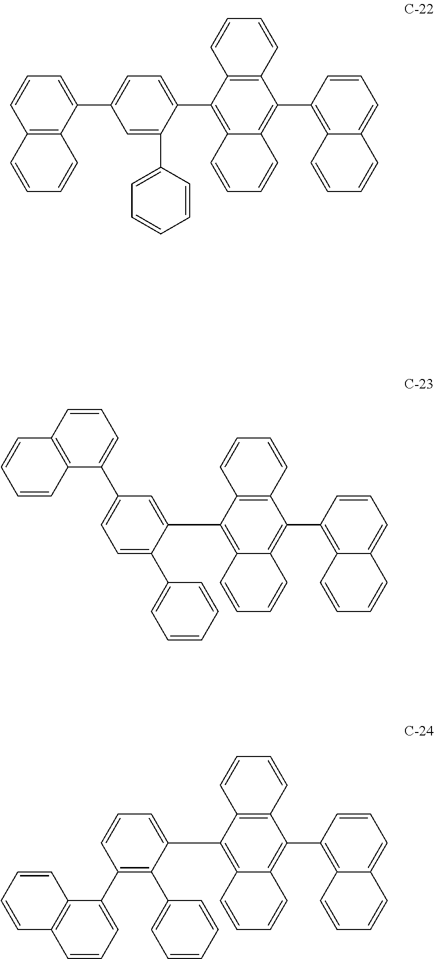

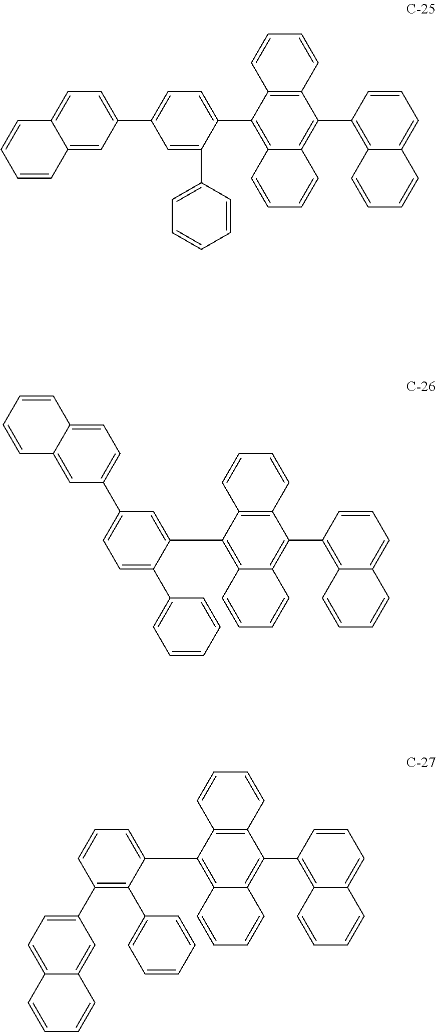

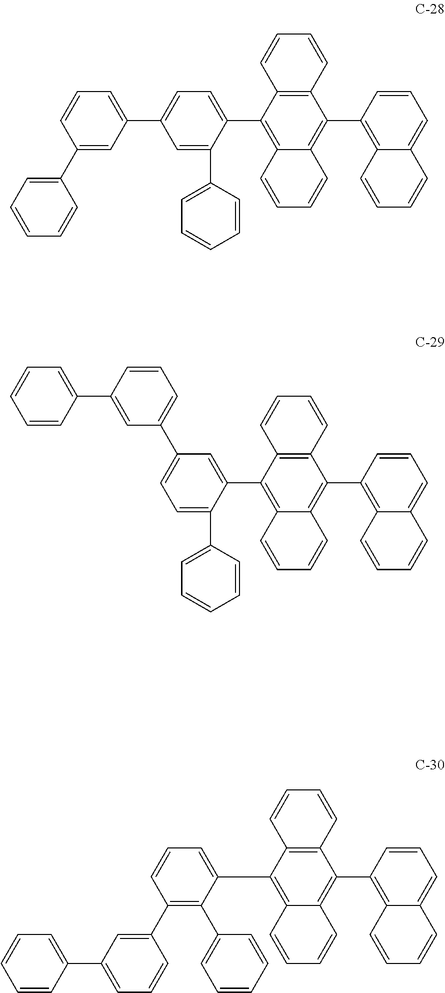

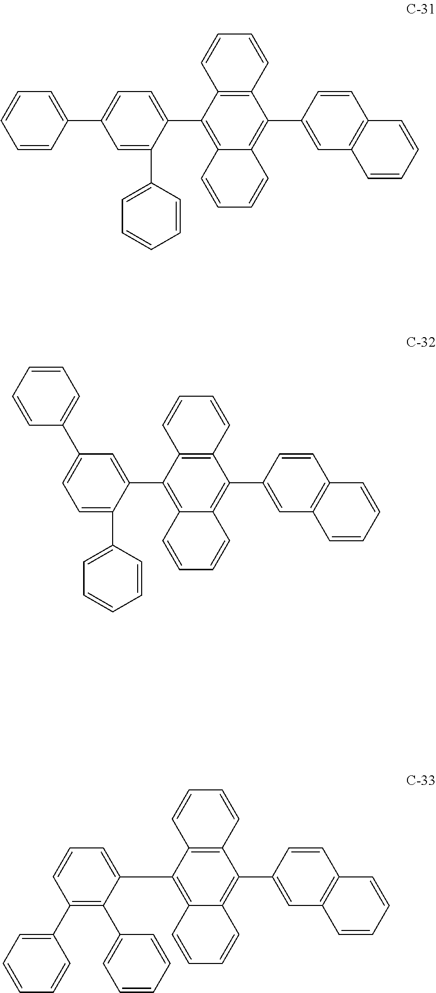

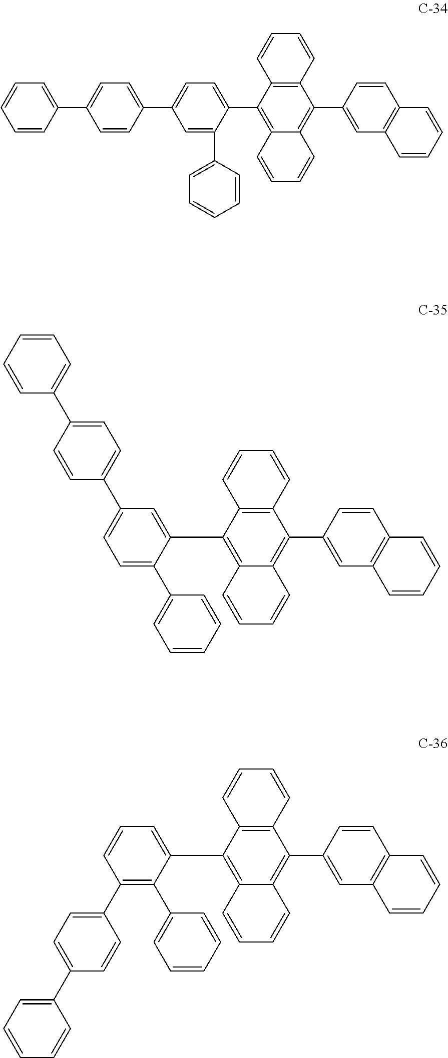

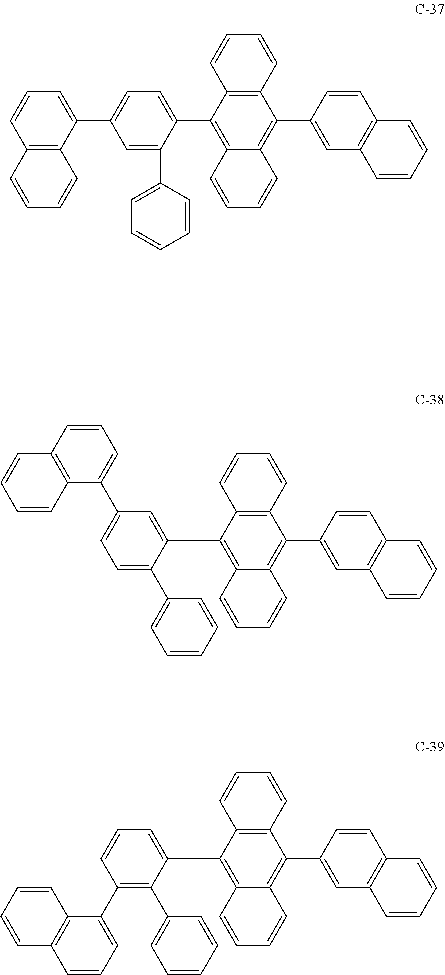

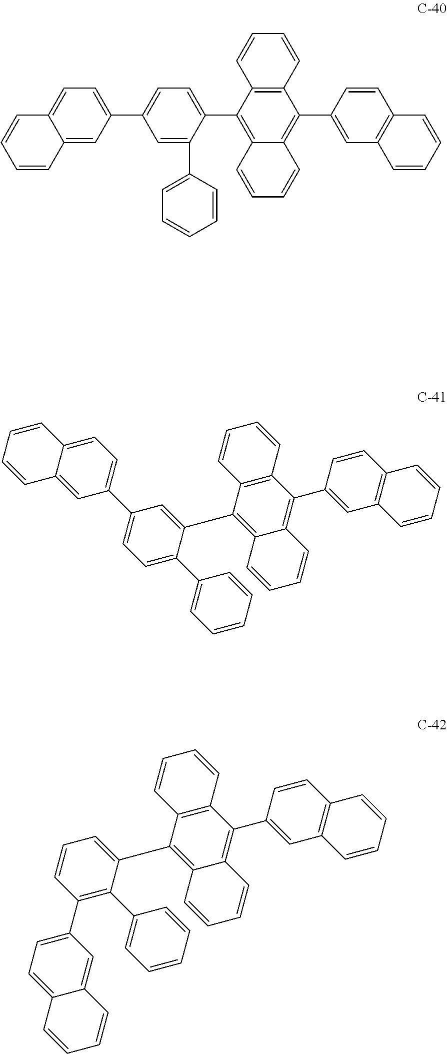

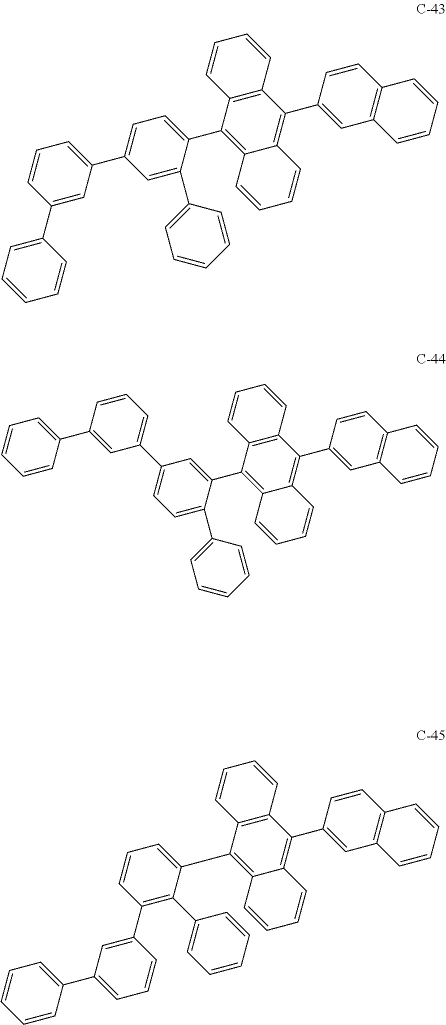

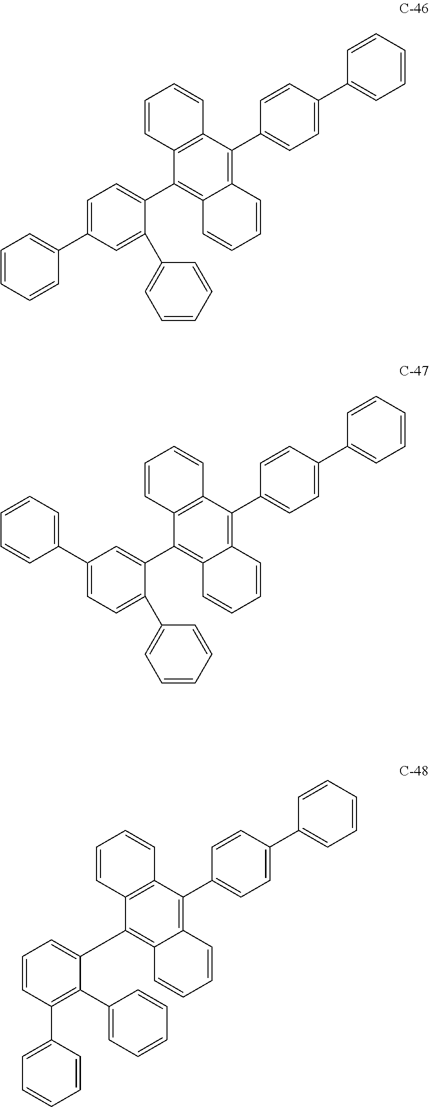

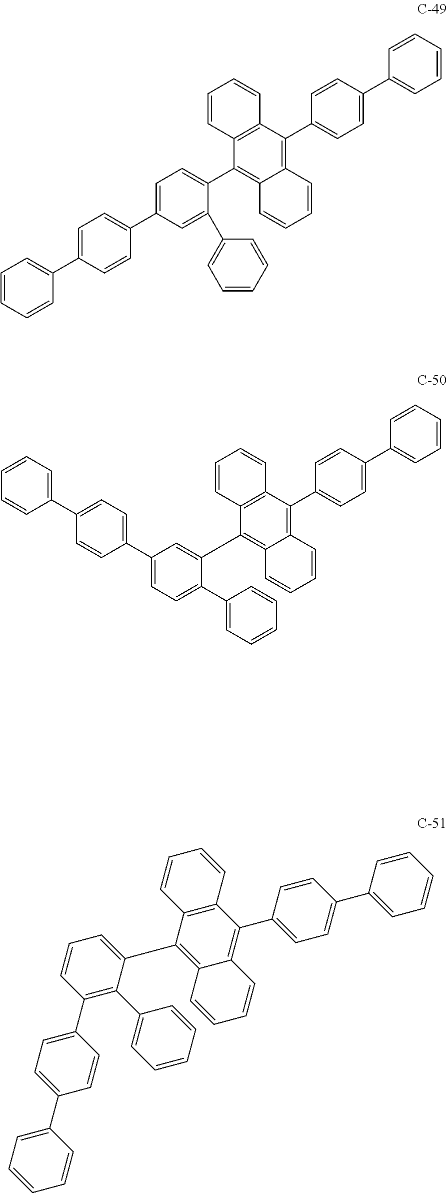

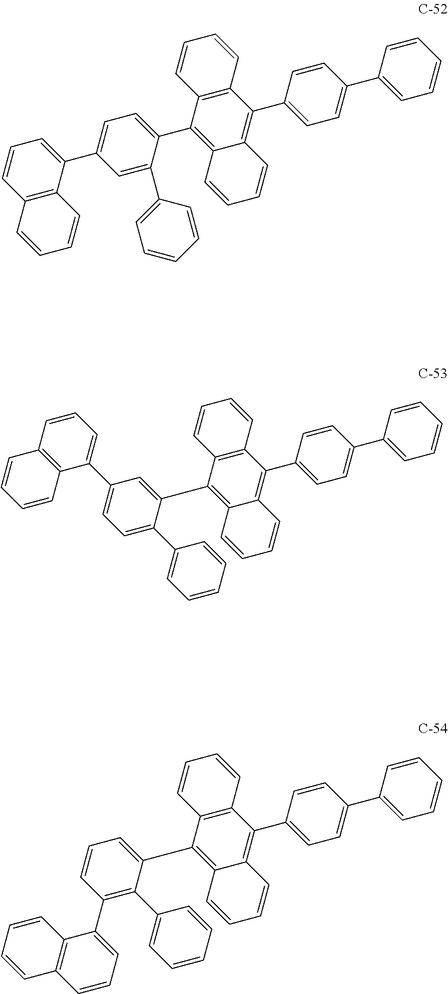

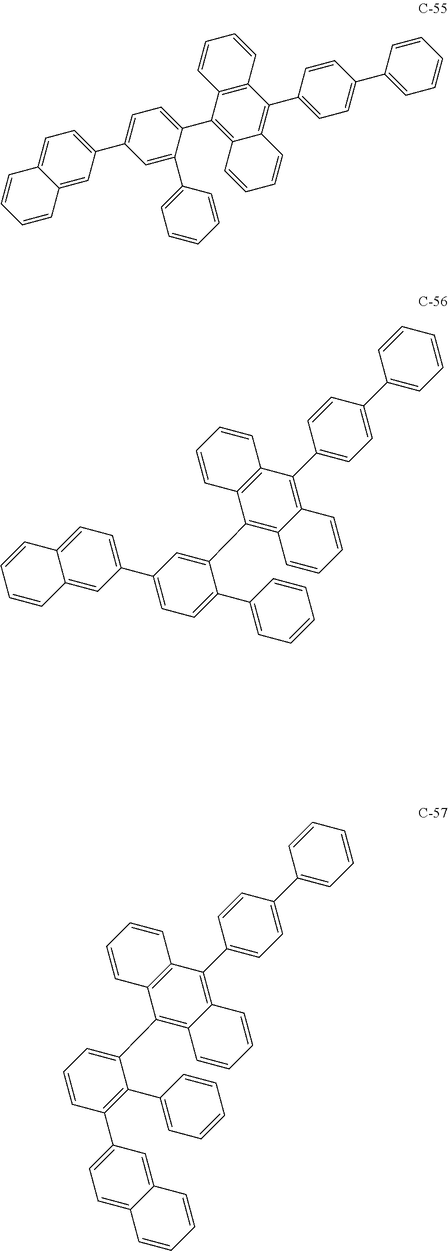

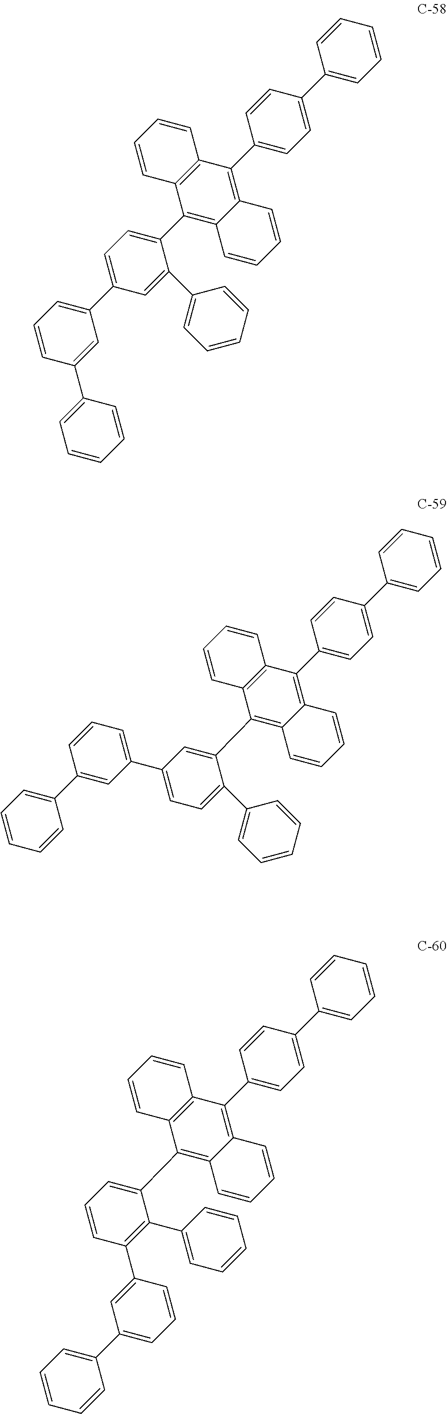

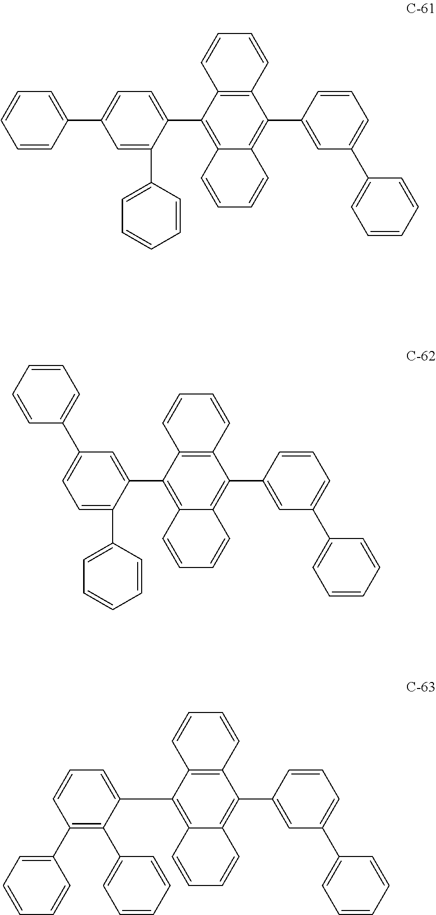

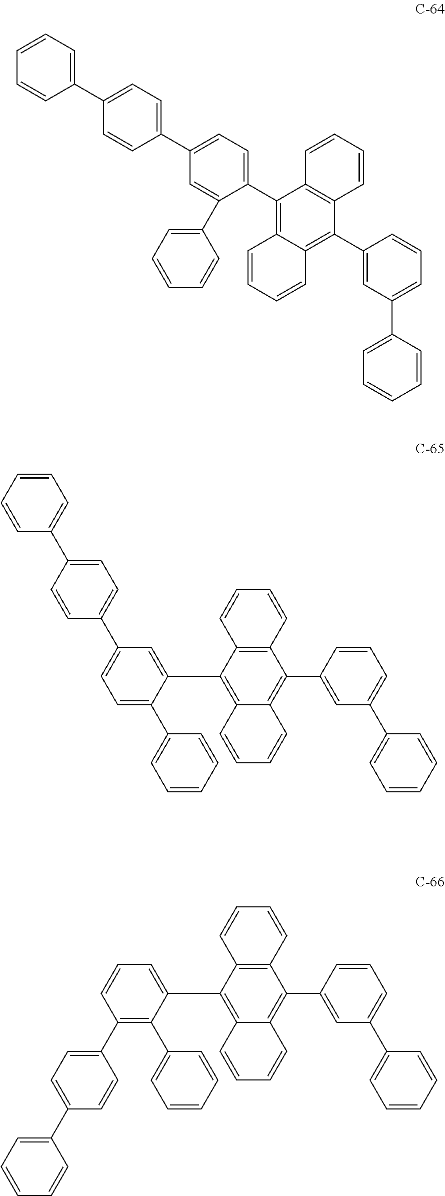

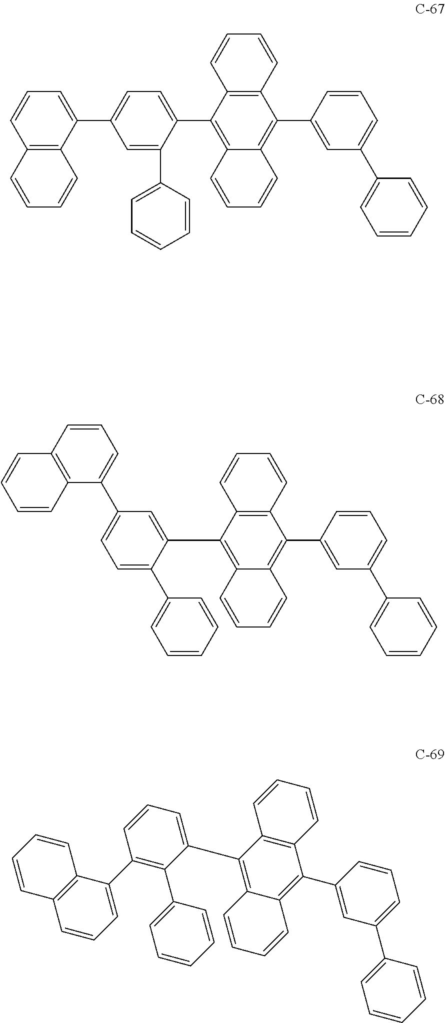

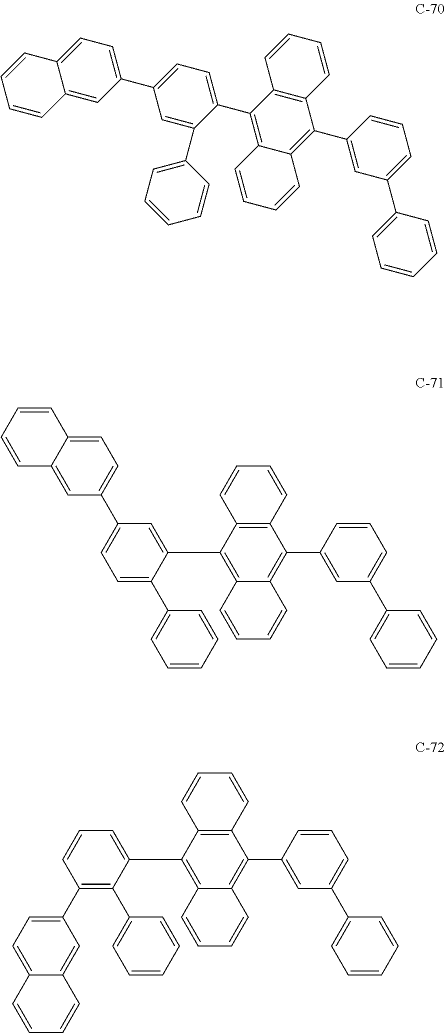

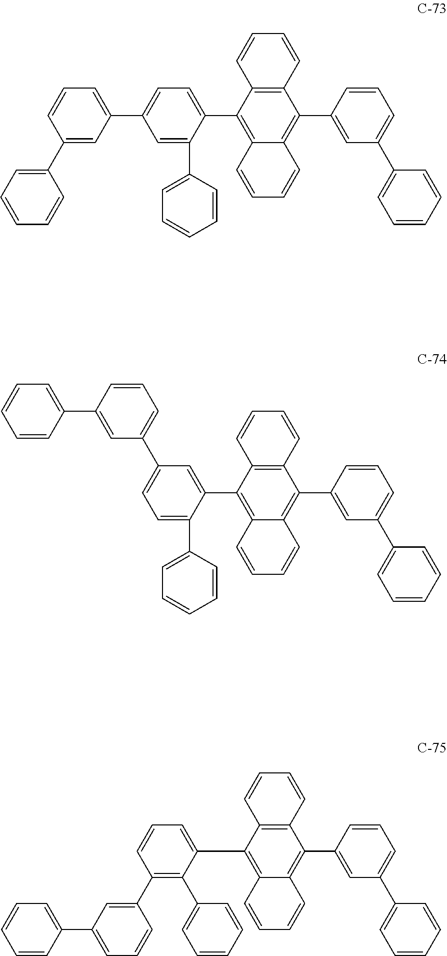

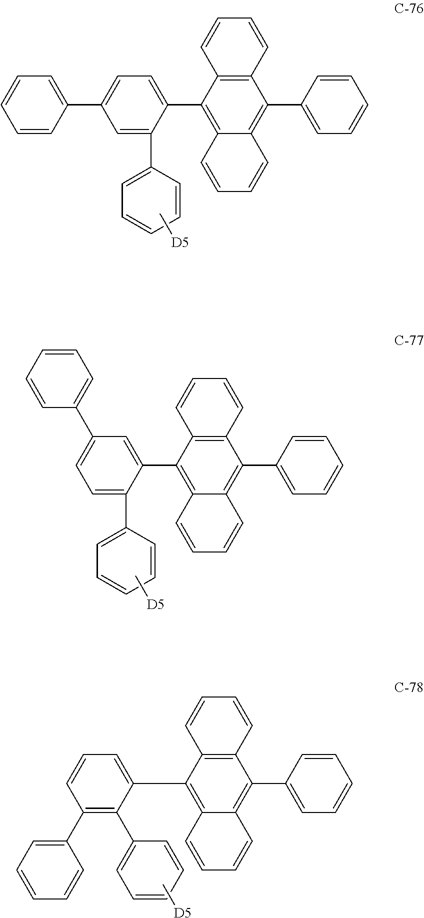

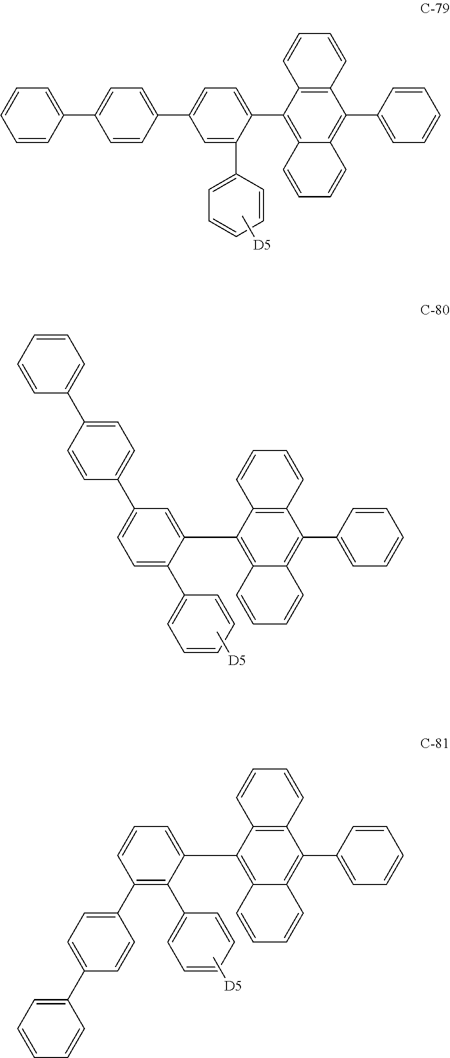

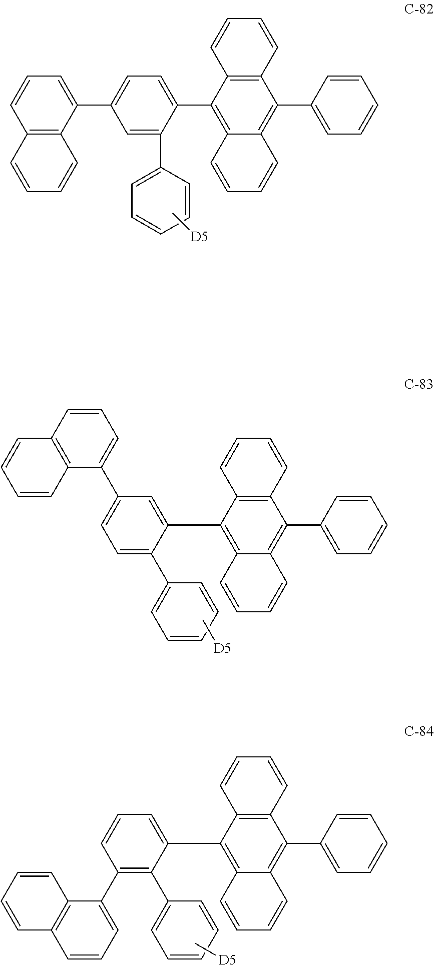

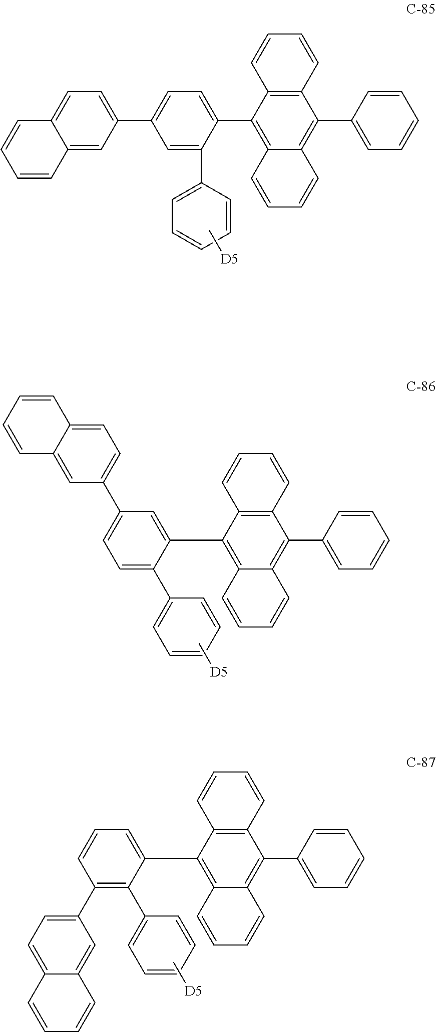

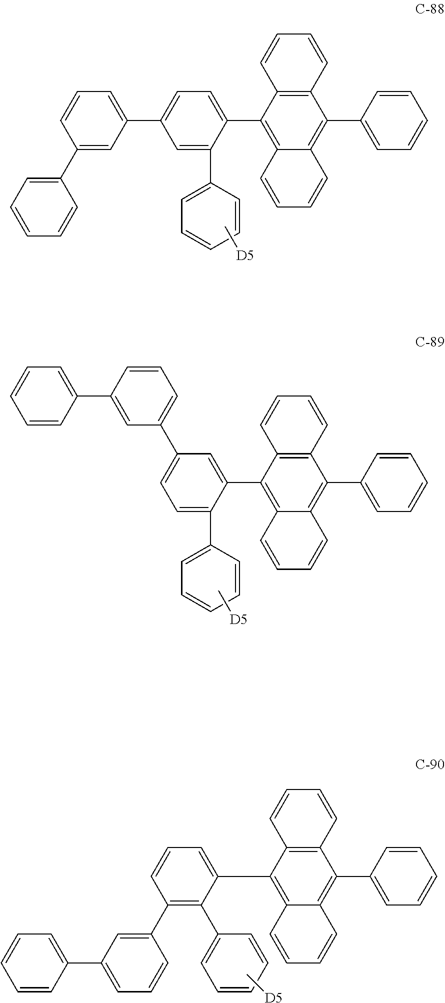

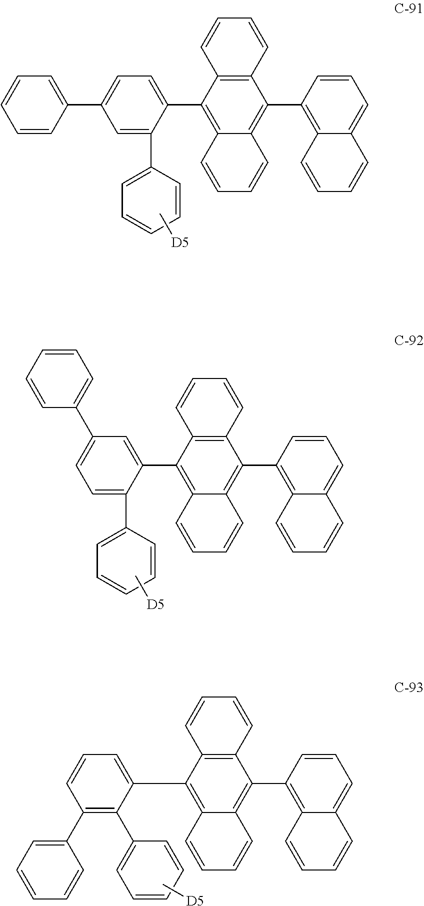

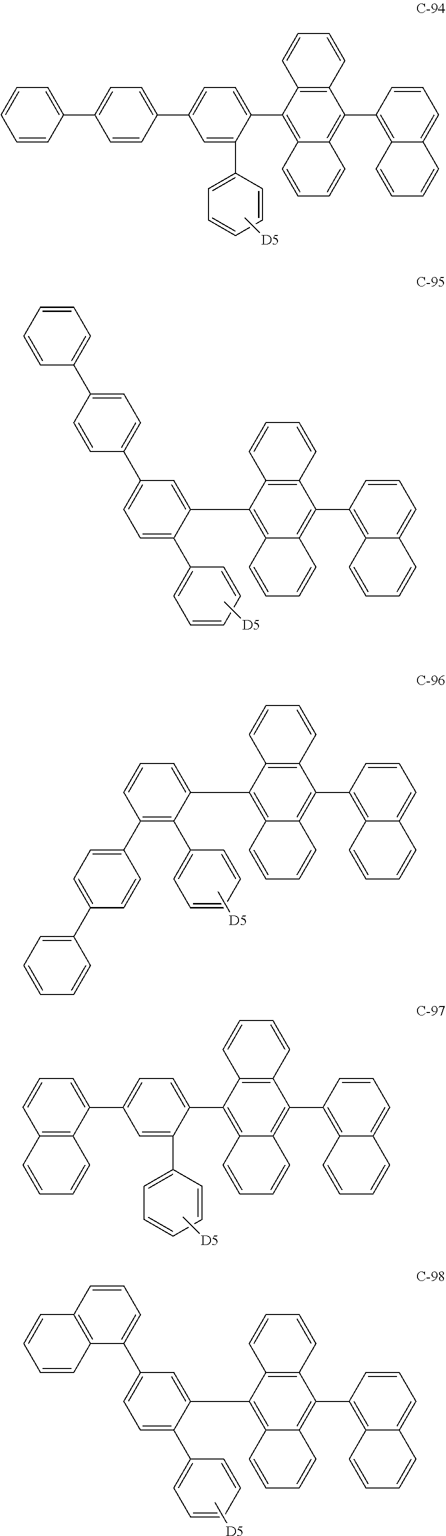

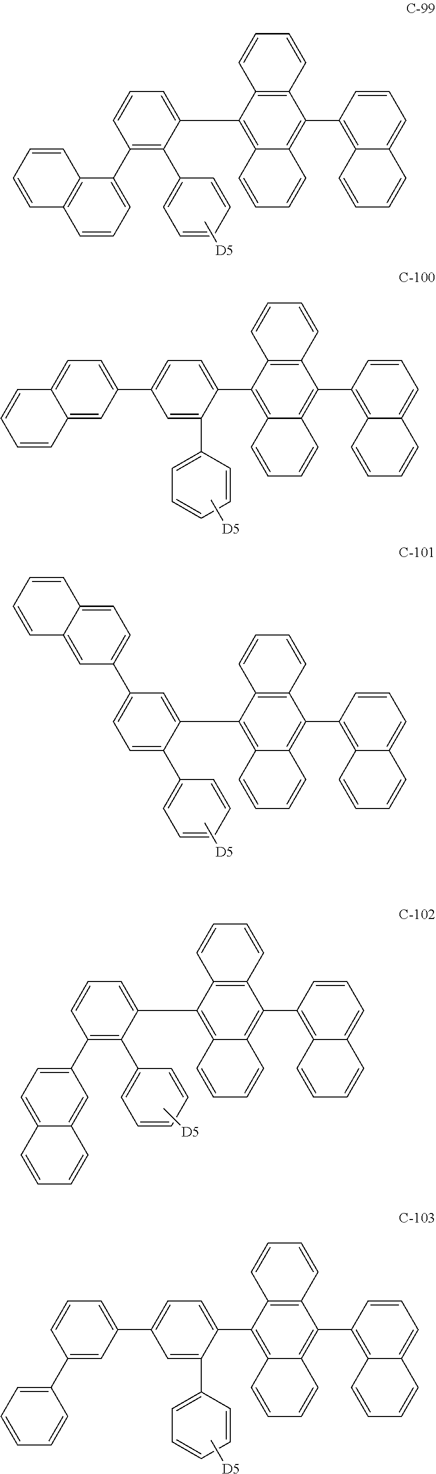

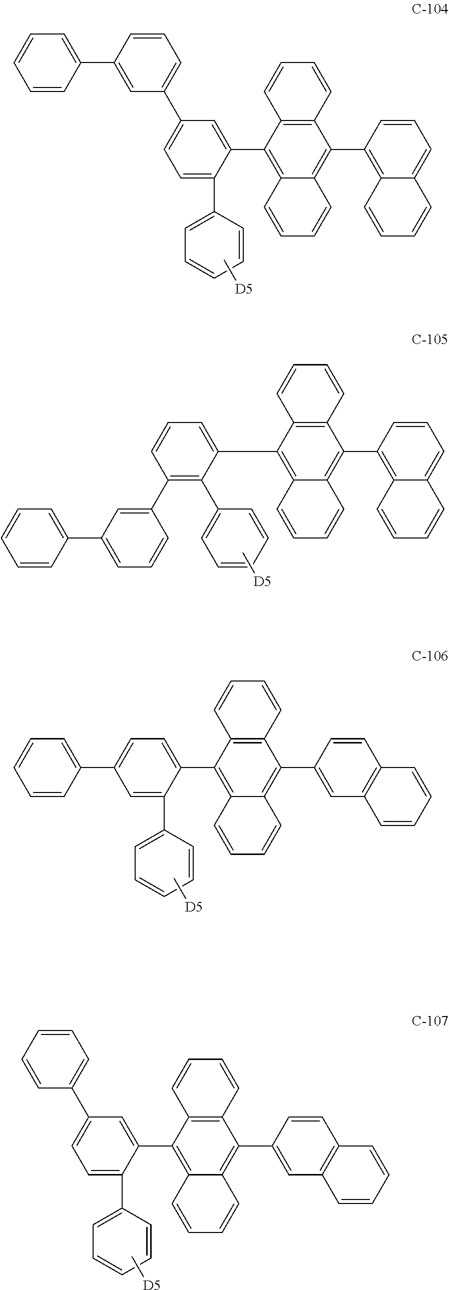

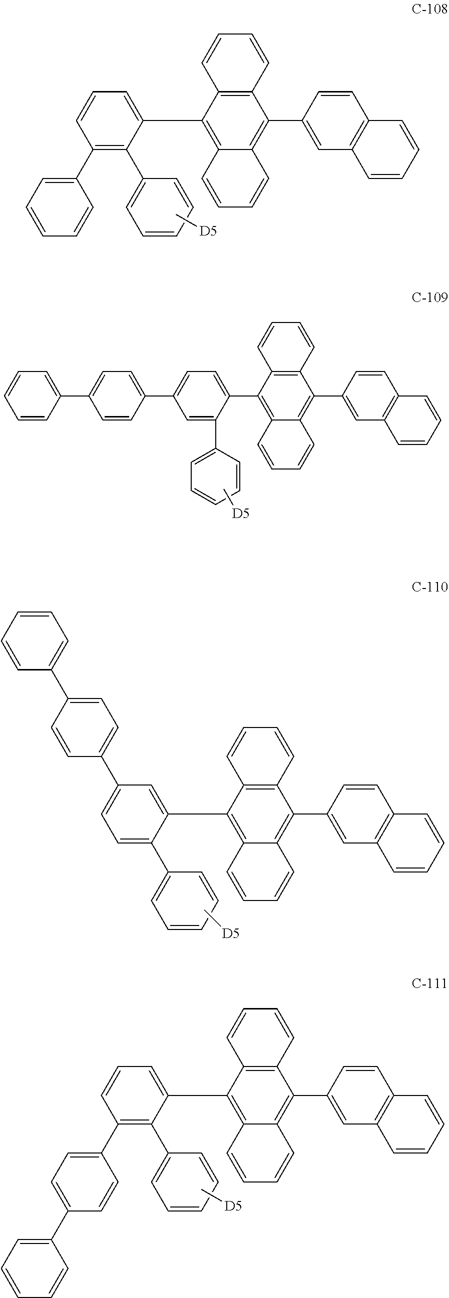

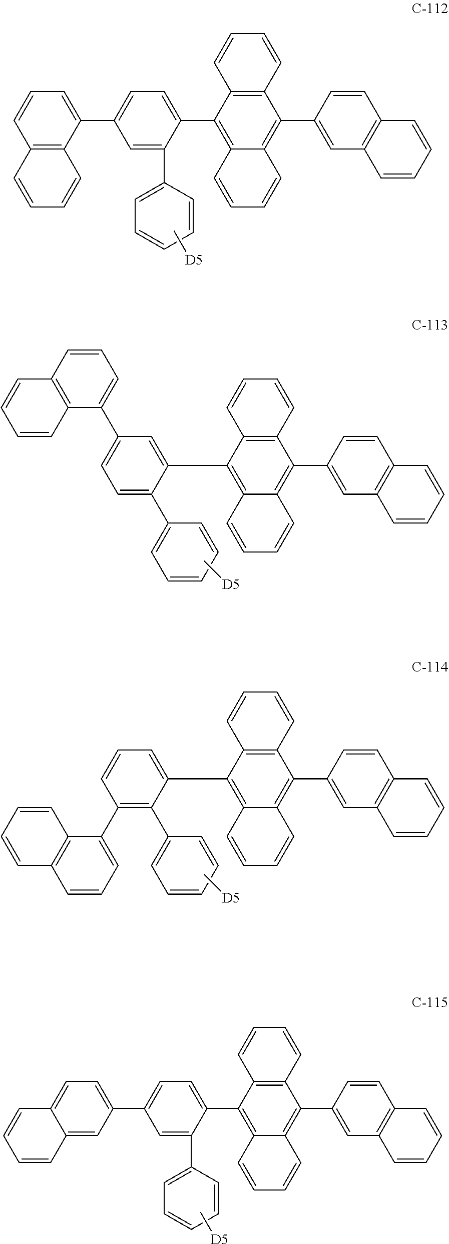

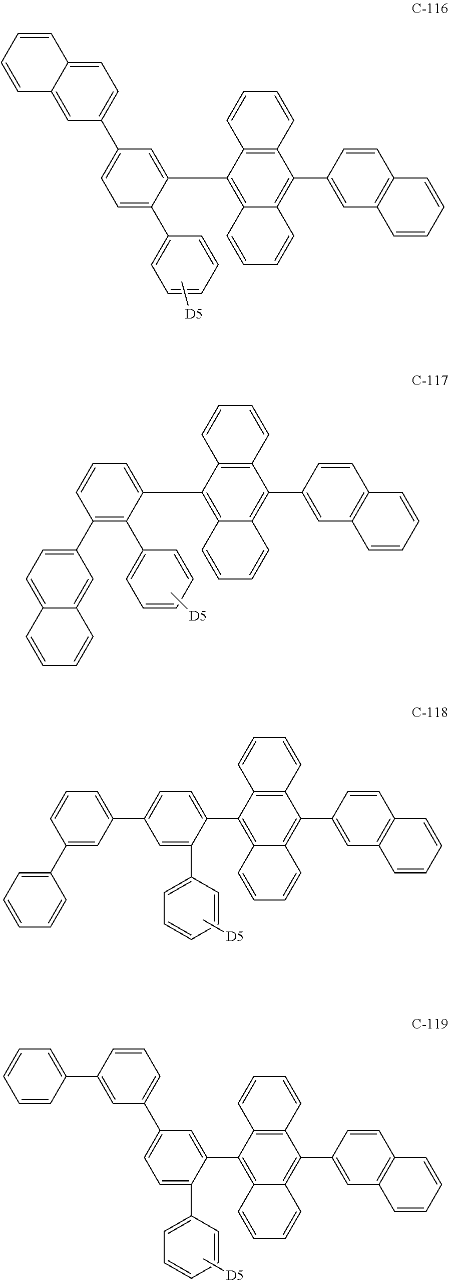

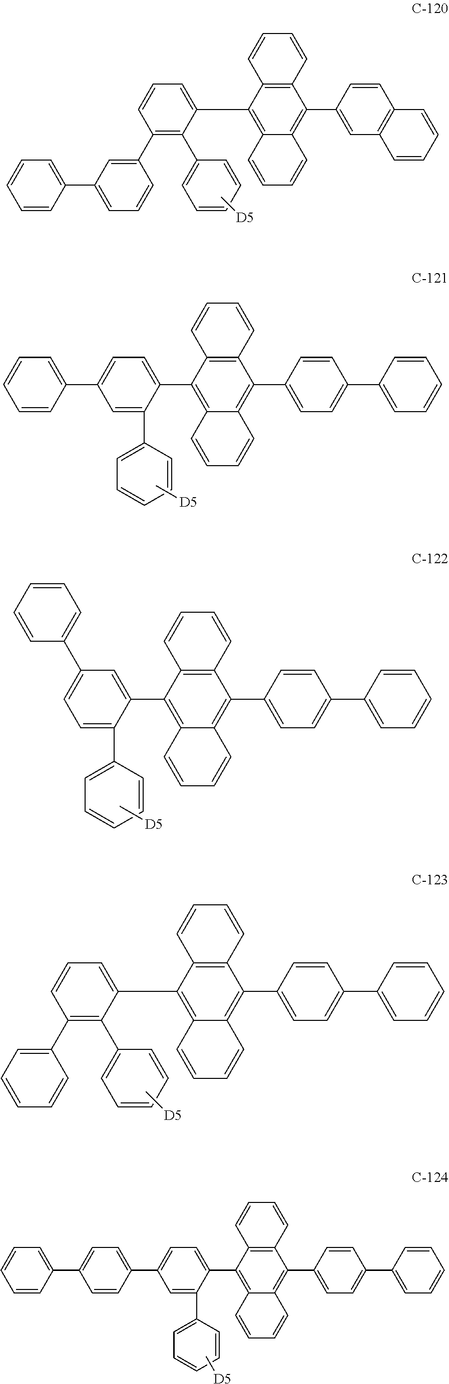

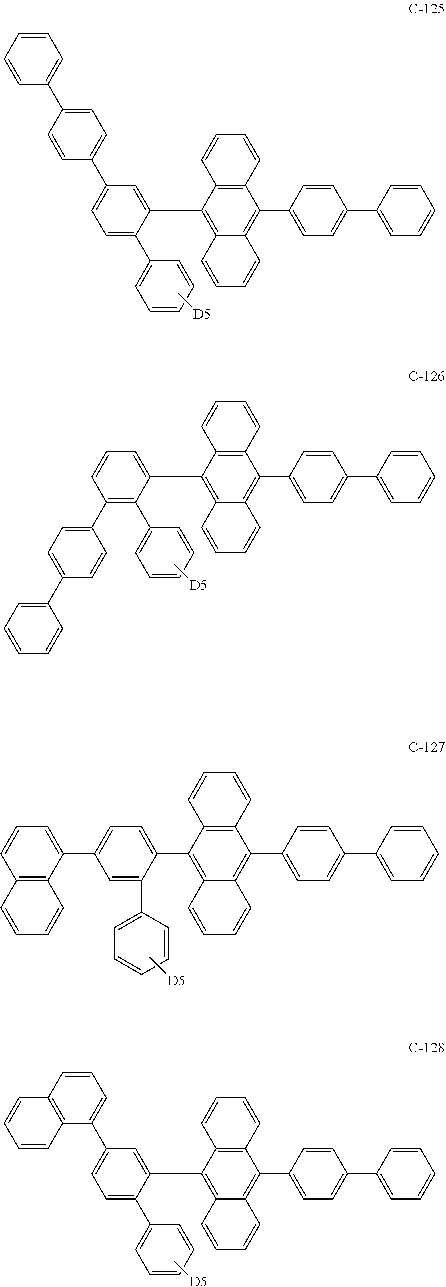

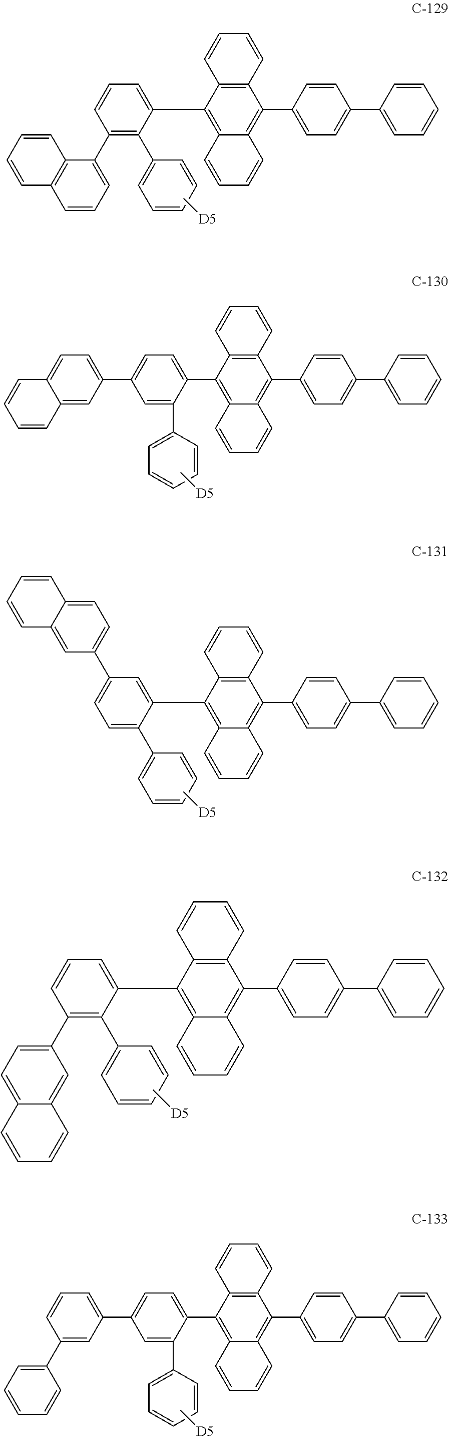

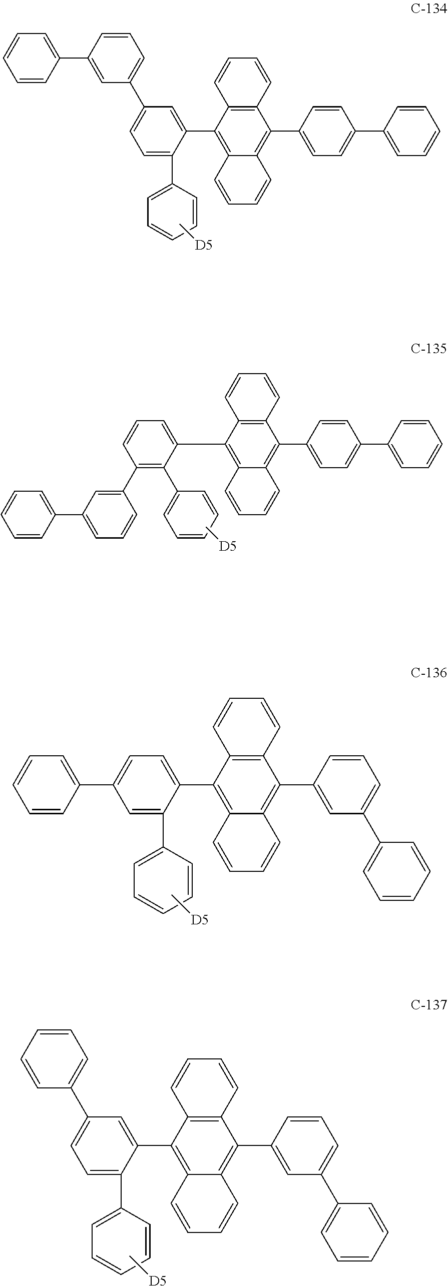

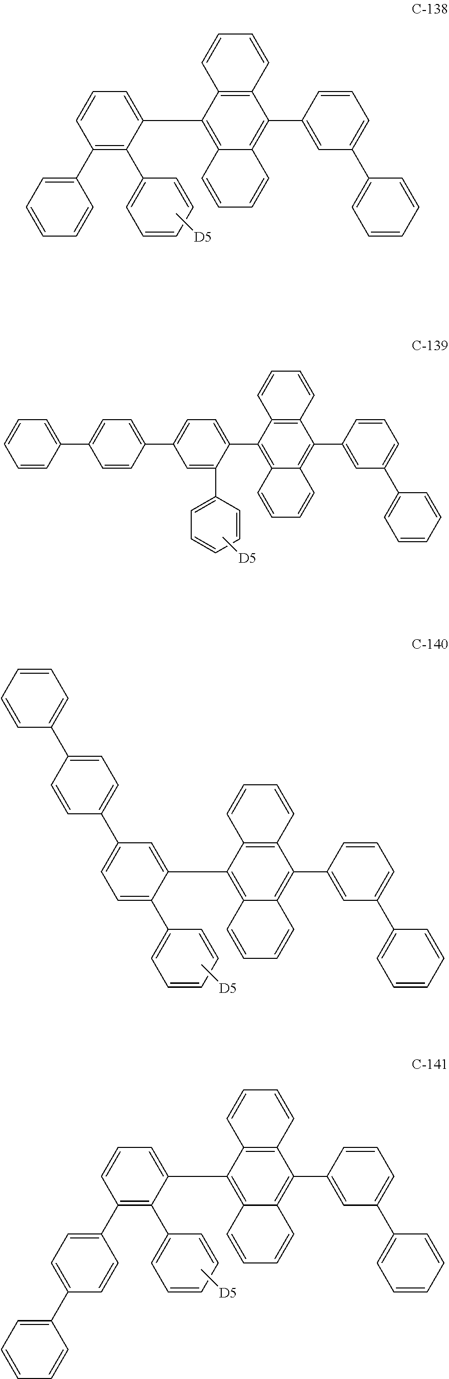

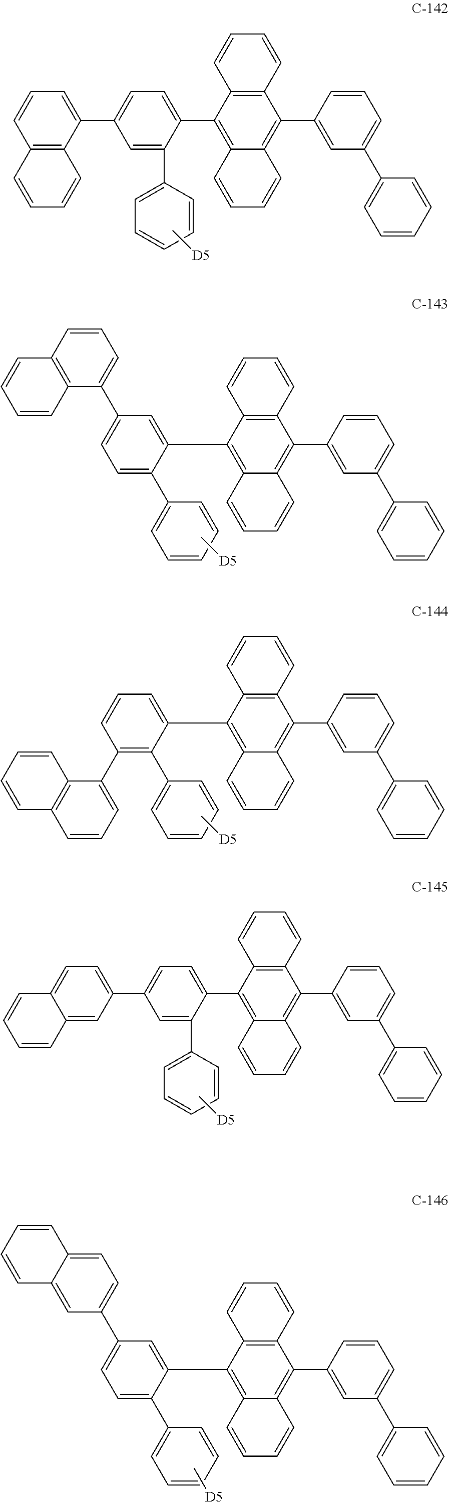

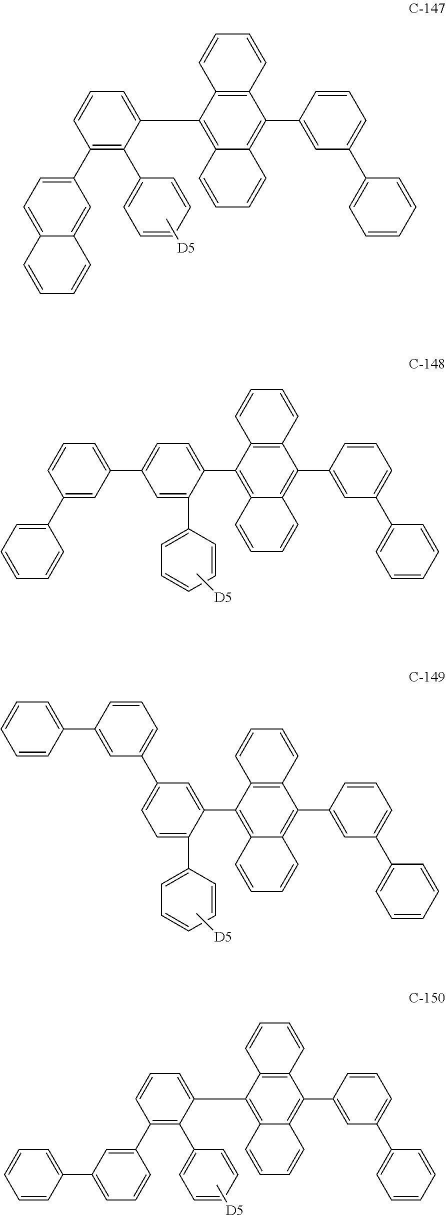

wherein,

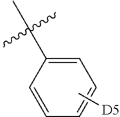

indicates

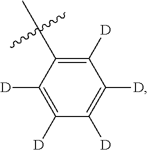

and wherein D indicates deuterium

The organic compound having the structure of Chemical Formulae 1 to 6 has at least one phenyl linked or substituted to 9-position of the anthracene ring. In addition, one phenyl linked to the anthracene ring is substituted with another phenyl at ortho-position thereof with respect to the anthracene ring. The steric effect of the anthracene backbone is controlled to reduce the reactivity of the anthracene-containing compound. Therefore, the organic compound having the structure of Chemical Formulae 1 to 6 can have beneficial luminous lifespan.

For example, the organic compound having the structure of Chemical Formulae 1 to 6 can be applied to an organic electroluminescent material. In one embodiment, the organic compound can be applied to a host in an emitting material layer. In another embodiment, the organic compound having the structure of Chemical Formulae 1 to 6 has proper energy level, so that it can be applied to a charge transport layer or a charge blocking layer.

### Organic Light Emitting Diode and Organic Light Emitting Device

Organic electroluminescent material applying the organic compound, an organic light emitting diode (OLED) and an organic light emitting device including the organic compound will be explained. In one embodiment, the organic electroluminescent material can be applied to an emissive layer that includes such as an emitting material layer, a hole transport layer, an electron blocking layer, a hole blocking layer and/or an electron transport layer in the OLED. For example, when the OLED comprises two or more hole transport layers, the organic compound can be included in a composition for fabricating a hole transport layer (hole auxiliary layer) adjacent to the emitting material layer.

The emissive layer can comprise one or more emitting material layer, and at least one emitting material layer can comprise one or more host material comprising the organic compound having the structure of Chemical Formulae 1 to 6. The emissive layer can additionally comprise at least one of a hole injection layer, a hole transport layer, a hole auxiliary layer, an emitting auxiliary layer, an electron transport layer, an electron injection layer, an interlayer, a hole blocking layer, an electron blocking layer, a hole buffer layer and an electron buffer layer.

In one embodiment, the organic light emitting diode can have one emitting part, or have plural emitting parts to form a tandem structure. Each of plural emitting parts can emit identical color light or different color lights.

As an example, in one or more embodiments of the present disclosure, the organic light emitting diode that includes the organic compound having the structure of Chemical Formulae 1 to 6 can be applied to an organic light emitting device such as an organic light emitting display device or an organic light emitting illumination device. As an example, an organic light emitting display device will be described.

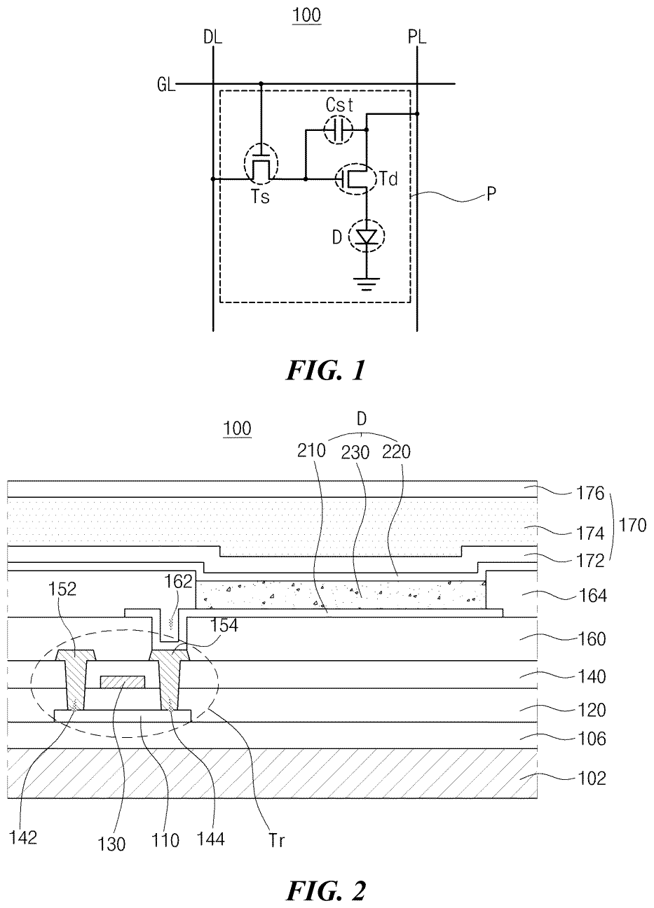

FIG. 1 illustrates a schematic circuit diagram of an organic light emitting display device in accordance with one or more embodiments of the present disclosure. As illustrated in FIG. 1, a gate line GL, a data line DL and power line PL, each of which crosses each other to define a pixel region P, are provided in an organic light emitting display device 100. A switching thin film transistor Ts, a driving thin film transistor Td, a storage capacitor Cst and an organic light emitting diode D are disposed within the pixel region P. The pixel region P can comprise a first pixel region, a second pixel region and a third pixel region. However, embodiments of the present disclosure are not limited to such examples. The organic light emitting display device 100 can comprise a plurality of such pixel regions P which can be arranged in a matrix configuration or other configurations.

The switching thin film transistor Ts is connected to the gate line GL and the data line DL. The driving thin film transistor Td and the storage capacitor Cst are connected between the switching thin film transistor Ts and the power line PL. The organic light emitting diode D is connected to the driving thin film transistor Td. When the switching thin film transistor Ts is turned on by a gate signal applied to the gate line GL, a data signal applied to the data line DL is applied to a gate electrode of the driving thin film transistor Td and one electrode of the storage capacitor Cst through the switching thin film transistor Ts.

The driving thin film transistor Td is turned on by the data signal applied to a gate electrode 130 (FIG. 2) so that a current proportional to the data signal is supplied from the power line PL to the organic light emitting diode D through the driving thin film transistor Td. And then, the organic light emitting diode D emits light having a luminance proportional to the current flowing through the driving thin film transistor Td. In this case, the storage capacitor Cst is charged with a voltage proportional to the data signal so that the voltage of the gate electrode in the driving thin film transistor Td is kept constant during one frame. Therefore, the organic light emitting display device can display a desired image.

FIG. 2 illustrates a schematic cross-sectional view of an organic light emitting display device in accordance with an embodiment of the present disclosure. The pixel circuit configuration of FIG. 1 can be used in the display device of FIG. 2 or other figures of the present application.

As illustrated in FIG. 2, the organic light emitting display device 100 comprises a substrate 102, a thin-film transistor Tr on the substrate 102, and an organic light emitting diode D connected to the thin film transistor Tr.

As an example, the substrate 102 can comprise a red pixel region, a green pixel region and a blue pixel region and an organic light emitting diode D can be located in each pixel region. Each of the organic light emitting diodes D emitting red, green and blue light, respectively, is located correspondingly in the red pixel region, the green pixel region and the blue pixel region.

The substrate 102 can comprise, but is not limited to, glass, thin flexible material and/or polymer plastics. For example, the flexible material can be selected from the group consisting of, but is not limited to, polyimide (PI), polyethersulfone (PES), polyethylenenaphthalate (PEN), polyethylene terephthalate (PET), polycarbonate (PC) and/or combinations thereof. The substrate 102, on which the thin film transistor Tr and the organic light emitting diode D are arranged, forms an array substrate.

A buffer layer 106 can be disposed on the substrate 102. The thin film transistor Tr can be disposed on the buffer layer 106. In certain embodiments, the buffer layer 106 can be omitted.

A semiconductor layer 110 is disposed on the buffer layer 106. In one embodiment, the semiconductor layer 110 can comprise, but is not limited to, oxide semiconductor materials. In this case, a light-shield pattern can be disposed under the semiconductor layer 110, and the light-shield pattern can prevent light from being incident toward the semiconductor layer 110, thereby, preventing or reducing the semiconductor layer 110 from being degraded by the light. Alternatively, the semiconductor layer 110 can comprise polycrystalline silicon. In this case, opposite edges of the semiconductor layer 110 can be doped with impurities.

A gate insulating layer 120 including an insulating material is disposed on the semiconductor layer 110. The gate insulating layer 120 can comprise, but is not limited to, an inorganic insulating material such as silicon oxide (SiOₓ, wherein 0<x≤2) or silicon nitride (SiNₓ, wherein 0<x≤2).

A gate electrode 130 made of a conductive material such as a metal is disposed on the gate insulating layer 120 so as to correspond to a center of the semiconductor layer 110. While the gate insulating layer 120 is disposed on the entire area of the substrate 102 as shown in FIG. 2, the gate insulating layer 120 can be patterned identically as the gate electrode 130.

An interlayer insulating layer 140 including an insulating material is disposed on the gate electrode 130 and covers an entire surface of the substrate 102. The interlayer insulating layer 140 can comprise, but is not limited to, an inorganic insulating material such as silicon oxide (SiOₓ, wherein 0<x≤2) or silicon nitride (SiNₓ, wherein 0<x≤2), or an organic insulating material such as benzocyclobutene or photo-acryl.

The interlayer insulating layer 140 has first and second semiconductor layer contact holes 142 and 144 that expose or do not cover a portion of the surface nearer to the opposing ends than to a center of the semiconductor layer 110. The first and second semiconductor layer contact holes 142 and 144 are disposed on opposite sides of the gate electrode 130 and spaced apart from the gate electrode 130. The first and second semiconductor layer contact holes 142 and 144 are formed within the gate insulating layer 120 and the interlayer insulating layer 140 in FIG. 2. Alternatively, in certain embodiments, the first and second semiconductor layer contact holes 142 and 144 can be formed only within the interlayer insulating layer 140 when the gate insulating layer 120 is patterned identically as the gate electrode 130.

A source electrode 152 and a drain electrode 154, which are made of conductive material such as a metal, are disposed on the interlayer insulating layer 140. The source electrode 152 and the drain electrode 154 are spaced apart from each other on opposing sides of the gate electrode 130, and contact both sides of the semiconductor layer 110 through the first and second semiconductor layer contact holes 142 and 144, respectively.

The semiconductor layer 110, the gate electrode 130, the source electrode 152 and the drain electrode 154 constitute the thin film transistor Tr, which acts as a driving element. The thin film transistor Tr in FIG. 2 has a coplanar structure in which the gate electrode 130, the source electrode 152 and the drain electrode 154 are disposed on the semiconductor layer 110. Alternatively, the thin film transistor Tr can have an inverted staggered structure in which a gate electrode is disposed under a semiconductor layer and a source and drain electrodes are disposed on the semiconductor layer. In this case, the semiconductor layer can comprise amorphous silicon.

The gate line GL and the data line DL, which cross each other to define a pixel region P, and a switching thin film transistor Ts, which is connected to the gate line GL and the data line DL, can be further formed in the pixel region P. The switching thin film transistor Ts is connected to the thin film transistor Tr, which is a driving element. In addition, the power line PL is spaced apart in parallel from the gate line GL or the data line DL. The thin film transistor Tr can further comprise a storage capacitor Cst configured to constantly keep a voltage of the gate electrode 130 for one frame.

A passivation layer 160 is disposed on the source and drain electrodes 152 and 154. The passivation layer 160 covers the thin film transistor Tr on the entire substrate 102. The passivation layer 160 has a flat top surface and a drain contact hole (or a contact hole) 162 that exposes or does not cover the drain electrode 154 of the thin film transistor Tr. While the drain contact hole 162 is disposed on the second semiconductor layer contact hole 144, it can be spaced apart from the second semiconductor layer contact hole 144.

The organic light emitting diode (OLED) D comprises a first electrode 210 that is disposed on the passivation layer 160 and connected to the drain electrode 154 of the thin film transistor Tr. The OLED D further comprises an emissive layer 230 and a second electrode 220 each of which is disposed sequentially on the first electrode 210.

One of the first electrode 210 and the second electrode 220 can be an anode, and the other of the first electrode 210 and the second electrode 220 can be a cathode. One of the first electrode 210 and the second electrode 220 can be a reflective electrode, and the other of the first electrode 210 and the second electrode 220 can be a transmissive electrode.

The first electrode 210 is disposed separately in each pixel region P. In one embodiment, the first electrode 210 can be an anode and comprise conductive material having relatively high work function value. For example, the first electrode 210 can comprise a transparent conductive oxide (TCO).

In one embodiment, when the organic light emitting display device 100 is a bottom-emission type, the first electrode 210 can have a single-layered structure of the TCO. Alternatively, when the organic light emitting display device 100 is a top-emission type, a reflective electrode or a reflective layer can be disposed under the first electrode 210. For example, the reflective electrode or the reflective layer can comprise, but is not limited to, silver (Ag) or aluminum-palladium-copper (APC) alloy. As an example, in the OLED D of the top-emission type, the first electrode 210 can have a triple-layered structure of ITO/Ag/ITO or ITO/APC/ITO.

In addition, a bank layer 164 is disposed on the passivation layer 160 in order to cover edges of the first electrode 210. The bank layer 164 exposes or does not cover a center of the first electrode 210 corresponding to each pixel region. In certain embodiments, the bank layer 164 can be omitted.

An emissive layer 230 is disposed on the first electrode 210. In one embodiment, the emissive layer 230 can have a single-layered structure of an emitting material layer (EML). Alternatively, the emissive layer 230 can have a multiple-layered structure of a hole injection layer (HIL), a hole transport layer (HTL), an electron blocking layer (EBL), an EML, a hole blocking layer (HBL), an electron transport layer (ETL), an electron injection layer (EIL) and/or a charge generation layer (CGL).

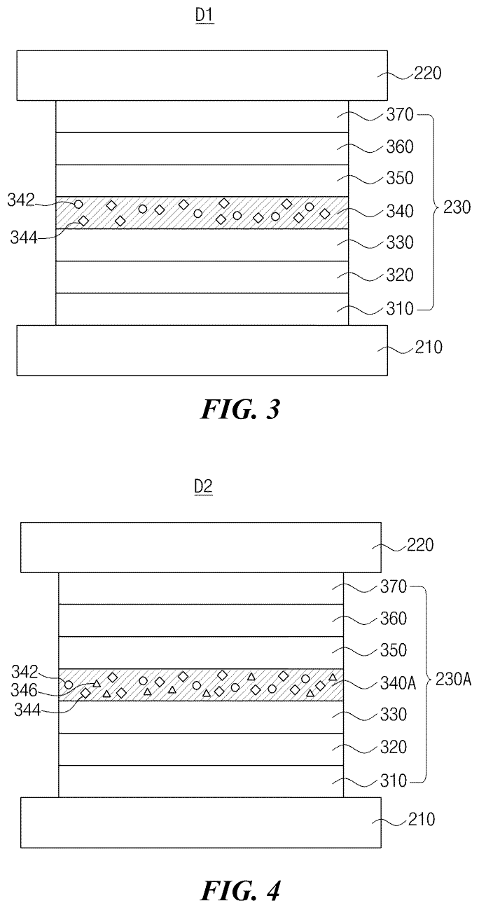

In one embodiment, the emissive layer 230 can have a single emitting part (FIGS. 3 and 4). Alternatively, the emissive layer 230 can have multiple emitting parts to form a tandem structure. For example, the emissive layer 230 can be applied to an OLED with a single emitting part located in each of the red pixel region, the green pixel region and the blue pixel region. Alternatively, the emissive layer 230 can be applied to a tandem-type OLED where at least two emitting parts are stacked.

The emissive layer 230 can comprise the organic compound having the structure of Chemical Formulae 1 to 6. The luminous lifespan of the OLED D and the organic light emitting display device 100 comprising the organic compound can be improved.

The second electrode 220 is disposed on the substrate 102 above which the emissive layer 230 is disposed. The second electrode 220 can be disposed on the entire display area. The second electrode 220 can comprise a conductive material with a relatively low work function value compared to the first electrode 210. The second electrode 220 can be a cathode providing electrons. When the organic light emitting display device 100 is a top-emission type, the second electrode 220 is thin so as to have light-transmissive (semi-transmissive) property.

In addition, an encapsulation film 170 can be disposed on the second electrode 220 in order to prevent or reduce outer moisture from penetrating into the OLED D. The encapsulation film 170 can have, but is not limited to, a laminated structure of a first inorganic insulating film 172, an organic insulating film 174 and a second inorganic insulating film 176. In certain embodiments, the encapsulation film 170 can be omitted.

A polarizing plate can be attached onto the encapsulation film 170 to reduce reflection of external light. For example, the polarizing plate can be a circular polarizing plate. When the organic light emitting display device 100 is a bottom-emission type, the polarizing plate can be disposed under the substrate 102. Alternatively, when the organic light emitting display device 100 is a top-emission type, the polarizing plate can be disposed on the encapsulation film 170. In addition, a cover window can be attached to the encapsulation film 170 or the polarizing plate. In this case, the substrate 102 and the cover window can have a flexible property, thus the organic light emitting display device 100 can be a flexible display device.

The OLED D is described in more detail. FIG. 3 illustrates a schematic cross-sectional view of an organic light emitting diode having a single emitting part in accordance with an embodiment of the present disclosure. For instance, FIG. 3 shows an example (OLED D1) of the OLED D in FIGS. 1 and 2.

As illustrated in FIG. 3, the organic light emitting diode (OLED) D1 in accordance with an example of the present disclosure comprises first and second electrodes 210 and 220 facing each other and an emissive layer 230 disposed between the first and second electrodes 210 and 220. The organic light emitting display device 100 comprises a red pixel region, a green pixel region and a blue pixel region, and the OLED D1 can be disposed in the red pixel region, the green pixel region and/or the blue pixel region. As an example, the OLED D1 can be disposed in the blue pixel region.

In one embodiment, the emissive layer 230 comprises an emitting material layer (EML) 340 disposed between the first and second electrodes 210 and 220. The EML 340 can be a blue emitting material layer.

The emissive layer 230 can comprise at least one of a hole transport layer (HTL) 320 disposed between the first electrode 210 and the EML 340 and an electron transport layer (ETL) 360 disposed between the second electrode 220 and the EML 340. In certain embodiments, the emissive layer 230 can further comprise at least one of a hole injection layer (HIL) 310 disposed between the first electrode 210 and the HTL 320 and an electron injection layer (EIL) 370 disposed between the second electrode 220 and the ETL 360. Alternatively or additionally, the emissive layer 230 can further comprise a first exciton blocking layer, i.e., an electron blocking layer (EBL) 330 disposed between the HTL 320 and the EML 340 and/or a second exciton blocking layer, i.e., a hole blocking layer (HBL) 350 disposed between the EML 340 and the ETL 360.

The first electrode 210 can be an anode that provides holes into the EML 340. The first electrode 210 can comprise a conductive material having a relatively high work function value, for example, a transparent conductive oxide (TCO). As an example, the first electrode 210 can include, but is not limited to, indium tin oxide (ITO), indium zinc oxide (IZO), indium tin zinc oxide (ITZO), tin oxide (SnO), zinc oxide (ZnO), indium cerium oxide (ICO), aluminum doped zinc oxide (AZO), and/or the like.

The second electrode 220 can be a cathode that provides electrons into the EML 340. The second electrode 220 can comprise a conductive material having a relatively low work function values, i.e., a highly reflective material. As an example, the second electrode 220 can comprise, but is not limited to, aluminum (Al), magnesium (Mg), calcium (Ca), silver (Ag), alloy thereof and/or combinations thereof such as aluminum-magnesium alloy (Al—Mg).

The EML 340 can comprise a host 342 and a dopant (emitter) 344 where ultimate light emission is occurred. The EML 340 can emit blue color light. The host 342 can be one or more. For example, when the EML 340 comprises two or more hosts, the host 342 can comprise a P-type (hole-type) host with beneficial hole affinity property and/or hole transporting property, and an N-type (electron type) host with beneficial electron affinity property and/or electron transporting affinity. As an example, the host 342 can comprise the anthracene-containing organic compound having the structure of Chemical Formulae 1 to 6.

The dopant 344 can emit blue color light. As an example, the dopant 344 can be blue phosphorescent material, blue fluorescent material or blue delayed fluorescent material. In one embodiment, the dopant 344 of blue phosphorescent material can comprise, but is not limited to, an organometallic compound having the following structure of Chemical Formula 7:

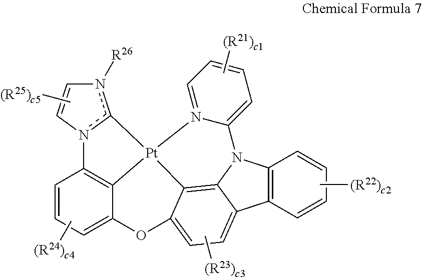

wherein, in Chemical Formula 7,
each of R²¹ to R²⁶ is independently protium, deuterium, tritium, halogen, a cyan group, an unsubstituted or substituted C₁-C₂₀ alkyl group, an unsubstituted or substituted C₂-C₂₀ alkenyl group, an unsubstituted or substituted C₂-C₂₀ alkynyl group, an unsubstituted or substituted C₁-C₂₀ alkoxy group, an amino group, an unsubstituted or substituted C₁-C₂₀ alkyl amino group, an unsubstituted or substituted C₁-C₂₀ alkyl silyl group, an unsubstituted or substituted C₃-C₃₀ cycloalkyl group, an unsubstituted or substituted C₃-C₃₀ hetero cycloalkyl group, an unsubstituted or substituted C₆-C₃₀ aryl group, an unsubstituted or substituted C₂-C₃₀ hetero aryl group, an unsubstituted or substituted C₇-C₃₀ aralkyl group, an unsubstituted or substituted C₃-C₃₀ hetero aralkyl group, an unsubstituted or substituted C₆-C₃₀ aryloxy group, an unsubstituted or substituted C₂-C₃₀ hetero aryloxy group, an unsubstituted or substituted C₆-C₃₀ aryl amino group, an unsubstituted or substituted C₂-C₃₀ hetero aryl amino group, an unsubstituted or substituted C₆-C₃₀ aryl silyl group, an unsubstituted or substituted C₂-C₃₀ hetero aryl silyl group, an unsubstituted or substituted C₁-C₂₀ alkyl germanyl group, an unsubstituted or substituted C₆-C₃₀ aryl germanyl group, an unsubstituted or substituted C₂-C₃₀ hetero aryl germanyl group, an unsubstituted or substituted C₆-C₃₀ tri-aryl methyl group, or an unsubstituted or substituted C₂-C₃₀ tri-hetero aryl methyl group, where each R²¹ is identical to or different from each other when c1 is 2, 3 or 4, each R²² is identical to or different from each other when c2 is 2, 3 or 4, each R²³ is identical to or different from each other when c3 is 2, each R²⁴ is identical to or different from each other when c4 is 2 or 3, and each R²¹ is identical to different from each other when c5 is 2, or
optionally,
two adjacent R²¹, two adjacent R²², two adjacent R²³, two adjacent R²⁴ and/or two adjacent R²¹ are further linked to form an unsubstituted or substituted benzene ring;
each of c1 and c2 is independently 0, 1, 2, 3 or 4;
each of c3 and c5 is independently 0, 1 or 2; and
c4 is 0, 1, 2 or 3.

For example, the dopant 344 of blue phosphorescent material can be at least one of or selected from, but is not limited to, the following compounds of Chemical Formula 8:

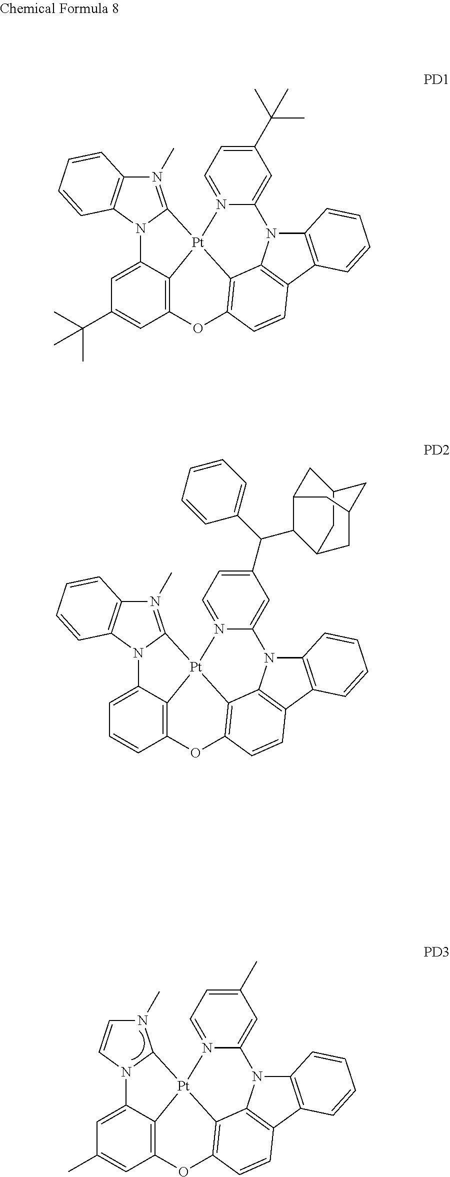

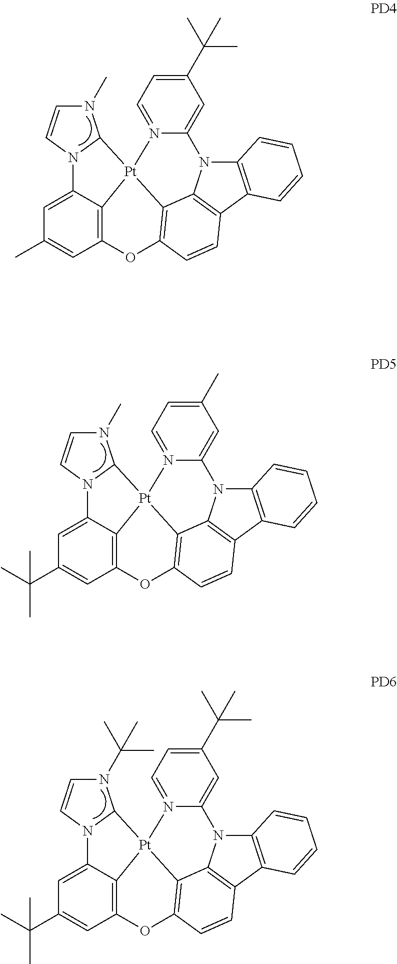

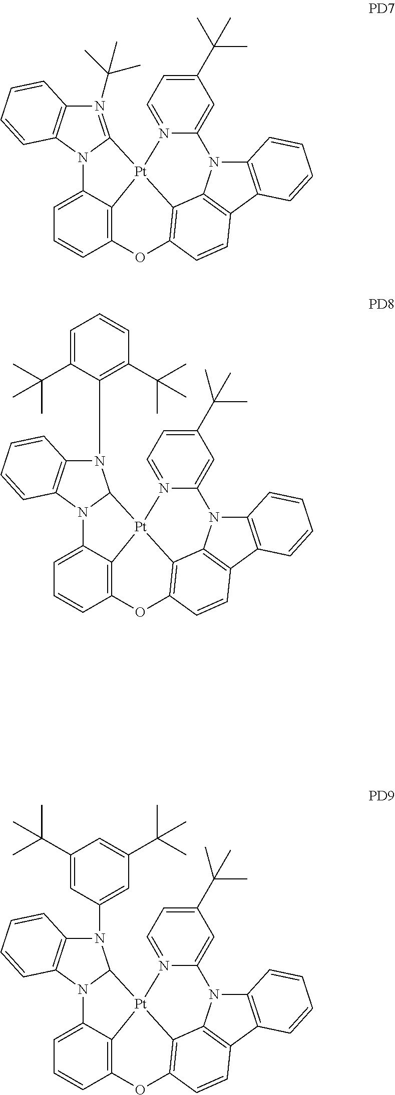

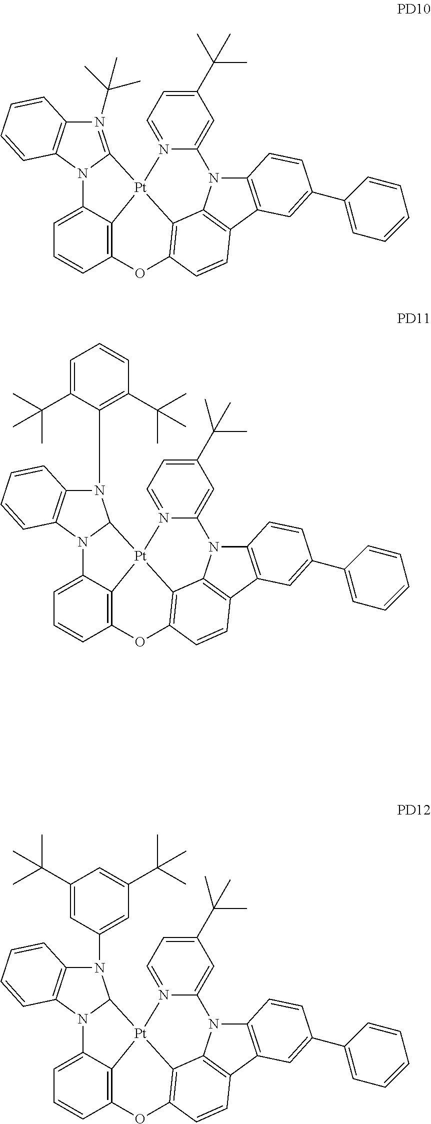

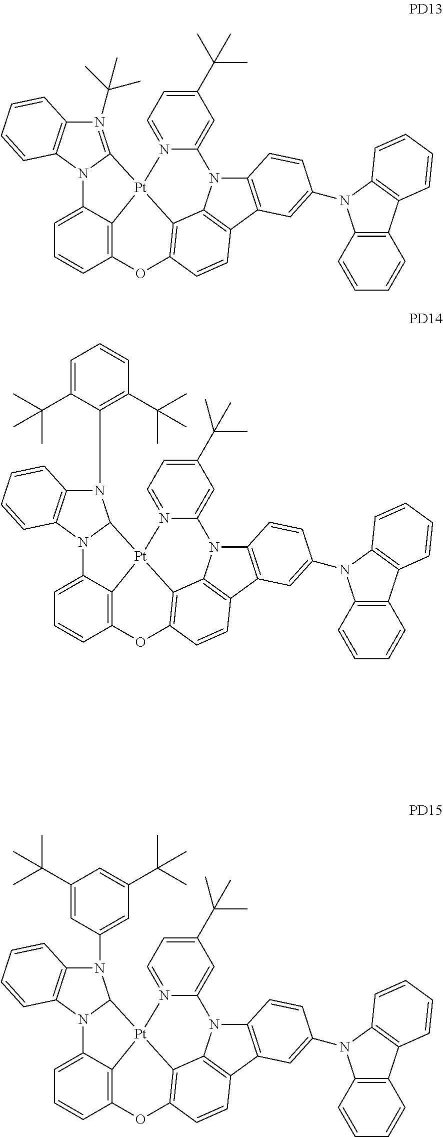

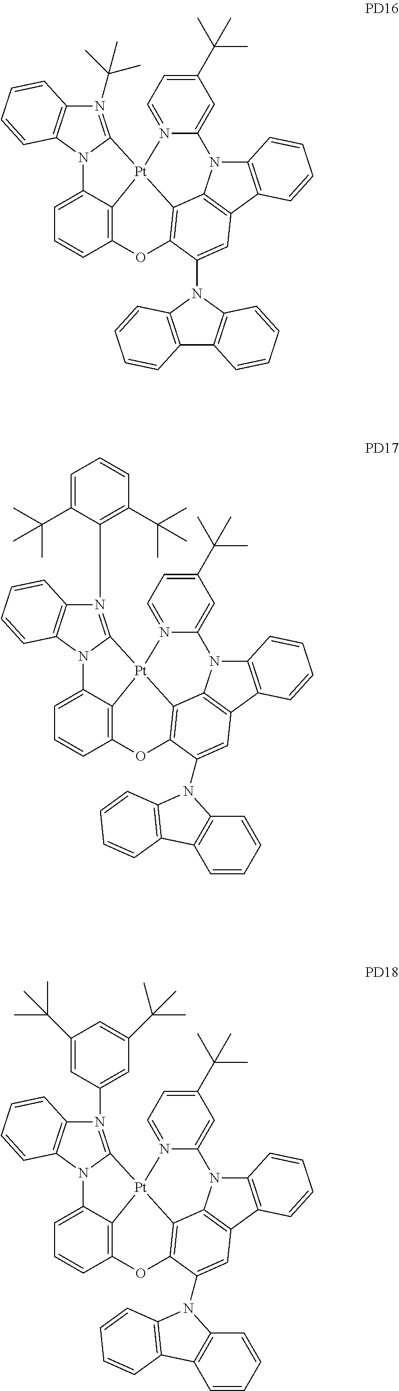

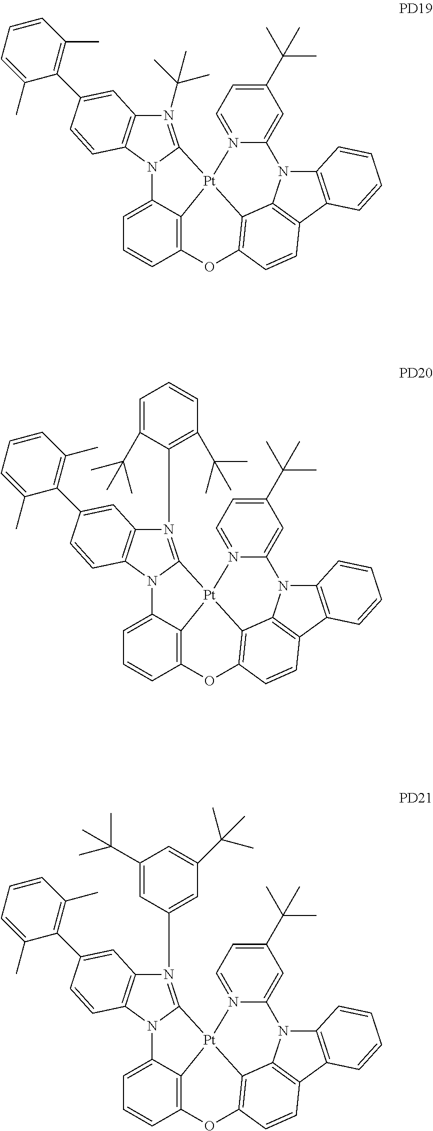

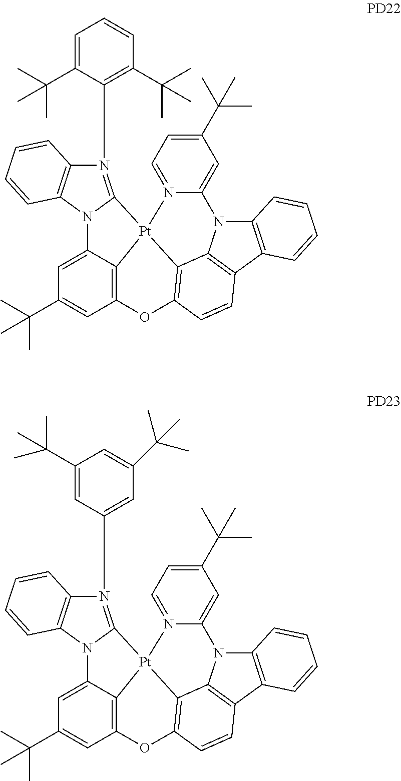

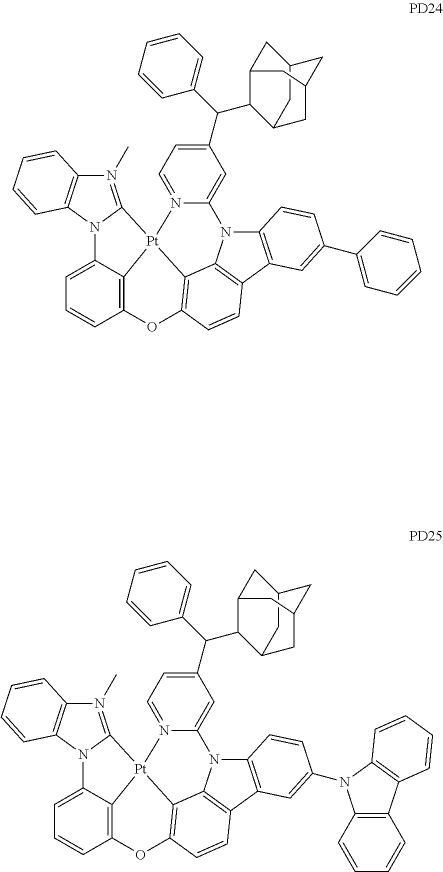

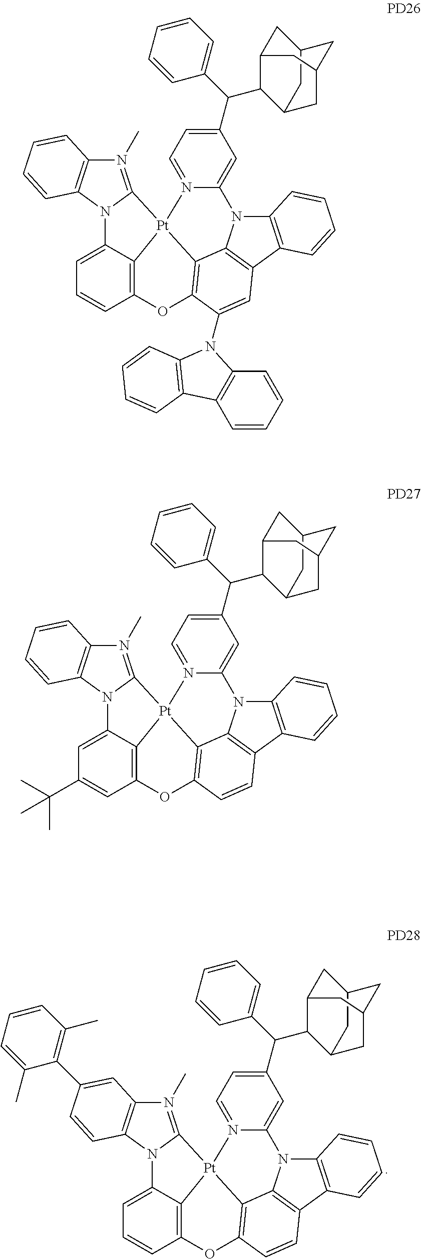

In another embodiment, the dopant 344 of blue fluorescent material can be a polycyclic organic compound with boron atom and nitrogen atom. As an example, the dopant 344 of blue fluorescent material can have the following structure of Chemical Formula 9:

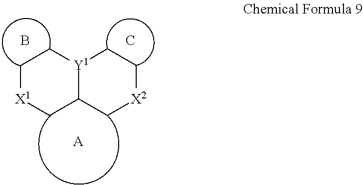

wherein, in Chemical Formula 9,
each of ring A, ring B and ring C is independently an unsubstituted or substituted C₆-C₃₀ aromatic ring or an unsubstituted or substituted C₃-C₃₀ hetero aromatic ring;
Y¹ is boron;
each of X¹ and X² is independently NR^{A}, O or S;
R^{A} is protium, deuterium, tritium, halogen, a cyan group, an unsubstituted or substituted C₁-C₂₀ alkyl group, an unsubstituted or substituted C₂-C₂₀ alkenyl group, an unsubstituted or substituted C₂-C₂₀ alkynyl group, an unsubstituted or substituted C₁-C₂₀ alkoxy group, an amino group, an unsubstituted or substituted C₁-C₂₀ alkyl amino group, an unsubstituted or substituted C₁-C₂₀ alkyl silyl group, an unsubstituted or substituted C₃-C₃₀ cycloalkyl group, an unsubstituted or substituted C₃-C₃₀ hetero cycloalkyl group, an unsubstituted or substituted C₆-C₃₀ aryl group, an unsubstituted or substituted C₂-C₃₀ hetero aryl group, an unsubstituted or substituted C₇-C₃₀ aralkyl group, an unsubstituted or substituted C₃-C₃₀ hetero aralkyl group, an unsubstituted or substituted C₆-C₃₀ aryloxy group, an unsubstituted or substituted C₂-C₃₀ hetero aryloxy group, an unsubstituted or substituted C₆-C₃₀ aryl amino group, an unsubstituted or substituted C₂-C₃₀ hetero aryl amino group, an unsubstituted or substituted tri-phenyl methyl group, an unsubstituted or substituted C₆-C₃₀ aryl silyl group, an unsubstituted or substituted C₂-C₃₀ hetero aryl silyl group, an unsubstituted or substituted C₁-C₂₀ alkyl germanyl group, an unsubstituted or substituted C₆-C₃₀ aryl germanyl group, an unsubstituted or substituted C₂-C₃₀ hetero aryl germanyl group, or -L¹-N—(R^{B})(R^{C}), or
optionally,
R^{A} is linked to at least one of ring A, ring B and ring C to form an unsubstituted or substituted C₃-C₃₀ alicyclic ring, an unsubstituted or substituted C₃-C₃₀ hetero alicyclic ring, an unsubstituted or substituted C₆-C₃₀ aromatic ring or an unsubstituted or substituted C₂-C₃₀ hetero aromatic ring;
L¹ is a single bond, an unsubstituted or substituted C₆-C₃₀ arylene group, an unsubstituted or substituted C₂-C₃₀ hetero arylene group, an unsubstituted or substituted divalent C₁-C₃₀ aliphatic group, or a divalent fused ring group of an unsubstituted or substituted C₃-C₃₀ alicyclic ring and an unsubstituted or substituted C₆-C₃₀ aromatic ring; and
each of R^{B} and R^{C} is independently an unsubstituted or substituted C₁-C₂₀ alkyl group, an unsubstituted or substituted C₂-C₂₀ alkenyl group, an unsubstituted or substituted C₆-C₃₀ aryl group or an unsubstituted or substituted C₂-C₃₀ hetero aryl group.

For example, each of ring A, ring B and ring C in Chemical Formula 9 can be independently a C₆-C₂₀ aromatic ring. As an example, the dopant 344 of blue fluorescent material can have the following structure of Chemical Formula 10:

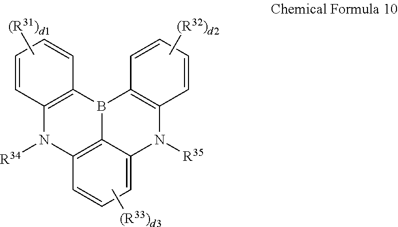

wherein, in Chemical Formula 10,
each of R³¹ to R³⁵ is independently protium, deuterium, tritium, halogen, a cyan group, an unsubstituted or substituted C₁-C₂₀ alkyl group, an unsubstituted or substituted C₂-C₂₀ alkenyl group, an unsubstituted or substituted C₂-C₂₀ alkynyl group, an unsubstituted or substituted C₁-C₂₀ alkoxy group, an amino group, an unsubstituted or substituted C₁-C₂₀ alkyl amino group, an unsubstituted or substituted C₁-C₂₀ alkyl silyl group, an unsubstituted or substituted C₃-C₃₀ cycloalkyl group, an unsubstituted or substituted C₃-C₃₀ hetero cycloalkyl group, an unsubstituted or substituted C₆-C₃₀ aryl group, an unsubstituted or substituted C₂-C₃₀ hetero aryl group, an unsubstituted or substituted C₇-C₃₀ aralkyl group, an unsubstituted or substituted C₃-C₃₀ hetero aralkyl group, an unsubstituted or substituted C₆-C₃₀ aryloxy group, an unsubstituted or substituted C₂-C₃₀ hetero aryloxy group, an unsubstituted or substituted C₆-C₃₀ aryl amino group, an unsubstituted or substituted C₂-C₃₀ hetero aryl amino group, an unsubstituted or substituted C₆-C₃₀ aryl silyl group, an unsubstituted or substituted C₂-C₃₀ hetero aryl silyl group, an unsubstituted or substituted C₁-C₂₀ alkyl germanyl group, an unsubstituted or substituted C₆-C₃₀ aryl germanyl group or an unsubstituted or substituted C₂-C₃₀ hetero aryl germanyl group, where each R³¹ is identical to or different from each other when d1 is 2, 3 or 4, each R³² is identical to or different from each other when d2 is 2, 3 or 4, and each R³³ is identical to or different from each other when d3 is 2 or 3,
optionally,
two adjacent R³¹, two adjacent R³² and/or two adjacent R³³ are further linked to form an unsubstituted or substituted C₃-C₃₀ alicyclic ring, an unsubstituted or substituted C₃-C₃₀ hetero alicyclic ring, an unsubstituted or substituted C₆-C₃₀ aromatic ring or an unsubstituted or substituted C₂-C₃₀ hetero aromatic ring, or
R⁴ can be linked to at least one ring of a benzene ring where R³¹ is linked and a benzene ring where R³³ is linked, and R³⁵ can be linked to at least one ring of a benzene ring where R³² is linked and a benzene ring where R³³ is linked;
each of d1 and d2 is independently 0, 1, 2, 3 or 4; and
d3 is 0, 1, 2 or 3.

As an example, the dopant 344 of blue fluorescent material can be at least one of or selected from, but is not limited to, the following compounds of Chemical Formula 11:

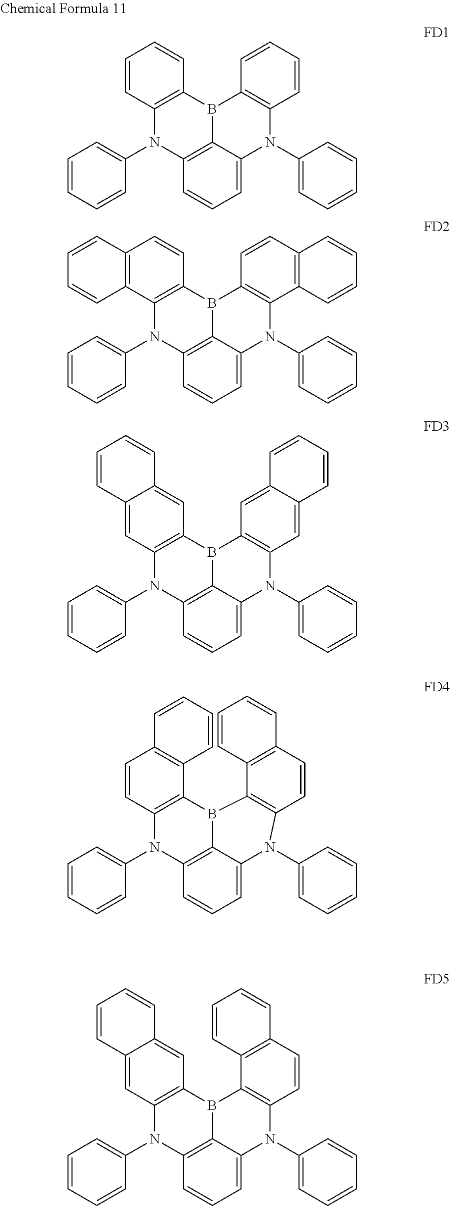

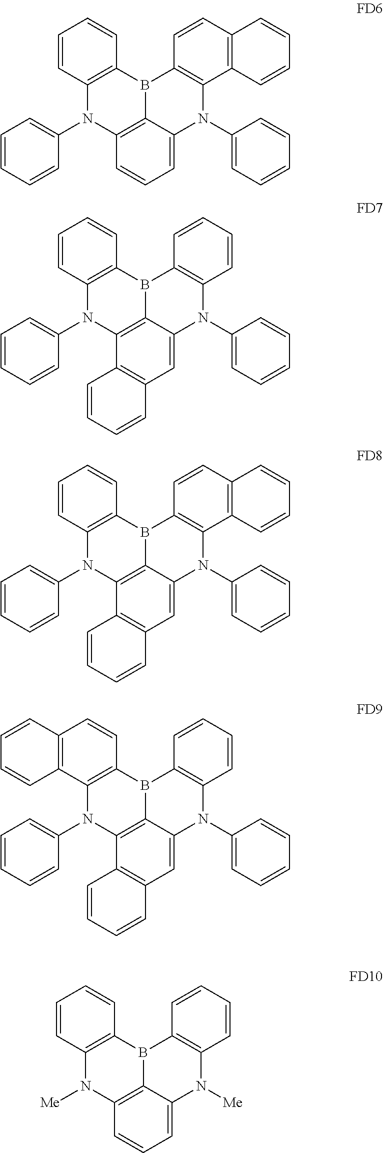

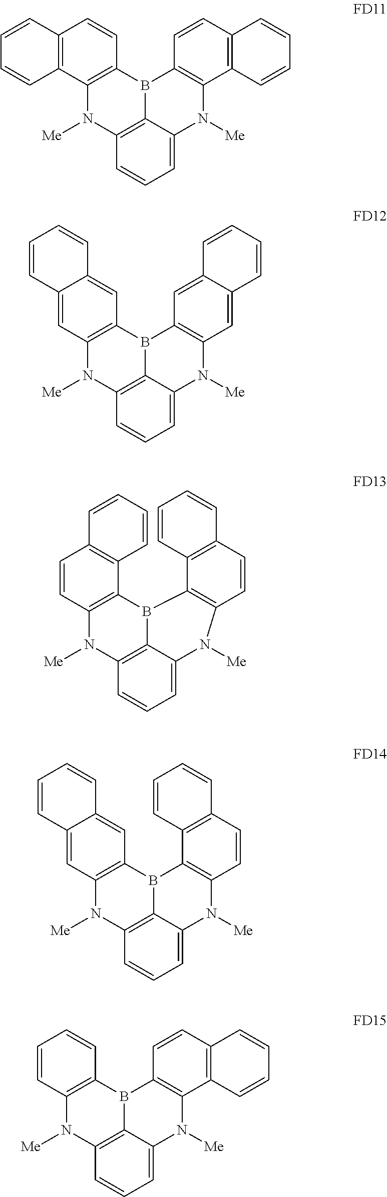 [SMILES: CN1c2cccc3c2B(c2ccc4ccccc4c21)c1ccc2ccccc2c1N3C ; CN1c2cc3ccccc3cc2B2c3cc4ccccc4cc3N(C)c3cccc1c32 ; CN1c2cccc3c2B(c2c1ccc1ccccc21)c1c(ccc2ccccc12)N3C ; CN1c2cc3ccccc3cc2B2c3c1cccc3N(C)c1ccc3ccccc3c12 ; CN1c2ccccc2B2c3ccc4ccccc4c3N(C)c3cccc1c32]

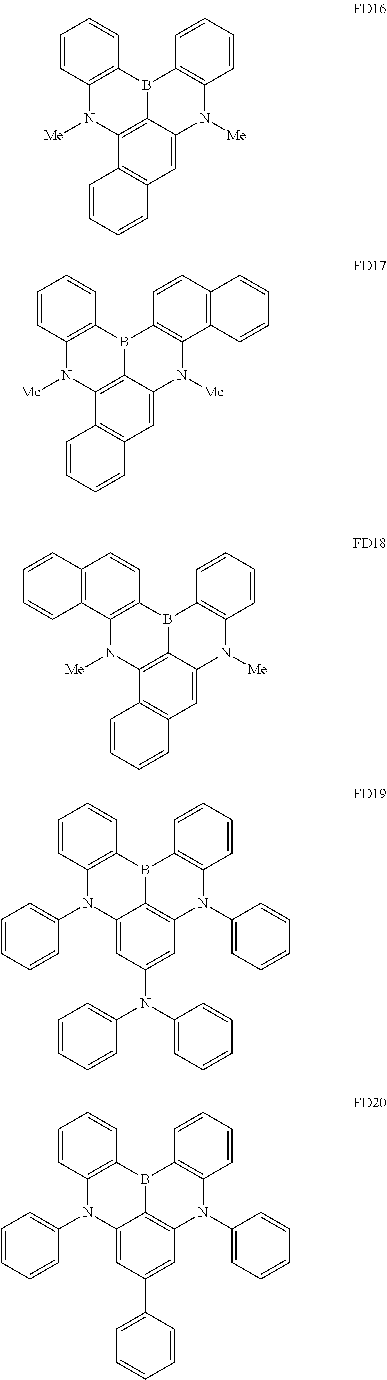

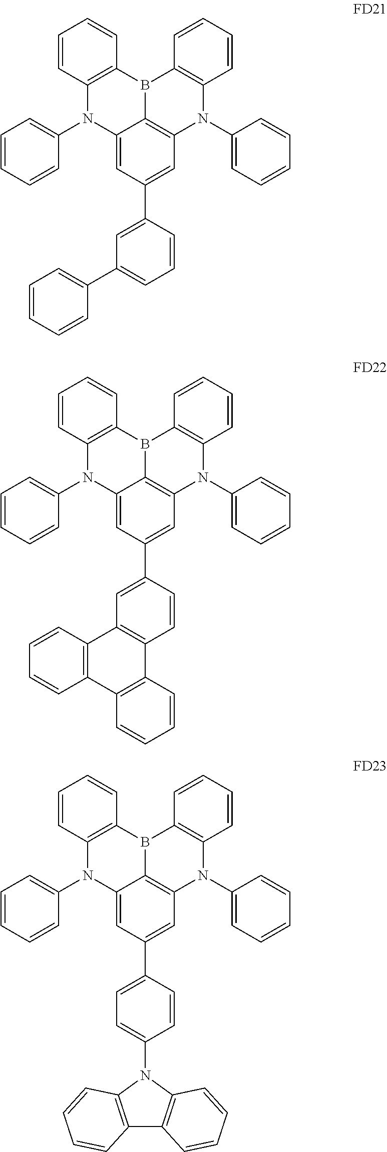 [SMILES: c1ccc(-c2cccc(-c3cc4c5c(c3)N(c3ccccc3)c3ccccc3B5c3ccccc3N4c3ccccc3)c2)cc1 ; c1ccc(N2c3ccccc3B3c4ccccc4N(c4ccccc4)c4cc(-c5ccc6c7ccccc7c7ccccc7c6c5)cc2c43)cc1 ; c1ccc(N2c3ccccc3B3c4ccccc4N(c4ccccc4)c4cc(-c5ccc(-n6c7ccccc7c7ccccc76)cc5)cc2c43)cc1]

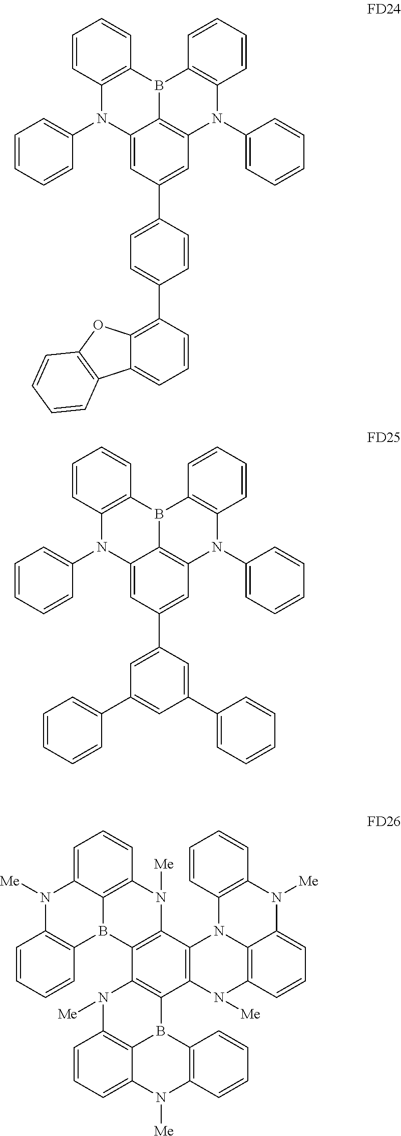

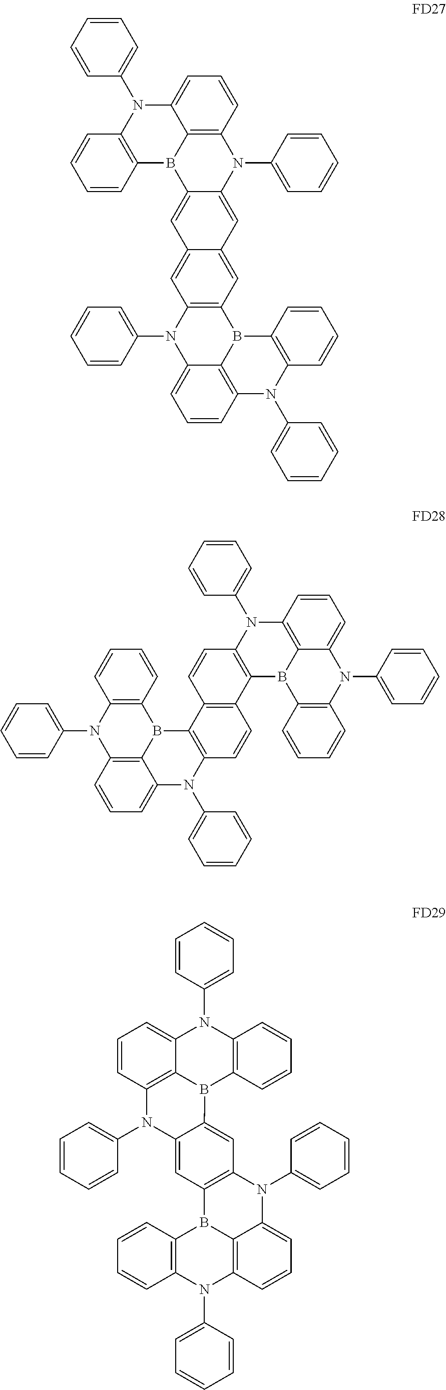 [SMILES: c1ccc(N2c3ccccc3B3c4cc5cc6c(cc5cc4N(c4ccccc4)c4cccc2c43)B2c3ccccc3N(c3ccccc3)c3cccc(c32)N6c2ccccc2)cc1 ; c1ccc(N2c3ccccc3B3c4c2cccc4N(c2ccccc2)c2ccc4c5c(ccc4c23)N(c2ccccc2)c2cccc3c2B5c2ccccc2N3c2ccccc2)cc1 ; c1ccc(N2c3ccccc3B3c4cc5c(cc4N(c4ccccc4)c4cccc2c43)B2c3ccccc3N(c3ccccc3)c3cccc(c32)N5c2ccccc2)cc1]

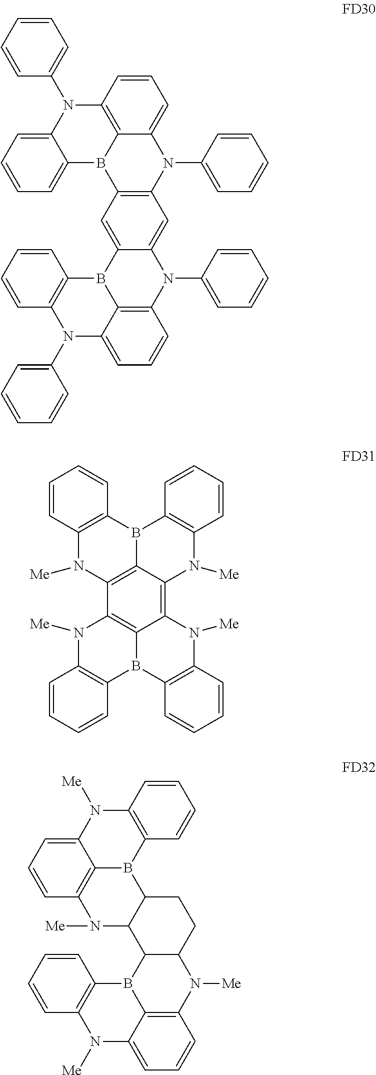 [SMILES: c1ccc(N2c3ccccc3B3c4cc5c(cc4N(c4ccccc4)c4cccc2c43)N(c2ccccc2)c2cccc3c2B5c2ccccc2N3c2ccccc2)cc1 ; CN1c2ccccc2B2c3ccccc3N(C)c3c2c1c1c2c3N(C)c3ccccc3B2c2ccccc2N1C ; CN1c2ccccc2B2c3c1cccc3N(C)C1CCC3B4c5ccccc5N(C)c5cccc(c54)N(C)C3C21]

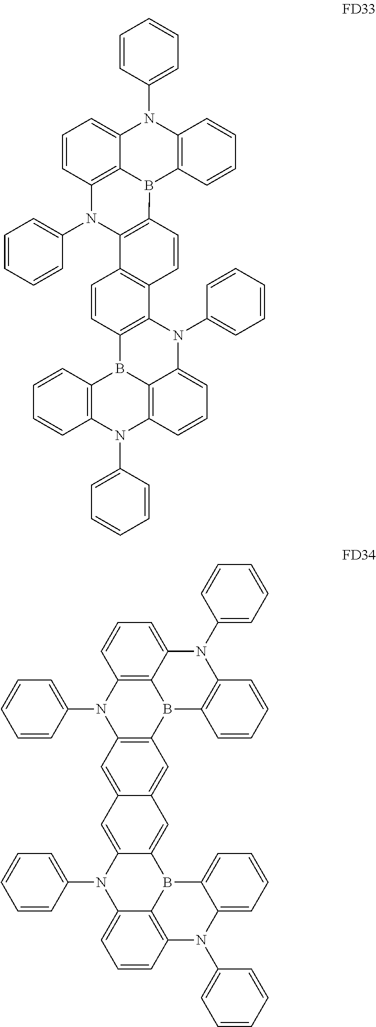 [SMILES: c1ccc(N2c3ccccc3B3c4ccc5c6c(ccc5c4N(c4ccccc4)c4cccc2c43)B2c3ccccc3N(c3ccccc3)c3cccc(c32)N6c2ccccc2)cc1 ; c1ccc(N2c3ccccc3B3c4cc5cc6c(cc5cc4N(c4ccccc4)c4cccc2c43)N(c2ccccc2)c2cccc3c2B6c2ccccc2N3c2ccccc2)cc1]

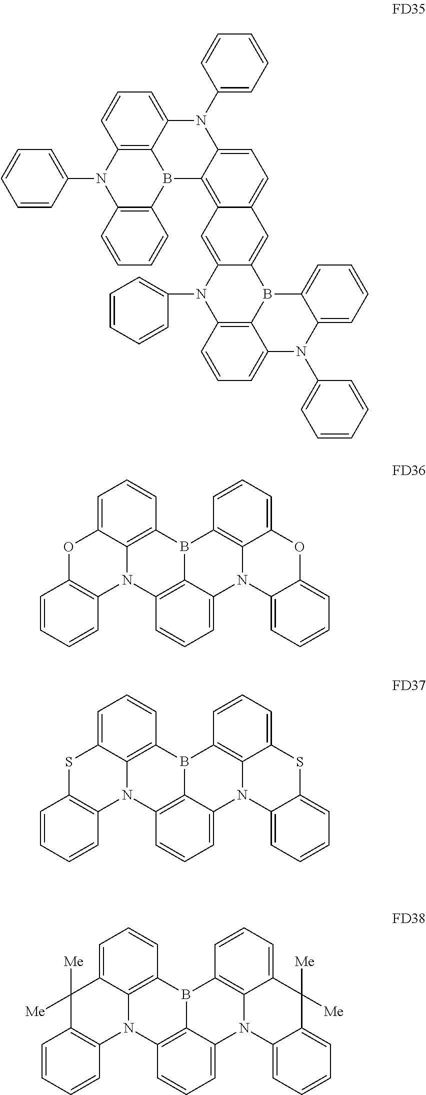 [SMILES: c1ccc(N2c3ccccc3B3c4cc5ccc6c(c5cc4N(c4ccccc4)c4cccc2c43)B2c3ccccc3N(c3ccccc3)c3cccc(c32)N6c2ccccc2)cc1 ; c1ccc2c(c1)Oc1cccc3c1N2c1cccc2c1B3c1cccc3c1N2c1ccccc1O3 ; c1ccc2c(c1)Sc1cccc3c1N2c1cccc2c1B3c1cccc3c1N2c1ccccc1S3 ; CC1(C)c2ccccc2N2c3cccc4c3B(c3cccc1c32)c1cccc2c1N4c1ccccc1C2(C)C]

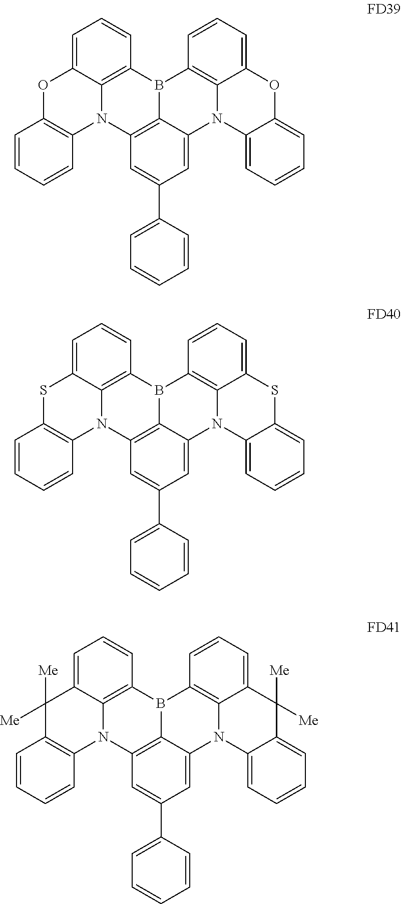 [SMILES: c1ccc(-c2cc3c4c(c2)N2c5ccccc5Oc5cccc(c52)B4c2cccc4c2N3c2ccccc2O4)cc1 ; c1ccc(-c2cc3c4c(c2)N2c5ccccc5Sc5cccc(c52)B4c2cccc4c2N3c2ccccc2S4)cc1 ; CC1(C)c2ccccc2N2c3cc(-c4ccccc4)cc4c3B(c3cccc1c32)c1cccc2c1N4c1ccccc1C2(C)C]

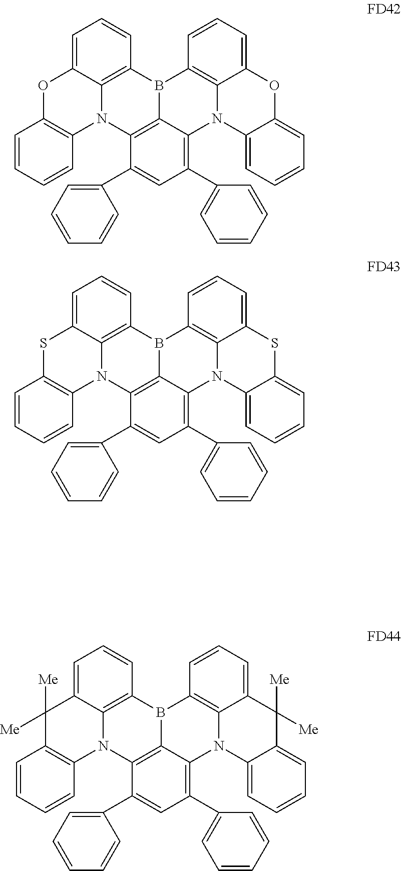

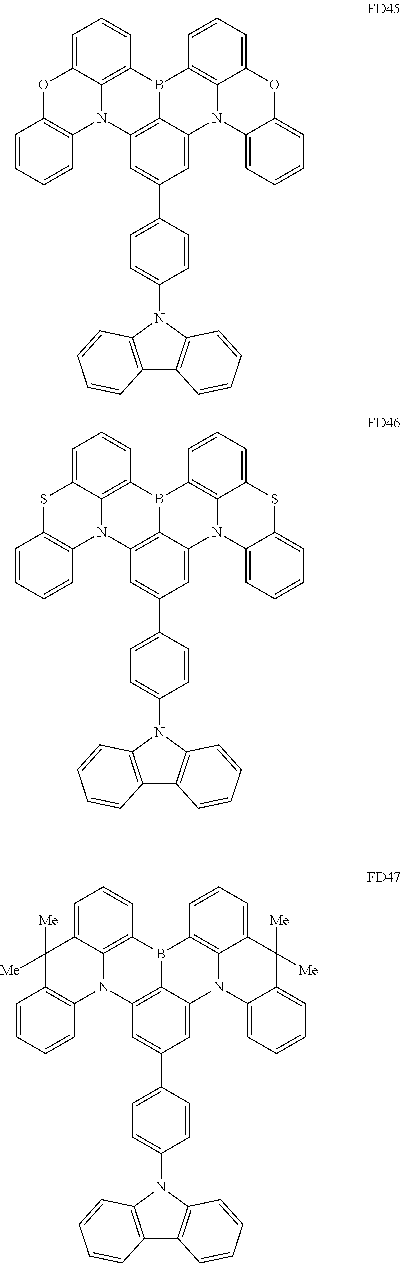

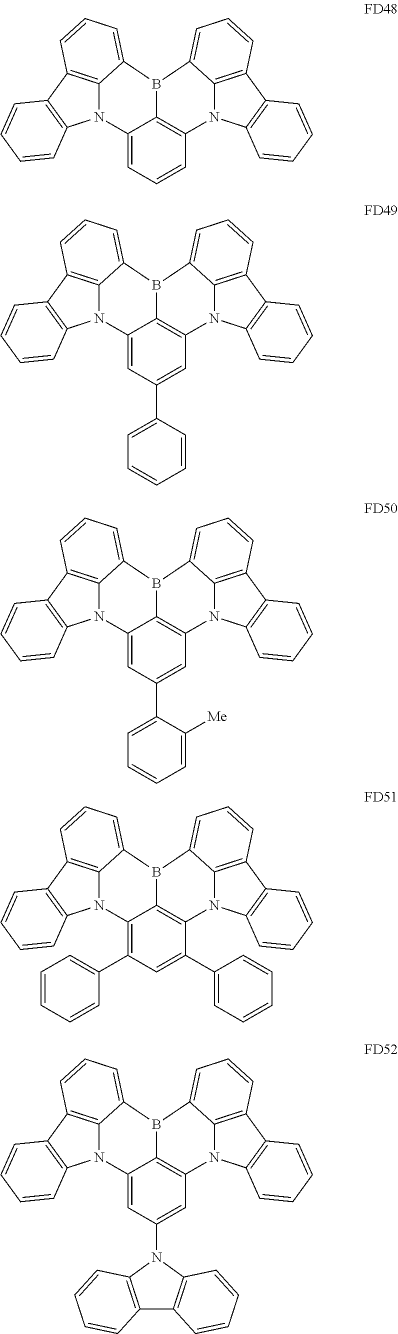

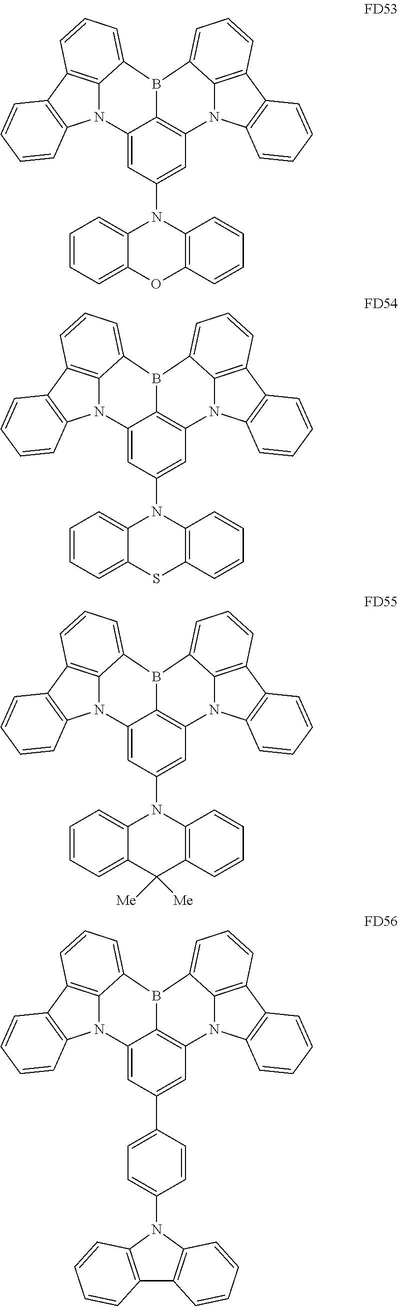

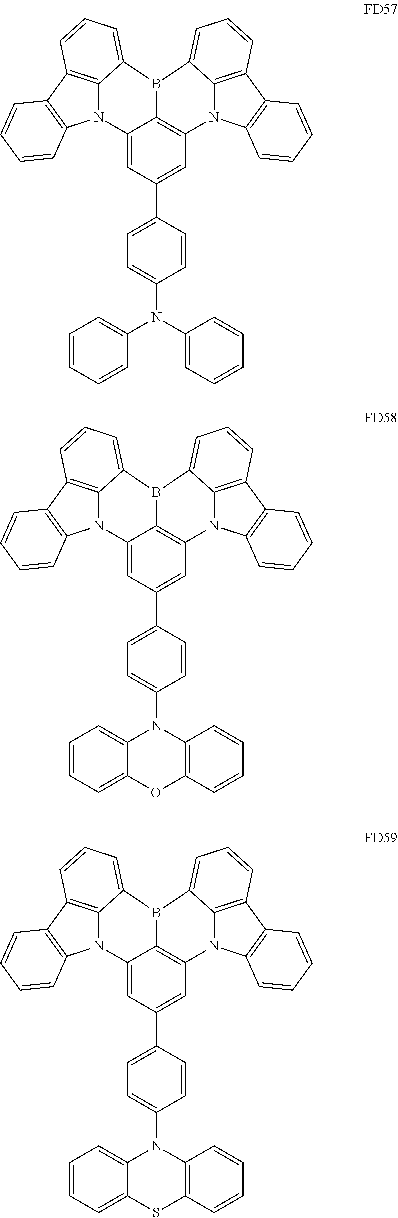

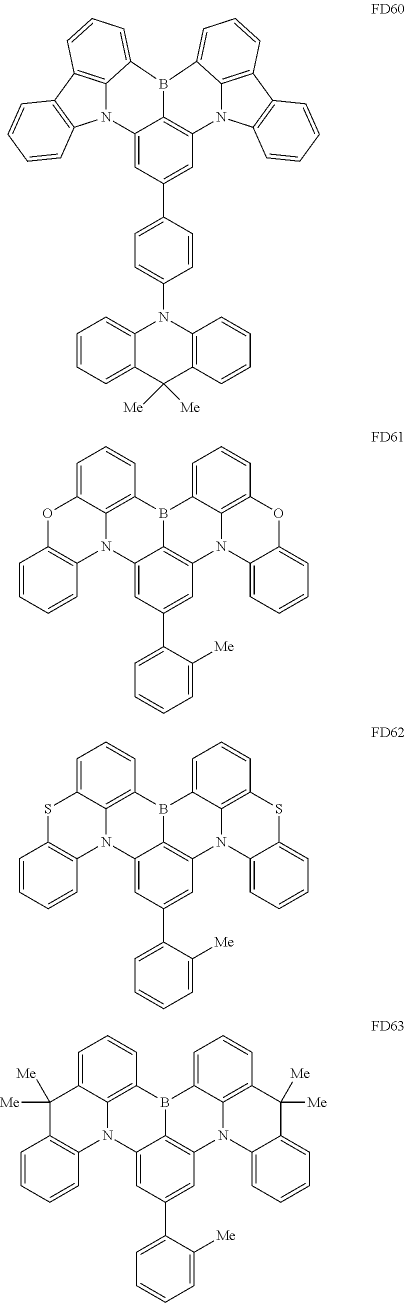 [SMILES: CC1(C)c2ccccc2N(c2ccc(-c3cc4c5c(c3)-n3c6ccccc6c6cccc(c63)B5c3cccc5c6ccccc6n-4c35)cc2)c2ccccc21 ; Cc1ccccc1-c1cc2c3c(c1)N1c4ccccc4Oc4cccc(c41)B3c1cccc3c1N2c1ccccc1O3 ; Cc1ccccc1-c1cc2c3c(c1)N1c4ccccc4Sc4cccc(c41)B3c1cccc3c1N2c1ccccc1S3 ; Cc1ccccc1-c1cc2c3c(c1)N1c4ccccc4C(C)(C)c4cccc(c41)B3c1cccc3c1N2c1ccccc1C3(C)C]

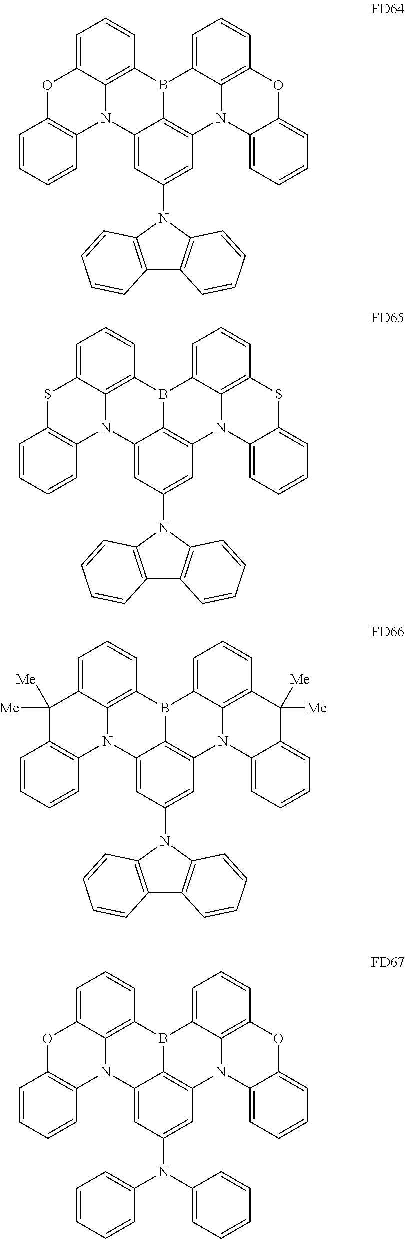

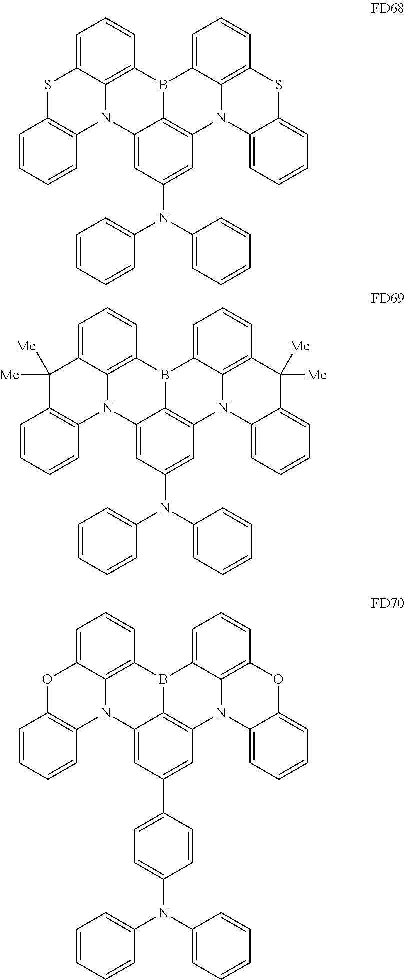

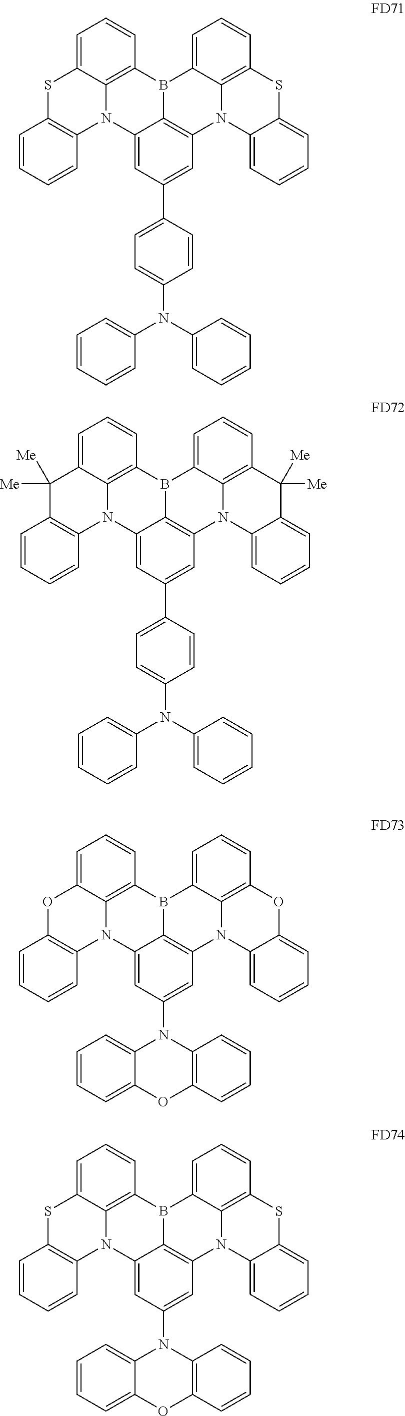

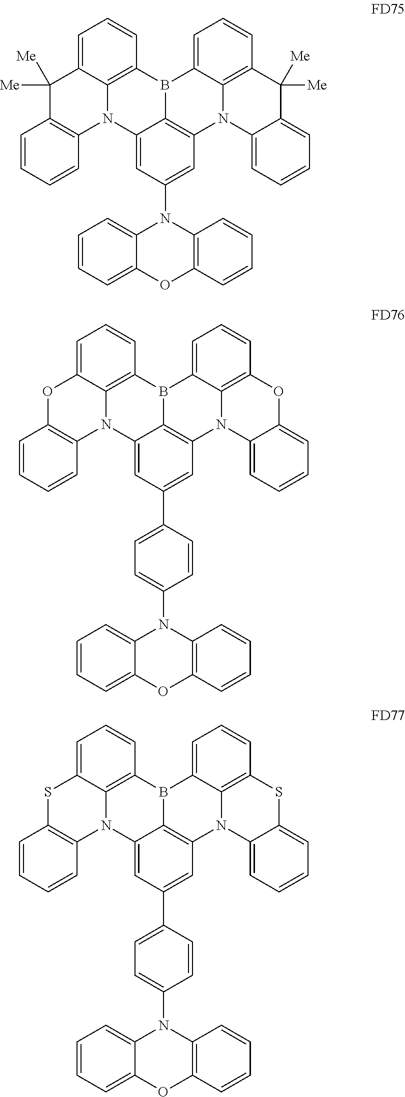

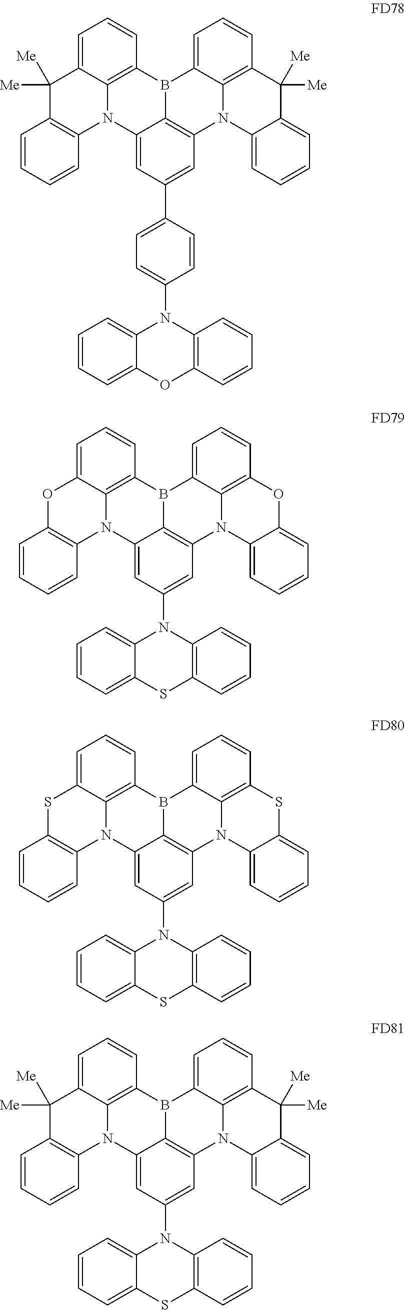

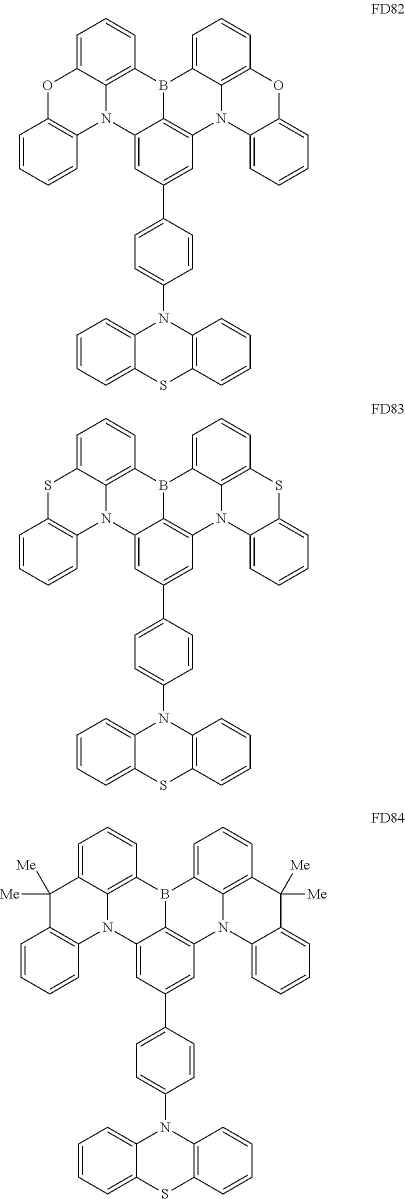

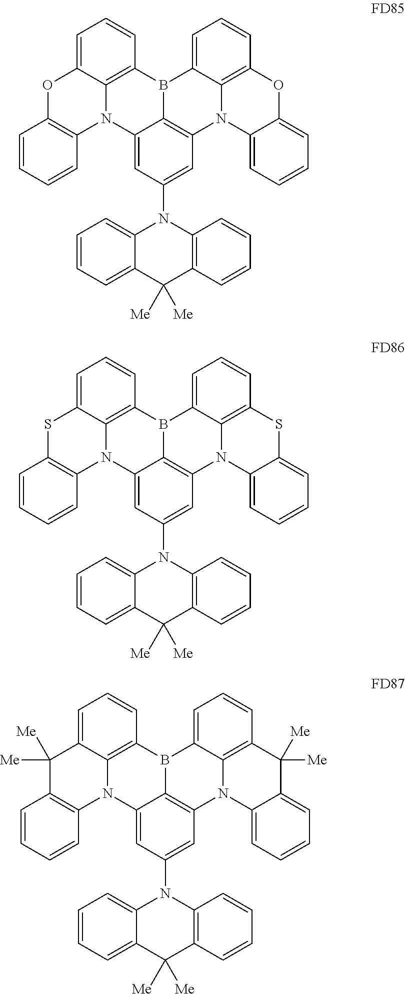 [SMILES: CC1(C)c2ccccc2N(c2cc3c4c(c2)N2c5ccccc5Oc5cccc(c52)B4c2cccc4c2N3c2ccccc2O4)c2ccccc21 ; CC1(C)c2ccccc2N(c2cc3c4c(c2)N2c5ccccc5Sc5cccc(c52)B4c2cccc4c2N3c2ccccc2S4)c2ccccc21 ; CC1(C)c2ccccc2N(c2cc3c4c(c2)N2c5ccccc5C(C)(C)c5cccc(c52)B4c2cccc4c2N3c2ccccc2C4(C)C)c2ccccc21]

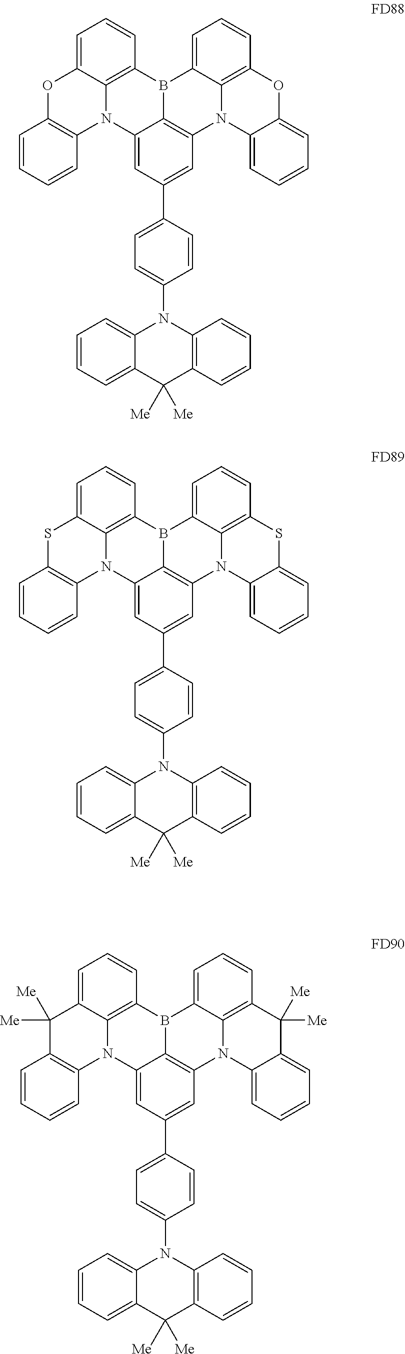 [SMILES: CC1(C)c2ccccc2N(c2ccc(-c3cc4c5c(c3)N3c6ccccc6Oc6cccc(c63)B5c3cccc5c3N4c3ccccc3O5)cc2)c2ccccc21 ; CC1(C)c2ccccc2N(c2ccc(-c3cc4c5c(c3)N3c6ccccc6Sc6cccc(c63)B5c3cccc5c3N4c3ccccc3S5)cc2)c2ccccc21 ; CC1(C)c2ccccc2N(c2ccc(-c3cc4c5c(c3)N3c6ccccc6C(C)(C)c6cccc(c63)B5c3cccc5c3N4c3ccccc3C5(C)C)cc2)c2ccccc21]

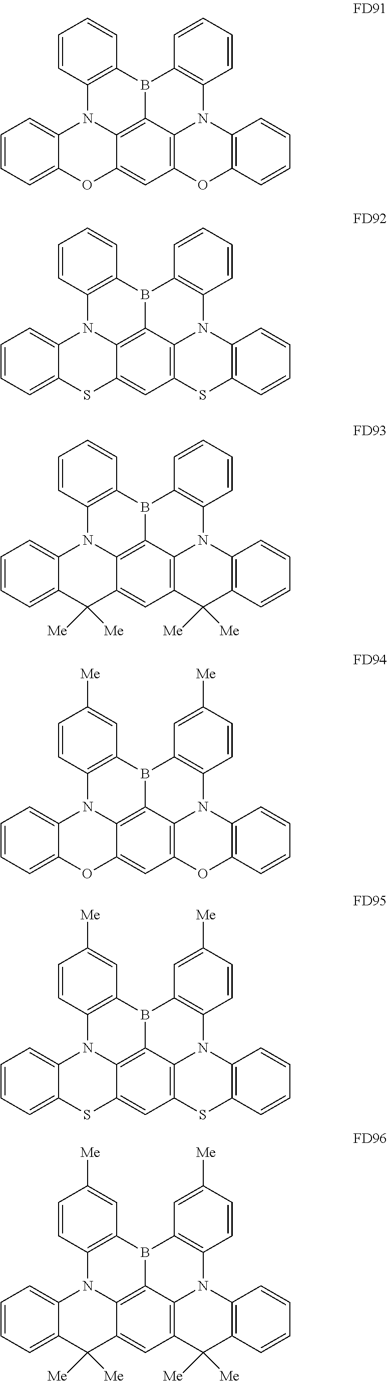

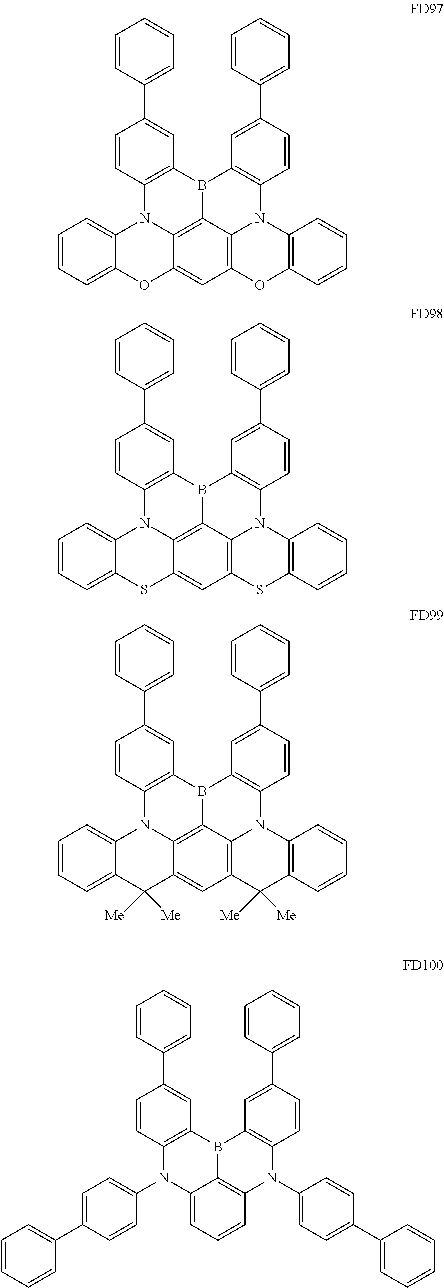

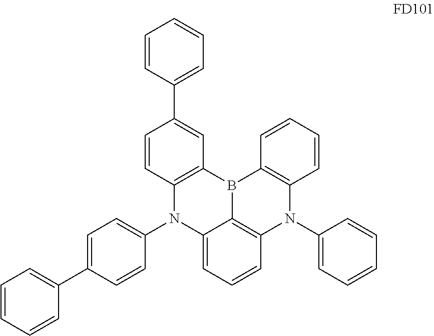 [SMILES: c1ccc(-c2ccc(N3c4ccc(-c5ccccc5)cc4B4c5ccccc5N(c5ccccc5)c5cccc3c54)cc2)cc1]

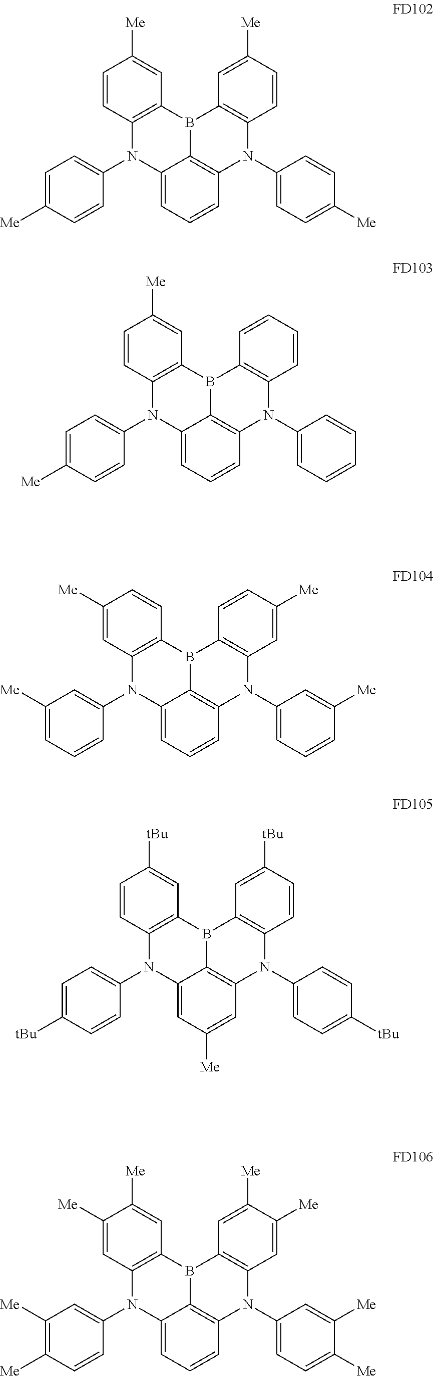 [SMILES: Cc1ccc(N2c3ccc(C)cc3B3c4cc(C)ccc4N(c4ccc(C)cc4)c4cccc2c43)cc1 ; Cc1ccc(N2c3ccc(C)cc3B3c4ccccc4N(c4ccccc4)c4cccc2c43)cc1 ; Cc1cccc(N2c3cc(C)ccc3B3c4ccc(C)cc4N(c4cccc(C)c4)c4cccc2c43)c1 ; Cc1cc2c3c(c1)N(c1ccc(C(C)(C)C)cc1)c1ccc(C(C)(C)C)cc1B3c1cc(C(C)(C)C)ccc1N2c1ccc(C(C)(C)C)cc1 ; Cc1ccc(N2c3cc(C)c(C)cc3B3c4cc(C)c(C)cc4N(c4ccc(C)c(C)c4)c4cccc2c43)cc1C]

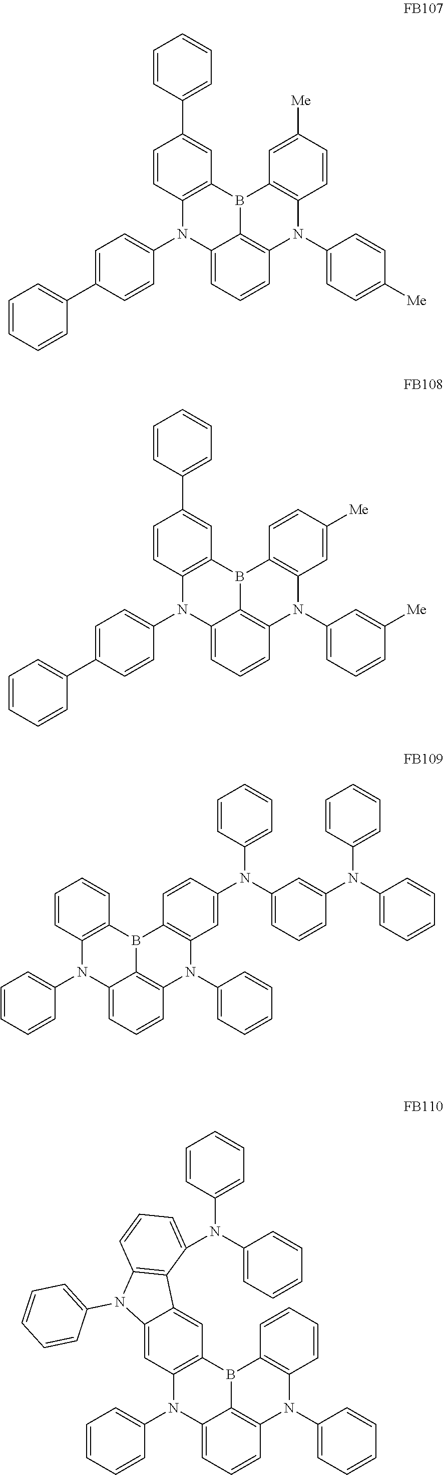 [SMILES: Cc1ccc(N2c3ccc(C)cc3B3c4cc(-c5ccccc5)ccc4N(c4ccc(-c5ccccc5)cc4)c4cccc2c43)cc1 ; Cc1cccc(N2c3cc(C)ccc3B3c4cc(-c5ccccc5)ccc4N(c4ccc(-c5ccccc5)cc4)c4cccc2c43)c1 ; c1ccc(N(c2ccccc2)c2cccc(N(c3ccccc3)c3ccc4c(c3)N(c3ccccc3)c3cccc5c3B4c3ccccc3N5c3ccccc3)c2)cc1 ; c1ccc(N2c3ccccc3B3c4cc5c6c(N(c7ccccc7)c7ccccc7)cccc6n(-c6ccccc6)c5cc4N(c4ccccc4)c4cccc2c43)cc1]

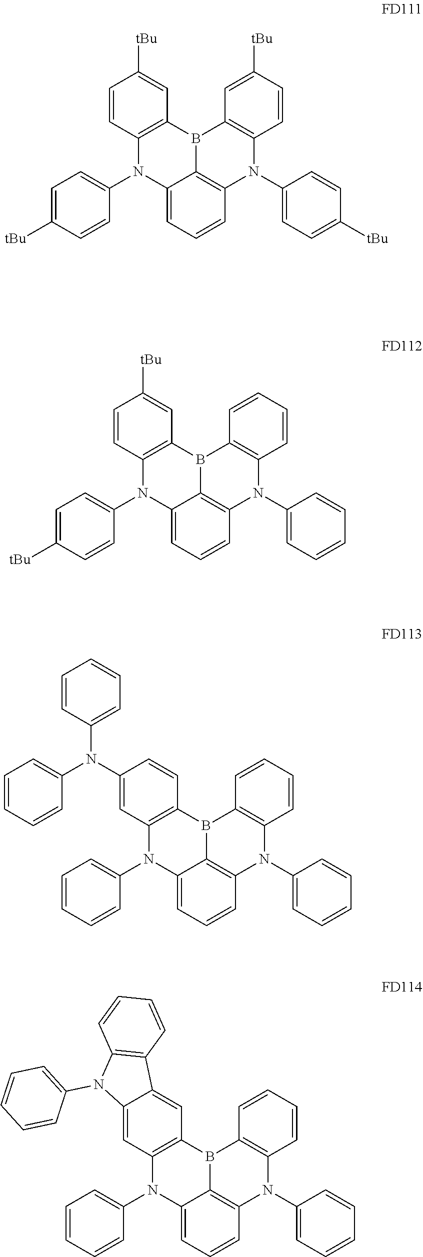 [SMILES: CC(C)(C)c1ccc(N2c3ccc(C(C)(C)C)cc3B3c4cc(C(C)(C)C)ccc4N(c4ccc(C(C)(C)C)cc4)c4cccc2c43)cc1 ; CC(C)(C)c1ccc(N2c3ccc(C(C)(C)C)cc3B3c4ccccc4N(c4ccccc4)c4cccc2c43)cc1 ; c1ccc(N(c2ccccc2)c2ccc3c(c2)N(c2ccccc2)c2cccc4c2B3c2ccccc2N4c2ccccc2)cc1 ; c1ccc(N2c3ccccc3B3c4cc5c6ccccc6n(-c6ccccc6)c5cc4N(c4ccccc4)c4cccc2c43)cc1]

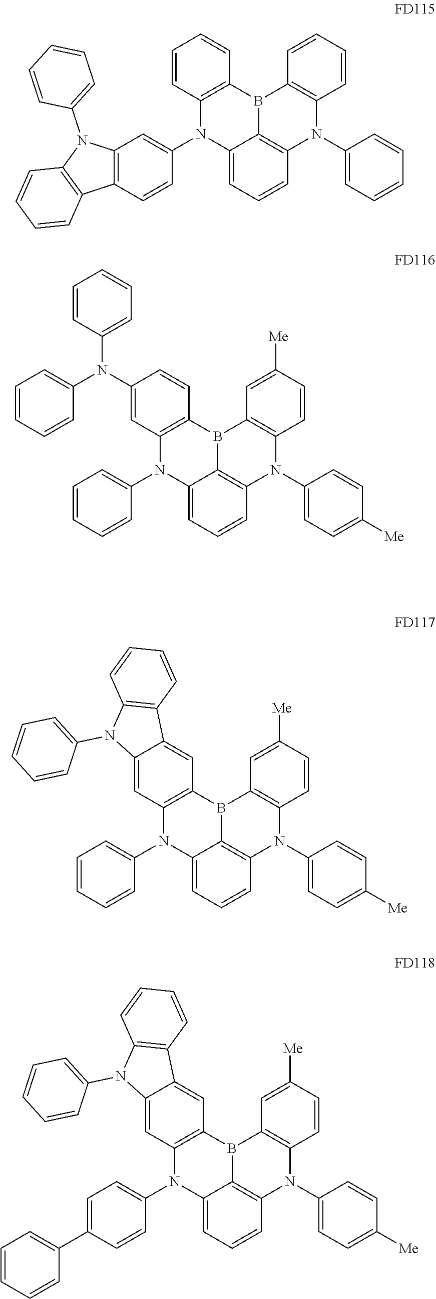 [SMILES: c1ccc(N2c3ccccc3B3c4ccccc4N(c4ccc5c6ccccc6n(-c6ccccc6)c5c4)c4cccc2c43)cc1 ; Cc1ccc(N2c3ccc(C)cc3B3c4ccc(N(c5ccccc5)c5ccccc5)cc4N(c4ccccc4)c4cccc2c43)cc1 ; Cc1ccc(N2c3ccc(C)cc3B3c4cc5c6ccccc6n(-c6ccccc6)c5cc4N(c4ccccc4)c4cccc2c43)cc1 ; Cc1ccc(N2c3ccc(C)cc3B3c4cc5c6ccccc6n(-c6ccccc6)c5cc4N(c4ccc(-c5ccccc5)cc4)c4cccc2c43)cc1]

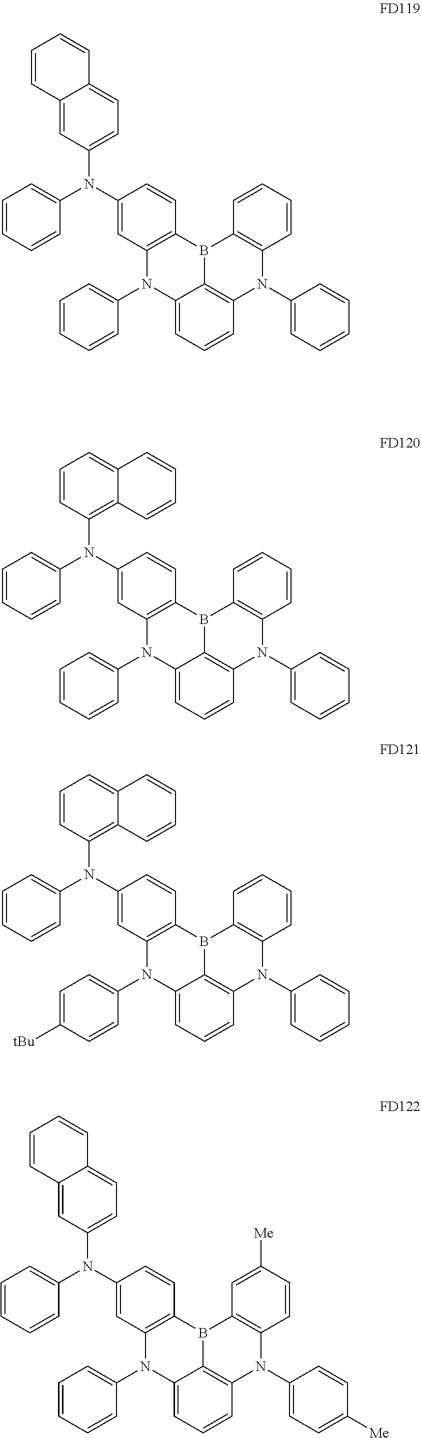 [SMILES: c1ccc(N(c2ccc3c(c2)N(c2ccccc2)c2cccc4c2B3c2ccccc2N4c2ccccc2)c2ccc3ccccc3c2)cc1 ; c1ccc(N2c3ccccc3B3c4ccc(N(c5ccccc5)c5cccc6ccccc56)cc4N(c4ccccc4)c4cccc2c43)cc1 ; CC(C)(C)c1ccc(N2c3cc(N(c4ccccc4)c4cccc5ccccc45)ccc3B3c4ccccc4N(c4ccccc4)c4cccc2c43)cc1 ; Cc1ccc(N2c3ccc(C)cc3B3c4ccc(N(c5ccccc5)c5ccc6ccccc6c5)cc4N(c4ccccc4)c4cccc2c43)cc1]

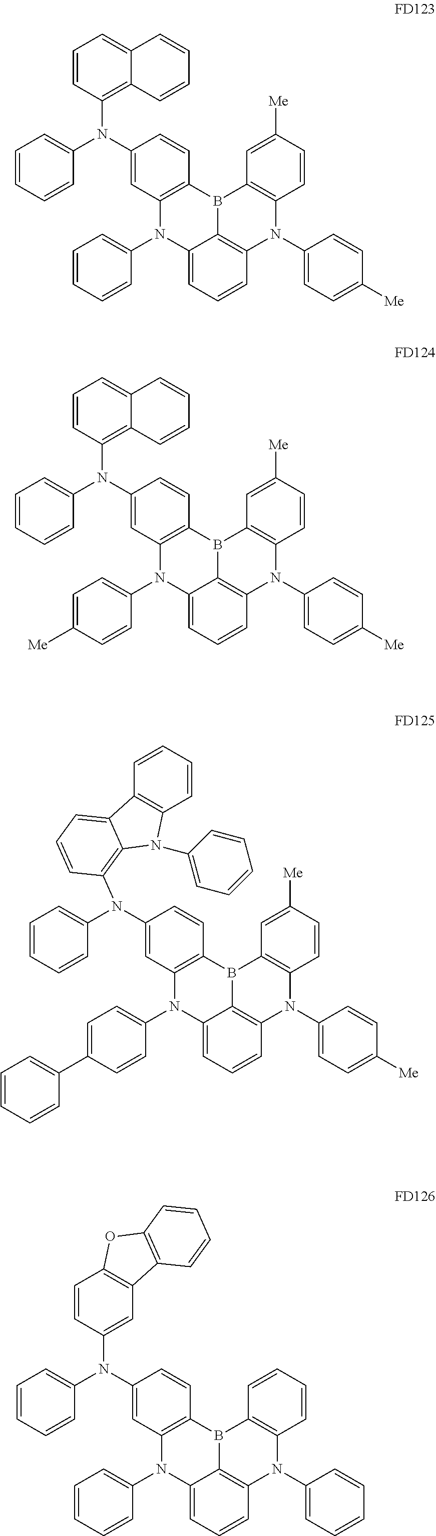

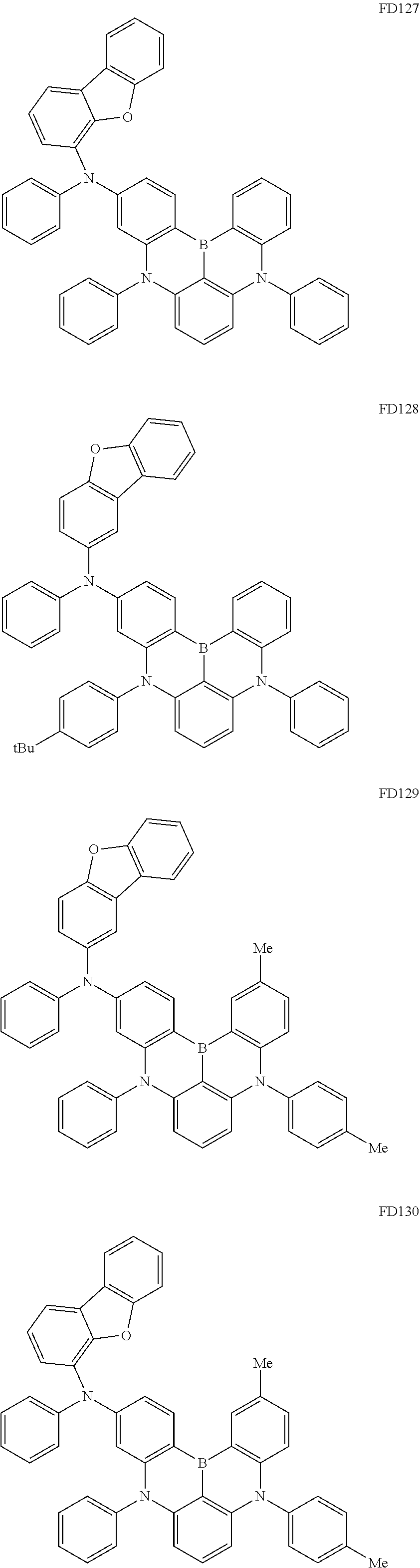

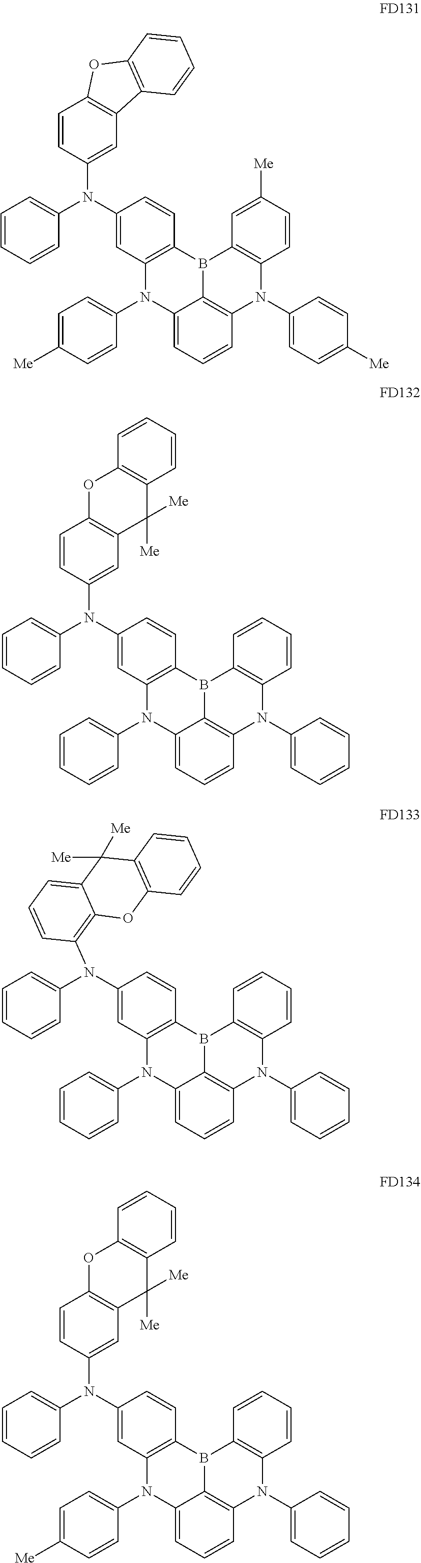

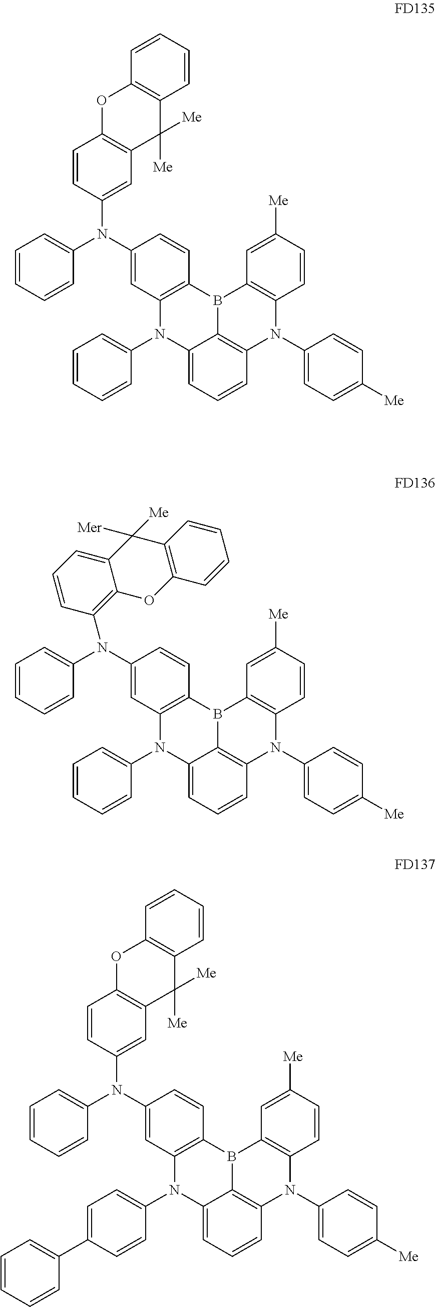

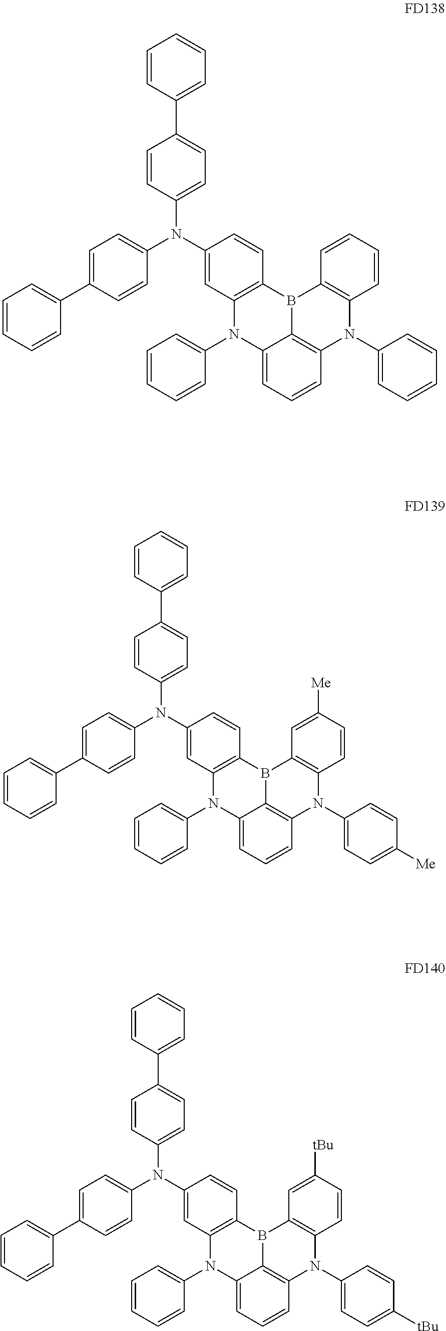

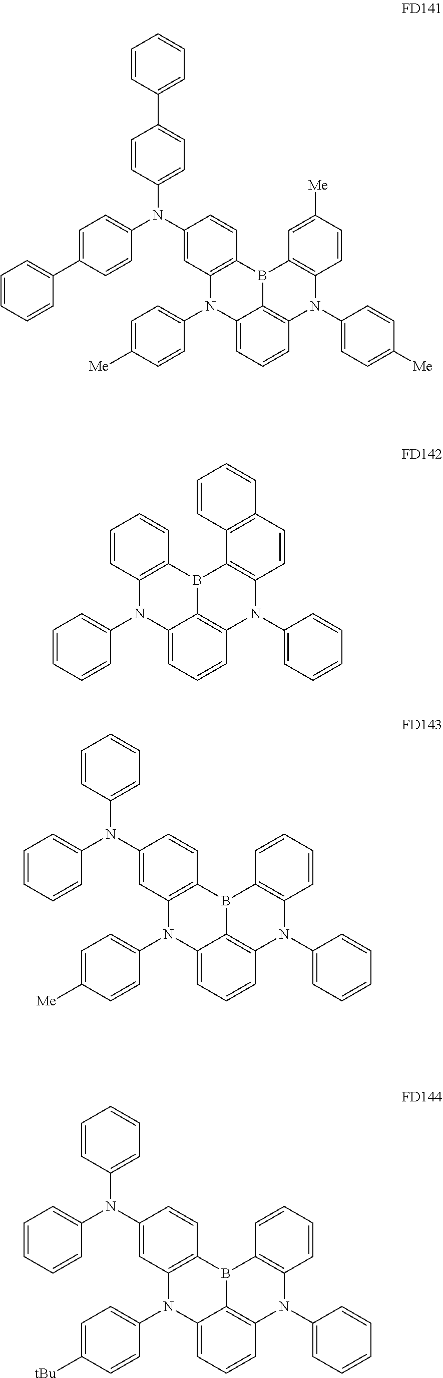 [SMILES: Cc1ccc(N2c3ccc(C)cc3B3c4ccc(N(c5ccc(-c6ccccc6)cc5)c5ccc(-c6ccccc6)cc5)cc4N(c4ccc(C)cc4)c4cccc2c43)cc1 ; c1ccc(N2c3ccccc3B3c4c2cccc4N(c2ccccc2)c2ccc4ccccc4c23)cc1 ; Cc1ccc(N2c3cc(N(c4ccccc4)c4ccccc4)ccc3B3c4ccccc4N(c4ccccc4)c4cccc2c43)cc1 ; CC(C)(C)c1ccc(N2c3cc(N(c4ccccc4)c4ccccc4)ccc3B3c4ccccc4N(c4ccccc4)c4cccc2c43)cc1]

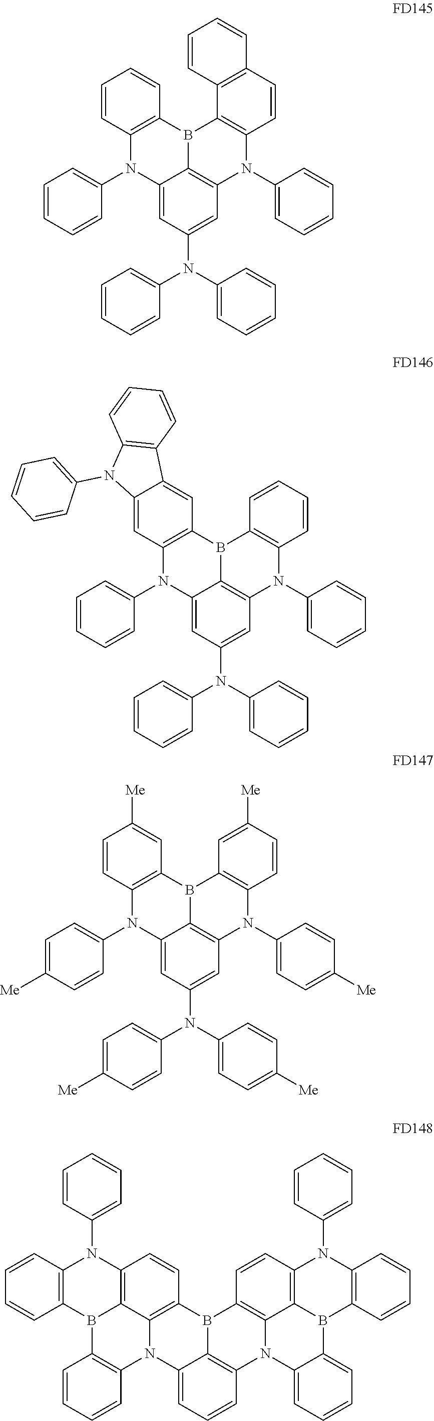 [SMILES: c1ccc(N(c2ccccc2)c2cc3c4c(c2)N(c2ccccc2)c2ccc5ccccc5c2B4c2ccccc2N3c2ccccc2)cc1 ; c1ccc(N(c2ccccc2)c2cc3c4c(c2)N(c2ccccc2)c2cc5c(cc2B4c2ccccc2N3c2ccccc2)c2ccccc2n5-c2ccccc2)cc1 ; Cc1ccc(N(c2ccc(C)cc2)c2cc3c4c(c2)N(c2ccc(C)cc2)c2ccc(C)cc2B4c2cc(C)ccc2N3c2ccc(C)cc2)cc1 ; c1ccc(N2c3ccccc3B3c4ccccc4N4c5cccc6c5B(c5ccc2c3c54)c2ccc3c4c2N6c2ccccc2B4c2ccccc2N3c2ccccc2)cc1]

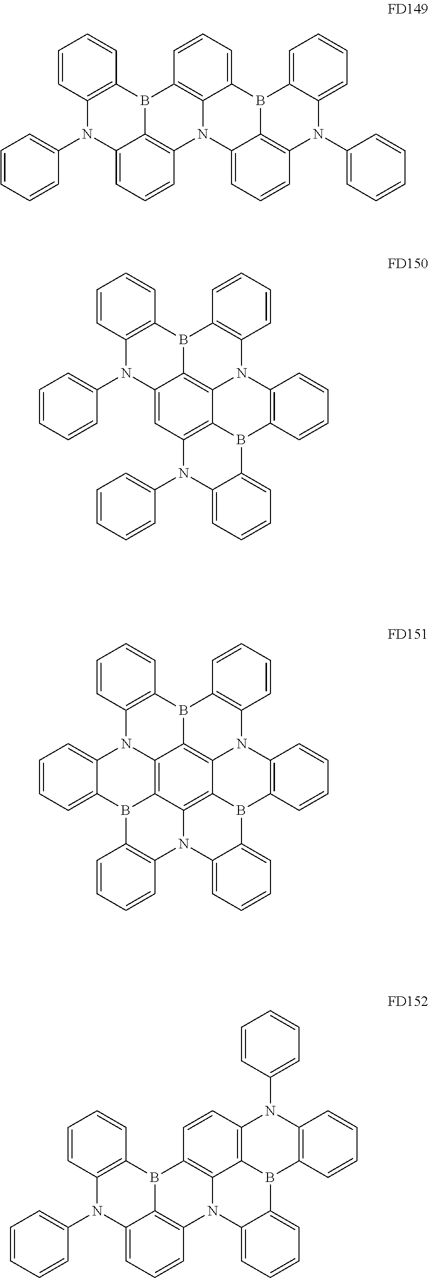 [SMILES: c1ccc(N2c3ccccc3B3c4cccc5c4N(c4cccc2c43)c2cccc3c2B5c2ccccc2N3c2ccccc2)cc1 ; c1ccc(N2c3ccccc3B3c4ccccc4N4c5ccccc5B5c6ccccc6N(c6ccccc6)c6cc2c3c4c65)cc1 ; c1ccc2c(c1)B1c3ccccc3N3c4ccccc4B4c5ccccc5N5c6ccccc6B6c7ccccc7N2c2c1c3c4c5c26 ; c1ccc(N2c3ccccc3B3c4ccc5c6c4N(c4ccccc4B6c4ccccc4N5c4ccccc4)c4cccc2c43)cc1]

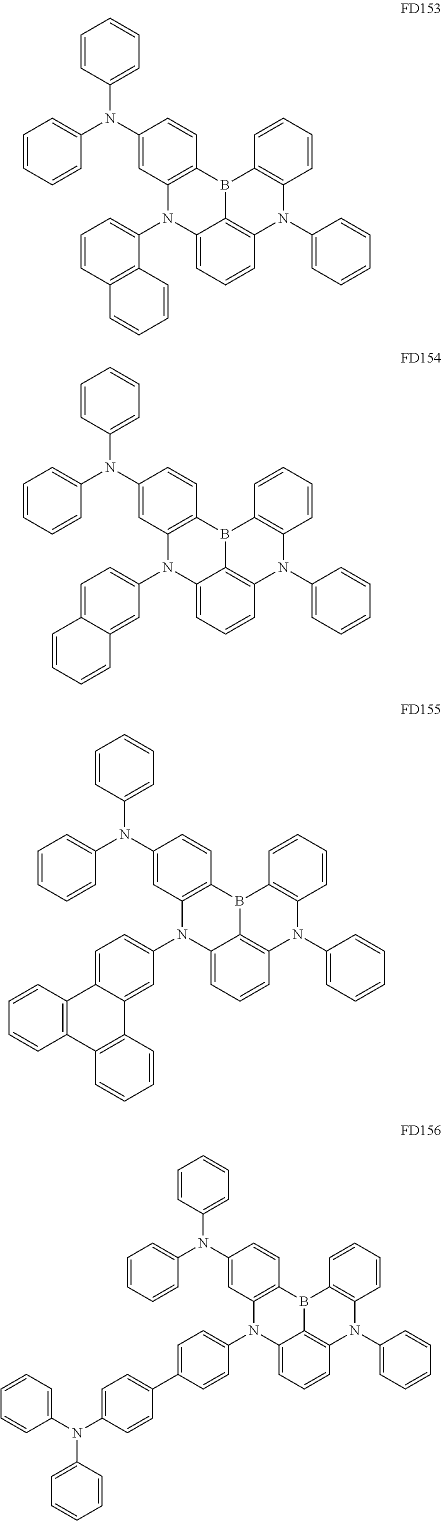

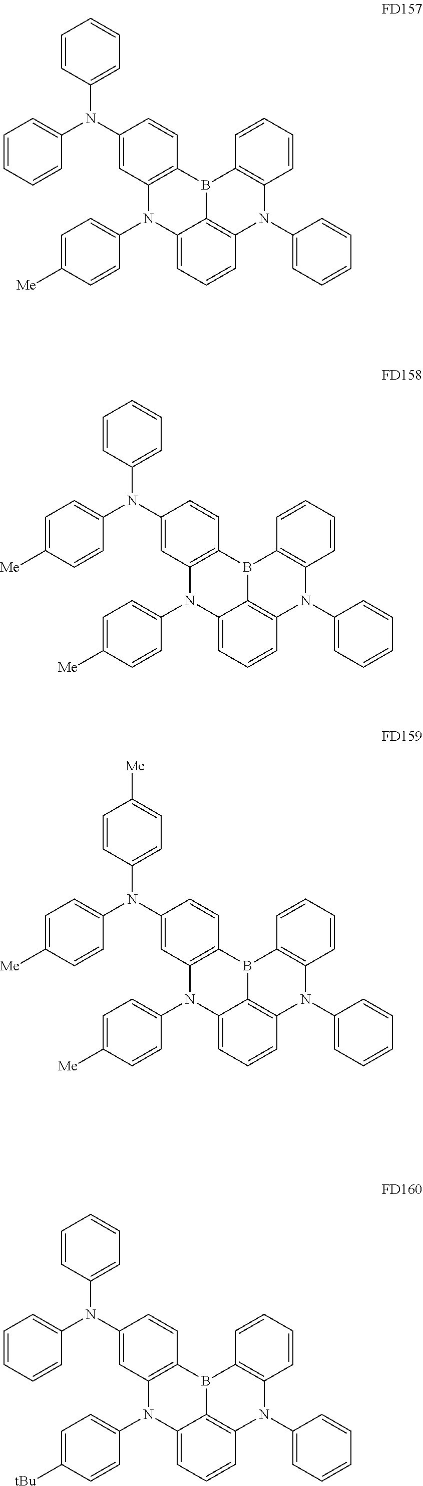 [SMILES: Cc1ccc(N2c3cc(N(c4ccccc4)c4ccccc4)ccc3B3c4ccccc4N(c4ccccc4)c4cccc2c43)cc1 ; Cc1ccc(N(c2ccccc2)c2ccc3c(c2)N(c2ccc(C)cc2)c2cccc4c2B3c2ccccc2N4c2ccccc2)cc1 ; Cc1ccc(N(c2ccc(C)cc2)c2ccc3c(c2)N(c2ccc(C)cc2)c2cccc4c2B3c2ccccc2N4c2ccccc2)cc1 ; CC(C)(C)c1ccc(N2c3cc(N(c4ccccc4)c4ccccc4)ccc3B3c4ccccc4N(c4ccccc4)c4cccc2c43)cc1]

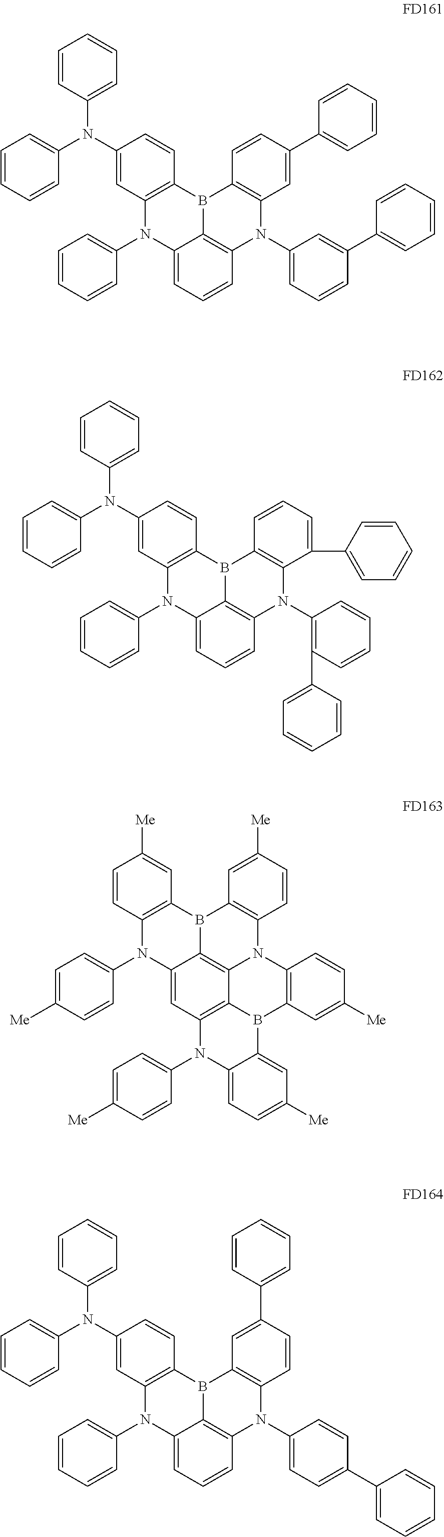 [SMILES: c1ccc(-c2cccc(N3c4cc(-c5ccccc5)ccc4B4c5ccc(N(c6ccccc6)c6ccccc6)cc5N(c5ccccc5)c5cccc3c54)c2)cc1 ; c1ccc(-c2ccccc2N2c3cccc4c3B(c3ccc(N(c5ccccc5)c5ccccc5)cc3N4c3ccccc3)c3cccc(-c4ccccc4)c32)cc1 ; Cc1ccc(N2c3ccc(C)cc3B3c4cc(C)ccc4N4c5ccc(C)cc5B5c6cc(C)ccc6N(c6ccc(C)cc6)c6cc2c3c4c65)cc1 ; c1ccc(-c2ccc(N3c4ccc(-c5ccccc5)cc4B4c5ccc(N(c6ccccc6)c6ccccc6)cc5N(c5ccccc5)c5cccc3c54)cc2)cc1]

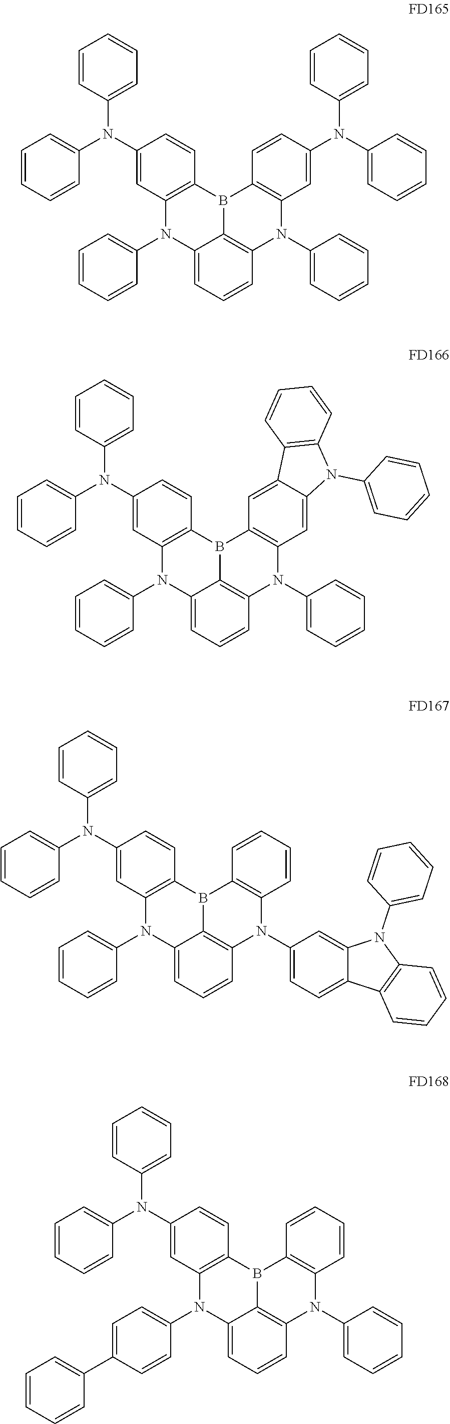

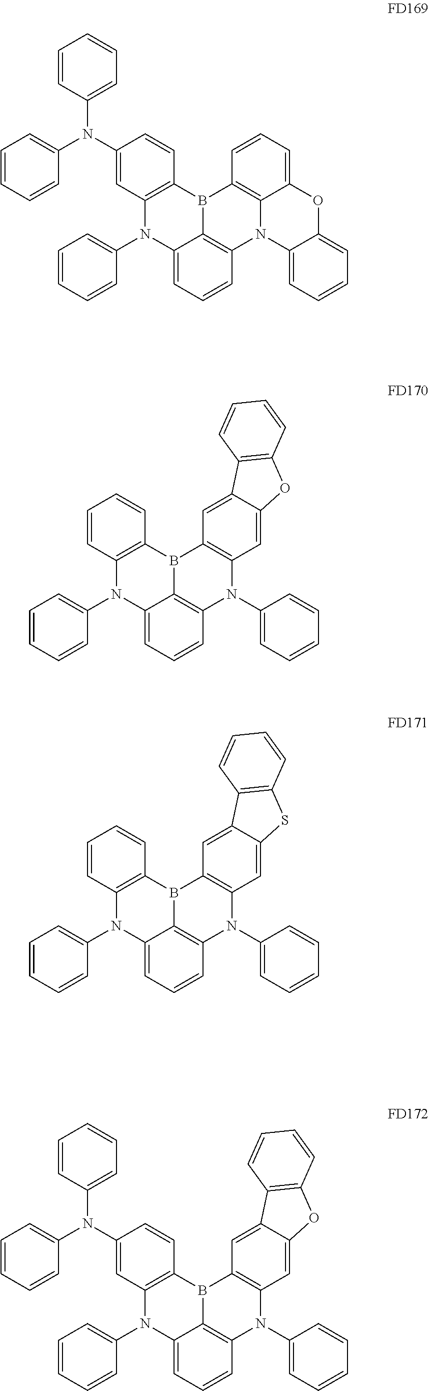

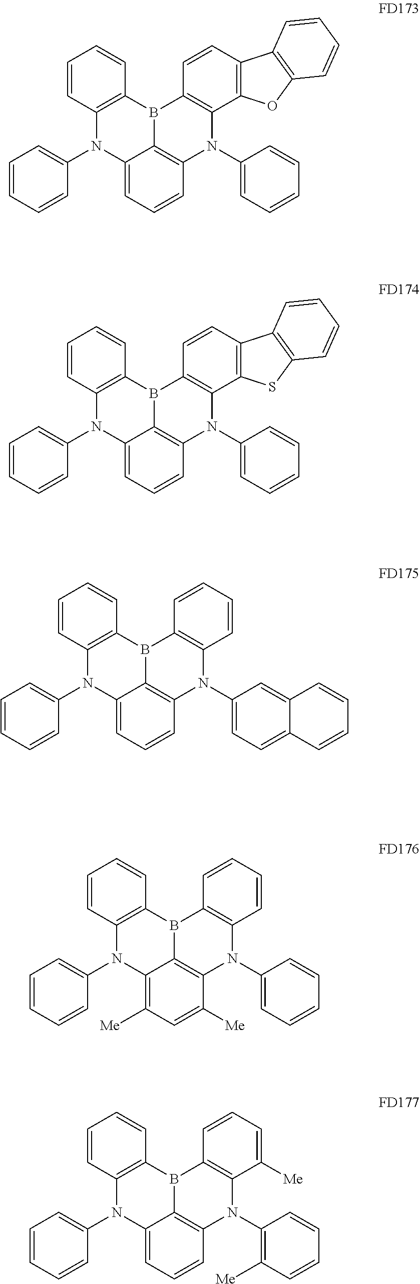

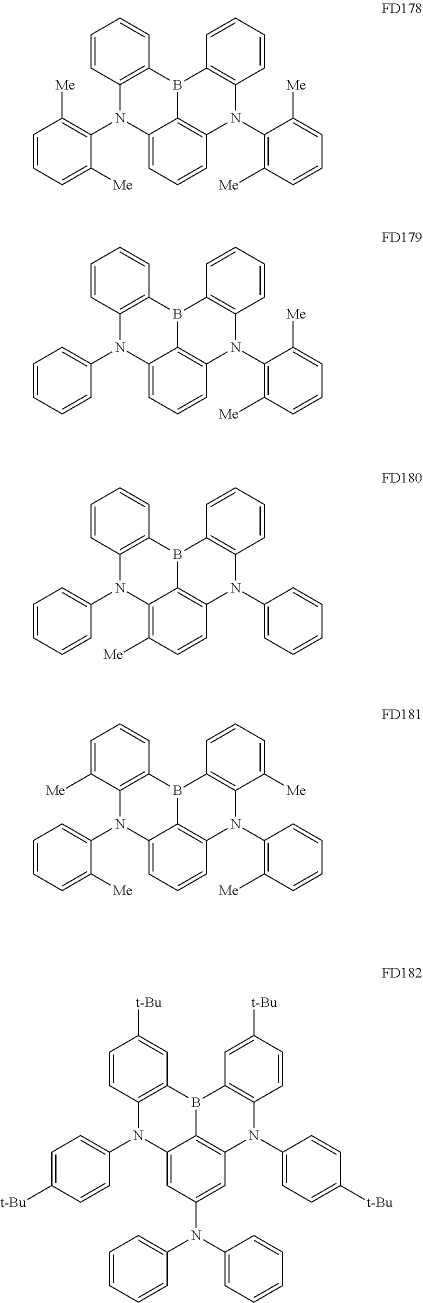 [SMILES: Cc1cccc(C)c1N1c2ccccc2B2c3ccccc3N(c3c(C)cccc3C)c3cccc1c32 ; Cc1cccc(C)c1N1c2ccccc2B2c3ccccc3N(c3ccccc3)c3cccc1c32 ; Cc1ccc2c3c1N(c1ccccc1)c1ccccc1B3c1ccccc1N2c1ccccc1 ; Cc1ccccc1N1c2cccc3c2B(c2cccc(C)c21)c1cccc(C)c1N3c1ccccc1C ; CC(C)(C)c1ccc(N2c3ccc(C(C)(C)C)cc3B3c4cc(C(C)(C)C)ccc4N(c4ccc(C(C)(C)C)cc4)c4cc(N(c5ccccc5)c5ccccc5)cc2c43)cc1]

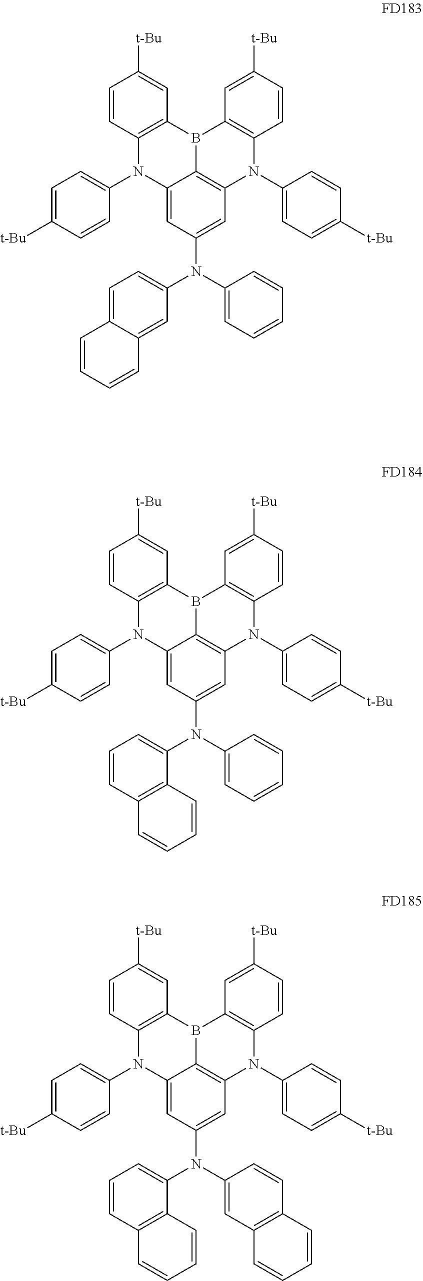 [SMILES: CC(C)(C)c1ccc(N2c3ccc(C(C)(C)C)cc3B3c4cc(C(C)(C)C)ccc4N(c4ccc(C(C)(C)C)cc4)c4cc(N(c5ccccc5)c5ccc6ccccc6c5)cc2c43)cc1 ; CC(C)(C)c1ccc(N2c3ccc(C(C)(C)C)cc3B3c4cc(C(C)(C)C)ccc4N(c4ccc(C(C)(C)C)cc4)c4cc(N(c5ccccc5)c5cccc6ccccc56)cc2c43)cc1 ; CC(C)(C)c1ccc(N2c3ccc(C(C)(C)C)cc3B3c4cc(C(C)(C)C)ccc4N(c4ccc(C(C)(C)C)cc4)c4cc(N(c5ccc6ccccc6c5)c5cccc6ccccc56)cc2c43)cc1]

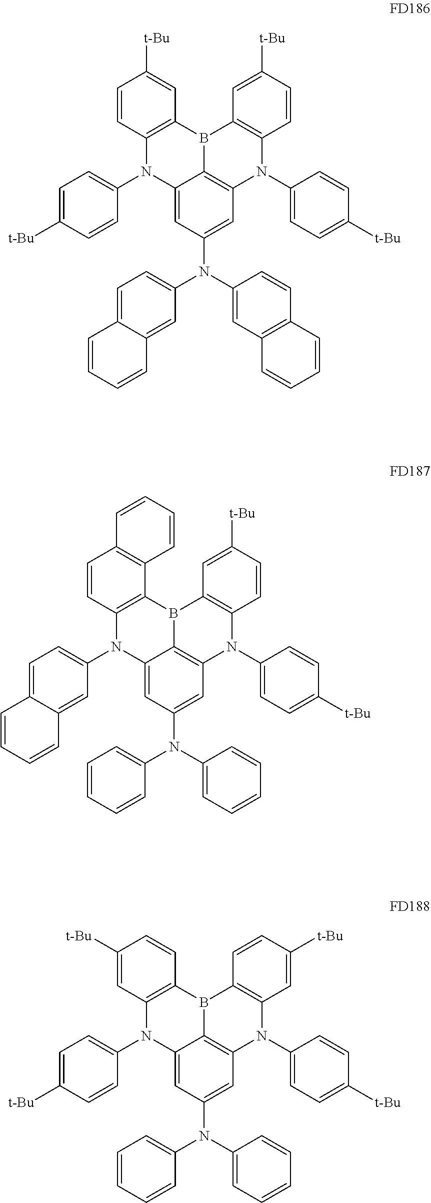 [SMILES: CC(C)(C)c1ccc(N2c3ccc(C(C)(C)C)cc3B3c4cc(C(C)(C)C)ccc4N(c4ccc(C(C)(C)C)cc4)c4cc(N(c5ccc6ccccc6c5)c5ccc6ccccc6c5)cc2c43)cc1 ; CC(C)(C)c1ccc(N2c3ccc(C(C)(C)C)cc3B3c4c2cc(N(c2ccccc2)c2ccccc2)cc4N(c2ccc4ccccc4c2)c2ccc4ccccc4c23)cc1 ; CC(C)(C)c1ccc(N2c3cc(C(C)(C)C)ccc3B3c4ccc(C(C)(C)C)cc4N(c4ccc(C(C)(C)C)cc4)c4cc(N(c5ccccc5)c5ccccc5)cc2c43)cc1]

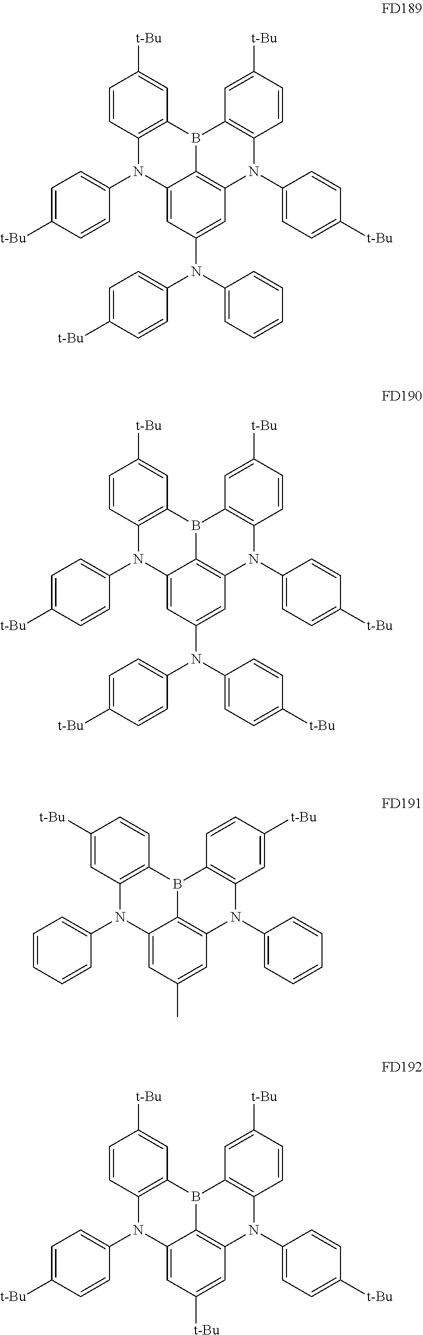 [SMILES: CC(C)(C)c1ccc(N(c2ccccc2)c2cc3c4c(c2)N(c2ccc(C(C)(C)C)cc2)c2ccc(C(C)(C)C)cc2B4c2cc(C(C)(C)C)ccc2N3c2ccc(C(C)(C)C)cc2)cc1 ; CC(C)(C)c1ccc(N(c2ccc(C(C)(C)C)cc2)c2cc3c4c(c2)N(c2ccc(C(C)(C)C)cc2)c2ccc(C(C)(C)C)cc2B4c2cc(C(C)(C)C)ccc2N3c2ccc(C(C)(C)C)cc2)cc1 ; Cc1cc2c3c(c1)N(c1ccccc1)c1cc(C(C)(C)C)ccc1B3c1ccc(C(C)(C)C)cc1N2c1ccccc1 ; CC(C)(C)c1ccc(N2c3ccc(C(C)(C)C)cc3B3c4cc(C(C)(C)C)ccc4N(c4ccc(C(C)(C)C)cc4)c4cc(C(C)(C)C)cc2c43)cc1]

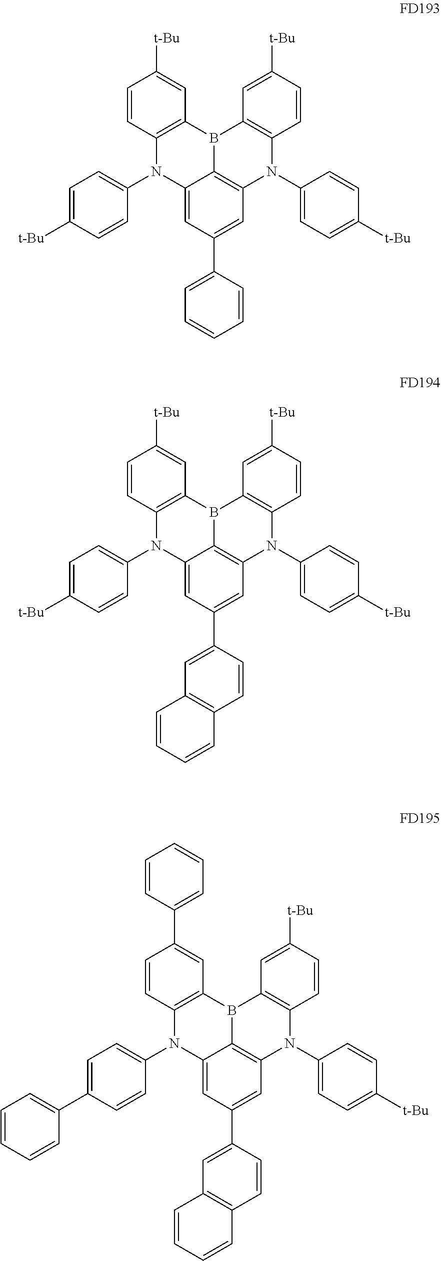 [SMILES: CC(C)(C)c1ccc(N2c3ccc(C(C)(C)C)cc3B3c4cc(C(C)(C)C)ccc4N(c4ccc(C(C)(C)C)cc4)c4cc(-c5ccccc5)cc2c43)cc1 ; CC(C)(C)c1ccc(N2c3ccc(C(C)(C)C)cc3B3c4cc(C(C)(C)C)ccc4N(c4ccc(C(C)(C)C)cc4)c4cc(-c5ccc6ccccc6c5)cc2c43)cc1 ; CC(C)(C)c1ccc(N2c3ccc(C(C)(C)C)cc3B3c4cc(-c5ccccc5)ccc4N(c4ccc(-c5ccccc5)cc4)c4cc(-c5ccc6ccccc6c5)cc2c43)cc1]

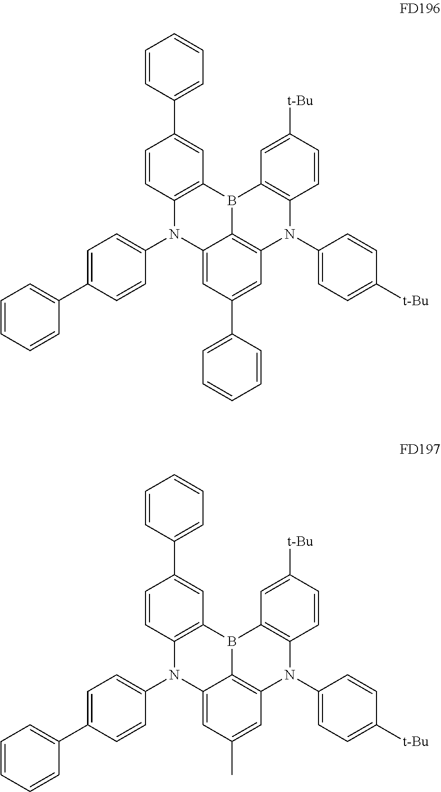 [SMILES: CC(C)(C)c1ccc(N2c3ccc(C(C)(C)C)cc3B3c4cc(-c5ccccc5)ccc4N(c4ccc(-c5ccccc5)cc4)c4cc(-c5ccccc5)cc2c43)cc1 ; Cc1cc2c3c(c1)N(c1ccc(C(C)(C)C)cc1)c1ccc(C(C)(C)C)cc1B3c1cc(-c3ccccc3)ccc1N2c1ccc(-c2ccccc2)cc1]

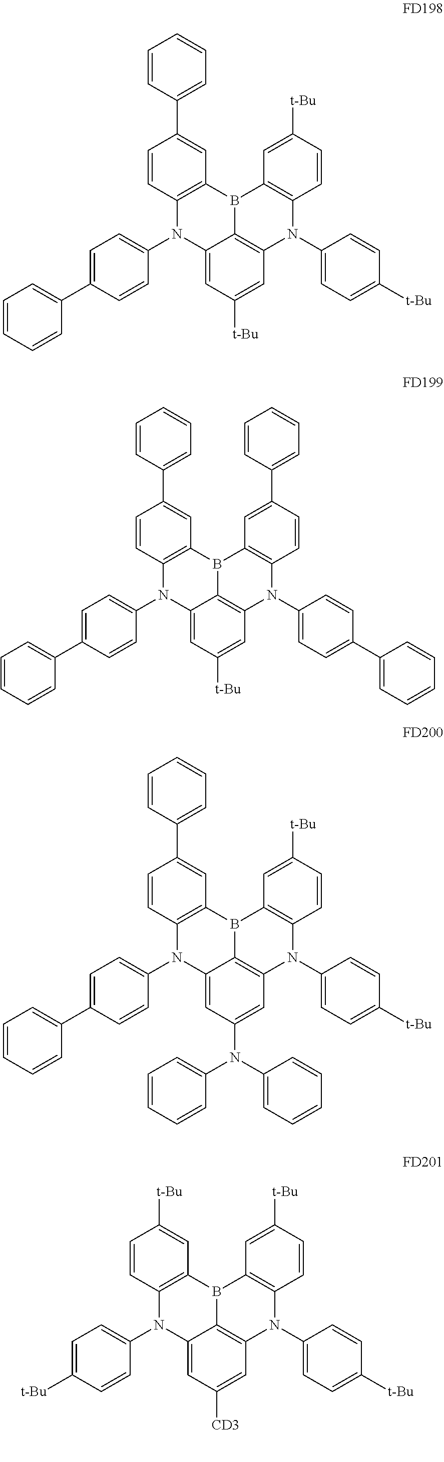

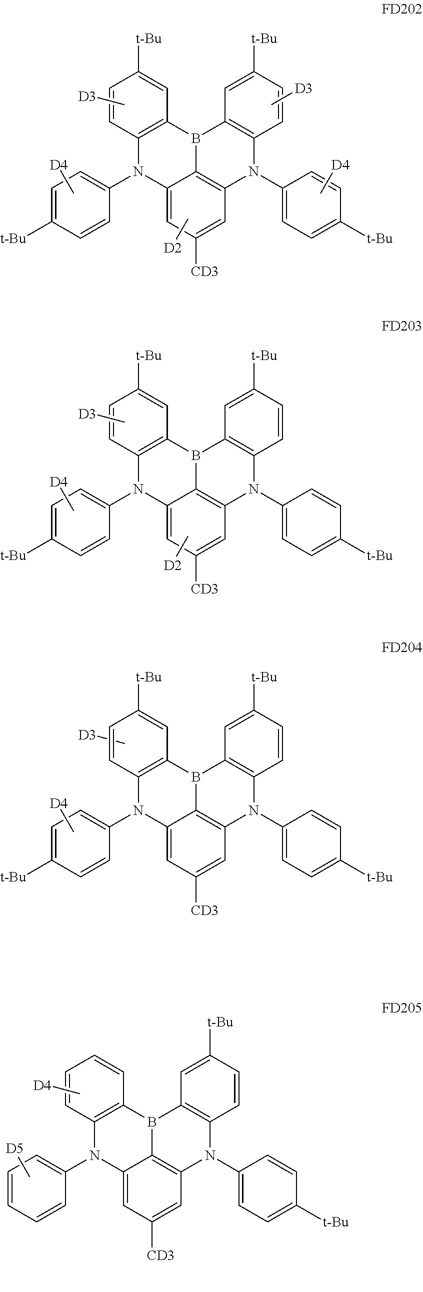

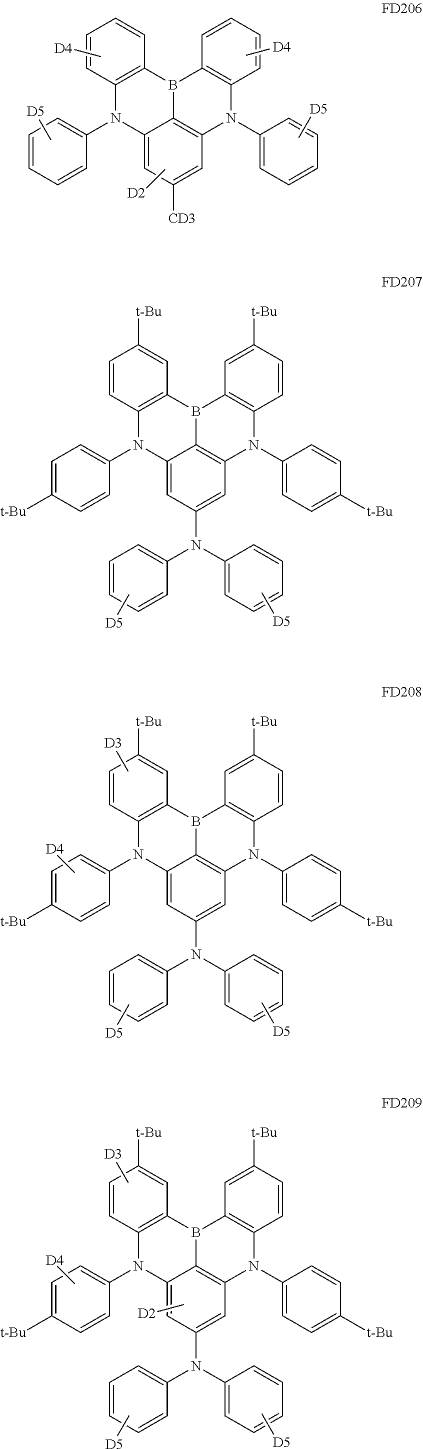

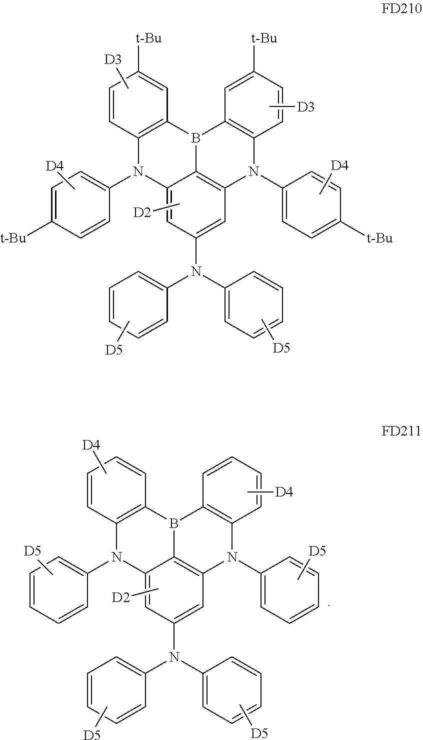

In another embodiment, the dopant 344 of blue delayed fluorescent material can comprise, but is not limited to, 4,4,6,6-tetra(9H-carbazol-9-yl)-[1,1-biphenyl]-3,3-dicarbonitrile (CzBPCN), 4,6-di(carbazol-9-yl)benzene-1,3-dicarbonitrile (DCzIPN), 10-(4-(diphenylphosphoryl)phenyl)-10H-phenoxazine (SPXZPO), 10,10′-(4,4′-(phenylphosphoryl)bis(4,1-phenylene))bis(10H-phenoxazine) (DPXZPO), 10,10′,10″-(4,4′,4″-phosphoryltris(benzene-4,1-diyl))tris(10H-phenoxazine) (TPXZPO), 9,9′-(5-(4,6-diphenyl-1,3,5-triazin-2-yl)-1,3-phenylene)bis(9H-carbazole) (DcZTrz), 9,9′,9″,9′″-((6-phenyl-1,3,5-triazin-2,4-diyl)bis(benzene-5,3,1-triyl))tetrakis(9H-carbazole) (DDczTrz), 10,10′-(4,4′-(4-Phenyl-4H-1,2,4-triazole-3,5-diyl)bis(4,1-phenylene))bis(10H-phenoxazine) (2PXZ-TAZ), 2,7-bis(9,9-dimethylacridin-10(9H)-yl)-9,9-dimethyl-9H-thioxanthene-10,10-dioxide (DMTDAc), 9,9′-(4,4′-sulfonylbis(4,1-phenylene))bis(3,6-dimethoxyl-9H-carbazole) (DMOC-DPS), 10,10′-(4,4′-Sulfonylbis(4,1-phenylene))bis(9,9-dimethyl-9,10-dihydroacridine) (DMAC-DPS), 10-(4-(4,6-diphenyl-1,3,5-triazin-2-yl)phenyl)-9,9-dimethyl-9,10-dihydroacridine (DMAC-TRZ), 10-phenyl-10H,10′H-spiro[acridine-9,9′-anthracen]-10′-one (ACRSA), 3,6-dibenzoyl-4,5-di(1-methyl-9-phenyl-9H-carbazoyl)-2-ethynylbenzonitrile (Cz-VPN), 9,9′,9″-(5-(4,6-diphenyl-1,3,5-triazin-2-yl)benzene-1,2,3-triyl) tris(9H-carbazole) (TcZTrz), 4,4,6,6-tetra(9H-carbazol-9-yl)-[1,1-biphenyl]-3,3-dicarbonitrile (CzBPCN), 2′-(10H-phenoxazin-10-yl)-[1,1′:3′,1″-terphenyl]-5′-carbonitrile (mPTC), bis(4-(9H-3,9′-bicarbazol-9-yl)phenyl)methanone (CC2BP), 9′-[4-(4,6-diphenyl-1,3,5-triazin-2-yl)phenyl]-3,3″,6,6″-tetraphenyl-9,3′:6′,9″-ter-9H-carbazole (BDPCC-TPTA), 9′-[4-(4,6-diphenyl-1,3,5-triazin-2-yl)phenyl]-9,3′:6′,9″-ter-9H-carbazole (BCC-TPTA), 9-(4-(4,6-diphenyl-1,3,5-triazin-2-yl)phenyl)-3′,6′-diphenyl-9H-3,9′-bicarbazole (DPCC-TPTA), 10-(4,6-diphenyl-1,3,5-triazin-2-yl)-10H-phenoxazine (Phen-TRZ), 9-(4-(4,6-diphenyl-1,3,5-triazin-2-yl)phenyl)-9H-carbazole (Cab-Ph-TRZ), 10-(4-(4,6-diphenyl-1,3,5-triazin-2-yl)phenyl)-10H-spiro[acridine-9,9′-fluorene](SpiroAC-TRZ), 2,4,6-tri(9H-carbazol-9-yl)-3,5-difluorobezonitrile (3CzFCN), 2,3,4,6-tetra(9H-carbazol-9-yl)-5-fluorobenzonitrile (4CzFCN), 5,9-diphenyl-13b-bora-9,13b-dihydro-5H-quino[2,3,4-kl]acridine (DABNA-1) and/or combinations thereof.

The contents of the host 342 in the EML 340 can be about 50 wt. % to about 99 wt. %, for example, about 50 wt. % to about 95 wt. % or about 60 wt. % to about 90 wt. %, and the contents of the dopant 344 in the EML 340 can be about 1 wt. % to about 50 wt. %, for example, about 5 wt. % to about 50 wt. % or about 10 wt. % to about 40 wt. %, but is not limited thereto. When the EML 340 includes both the P-type host and the N-type host, the P-type host and the N-type host can be mixed, but is not limited to, with a weight ratio of about 4:1 to about 1:4, for example about 3:1 to about 1:3.

The HIL 310 is disposed between the first electrode 210 and the HTL 320 and can improve an interface property between the inorganic first electrode 210 and the organic HTL 320. In one embodiment, hole injecting material in the HIL 310 can include, but is not limited to, 4,4′,4″-tris(3-methylphenylamino)triphenylamine (MTDATA), 4,4′,4″-tris(N,N-diphenyl-amino)triphenylamine (NATA), 4,4′,4″-tris(N-(naphthalene-1-yl)-N-phenyl-amino)triphenylamine (1T-NATA), 4,4′,4″-tris(N-(naphthalene-2-yl)-N-phenyl-amino)triphenylamine (2T-NATA), copper phthalocyanine (CuPc), tris(4-carbazoyl-9-yl-phenyl)amine (TCTA), N,N′-diphenyl-N,N′-bis(1-naphthyl)-1,1′-biphenyl-4,4″-diamine (NPB; NPD), N,N′-bis{4-[bis(3-methylphenyl)amino]phenyl}-N,N′-diphenyl-4,4′-biphenyldiamine (DNTPD), 1,4,5,8,9,11-hexaazatriphenylenehexacarbonitrile (dipyrazino[2,3-f:2′3′-h]quinoxaline-2,3,6,7,10,11-hexacarbonitrile; HAT-CN), 2,3,5,6-tetrafluoro-7,7,8,8-tetracyanoquinodimethane (F4-TCNQ), 1,3,4,5,7,8-hexafluorotetracaynonaphthoquinodimethane (F6-TCNNQ), 1,3,5-tris[4-(diphenylamino)phenyl]benzene (TDAPB), poly(3,4-ethylenedioxythiphene)polystyrene sulfonate (PEDOT/PSS), N-(biphenyl-4-yl)-9,9-dimethyl-N-(4-(9-phenyl-9H-carbazol-3-yl)phenyl)-9H-fluoren-2-amine, N,N′-diphenyl-N,N′-di[4-(N,N′-diphenyl-amino)phenyl]benzidine (NPNPB), following hole injecting material of Chemical Formula 12 and/or combinations thereof:

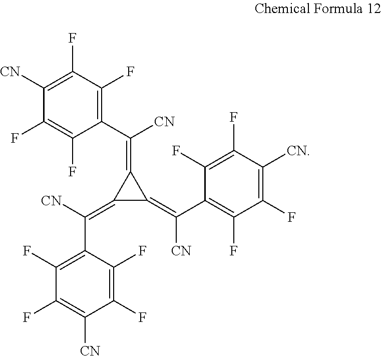

In another embodiment, the HIL 310 can include hole injection host of the following hole transporting material and hole injection dopant of the above hole injecting material (P-type dopant, e.g., HAT-CN, F4-TCNQ, F6-TCNNQ and/or material of Chemical Formula 12). In this case, the contents of the hole injection dopant in the HIL 310 can be, but is not limited to, about 1 wt. % to about 10 wt. %. In certain embodiments, the HIL 310 can be omitted in compliance of the OLED D1 property.

The HTL 320 is disposed adjacently to the EML 340 between the first electrode 210 and the EML 340. In one embodiment, hole transporting material in the HTL 320 can include, but is not limited to, N,N′-diphenyl-N,N′-bis(3-methylphenyl)-1,1′-biphenyl-4,4′-diamine (TPD), NPB(NPD), DNTPD, 4,4′-bis(N-carbazolyl)-1,1′-biphenyl (CBP), Poly[N,N′-bis(4-butylphenyl)-N,N′-bis(phenyl)-benzidine](Poly-TPD), Poly[(9,9-dioctylfluorenyl-2,7-diyl)-co-(4,4′-(N-(4-sec-butylphenyl)diphenylamine))](TFB), di-[4-(N,N-di-p-tolyl-amino)-phenyl]cyclohexane (TAPC), 3,5-di(9H-carbazol-9-yl)-N,N-diphenylaniline (DCDPA), N-(biphenyl-4-yl)-9,9-dimethyl-N-(4-(9-phenyl-9H-carbazol-3-yl)phenyl)-9H-fluoren-2-amine, N-(biphenyl-4-yl)-N-(4-(9-phenyl-9H-carbazol-3-yl)phenyl)biphenyl-4-amine), N-([1,1′-biphenyl]-4-yl)-9,9-dimethyl-N-(4-(9-phenyl-9H-carbazol-3-yl)phenyl)-9H-fluoren-2-amine, following hole transporting material of Chemical Formula 13 and/or combinations thereof:

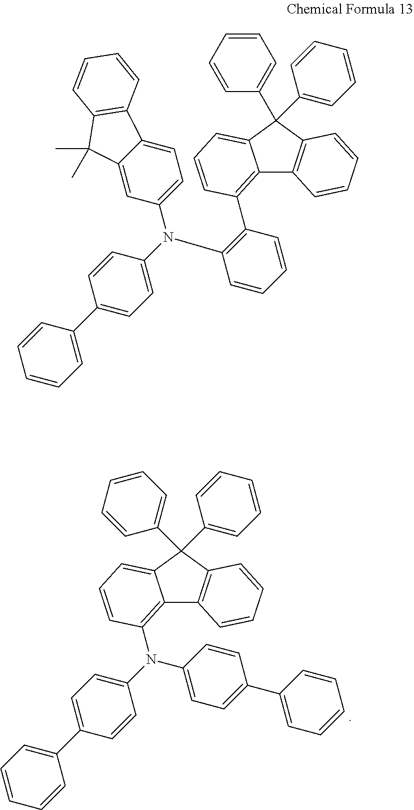 [SMILES: CC1(C)c2ccccc2-c2ccc(N(c3ccc(-c4ccccc4)cc3)c3ccccc3-c3cccc4c3-c3ccccc3C4(c3ccccc3)c3ccccc3)cc21 ; c1ccc(-c2ccc(N(c3ccc(-c4ccccc4)cc3)c3cccc4c3-c3ccccc3C4(c3ccccc3)c3ccccc3)cc2)cc1]

In another embodiment, the hole transporting material can comprise the organic compound having the structure of Chemical Formulae 1 to 6.

The ETL 360 and the EIL 370 can be laminated sequentially between the EML 340 and the second electrode 220. An electron transporting material included in the ETL 360 has high electron mobility so as to provide electrons stably with the EML 340 by fast electron transportation.

The electron transporting material in the ETL 360 can include at least one of oxadiazole-containing compounds, triazole-containing compounds, phenanthroline-containing compounds, benzoxazole-containing compounds, benzothiazole-containing compounds, benzimidazole-containing compounds and triazine-containing compounds.

For example, the electron transporting material in the ETL 360 can include, but is not limited to, tris-(8-hydroxyquinoline aluminum) (Alq₃), 2-biphenyl-4-yl-5-(4-t-butylphenyl)-1,3,4-oxadiazole (PBD), spiro-PBD, lithium 8-quinolinolate (Liq), 1,3,5-tris(N-phenylbenzimidazol-2-yl)benzene (TPBi), bis(2-methyl-8-quinolinolato-N1,O8)-(1,1′-biphenyl-4-olato)aluminum (BAlq), 4,7-diphenyl-1,10-phenanthroline (Bphen), 2,9-bis(naphthalene-2-yl)4,7-diphenyl-1,10-phenanthroline (NBphen), 2,9-dimethyl-4,7-diphenyl-1,10-phenathroline (BCP), 3-(4-biphenyl)-4-phenyl-5-tert-butylphenyl-1,2,4-triazole (TAZ), 4-(naphthalen-1-yl)-3,5-diphenyl-4H-1,2,4-triazole (NTAZ), 1,3,5-tri(p-pyrid-3-yl-phenyl)benzene (TpPyPB), 2,4,6-tris(3′-(pyridin-3-yl)biphenyl-3-yl)1,3,5-triazine (TmPPPyTz), Poly[9,9-bis(3′-((N,N-dimethyl)-N-ethylammonium)-propyl)-2,7-fluorene-alt-2,7-(9,9-dioctylfluorene)](PFNBr), tris(phenylquinoxaline) (TPQ), TSPO1, 2-[4-(9,10-Di-2-naphthalen2-yl-2-anthracen-2-yl)phenyl]-1-phenyl-1H-benzimidazole (ZADN), the following electron transporting material of Chemical Formula 14 and/or combinations thereof:

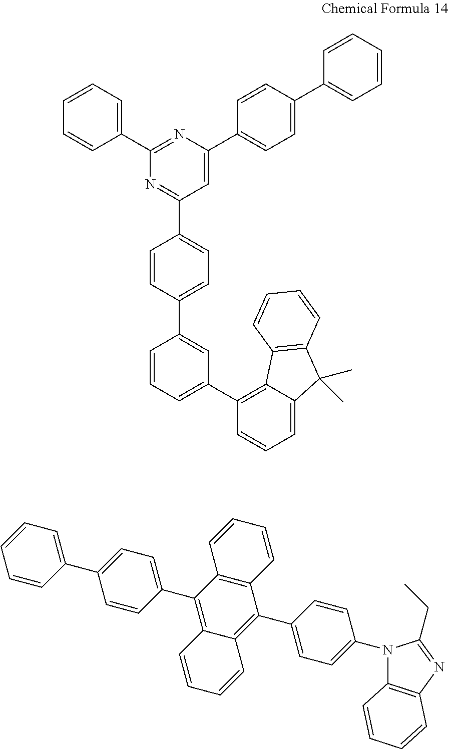 [SMILES: CC1(C)c2ccccc2-c2c(-c3cccc(-c4ccc(-c5cc(-c6ccc(-c7ccccc7)cc6)nc(-c6ccccc6)n5)cc4)c3)cccc21 ; CCc1nc2ccccc2n1-c1ccc(-c2c3ccccc3c(-c3ccc(-c4ccccc4)cc3)c3ccccc23)cc1]

In another embodiment, the electron transporting material can comprise the organic compound having the structure of Chemical Formulae 1 to 6.

The EIL 370 is disposed between the second electrode 220 and the ETL 360, and can improve physical properties of the second electrode 220 and therefore, can enhance the lifespan of the OLED D1. In one embodiment, electron injecting material in the EIL 370 can include, but is not limited to, an alkali metal halide or an alkaline earth metal halide such as LiF, CsF, NaF, BaF₂ and the like, and/or an organometallic compound such as Liq, lithium benzoate, sodium stearate, and the like. In certain embodiments, the EIL 370 can be omitted.

In another embodiment, the ETL 360 and the EIL 370 can have a single layered structure. In this case, the above electron transporting material and/or the electron injecting material can be mixed with each other. As an example, the ETL/EIL with a single layered structure can include two or more different electron transporting materials. For example, two electron transporting materials in the ETL/EIL are mixed with a weight ratio of about 3:7 to about 7:3, but is not limited thereto.

When holes are transferred to the second electrode 220 via the EML 340 and/or electrons are transferred to the first electrode 210 via the EML 340, the OLED D1 can have short lifespan and reduced luminous efficiency. In order to prevent those phenomena, the OLED D1 in accordance with this aspect of the present disclosure can have at least one exciton blocking layer adjacent to the EML 340.

As an example, the OLED D1 can include the EBL 330 between the HTL 320 and the EML 340 so as to control and prevent electron transfers. In one embodiment, electron blocking material in the EBL 330 can include, but is not limited to, TCTA, tris[4-(diethylamino)phenyl]amine, N-(biphenyl-4-yl)-9,9-dimethyl-N-(4-(9-phenyl-9H-carbazol-3-yl)phenyl)-9H-fluorene-2-amine, TAPC, MTDATA, mCP, mCBP, CuPc, DNTPD, TDAPB, DCDPA, 2,8-bis(9-phenyl-9H-carbazol-3-yl)dibenzo[b,d]thiophene and/or combinations thereof. In another embodiment, the electron blocking material can comprise the organic compound having the structure of Chemical Formulae 1 to 6.

In addition, the OLED D1 can further include the HBL 350 as a second exciton blocking layer between the EML 340 and the ETL 360 so that holes cannot be transferred from the EML 340 to the ETL 360. In one embodiment, hole blocking material in the HBL 350 can include, but is not limited to, at least one of oxadiazole-containing compounds, triazole-containing compounds, phenanthroline-containing compounds, benzoxazole-containing compounds, benzothiazole-containing compounds, benzimidazole-containing compounds, and triazine-containing compounds.

As an example, the hole blocking material in the HBL 350 can include material having a relatively low HOMO energy level compared to the luminescent materials in EML 340. For example, the hole blocking material in the HBL 350 can include, but is not limited to, BCP, BAlq, Alq₃, PBD, spiro-PBD, Liq, bis-4,5-(3,5-di-3-pyridylphenyl)-2-methylpyrimidine (B3PYMPM), DPEPO, 9-(6-(9H-carbazol-9-yl)pyridine-3-yl)-9H-3,9′-bicarbazole, TSPO1 and/or combinations thereof.

In another embodiment, the hole blocking material can comprise the organic compound having the structure of Chemical Formulae 1 to 6.

As described above, the EML 340 comprises the host 342 and the dopant 344, and the host 342 comprises the organic compound having the structure of Chemical Formulae 1 to 6. The luminous lifespan of the OLED D1 can be improved by applying the organic compound having the structure of Chemical Formulae 1 to 6 to the EML 340.

An emitting material layer can comprise plural dopants (emitters). FIG. 4 illustrates a cross-sectional view of an organic light emitting diode having a single emitting part in accordance with another embodiment of the present disclosure.

As illustrated in FIG. 4, the organic light emitting diode D2 comprises a first electrode 210, a second electrode 220 facing to the first electrode 210, and an emissive layer 230A disposed between the first electrode 210 and the second electrode 220. The organic light emitting display device 100 (FIG. 2) comprises the red pixel region, the green pixel region and the blue pixel region, and the OLED D2 can be located in the blue pixel region.

The emissive layer 230A comprises an emitting material layer (EML) 340A disposed between the first and second electrodes 210 and 220. The emissive layer 230A can comprise at least one of a hole transport layer (HTL) 320 disposed between the first electrode 210 and the EML 340A and an electron transport layer (ETL) 360 disposed between the second electrode 220 and the EML 340A. In certain embodiments, the emissive layer 230A can further comprise at least one of a hole injection layer (HIL) 310 disposed between the first electrode 210 and the HTL 320 and an electron injection layer (EIL) 370 disposed between the second electrode 220 and the ETL 360. Alternatively, the emissive layer 230A can further comprise a first exciton blocking layer, i.e., an electron blocking layer (EBL) 330 disposed between the HTL 320 and the EML 340A and/or a second exciton blocking layer, i.e., a hole blocking layer (HBL) 350 disposed between the EML 340A and the ETL 360.

The configurations of first electrode 210, the second electrode 220, and the emissive layer 230A except the EML 340A can be identical to the configurations of corresponding components with referring to FIG. 3.

The EML 340A comprises a host 342 and plural dopants (emitters) 344 and 346. The host can be one or more, and can comprise the organic compound having the structure of Chemical Formulae 1 to 6.

In one embodiment, a first dopant 344 can be blue delayed fluorescent material and/or blue phosphorescent material. For example, when the first dopant 344 is blue phosphorescent material, the first dopant 344 can comprise, but is not limited to, the organometallic compound having the structure of Chemical Formulae 7 to 8. In another embodiment, when the first dopant 344 is blue delayed fluorescent material, the first dopant 344 can comprise, but is not limited to, CzBPCN, DCzIPN, SPXZPO, DPXZPO, TPXZPO, DcZTrz, DDczTrz, 2PXZ-TAZ, DMTDAc, DMOC-DPS, DMAC-DPS, DMAC-TRZ, ACRSA, Cz-VPN, TcZTrz, CzBPCN, mPTC, CC2BP, BDPCC-TPTA, BCC-TPTA, DPCC-TPTA, Phen-TRZ, Cab-Ph-TRZ, SpiroAC-TRZ, 3CzFCN, 4CzFCN, DABNA-1 and/or combinations thereof.

The second dopant 346 can be blue fluorescent material. For example, the second dopant 346 can comprise the polycyclic organic compound having the structure of Chemical Formulae 9 to 11 and including boron and nitrogen.

The contents of the host 342 in the EML 340A can be about 50 wt. % to about 99 wt. %, for example, about 50 wt. % to about 95 wt. % or about 60 wt. % to about 90 wt. %, the contents of the first dopant 344 in the EML 340A can be about 3 wt. % to about 50 wt. %, for example, about 5 wt. % to about 30 wt. % or about 5 wt. % to about 20 wt. %, and the contents of the second dopant 346 in the EML 340A can be about 0.5 wt. % to about 20 wt. %, for example, about 1 wt. % to about 10 wt. % or about 1 wt. % to about 5 wt. %, but is not limited thereto. When the EML 340A includes both the P-type host and the N-type host, the P-type host and the N-type host can be mixed, but is not limited to, with a weight ratio of about 4:1 to about 1:4, for example about 3:1 to about 1:3.

The EML 340A comprises the second dopant 346 of fluorescent material to maximize the luminous property of the first dopant 344 of delayed fluorescent material or phosphorescent material. The first dopant 344 of phosphorescent material and/or delayed fluorescent material can utilize both the singlet exciton energy and the triplet exciton energy by Intersystem crossing (ISC) or reverse intersystem crossing (RISC) mechanism.

When the EML 340A comprises the second dopant 346 of fluorescent material with appropriate energy level compared to the energy level of the first dopant 344 of phosphorescent material and/or delayed fluorescent material, the second dopant 346 absorbs the exciton energy released from the first dopant 344, the second dopant 346 can maximize its luminous efficiency with generating 100% singlet excitons using the absorbed energy.

In one embodiment, the excited singlet exciton energy, including the singlet exciton energy converted upwardly from the triplet exciton energy and the initial singlet exciton energy, of the first dopant 344 of delayed fluorescent material in the EML 340A, is mainly transferred to the second dopant 346, which is fluorescent material in the same EML 340A, by Forster resonance energy transfer (FRET) mechanism, and the ultimate light emission is occurred at the second dopant 346. In another embodiment, the EML 340A comprises the first dopant 344 of phosphorescent material with beneficial luminous efficiency and the second dopant 346 of fluorescent material with beneficial color purity so that the EML 340A can implement phosphor-sensitized fluorescence (PSF).

A compound with a large overlapping area of the absorption wavelength with respect to the luminescence wavelength of the first dopant 344 can be selected as the second dopant 346 so that the exciton energy generated at the first dopant 344 can be transferred efficiently to the second dopant 346. The second dopant 346 that ultimately emits light has a narrow full-width at half maximum (FWHM) so that the second dopant 346 can exhibit beneficial color purity.

The first dopant 344 with delayed fluorescence property and/or phosphorescence property can receive exciton energy from the host 342. The first dopant 344 can generate both the singlet exciton energy and the triplet exciton energy by ISC or RISC and can transfer efficiently the generated exciton energy to the second dopant 346. The exciton energy generated at the host 342 is transferred to the second dopant 346 with beneficial color purity via the first dopant 344 with beneficial luminous efficiency. Accordingly, the driving voltage of the OLED D2 can be lowered, and the luminous efficiency, luminous lifespan and/or the color purity of the OLED D2 can be further improved.

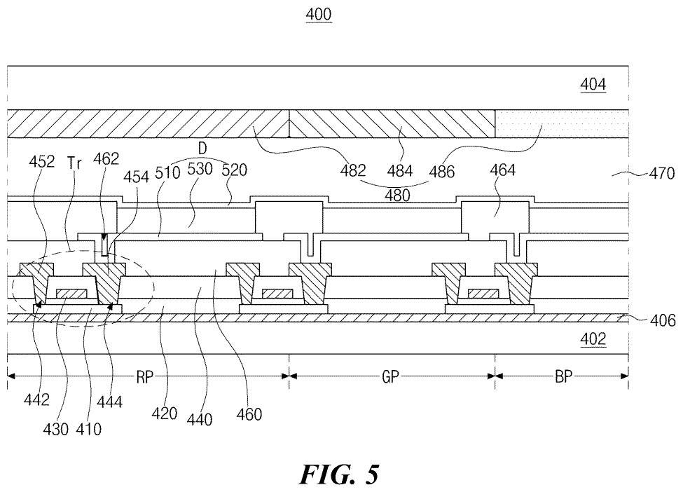

The OLED D1 and the OLED D2 with a single emitting part and emitting blue color light are shown in FIGS. 3 and 4, respectively. In another embodiment, an organic light emitting display device can implement full-color including white color. FIG. 5 illustrates a schematic cross-sectional view of an organic light emitting display device in accordance with another embodiment of the present disclosure.

As illustrated in FIG. 5, the organic light emitting display device 400 includes a first substrate 402 that defines each of a red pixel region RP, a green pixel region GP and a blue pixel region BP, a second substrate 404 facing the first substrate 402, a thin film transistor Tr on the first substrate 402, an OLED D disposed between the first and second substrates 402 and 404 and emitting white (W) light and a color filter layer 480 disposed between the OLED D and the second substrate 404.

Each of the first and second substrates 402 and 404 can include, but is not limited to, glass, flexible material and/or polymer plastics. For example, each of the first and second substrates 402 and 404 can be made of PI, PES, PEN, PET, PC and/or combinations thereof. In certain embodiments, the second substrate 404 can be omitted. The first substrate 402, on which a thin film transistor Tr and the OLED D are arranged, forms an array substrate.

A buffer layer 406 can be disposed on the first substrate 402. The thin film transistor Tr is disposed on the buffer layer 406 correspondingly to each of the red pixel region RP, the green pixel region GP and the blue pixel region BP. In certain embodiments, the buffer layer 406 can be omitted.

A semiconductor layer 410 is disposed on the buffer layer 406. The semiconductor layer 410 can be made of or include oxide semiconductor material or polycrystalline silicon.

A gate insulating layer 420 including an insulating material, for example, inorganic insulating material such as silicon oxide (SiOₓ, wherein 0<x≤2) or silicon nitride (SiNₓ, wherein 0<x≤2) is disposed on the semiconductor layer 410.

A gate electrode 430 made of a conductive material such as a metal is disposed over the gate insulating layer 420 so as to correspond to a center of the semiconductor layer 410. An interlayer insulating layer 440 including an insulating material, for example, inorganic insulating material such as SiOₓ (wherein 0<x≤2) or SiNₓ (wherein 0<x≤2), or an organic insulating material such as benzocyclobutene or photo-acryl, is disposed on the gate electrode 430.

The interlayer insulating layer 440 has first and second semiconductor layer contact holes 442 and 444 that expose or do not cover a portion of the surface nearer to the opposing ends than to a center of the semiconductor layer 410. The first and second semiconductor layer contact holes 442 and 444 are disposed on opposite sides of the gate electrode 430 with spacing apart from the gate electrode 430.

A source electrode 452 and a drain electrode 454, which are made of or include a conductive material such as a metal, are disposed on the interlayer insulating layer 440. The source electrode 452 and the drain electrode 454 are spaced apart from each other with respect to the gate electrode 430. The source electrode 452 and the drain electrode 454 contact both sides of the semiconductor layer 410 through the first and second semiconductor layer contact holes 442 and 444, respectively.

The semiconductor layer 410, the gate electrode 430, the source electrode 452 and the drain electrode 454 constitute the thin film transistor Tr, which acts as a driving element.

Although not shown in FIG. 5, the gate line GL and the data line DL, which cross each other to define the pixel region P, and a switching thin film transistor Ts, which is connected to the gate line GL and the data line DL, can be further formed in the pixel region P. The switching thin film transistor Ts is connected to the thin film transistor Tr, which is a driving element. In addition, the power line PL is spaced apart in parallel from the gate line GL or the data line DL, and the thin film transistor Tr can further include the storage capacitor Cst configured to constantly keep a voltage of the gate electrode 430 for one frame.

A passivation layer 460 is disposed on the source electrode 452 and the drain electrode 454 and covers the thin film transistor Tr over the entire first substrate 402. The passivation layer 460 has a drain contact hole 462 that exposes or does not cover the drain electrode 454 of the thin film transistor Tr.

The OLED D is located on the passivation layer 460. The OLED D includes a first electrode 510 that is connected to the drain electrode 454 of the thin film transistor Tr, a second electrode 520 facing the first electrode 510 and an emissive layer 530 disposed between the first and second electrodes 510 and 520.

The first electrode 510 formed for each pixel region RP, GP or BP can be an anode and can include a conductive material having relatively high work function value. Alternatively, a reflective electrode or a reflective layer can be disposed under the first electrode 510. For example, the reflective electrode or the reflective layer can include, but is not limited to, Ag or APC alloy.

A bank layer 464 is disposed on the passivation layer 460 in order to cover edges of the first electrode 510. The bank layer 464 exposes or does not cover a center of the first electrode 510 corresponding to each of the red pixel RP, the green pixel GP and the blue pixel BP. In certain embodiments, the bank layer 464 can be omitted.

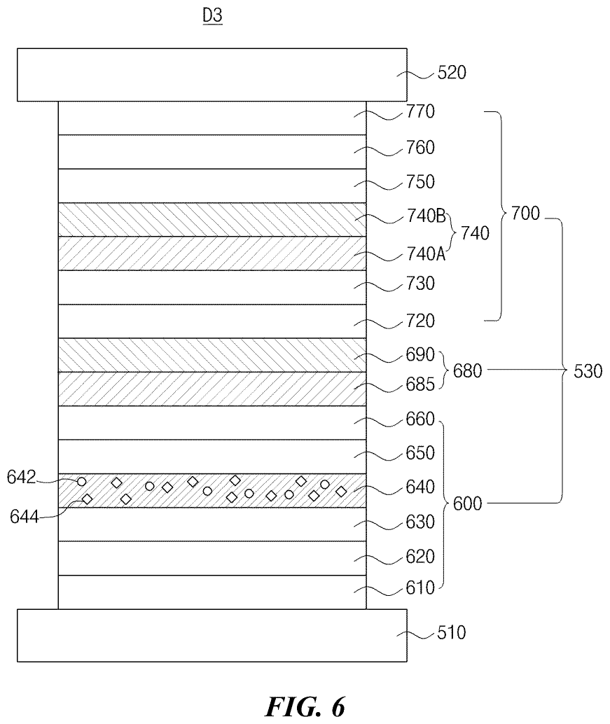

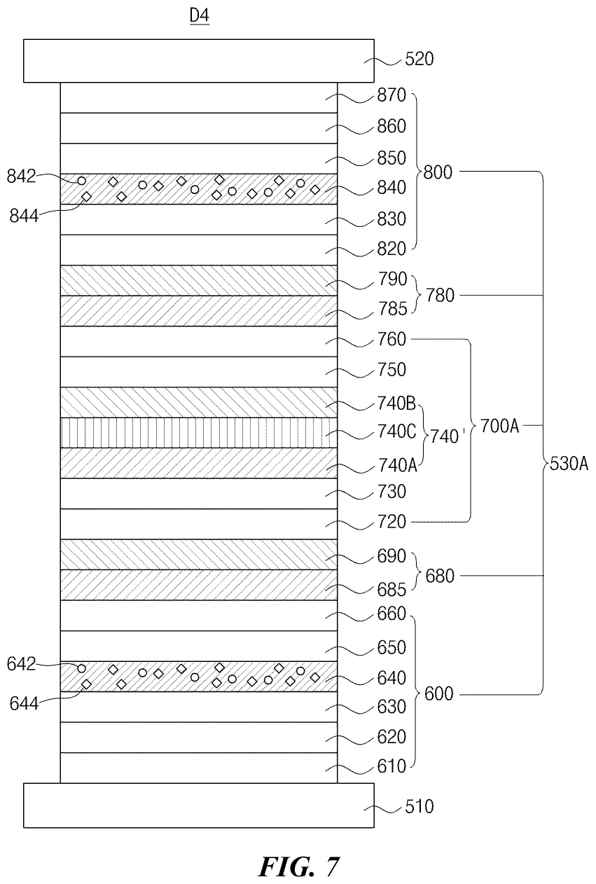

An emissive layer 530 that can include multiple emitting parts is disposed on the first electrode 510. As illustrated in FIGS. 6 and 7, the emissive layer 530 or the emissive layer 530A can include multiple emitting parts 600, 700, 700A, and 800 and at least one charge generation layer 680 and 780. Each of the emitting parts 600, 700, 700A and 800 includes at least one emitting material layer and can further include an HIL, an HTL, an EBL, an HBL, an ETL and/or an EIL.

The second electrode 520 can be disposed on the first substrate 402 above which the emissive layer 530 can be disposed. The second electrode 520 can be disposed over an entire display area, can include a conductive material with a relatively low work function value compared to the first electrode 510, and can be a cathode. Since the light emitted from the emissive layer 530 is incident to the color filter layer 480 through the second electrode 520 in the organic light emitting display device 400 in accordance with the second embodiment of the present disclosure, the second electrode 520 has a thin thickness so that the light can be transmitted.

The color filter layer 480 is disposed on the OLED D and includes a red color filter pattern 482, a green color filter pattern 484 and a blue color filter pattern 486 each of which is disposed correspondingly to the red pixel RP, the green pixel GP and the blue pixel BP, respectively. Although not shown in FIG. 5, the color filter layer 480 can be attached to the OLED D through an adhesive layer. Alternatively, the color filter layer 480 can be disposed directly on the OLED D.

In addition, an encapsulation film 470 can be disposed on the second electrode 520 in order to prevent or reduce outer moisture from penetrating into the OLED D. The encapsulation film 470 can have, but is not limited to, a laminated structure including a first inorganic insulating film, an organic insulating film and a second inorganic insulating film (170 in FIG. 2). In addition, a polarizing plate can be attached onto the second substrate 404 to reduce reflection of external light. For example, the polarizing plate can be a circular polarizing plate.

In FIG. 5, the light emitted from the OLED D is transmitted through the second electrode 520 and the color filter layer 480 is disposed on the OLED D. In this case, the organic light emitting display device 400 can be a top-emission type. Alternatively, when the organic light emitting display device 400 is a bottom-emission type, the light emitted from the OLED D is transmitted through the first electrode 510 and the color filter layer 480 can be disposed between the OLED D and the first substrate 402.

In addition, a color conversion layer may be formed or disposed between the OLED D and the color filter layer 480. The color conversion layer may include a red color conversion layer, a green color conversion layer and a blue color conversion layer each of which is disposed correspondingly to each pixel (RP, GP and BP), respectively, so as to convert the white (W) color light to each of a red, green and blue color lights, respectively. Alternatively, the organic light emitting display device 400 can comprise the color conversion layer instead of the color filter layer 480.

As described above, the white (W) color light emitted from the OLED D is transmitted through the red color filter pattern 482, the green color filter pattern 484 and the blue color filter pattern 486 each of which is disposed correspondingly to the red pixel region RP, the green pixel region GP and the blue pixel region BP, respectively, so that red, green and blue color lights are displayed in the red pixel region RP, the green pixel region GP and the blue pixel region BP.

An OLED that can be applied into the organic light emitting display device will be described in more detail. FIG. 6 illustrates a schematic cross-sectional view of an organic light emitting diode having a tandem structure of two emitting parts.

As illustrated in FIG. 6, the OLED D3 in accordance with the embodiment of the present disclosure includes first and second electrodes 510 and 520 facing each other and an emissive layer 530 disposed between the first and second electrodes 510 and 520. The emissive layer 530 includes a first emitting part 600 disposed between the first and second electrodes 510 and 520, a second emitting part 700 disposed between the first emitting part 600 and the second electrode 520 and a charge generation layer (CGL) 680 disposed between the first and second emitting parts 600 and 700.

The first electrode 510 can be an anode and can include a conductive material having relatively high work function value such as TCO. For example, the first electrode 510 can include, but is not limited to, ITO, IZO, ITZO, SnO, ZnO, ICO, AZO, and/or the like. The second electrode 520 can be a cathode and can include a conductive material with a relatively low work function value. For example, the second electrode 520 can include, but is not limited to, highly reflective material such as Al, Mg, Ca, Ag, alloy thereof and/or combination thereof such as Al—Mg.

The first emitting part 600 includes a first EML (EML1) 640. The first emitting part 600 can further include at least one of a hole injection layer (HIL) 610 disposed between the first electrode 510 and the EML1 640, a first hole transport layer (HTL1) 620 disposed between the HIL 610 and the EML1 640, and a first electron transport layer (ETL1) 660 disposed between the EML1 640 and the CGL 680. Alternatively or additionally, the first emitting part 600 can further include a first electron blocking layer (EBL1) 630 disposed between the HTL1 620 and the EML1 640 and/or a first hole blocking layer (HBL1) 650 disposed between the EML1 640 and the ETL1 660.

The second emitting part 700 includes a second EML (EML2) 740. The second emitting part 700 can further include at least one of a second hole transport layer (HTL2) 720 disposed between the CGL 680 and the EML2 740, an second electron transport layer (ETL2) 760 disposed between the second electrode 520 and the EML2 740 and an electron injection layer (EIL) 770 disposed between the second electrode 520 and the ETL2 760. Alternatively or additionally, the second emitting part 700 can further include a second electron blocking layer (EBL2) 730 disposed between the HTL2 720 and the EML2 740 and/or a second hole blocking layer (HBL2) 750 disposed between the EML2 740 and the ETL2 760.

One of the EML1 640 and the EML2 740 can include the organic compound having the structure of Chemical Formulae 1 to 6. One of the EML1 640 and the EML2 740 can emit blue color light and the other of the EML1 640 and the EML2 740 can emit red to green color light, so that the OLED D3 can realize white (W) emission. Hereinafter, the OLED D3 where the EML1 640 comprises the organic compound having the structure of Chemical Formulae 1 to 6 to emit blue color light, and the EML2 740 emits red to green color light will be described in detail.

The HIL 610 is disposed between the first electrode 510 and the HTL1 620 and improves an interface property between the inorganic first electrode 510 and the organic HTL1 620. In one exemplary embodiment, hole injecting material in the HIL 610 can include, but is not limited to, MTDATA, NATA, 1T-NATA, 2T-NATA, CuPc, TCTA, NPB (NPD), DNDPT, HAT-CN, F4-TCNQ, F6-TCNNQ, TDAPB, PEDOT/PSS, N-(biphenyl-4-yl)-9,9-dimethyl-N-(4-(9-phenyl-9H-carbazol-3-yl)phenyl)-9H-fluoren-2-amine, NPNPB, the hole injection material of Chemical Formula 12 and/or combinations thereof. In another embodiment, the HIL 610 can include hole injection host of hole transporting material and hole injection dopant of the hole injecting material. In certain embodiments, the HIL 610 can be omitted in compliance of the OLED D3 property.

In one embodiment, hole transporting material in each of the HTL1 620 and the HTL2 720 can independently include, but is not limited to, TPD, NPB (NPD), DNTPD, CBP, poly-TPD, TFB, TAPC, DCDPA, N-(biphenyl-4-yl)-9,9-dimethyl-N-(4-(9-phenyl-9H-carbazol-3-yl)phenyl)-9H-fluoren-2-amine, N-(biphenyl-4-yl)-N-(4-(9-phenyl-9H-carbazol-3-yl)phenyl)biphenyl-4-amine, N-([1,1′-biphenyl]-4-yl)-9,9-dimethyl-N-(4-(9-phenyl-9H-carbazol-3-yl)phenyl)-9H-fluoren-2-amine, the hole transporting material of Chemical Formula 13 and/or combinations thereof. In another embodiment, the hole transporting material can comprise the organic compound having the structure of Chemical Formulae 1 to 6.

Each of the ETL1 660 and the ETL2 760 facilitates electron transportation in each of the first emitting part 600 and the second emitting part 700, respectively. As an example, each of electron transporting materials in the ETL1 660 and the ETL2 760 can independently include at least one of oxadiazole-containing compounds, triazole-containing compounds, phenanthroline-containing compounds, benzoxazole-containing compounds, benzothiazole-containing compounds, benzimidazole-containing compound and triazine-containing compounds. For example, each of the electron transporting materials in the ETL1 660 and the ETL2 760 can include, but is not limited to, Alq₃, PBD, spiro-PBD, Liq, TPBi, BAlq, Bphen, NBphen, BCP, TAZ, NTAZ, TpPyPB, TmPPPyTz, PFNBr, TPQ, TSPO1, ZADN, the electrons transporting material of Chemical Formula 14 and/or combinations thereof. In another embodiment, the electron transporting material can comprise the organic compound having the structure of Chemical Formulae 1 to 6.

The EIL 770 is disposed between the second electrode 520 and the ETL2 760, and can improve physical properties of the second electrode 520 and therefore, can enhance the lifespan of the OLED D3. In one embodiment, electron injecting material in the EIL 770 can include, but is not limited to, an alkali metal halide or an alkaline earth metal halide such as LiF, CsF, NaF, BaF₂ and the like, and/or an organometallic compound such as Liq, lithium benzoate, sodium stearate, and the like.

Each of electron blocking materials in the EBL1 630 and the EBL2 730 can independently include, but is not limited to, TCTA, tris[4-(diethylamino)phenyl]amine, N-(biphenyl-4-yl)-9,9-dimethyl-N-(4-(9-phenyl-9H-carbazol-3-yl)phenyl)-9H-fluorene-2-amine, TAPC, MTDATA, mCP, mCBP, CuPc, DNTPD, TDAPB, DCDPA, 2,8-bis(9-phenyl-9H-carbazol-3-yl)dibenzo[b,d]thiophene and/or combinations thereof, respectively. In another embodiment, the electron blocking material can comprise the organic compound having the structure of Chemical Formulae 1 to 6.

Each of hole blocking materials in the HBL1 650 and the HBL2 750 can include, but is not limited to, at least one of oxadiazole-containing compounds, triazole-containing compounds, phenanthroline-containing compounds, benzoxazole-containing compounds, benzothiazole-containing compounds, benzimidazole-containing compounds, and triazine-containing compounds. For example, each of the hole blocking materials in the HBL1 650 and the HBL2 750 can independently include, but is not limited to, BCP, BAlq, Alq₃, PBD, spiro-PBD, Liq, B3PYMPM, DPEPO, 9-(6-(9H-carbazol-9-yl)pyridine-3-yl)-9H-3,9′-bicarbazole, TSPO1 and/or combinations thereof, respectively. In another embodiment, the hole blocking material can comprise the organic compound having the structure of Chemical Formulae 1 to 6.

The CGL 680 is disposed between the first emitting part 600 and the second emitting part 700. The CGL 680 includes an N-type CGL (N-CGL) 685 disposed adjacently to the first emitting part 600 and a P-type CGL (P-CGL) 690 disposed adjacently to the second emitting part 700. The N-CGL 685 injects electrons to the EML1 640 of the first emitting part 600 and the P-CGL 690 injects holes to the EML2 740 of the second emitting part 700.

The N-CGL 685 can be an organic layer doped with an alkali metal such as Li, Na, K and Cs and/or an alkaline earth metal such as Mg, Sr, Ba and Ra. For example, the host in the N-CGL 685 can include, but is not limited to, Bphen and MTDATA. The contents of the alkali metal or the alkaline earth metal in the N-CGL 685 can be, but is not limited to, between about 0.01 wt. % and about 30 wt. %.

The P-CGL 690 can include, but is not limited to, inorganic material selected from the group consisting of tungsten oxide (WOₓ), molybdenum oxide (MoOₓ), beryllium oxide (Be₂O₃), vanadium oxide (V₂O₅) and/or combinations thereof, and/or organic material selected from the group consisting of NPD, DNTPD, HAT-CN, F4-TCNQ, F6-TCNNQ, TPD, N,N,N′,N′-tetranaphthalenyl-benzidine (TNB), TCTA, N,N′-dioctyl-3,4,9,10-perylenedicarboximide (PTCDI-C8) and/or combinations thereof.

In one embodiment, the EML1 640 can be a blue EML. In this case, the EML1 640 can be a blue EML, a sky-blue EML or a deep-blue EML. The EML1 640 comprises a host 642 and a dopant (emitter) 644 where ultimate light emission occurs.

The host 642 can comprise the organic compound having the structure of Chemical Formulae 1 to 6. The EML1 640 can comprise one or more host 642.

The dopant 644 can comprise at least one of blue phosphorescent material, blue fluorescent material and blue delayed fluorescent material. As an example, the dopant 644 of blue phosphorescent material can comprise the organometallic compound having the structure of Chemical Formulae 7 to 8. The dopant 644 of blue fluorescent material can comprise the organic compound having the structure of Chemical Formulae 9 to 11. The dopant 644 of blue delayed fluorescent material can be identical to the blue delayed fluorescent material with referring to FIGS. 3 and 4.

In one embodiment, the dopant 644 can be single dopant. In another embodiment, the dopant 644 can comprise a first dopant of delayed fluorescent material and/or phosphorescent material and a second dopant of fluorescent material (FIG. 4). The contents of the host 642 and the dopant 644 in the EML1 640 can be identical to the contents of corresponding materials with referring to FIGS. 3 and 4.

The EML2 740 can include a lower emitting material layer (lower EML, first layer) 740A disposed between the EBL2 730 and the HBL2 750 and an upper emitting material layer (upper EML, second layer) 740B disposed between the lower EML 740A and the HBL2 750. One of the first layer 740A and the second layer 740B can emit red color light and the other of the first layer 740A and the second layer 740B can emitting green color light. Hereinafter, the EML2 740 where the first layer 740A emits red color light and the second layer 740B emits green color light will be described in detail.

The first layer 740A can include a red host and a red dopant (emitter). For example, the red host can comprise a bipolar red host, or comprise a P-type red host and an N-type red host.

For example, the P-type red host can include, but is not limited to, a biscarbazole-containing organic compound, an aryl amine- or a hetero aryl amine-containing organic compound with at least one fused aromatic and/or fused hetero aromatic moiety, and/or an aryl amine- or a hetero aryl amine-containing organic compound with a spirofluorene moiety. As an example, the N-type red host can include, but is not limited to, an azine-containing organic compound, a benzimidazole-containing organic compound and/or a quinazoline-containing organic compound.

For example, the red host can comprise, but is not limited to, mCP-CN, CBP, mCBP, mCP, DPEPO, 2,8-bis(diphenylphosphoryl)dibenzothiophene (PPT), 1,3,5-tri[(3-pyridyl)-phen-3-yl]benzene (TmPyPB), 2,6-di(9H-carbazol-9-yl)pyridine (PYD-2Cz), 2,8-di(9H-carbazol-9-yl)dibenzothiophene (DCzDBT), 3′,5′-di(carbazol-9-yl)-[1,1′-biphenyl]-3,5-dicarbonitrile (DCzTPA), 4′-(9H-carbazol-9-yl)biphenyl-3,5-dicarbonitrile (pCzB-2CN), 3′-(9H-carbazol-9-yl)biphenyl-3,5-dicarbonitrile (mCzB-2CN), TSPO1, 9-(9-phenyl-9H-carbazol-6-yl)-9H-carbazole (CCP), 4-(3-(triphenylen-2-yl)phenyl)dibenzo[b,d]thiophene, 9-(4-(9H-carbazol-9-yl)phenyl)-9H-3,9′-bicarbazole, 9-(3-(9H-carbazol-9-yl)phenyl)-9H-3,9′-bicarbazole, 9-(6-(9H-carbazol-9-yl)pyridin-3-yl)-9H-3,9′-bicabazole, 9,9′-diphenyl-9H,9′H-3,3′-bicarbazole (BCzPh), 1,3,5-tris(carbazole-9-yl)benzene (TCP), TCTA, 4,4′-bis(carbazole-9-yl)-2,2′-dimethylbiphenyl (CDBP), (2,7-bis(carbazole-9-yl)-9,9-dimethylfluorene (DMFL-CBP), 2,2′,7,7′-tetrakis(carbazole-9-yl)-9,9-spirofluorene (Spiro-CBP), 3,6-bis(carbazole-9-yl)-9-(2-ethyl-hexyl)-9H-carbazole (TCzl) and/or combinations thereof.

The red dopant can comprise at least one of red phosphorescent material, red fluorescent material and red delayed fluorescent material. For example, the red dopant can comprise, but is not limited to, bis[2-(4,6-dimethyl)phenylquinoline)](2,2,6,6-tetramethylheptane-3,5-dionate)iridium(III), bis[2-(4-n-hexylphenyl)quinoline](acetylacetonate)iridium(III) (Hex-Ir(phq)₂(acac)), tris[2-(4-n-hexylphenyl)quinoline]iridium(III) (Hex-Ir(phq)₃), tris[2-phenyl-4-methylquinoline]iridium(III) (Ir(Mphq)₃), bis(2-phenylquinoline)(2,2,6,6-tetramethylheptene-3,5-dionate)iridium(III) (Ir(dpm)PQ₂), bis(phenylisoquinoline)(2,2,6,6-tetramethylheptene-3,5-dionate)iridium(III) (Ir(dpm)(piq)₂), bis(1-phenylisoquinoline)(acetylacetonate)iridium(III) (Ir(piq)₂(acac)), bis[(4-n-hexylphenyl)isoquinoline](acetylacetonate)iridium(III) (Hex-Ir(piq)₂(acac)), tris[2-(4-n-hexylphenyl)quinoline]iridium(III) (Hex-Ir(piq)₃), tris(2-(3-methylphenyl)-7-methyl-quinolato)iridium (Ir(dmpq)₃), bis[2-(2-methylphenyl)-7-methylquinoline](acetylacetonate)iridium(III) (Ir(dmpq)₂(acac)), bis[2-(3,5-dimethylphenyl)-4-methylquinoline](acetylacetonate)iridium(III) (Ir(mphmq)₂(acac)), tris(dibenzoylmethane)mono(1,10-phenanthroline)europium(III) (Eu(dbm)₃(phen)) and/or combinations thereof.

As an example, the contents of the red host in the first layer 740A can be about 50 wt. % to about 99 wt. %, for example, about 60 wt. % to about 99 wt. % or about 80 wt. % to about 95 wt. %, and the contents of the red dopant in the first layer 740A can be about 1 wt. % to about 50 wt. %, for example, about 1 wt. % to about 40 wt. % or about 5 wt. % to about 20 wt. %, but is not limited thereto. When the first layer 740A includes both the P-type red host and the N-type red host, the P-type red host and the N-type red host can be mixed, but is not limited to, with a weight ratio of about 4:1 to about 1:4, for example about 3:1 to about 1:3.

The second layer 740B can include a green host, and a green dopant (emitter). For example, the green host can comprise a bipolar green host, or comprise a P-type green host and an N-type green host. The green host can be identical to the red host above.

The green dopant can include at least one of green phosphorescent material, green fluorescent material and green delayed fluorescent material. In one embodiment, the green dopant can include, but is not limited to, [bis(2-phenylpyridine)](pyridyl-2-benzofuro[2,3-b]pyridine)iridium, tris[2-phenylpyridine]iridium(III) (Ir(ppy)₃), fac-tris(2-phenylpyridine)iridium(III) (fac-Ir(ppy)₃), bis(2-phenylpyridine)(acetylacetonate)iridium(III) (Ir(ppy)₂(acac)), Tris[2-(p-tolyl)pyridine]iridium(III) (Ir(mppy)₃), bis(2-(naphthalene-2-yl)pyridine)(acetylacetonate)iridium(III) (Ir(npy)₂acac), tris(2-phenyl-3-methyl-pyridine)iridium (Ir(3mppy)₃), fac-tris(2-(3-p-xylyl)phenyl)pyridine iridium(III) (TEG) and/or combinations thereof.

In another embodiment, the green dopant with delayed fluorescence property can include, but is not limited to, DMAC-TRZ, DMAC-DPS, ACRSA, Cz-VPN, TcZTrz, 9,9′-(5-(4,6-diphenyl-1,3,5-triazin-2-yl)-1,3-phenylene)bis(9H-carbazole) (DcZTrz), 9,9′,9″,9′″-((6-phenyl-1,3,5-triazin-2,4-diyl)bis(benzene-5,3,1-triyl))tetrakis(9H-carbazole) (DDczTrz), CC2BP, BDPCC-TPTA, BCC-TPTA, DMOC-DPS, DPCC-TPTA, Phen-TRZ, Cab-Ph-TRZ, 1,2,3,5-Tetrakis(3,6-carbazol-9-yl)-4,6-dicyanobenzene (4CzIPN), 4CzFCN, 4,5-di(9H-carbazol-9-yl) phthalonitrile (2CzPN), 10-(4-(4,6-diphenyl-1,3,5-triazin-2-yl)phenyl)-10H-spiro[acridine-9,9′-xanthene], SpiroAC-TRZ and/or combinations thereof.

As an example, the contents of the green host in the second layer 740B can be about 50 wt. % to about 99 wt. %, for example, about 60 wt. % to about 99 wt. % or about 80 wt. % to about 95 wt. %, and the contents of the green dopant in the second layer 740B can be about 1 wt. % to about 50 wt. %, for example, about 1 wt. % to about 40 wt. % or about 5 wt. % to about 20 wt. %, but is not limited thereto. When the second layer 740B includes the P-type green host and the N-type green host, the P-type green host and the N-type green host can be mixed, but is not limited to, with a weight ratio of about 4:1 to about 1:4, for example about 3:1 to about 1:3.

Optionally, the EML2 740 can further include a third layer (740C in FIG. 7) that can emit yellow-green color light disposed between the first layer 740A of the red EML and the second layer 740B of the green EML.

The OLED D3 in accordance with this embodiment has a tandem structure and includes the organic compound having the structure of Chemical Formulae 1 to 6. The luminous lifespan of the OLED D3, which includes the anthracene-containing organic compound with reduced reactivity, can be improved.

An OLED can have three or more emitting parts to form a tandem structure. FIG. 7 is a schematic cross-sectional view illustrating an organic light emitting diode in accordance with yet another embodiment of the present disclosure.

As illustrated in FIG. 7, the OLED D4 includes first and second electrodes 510 and 520 facing each other and an emissive layer 530A disposed between the first and second electrodes 510 and 520. The emissive layer 530A includes a first emitting part 600 disposed between the first and second electrodes 510 and 520, a second emitting part 700A disposed between the first emitting part 600 and the second electrode 520, a third emitting part 800 disposed between the second emitting part 700A and the second electrode 520, a first charge generation layer (CGL1) 680 disposed between the first and second emitting parts 600 and 700A, and a second charge generation layer (CGL2) 780 disposed between the second and third emitting parts 700A and 800.

The first emitting part 600 includes a first emitting material layer (EML1) 640. The first emitting part 600 can further include at least one of a hole injection layer (HIL) 610 disposed between the first electrode 510 and the EML1 640, a first hole transport layer (HTL1) 620 disposed between the HIL 610 and the EML1 640, a first electron transport layer (ETL1) 660 disposed between the EML1 640 and the CGL1 680. Alternatively or additionally, the first emitting part 600 can further comprise a first electron blocking layer (EBL1) 630 disposed between the HTL1 620 and the EML1 640 and/or a first hole blocking layer (HBL1) 650 disposed between the EML1 640 and the ETL1 660.

The second emitting part 700A includes a second emitting material layer (EML2) 740′. The second emitting part 700A can further include at least one of a second hole transport layer (HTL2) 720 disposed between the CGL1 680 and the EML2 740′ and a second electron transport layer (ETL2) 760 disposed between the EML2 740′ and the CGL2 780. Alternatively or additionally, the second emitting part 700A can further include a second electron blocking layer (EBL2) 730 disposed between the HTL2 720 and the EML2 740′ and/or a second hole blocking layer (HBL2) 750 disposed between the EML2 740′ and the ETL2 760.

The third emitting part 800 includes a third emitting material layer (EML3) 840. The third emitting part 800 can further include at least one of a third hole transport layer (HTL3) 820 disposed between the CGL2 780 and the EML3 840, a third electron transport layer (ETL3) 860 disposed between the second electrode 520 and the EML3 840 and an electron injection layer (EIL) 870 disposed between the second electrode 520 and the ETL3 860. Alternatively or additionally, the third emitting part 800 can further comprise a third electron blocking layer (EBL3) 830 disposed between the HTL3 820 and the EML3 840 and/or a third hole blocking layer (HBL3) 850 disposed between the EML3 840 and the ETL3 860.

The CGL1 680 is disposed between the first emitting part 600 and the second emitting part 700A and the CGL2 780 is disposed between the second emitting part 700A and the third emitting part 800. The CGL1 680 includes a first N-type charge generation layer (N-CGL1) 685 disposed adjacently to the first emitting part 600 and a first P-type charge generation layer (P-CGL1) 690 disposed adjacently to the second emitting part 700A. The CGL2 780 includes a second N-type charge generation layer (N-CGL2) 785 disposed adjacently to the second emitting part 700A and a second P-type charge generation layer (P-CGL2) 790 disposed adjacently to the third emitting part 800. Each of the N-CGL1 685 and the N-CGL2 785 injects electrons to the EML1 640 of the first emitting part 600 and the EML2 740′ of the second emitting part 700A, respectively, and each of the P-CGL1 690 and the P-CGL2 790 injects holes to the EML2 740′ of the second emitting part 700A and the EML3 840 of the third emitting part 800, respectively.

The materials included in the HIL 610, the HTL1 to the HTL3 620, 720 and 820, the EBL1 to the EBL3 630, 730 and 830, the HBL1 to the HBL3 650, 750 and 850, the ETL1 to the ETL3 660, 760 and 860, the EIL 870, the CGL1 680, and the CGL2 780 can be identical to the materials with referring to FIGS. 3 and 6.

In one embodiment, at least one of the EML1 640, the EML2 740′ and the EML3 840 can include the organic compound having the structure of Chemical Formulae 1 to 6. For example, at least one of the EML1 640, the EML2 740′ and the EML3 840 can emit blue color light, and the other of the EML1 640, the EML2 740′ and the EML3 840 can emit red to green color light so that the OLED D4 can realize white (W) emission. Hereinafter, the OLED D4 where the EML2 740′ emits red to green color light, and each of the EML1 640 and the EML3 840 emits blue color light will be described in detail.

Each of the EML1 640 and the EML3 840 can be independently a blue EML. In this case, each of the EML1 640 and the EML3 840 can be independently a blue EML, a sky-blue EML or a deep-blue EML. Each of the EML1 640 and the EML3 840 can independently include at least one blue host and at least one blue dopant. For example, the EML1 640 can include a host 642 and a dopant (emitter) 644 where ultimate light emission occurs, and the EML3 840 can include a host 842 and a dopant 844 where ultimate light emission occurs.

In one embodiment, each of the hosts 642 and 842 can independently comprise the organic compound having the structure of Chemical Formulae 1 to 6. Each of the hosts 642 and 842 can be two or more.

Each of the dopants 644 and 844 can independently comprise at least one of blue phosphorescent material, blue florescent material and blue delayed fluorescent material. As an example, the dopants 644 and/or 844 of blue phosphorescent material can comprise the organometallic compound having the structure of Chemical Formulae 7 to 8. In another embodiment, the dopant 644 and/or 844 of blue fluorescent material can comprise the organic compound having the structure of Chemical Formulae 9 to 11.

In one embodiment, each of the dopants 644 and 844 can be independently a single dopant. In another embodiment, each of the dopants 644 and 844 can independently comprise a first dopant of delayed fluorescent material and/or phosphorescent material and a second dopant of fluorescent material (FIG. 4). The contents of the host 642 or 842 and the dopant 644 or 844 in the EML1 640 and the EML3 840 can be identical to the contents of corresponding component with referring to FIGS. 3 and 4.

Alternatively or additionally, the host 642 and/or the dopant 644 in the EML1 640 can be identical to or different from the host 842 and/or the dopant 844 in the EML3 840, respectively, in terms of color and/or luminous efficiency.

The EML2 740′ can include a lower emitting material layer (first layer) 740A disposed between the EBL2 730 and the HBL2 750, an upper emitting material layer (second layer) 740B disposed between the first layer 740A and the HBL2 750, and a middle emitting material layer (third layer) 740C disposed between the first layer 740A and the second layer 740B. One of the first layer 740A and the second layer 740B can emit red color light and the other of the first layer 740A and the second layer 740B can emit green color light. Hereinafter, the EML2 740′ where the first layer 740A emits a red color light and the second layer 740B emits a green color will be described in detail.

The first layer 740A can include a red host and a red dopant. The materials and the contents of the red host and the red dopant in the first layer 740A can be identical with referring to FIG. 6. The second layer 740B can include a green host and a green dopant. The materials and the contents of the green host and the green dopant in the second layer 740B can be identical with referring to FIG. 6.

The third layer 740C can be a yellow green EML. The third layer 740C can include a yellow green host and a yellow green dopant (emitter). For example, the yellow green host can comprise a bipolar yellow green host, or comprise a P-type yellow green host and an N-type yellow green host. For example, the yellow green host can be identical to the red host and/or the green host with referring to FIG. 6.

The yellow green dopant can include at least one of yellow green phosphorescent material, yellow green fluorescent material and yellow green delayed fluorescent material. For example, the yellow green dopant can include, but is not limited to, 5,6,11,12-Tetraphenylnaphthalene (Rubrene), 2,8-Di-tert-butyl-5,11-bis(4-tert-butylphenyl)-6,12-diphenyltetracene (TBRb), Bis(2-phenylbenzothiazolato)(acetylacetonate)iridium(III) (Ir(BT)₂(acac)), Bis(2-(9,9-diethyl-fluoren-2-yl)-1-phenyl-1H-benzo[d]imdiazolato)(acetylacetonate)iridium(III) (Ir(fbi)₂(acac)), Bis(2-phenylpyridine)(3-(pyridine-2-yl)-2H-chromen-2-onate)iridium(III) (fac-Ir(ppy)₂Pc), Bis(2-(2,4-difluorophenyl)quinoline)(picolinate)iridium(III) (FPQIrpic), Bis(4-phenylthieno[3,2-c]pyridinato-N,C2′) (acetylacetonate) iridium(III) (PO-01) and/or combinations thereof. In another embodiment, the yellow-green dopant can include the organometallic compound having the structure of Chemical Formulae 7 to 8. In certain embodiments, the third layer 740C can be omitted.

When the third layer 740C includes at least one yellow green host, the contents of the yellow green host in the third layer 740C can be about 50 wt. % to about 99 wt. %, for example, about 60 wt. % to about 99 wt. % or about 80 wt. % to about 95 wt. %, and the contents of the yellow green dopant in the third layer 740C can be about 1 wt. % to about 50 wt. %, for example, about 1 wt. % to about 40 wt. % or about 5 wt. % to about 20 wt. %, but is not limited thereto. When the third layer 740C includes the P-type yellow green host and the N-type yellow green host, the P-type yellow green host and the N-type yellow green host can be mixed, but is not limited to, with a weight ratio of about 4:1 to about 1:4, for example about 3:1 to about 1:3.

The OLED D4 in accordance with this embodiment has a tandem structure and includes the organic compound having the structure of Chemical Formulae 1 to 6. The OLED D4, which includes the organic compound and three emitting parts, enables its luminous lifespan to be improved with white emission.

In FIG. 7, the OLED D4 having three emitting parts is illustrated. If necessary, an organic light emitting diode can have four or more emitting parts. As an example, when the OLED has four emitting parts, two emitting part can emit blue color light, another emitting part can emit red color light, and the remaining emitting part can emit green color light, so that the OLED can implement white (W) emission.

### Synthesis Example 1

### Synthesis of Compound C-25

### (1) Synthesis of Intermediate 1-1

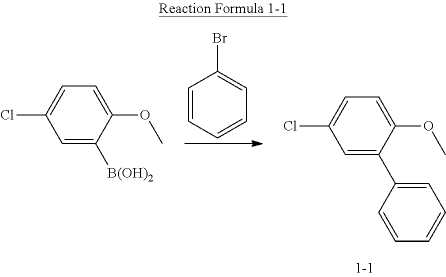

(5-chloro-2-methoxylphenyl)boronic acid (25 g, 134.12 mmol), bromobenzene (21.05 g, 201.18 mmol), palladium-tetrakis(triphenylphosphine) (Pd(PPh₃)₄, 6.19 g, 5.36 mmol), K₂CO₃ (37.07 g, 268.24 mmol), toluene (700 ml), distilled water (180 ml) and ethanol (80 ml) were mixed, then the solution was refluxed with stirring at 120° C. for 4 hours. The solution was cooled to room temperature, distilled water was added into the solution, and an organic layer was extracted with ethyl acetate. MgSO₄ was added to the extracted organic layer to be dried and the organic layer was filtered under reduced pressure. The organic layer was distilled under reduced pressure and purified with column chromatography to give an Intermediate 1-1 (28 g, 95.46%).

### (2) Synthesis of Intermediate 1-2

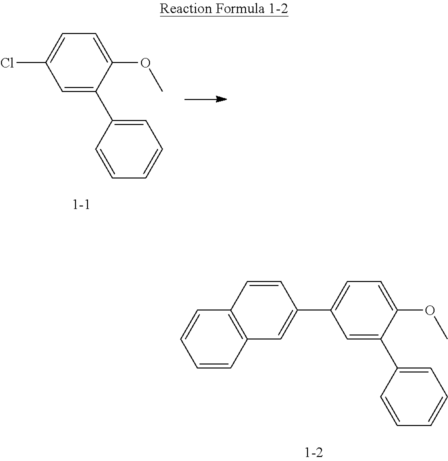 [SMILES: COc1ccc(Cl)cc1-c1ccccc1 ; COc1ccc(-c2ccc3ccccc3c2)cc1-c1ccccc1]

The Intermediate 1-1 (28 g, 128.04 mmol), 2-naphthylboronic acid (26.42 g, 153.6 mmol) palladium(II)acetate (Pd(OAc)₂, 1.43 g, 6.40 mmol), 2-Dicyclohexylphosphino-2′,6′-dimethoxybiphenyl (S phos, 6.30 g, 15.36 mmol), K₃PO₄ (54.35 g, 256.08 mmol), toluene (700 ml), distilled water (150 ml), isopropyl alcohol (80 ml) were mixed, and the solution was refluxed at 120° C. for 3 hours. The solution was cooled to room temperature, distilled water was added into the solution, and an organic layer was extracted with ethyl acetate. MgSO₄ was added to the extracted organic layer to be dried and the organic layer was filtered under reduced pressure. The organic layer was distilled under reduced pressure and purified with column chromatography to give an Intermediate 1-2 (17.5 g, 44.02%).

### (3) Synthesis of Intermediate 1-3

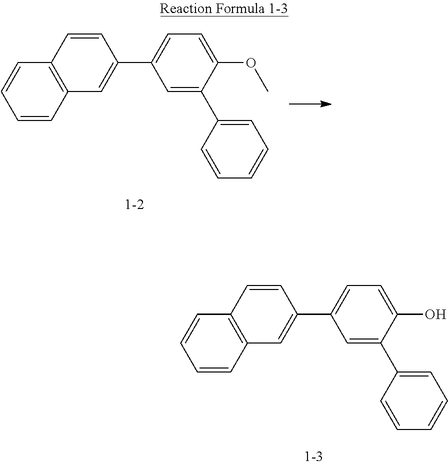 [SMILES: COc1ccc(-c2ccc3ccccc3c2)cc1-c1ccccc1 ; Oc1ccc(-c2ccc3ccccc3c2)cc1-c1ccccc1]

The Intermediate 1-2 (17.5 g, 56.37 mmol) was dissolved in methylene chloride (563 ml), and boron tribromide (BBr₃, 1M in methylene chloride, 84.56 ml) was added to the solution at 0° C. After 30 minutes, the solution was stirred at room temperature for 12 hours. The reaction solution was put into iced water and was stirred. An organic layer was extracted with methylene chloride and neutralized with Na₂CO₃ aqueous solution. MgSO₄ was added the neutralized organic layer to be dried and the organic layer was filtered under reduced pressure. The organic layer was distilled under reduced pressure and purified with column chromatography to give an Intermediate 1-3 (16.0 g, 95.77%).

### (4) Synthesis of Intermediate 1-4

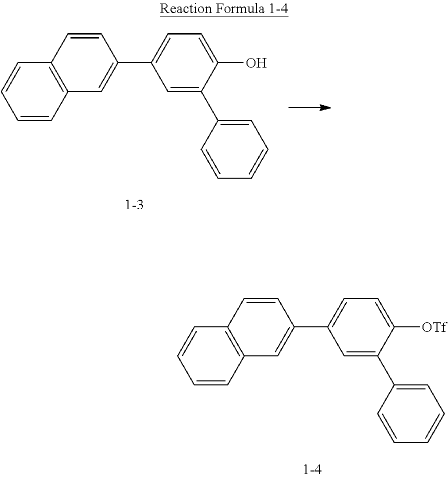 [SMILES: Oc1ccc(-c2ccc3ccccc3c2)cc1-c1ccccc1 ; O=S(=O)(Oc1ccc(-c2ccc3ccccc3c2)cc1-c1ccccc1)C(F)(F)F]

The Intermediate 1-3 (50.0 g, 168.70 mmol) was dissolved in CHCl₃ (1000 ml), and 4-dimethylaminopyridine (2.06 g, 16.87 mmol) was added into the solution. Triethylamine (35.56 ml, 253.06 mmol) was added into the solution at 0° C., and trifluoromethanesulfonic anhydride (42.2 ml, 253.06 mmol) was added into the solution 30 minutes later. The temperature of the solution was raised to room temperature, and the solution was stirred for 90 minutes. Distilled water was added into the solution and an organic layer was extracted with methylene chloride. MgSO₄ was added to the organic layer to be dried and the organic layer was filtered under reduced pressure. The organic layer was distilled under reduced pressure to obtain a solid, and the solid was purified with column chromatography to give an Intermediate 1-4 (65 g, 89.93%).

### (5) Synthesis of Compound C-25

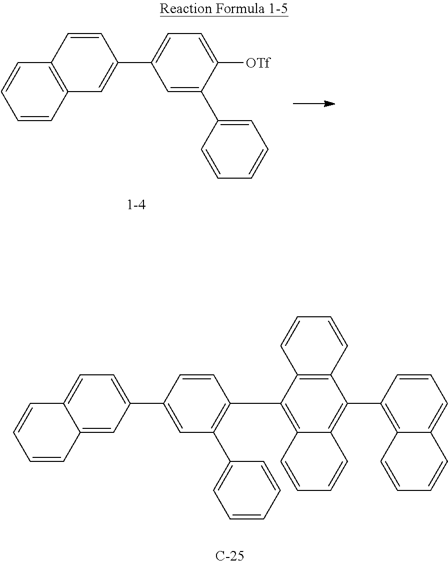

The Intermediate 1-4 (50.0 g, 116.70 mmol), (10-(naphthalene-1-yl)anthracen-9-yl)boronic acid (48.76 g, 140.04 mmol), Pd(OAc)₂ (1.31 g, 5.83 mmol), S phos (5.75 g, 14.0 mmol), K₃PO₄ (49.54 g, 233.41 mmol), toluene (800 ml), distilled water (150 ml) and ethanol (80 ml) were mixed, and the solution was refluxed with stirring at 120° C. for 2 hours. The solution was cooled to room temperature, distilled water was added into the solution, and an organic layer was extracted with ethyl acetate. MgSO₄ was added to the organic layer to be dried, and the organic layer was filtered under reduced pressure. The organic layer was distilled under reduced pressure and purified with column chromatography to give Compound C-25 (34.0 g, 49.99%, molecular weight 582.75, melting point 227.5° C.).

### Synthesis Example 2

### Synthesis of Compound C-100

### (1) Synthesis of Intermediate 2-1

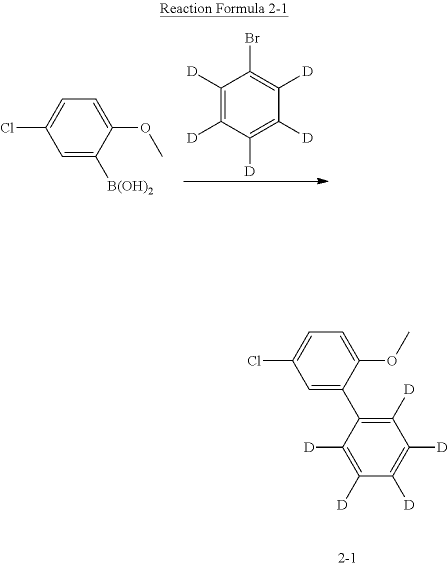

(5-chloro-2-methoxylphenyl)boronic acid (55 g, 295 mmol), bromobenzene-D5 (57.4 g, 354 mmol), Pd(PPh₃)₄ (13.6 g, 5.36 mmol), K₂CO₃ (81.5 g, 590 mmol), toluene (1200 ml), distilled water (300 ml) and ethanol (300 ml) were mixed, then the solution was refluxed with stirring at 120° C. for 4 hours. The solution was cooled to room temperature, distilled water was added into the solution, and an organic layer was extracted with ethyl acetate. MgSO₄ was added to the extracted organic layer to be dried and the organic layer was filtered under reduced pressure. The organic layer was distilled under reduced pressure and purified with column chromatography to give an Intermediate 2-1 (62.8 g, 95.25%).

### (2) Synthesis of Intermediate 2-2

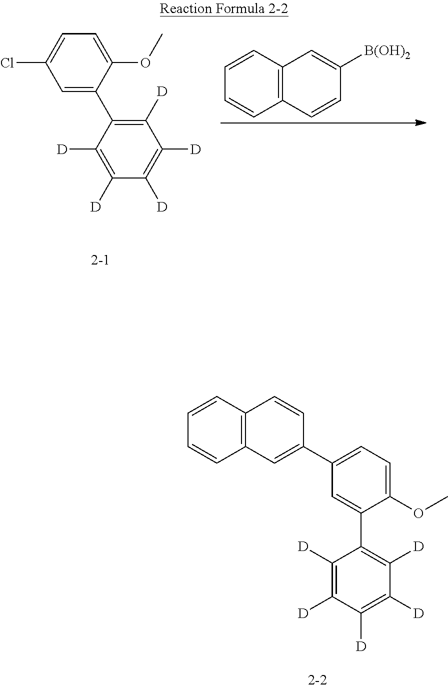 [SMILES: [2H]c1c([2H])c([2H])c(-c2cc(Cl)ccc2OC)c([2H])c1[2H] ; OB(O)c1ccc2ccccc2c1 ; [2H]c1c([2H])c([2H])c(-c2cc(-c3ccc4ccccc4c3)ccc2OC)c([2H])c1[2H]]

The Intermediate 2-1 (62 g, 281 mmol), 2-naphthylboronic acid (58 g, 337 mmol) Pd(OAc)₂ (3.2 g, 14.05 mmol), S phos (13.8 g, 33.72 mmol), K₃PO₄ (119 g, 562 mmol), toluene (1000 ml), distilled water (300 ml), isopropyl alcohol (150 ml) were mixed, and the solution was refluxed at 120° C. for 7 hours. The solution was cooled to room temperature, distilled water was added into the solution, and an organic layer was extracted with ethyl acetate. MgSO₄ was added to the extracted organic layer to be dried and the organic layer was filtered under reduced pressure. The organic layer was distilled under reduced pressure and purified with column chromatography to give an Intermediate 2-2 (71.1 g, 80.06%).

### (3) Synthesis of Intermediate 2-3

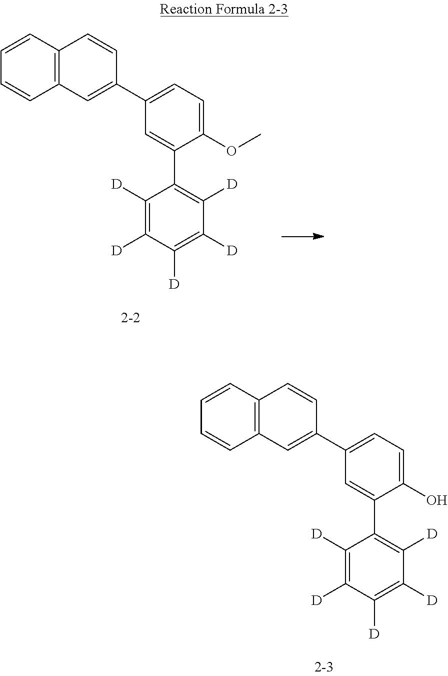 [SMILES: [2H]c1c([2H])c([2H])c(-c2cc(-c3ccc4ccccc4c3)ccc2OC)c([2H])c1[2H] ; [2H]c1c([2H])c([2H])c(-c2cc(-c3ccc4ccccc4c3)ccc2O)c([2H])c1[2H]]

The Intermediate 2-2 (71.1 g, 225 mmol) was dissolved in methylene chloride (1100 ml), and BBr₃ (1M in methylene chloride, 300 ml) was added to the solution at 0° C. After 30 minutes, the solution was stirred at room temperature for 5 hours. The reaction solution was put into iced water and was stirred. The reaction solution was added slowly to Na₂CO₃ aqueous solution at 0° C. and stirred. An organic layer was extracted with methylene chloride, MgSO₄ was added the organic layer to be dried and the organic layer was filtered under reduced pressure. The organic layer was distilled under reduced pressure and purified with column chromatography to give an Intermediate 2-3 (67.6 g, 99.55%).

### (4) Synthesis of Intermediate 2-4

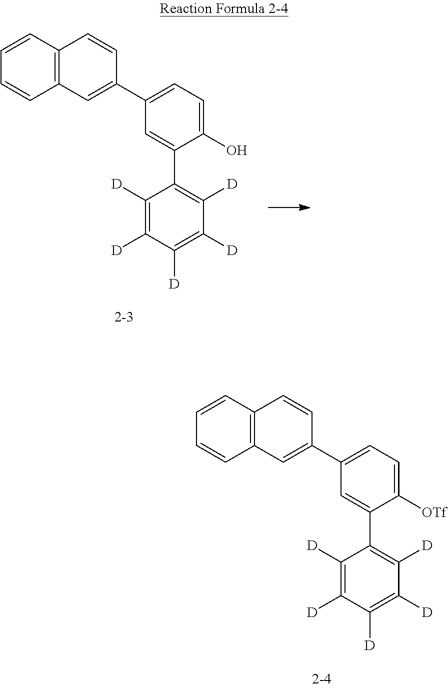 [SMILES: [2H]c1c([2H])c([2H])c(-c2cc(-c3ccc4ccccc4c3)ccc2O)c([2H])c1[2H] ; [2H]c1c([2H])c([2H])c(-c2cc(-c3ccc4ccccc4c3)ccc2OS(=O)(=O)C(F)(F)F)c([2H])c1[2H]]

The Intermediate 2-3 (67.6 g, 224 mmol) was dissolved in CHCl₃ (1100 ml), and 4-dimethylaminopyridine (2.7 g, 22.4 mmol) was added into the solution. Triethylamine (47 ml, 336 mmol) was added into the solution at 0° C., and trifluoromethanesulfonic anhydride (56 ml, 336 mmol) was added into the solution 30 minutes later. After 10 minutes, the temperature of the solution was raised to room temperature, and the solution was stirred for 4 hours. Distilled water was added into the solution and an organic layer was extracted with methylene chloride. MgSO₄ was added to the organic layer to be dried and the organic layer was filtered under reduced pressure. The organic layer was distilled under reduced pressure to obtain a solid, and the solid was purified with column chromatography to give an Intermediate 2-4 (65 g, 89.93%).

### (5) Synthesis of Compound C-100

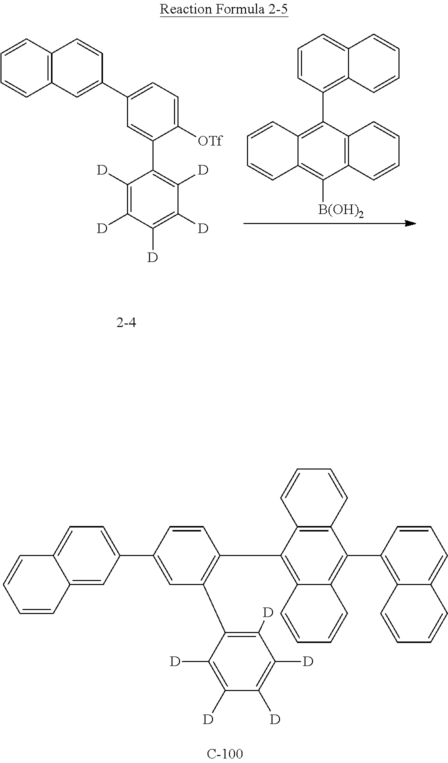 [SMILES: [2H]c1c([2H])c([2H])c(-c2cc(-c3ccc4ccccc4c3)ccc2OS(=O)(=O)C(F)(F)F)c([2H])c1[2H] ; OB(O)c1c2ccccc2c(-c2cccc3ccccc23)c2ccccc12 ; [2H]c1c([2H])c([2H])c(-c2cc(-c3ccc4ccccc4c3)ccc2-c2c3ccccc3c(-c3cccc4ccccc34)c3ccccc23)c([2H])c1[2H]]

The Intermediate 2-4 (50.0 g, 115 mmol), (10-(naphthalene-1-yl)anthracen-9-yl)boronic acid (44 g, 127 mmol), Pd(OAc)₂ (1.31 g, 5.75 mmol), S phos (5.7 g, 13.8 mmol), K₃PO₄ (49 g, 230 mmol), toluene (1100 ml), distilled water (170 ml) and ethanol (115 ml) were mixed, and the solution was refluxed with stirring at 120° C. for 3 hours. The solution was cooled to room temperature, distilled water was added into the solution, and an organic layer was extracted with ethyl acetate. MgSO₄ was added to the organic layer to be dried, and the organic layer was filtered under reduced pressure. The organic layer was distilled under reduced pressure and purified with column chromatography to give Compound C-100 (32.0 g, 47.34%, molecular weight 587.75, melting point 162° C.).

### Example 1 (Ex.1)

### Fabrication of OLED

An organic light emitting diode where an emitting material layer comprised the Compound C-25 synthesized in Synthesis Example 1 as a host was fabricated. A transparent electrode ITO film (10 ohm/sq.) on OLED glass substrate ultrasonically washed with acetone and isopropyl alcohol, and stored in isopropyl alcohol. The ITO substrate was transferred to a vacuum chamber for depositing emissive layer and a cathode. Subsequently, an emissive layer and a cathode were deposited by evaporation under about 10⁻⁶ torr as the following order:

A hole injection layer (HIL, HT-1 (95 wt. %) below, HI below (5 wt. %), 10 nm thickness); a first hole transport layer (HTL1, HT-1, 80 nm thickness); a second hole transporting layer (HTL2, HT-2 below, 15 nm thickness); an emitting material layer (EML, Compound C-25 (98 wt. %), Compound FD1 of Chemical Formula 11 (2 wt. %), 22.5 nm thickness); an electron buffer layer (ET-1 below, 5 nm thickness); an electron transporting layer (ETL, EI-1 below:EI-2 below=2:1 by volume, 25 nm thickness); an electron injection layer (EIL, Yb:LiF=2:1 by volume, 1 nm thickness); a cathode (Al, 80 nm thickness).

The structures of materials of hole injecting material, hole transporting material, electron buffer material, and electron transporting material are illustrated in the following:

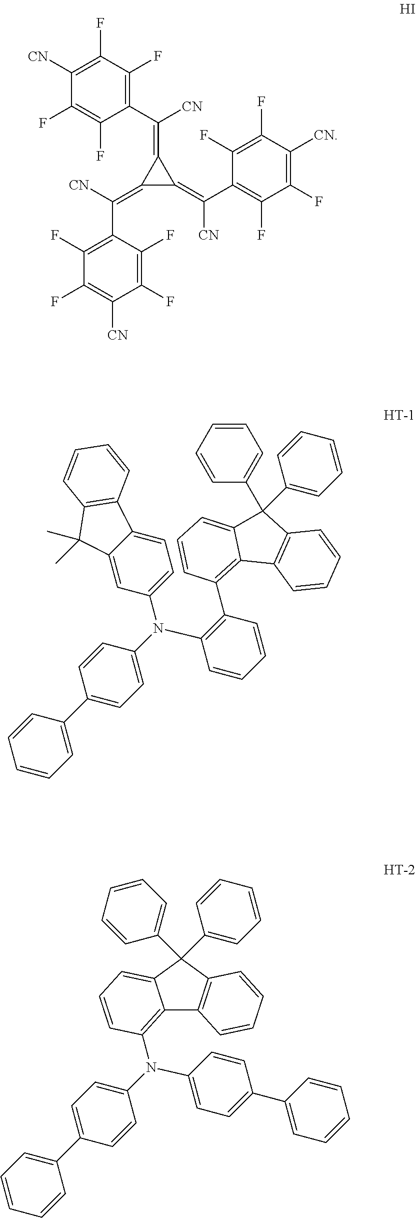

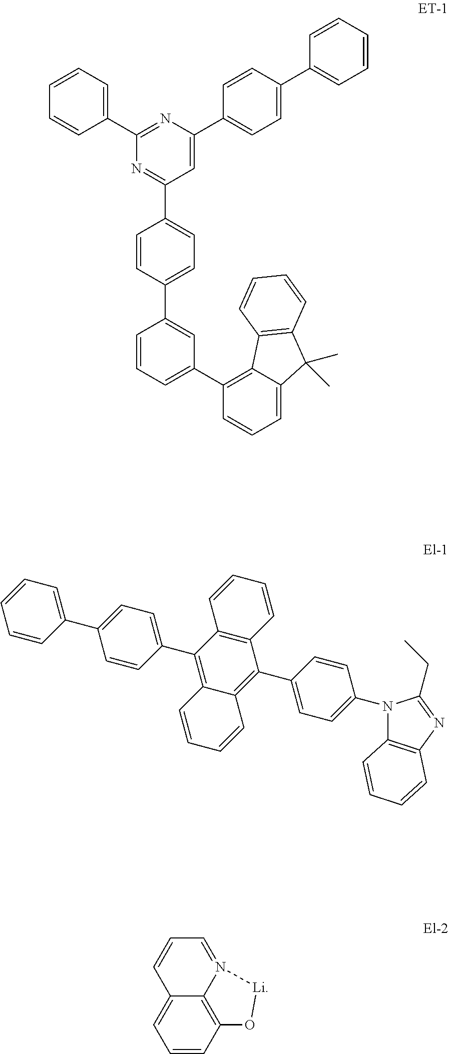

### Example 2 (Ex. 2)

### Fabrication of OLED

An OLED was fabricated using the same procedure and the same materials as Example 1, except that Compound C-100 instead Compound C-25 as the host in the EML was used.

### Comparative Example 1 (Ref. 1)

### Fabrication of OLED

An OLED was fabricated using the same procedure and the same materials as Example 1, except that the Compound H-Ref instead of Compound C-25 as the host in the EML was used.

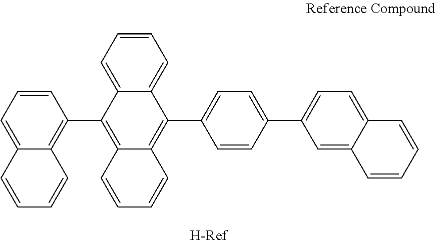 [SMILES: c1ccc2cc(-c3ccc(-c4c5ccccc5c(-c5cccc6ccccc56)c5ccccc45)cc3)ccc2c1]

### Experimental Example 1

### Measurement of Luminous Properties of OLEDs

Each of the OLEDs fabricated in Examples 1 to 2 and Comparative Example 1 was connected to an external power source and then luminous properties for all the OLEDs were evaluated. In particular, driving voltage and time period from initial luminance to 95% luminescence (T₉₅) by CIE color coordinates standard as luminous lifespan were measured at a luminance 1000 nits. The measurement results are illustrated in the following Table 1.

TABLE 1
Luminous Properties of OLED

| | | Driving Voltage | |
|---|---|---|---|
| Sample | Host | (V) | T₉₅ (hour) |
| Ex. 1 | C-25 | 3.9 | 54 |
| Ex. 2 | C-100 | 3.9 | 60 |
| Ref. 1 | H-Ref | 3.9 | 35 |

As indicated in Table 1, compared to the OLED fabricated in Comparative Example 1, in the OLEDs fabricated in Examples 1-2 where an anthracene-containing compound with specific molecular conformation was used as the host in the EML, the luminous lifespan was improved significantly. As an anthracene moiety in the anthracene-containing host is relatively high reactive, the luminous lifespan of the OLED including the anthracene-containing host tends to be reduced. When the reactivity of the anthracene moiety included in the host is lowered, the luminous lifespan of the OLED can be improved. The phenyl substituted to one side of the anthracene core includes another phenyl substituted to the ortho-position of the initial phenyl, so that the steric effect of the anthracene backbone can be adjusted and therefore, the reactivity of the anthracene-core can be reduced. The OLED where the anthracene-containing compound with the specific molecular conformation was used in an emissive layer can have more beneficial luminous lifespan compared to the conventional OLED.

It will be apparent to those skilled in the art that various modifications and variations can be made in the present disclosure without departing from the scope of the disclosure. Thus, it is intended that the present disclosure cover the modifications and variations of the present disclosure provided they come within the scope of the appended claims.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. An organic compound having the following structure of Chemical Formula 1: 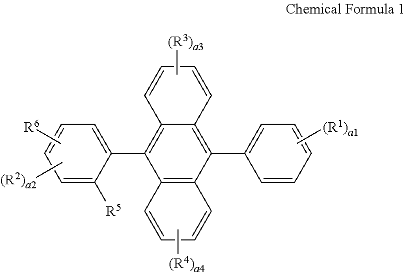
wherein, in Chemical Formula 1,
R¹ is protium, deuterium, tritium, an unsubstituted or substituted C₁-C₁₀ alkyl group or unsubstituted or substituted phenyl, where each R¹ is identical to or different from each other when a1 is 2, 3, 4 or 5, or
optionally,
two adjacent R¹ when a1 is 2, 3, 4 or 5 are further linked to form a benzene ring unsubstituted or substituted with at least one of deuterium, C₁-C₁₀ alkyl, and a group formed by connecting the two groups mentioned above;
each of R², R³ and R⁴ is independently protium, deuterium, tritium or an unsubstituted or substituted C₁-C₁₀ alkyl group, where each R² is identical to or different from each other when a2 is 2 or 3, each R³ is identical to or different from each other when a3 is 2, 3 or 4, and each R⁴ is identical to or different from each other when a4 is 2, 3 or 4;
each of R⁵ and R⁶ is independently phenyl, biphenyl or naphthyl, wherein each of the phenyl, biphenyl and the naphthyl is independently unsubstituted or substituted with at least one of deuterium, C₁-C₁₀ alkyl, and a group formed by connecting the two groups mentioned above;
a1 is 0, 1, 2, 3, 4 or 5;
a2 is 0, 1, 2 or 3; and
each of a3 and a4 is independently 0, 1, 2, 3 or 4.

2. The organic compound of claim 1, wherein the organic compound has the following structure of Chemical Formula 2 or Chemical Formula 3: 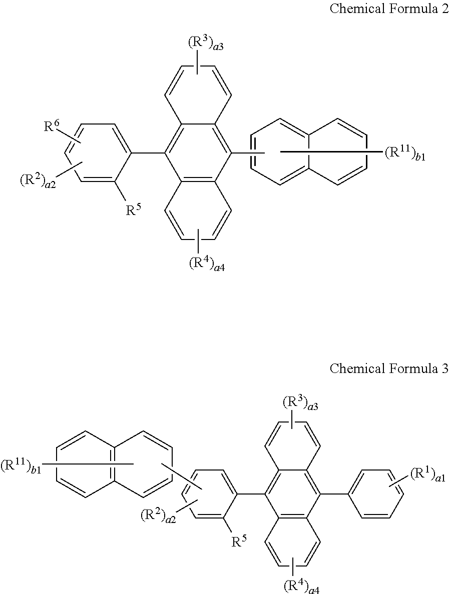
wherein, in Chemical Formulae 2 and 3,
each of R¹, R², R³, R⁴, R⁵, R⁶, a1, a2, a3 and a4 is a same as defined in Chemical Formula 1;
R¹¹ is protium, deuterium or an unsubstituted or deuterium-substituted C₁-C₁₀ alkyl group, where each R¹¹ is identical to or different from each other when b1 is 2, 3, 4, 5, 6 or 7; and
b1 is 0, 1, 2, 3, 4, 5, 6 or 7.

3. The organic compound of claim 1, wherein the organic compound has the following structure of Chemical Formula 4 or Chemical Formula 5: 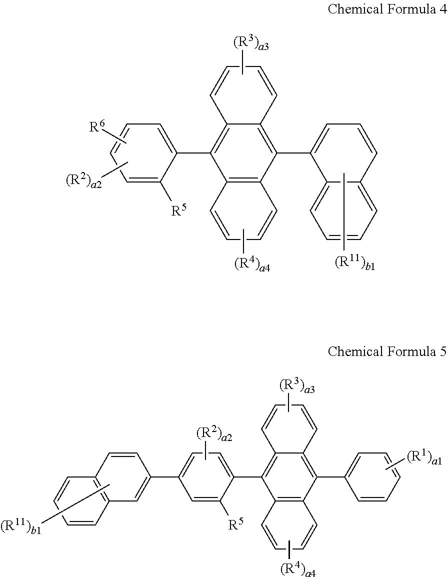
wherein, in Chemical Formulae 4 and 5,
each of R¹, R², R³, R⁴, R⁵, R⁶, a1, a2, a3 and a4 is a same as defined in Chemical Formula 1;
R¹¹ is protium, deuterium or an unsubstituted or deuterium-substituted C₁-C₁₀ alkyl group, where each R¹¹ is identical to or different from each other when b1 is 2, 3, 4, 5, 6 or 7; and
b1 is 0, 1, 2, 3, 4, 5, 6 or 7.

4. The organic compound of claim 1, wherein two adjacent R¹s are further linked to form a benzene ring, the rest three of R¹s are protium or deuterium, and R⁶ is naphthyl in Chemical Formula 1.

5. The organic compound of claim 1, wherein two adjacent R¹s are further linked to form a benzene ring, and thereby linked to the anthracene ring as 1-napthyl form, and R⁶ is 2-naphthyl.

6. The organic compound of claim 1, wherein R⁵ is phenyl, or phenyl where all hydrogens are substituted with deuterium.

7. The organic compound of claim 1, wherein the organic compound is at least one of: 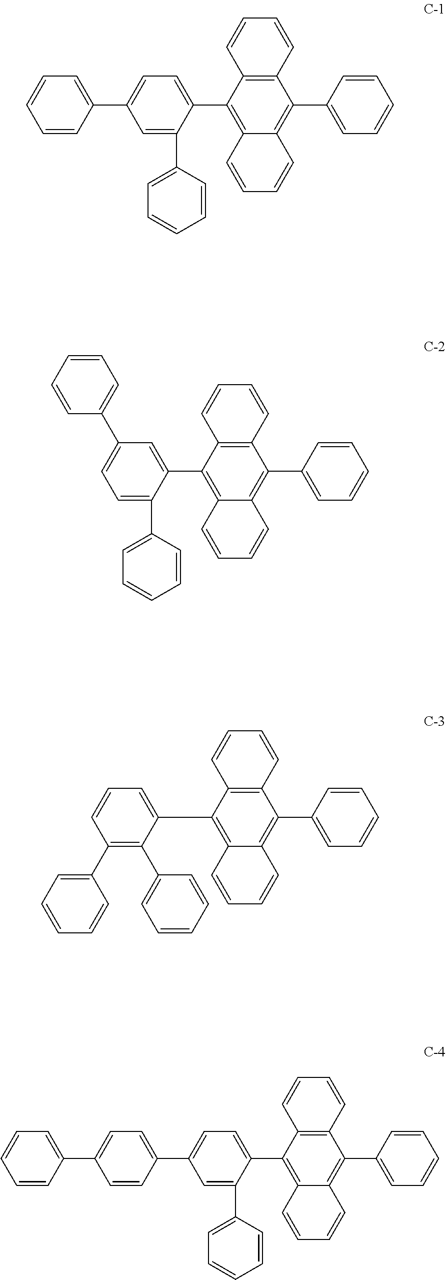 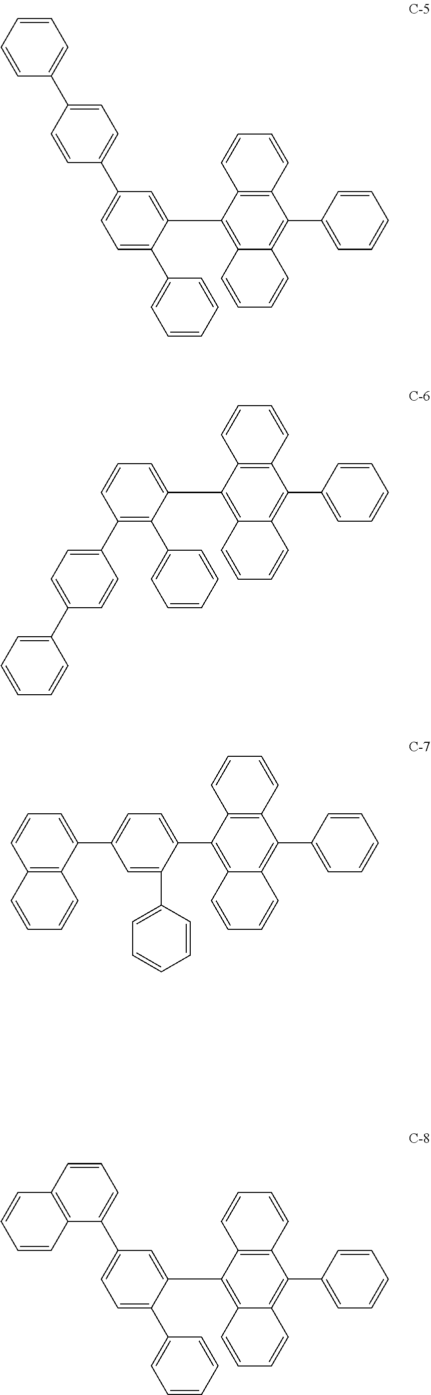 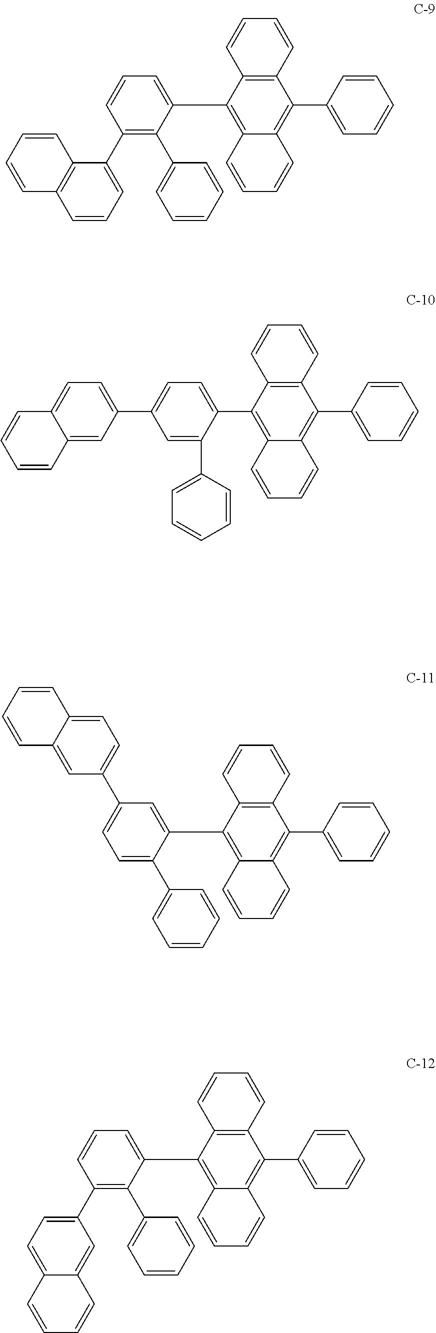 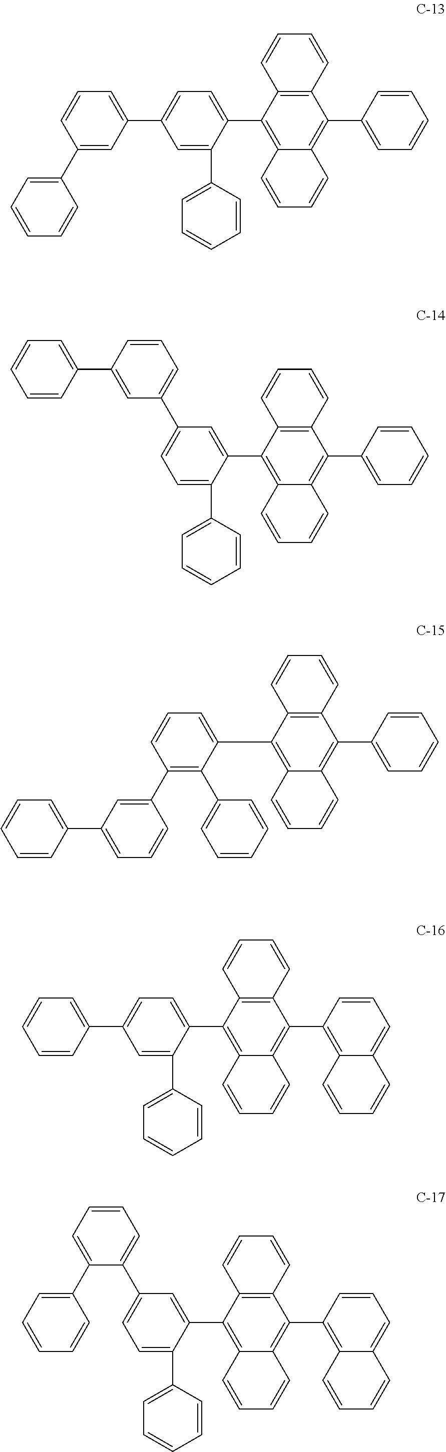 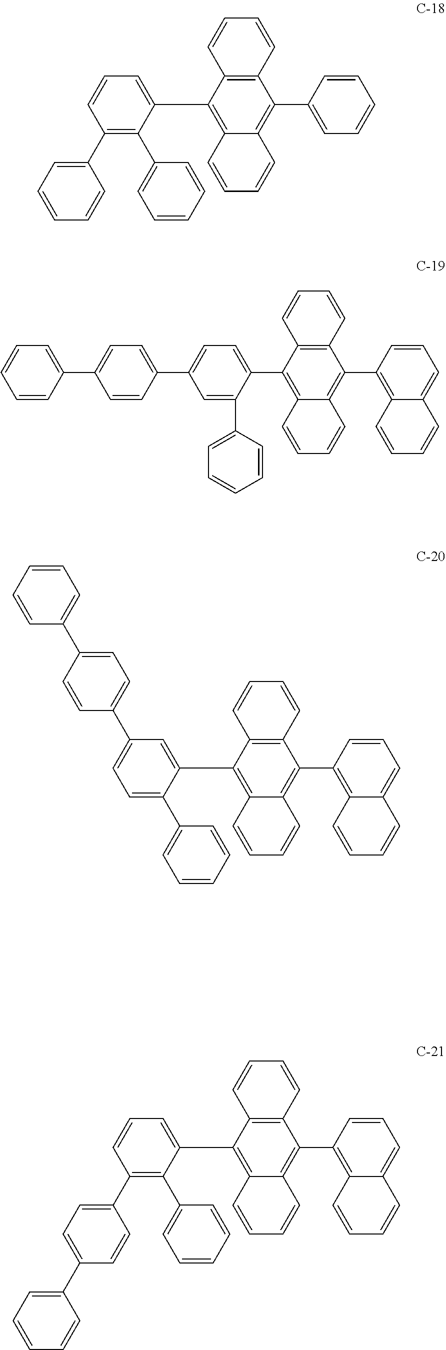 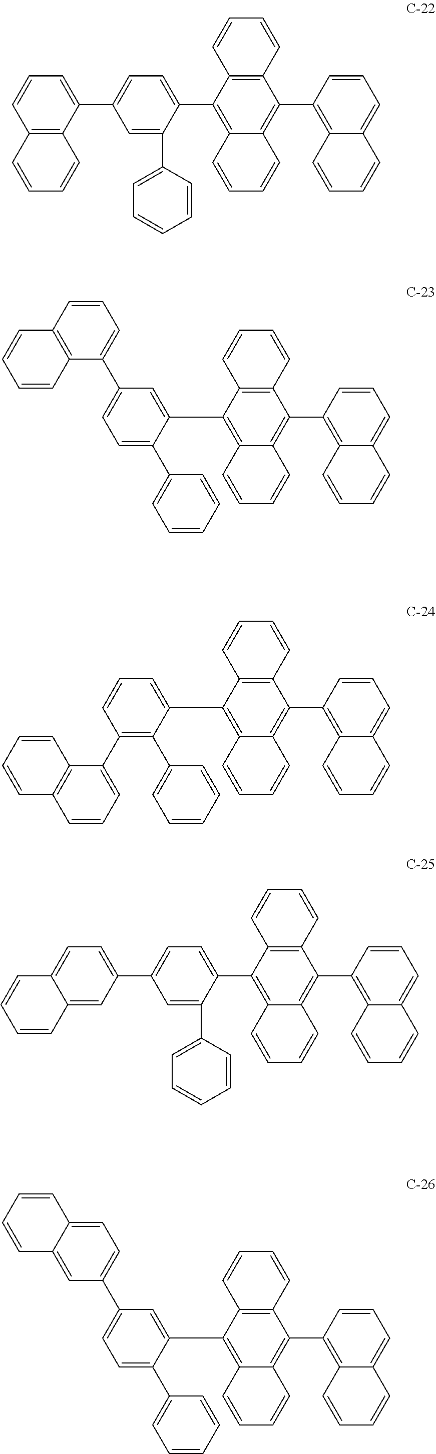 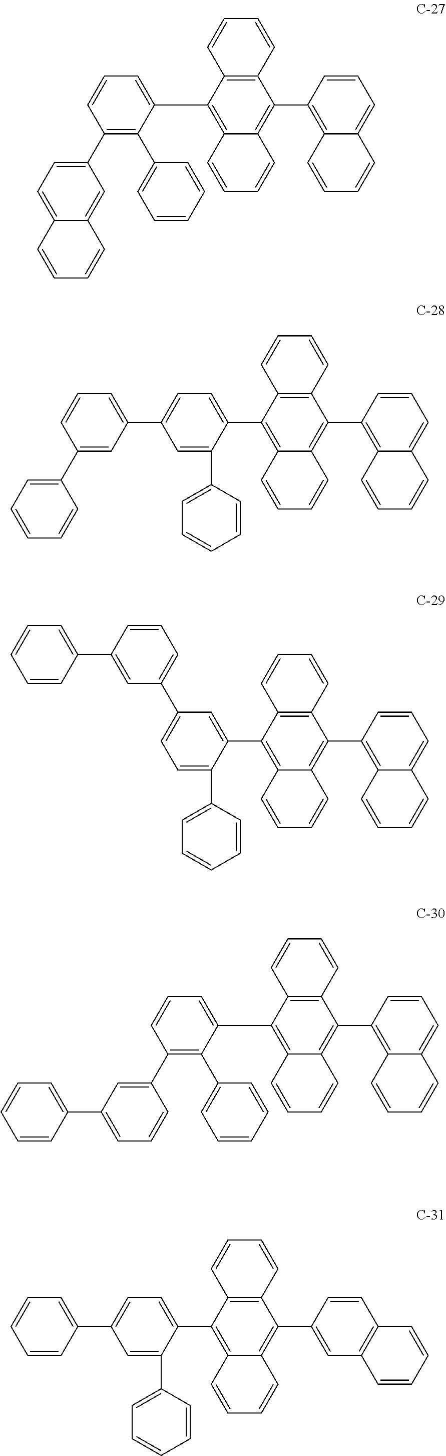 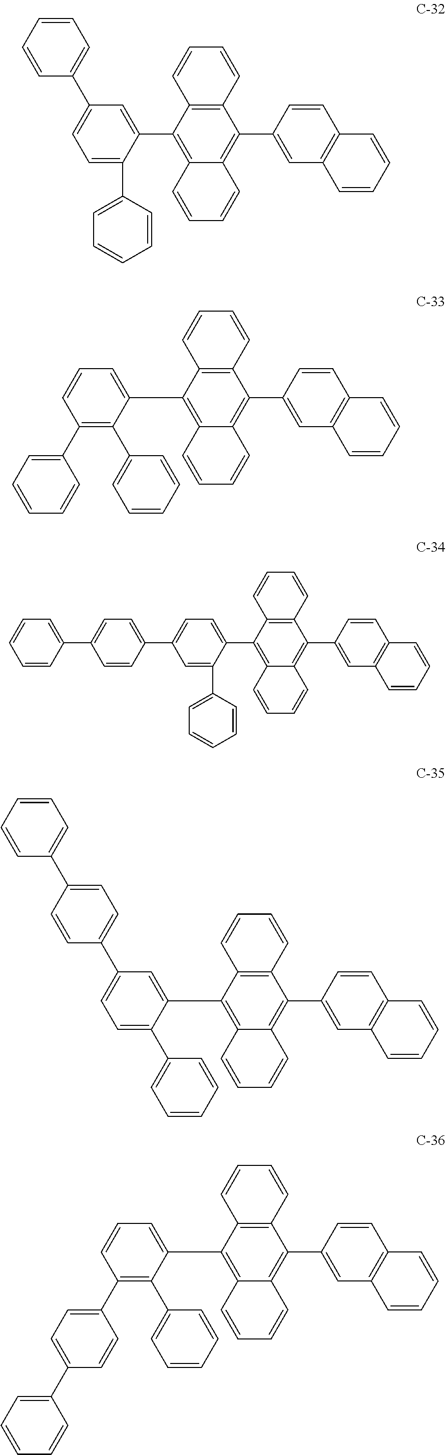 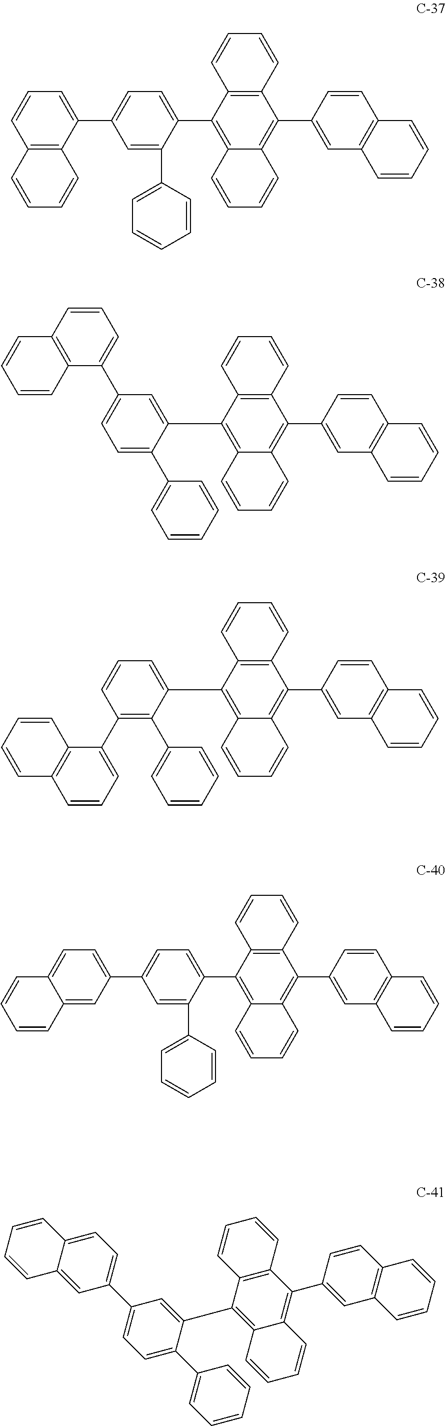 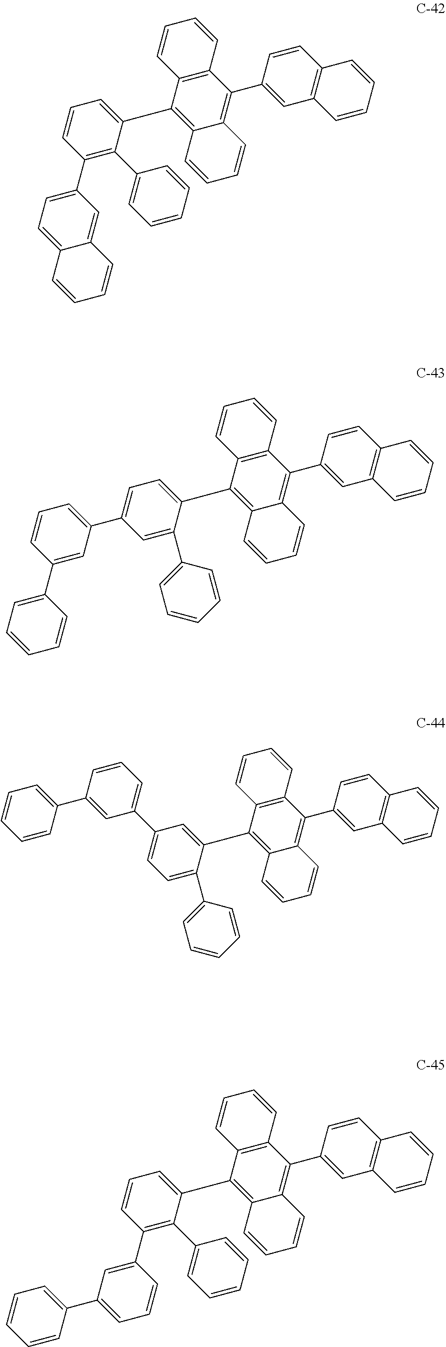 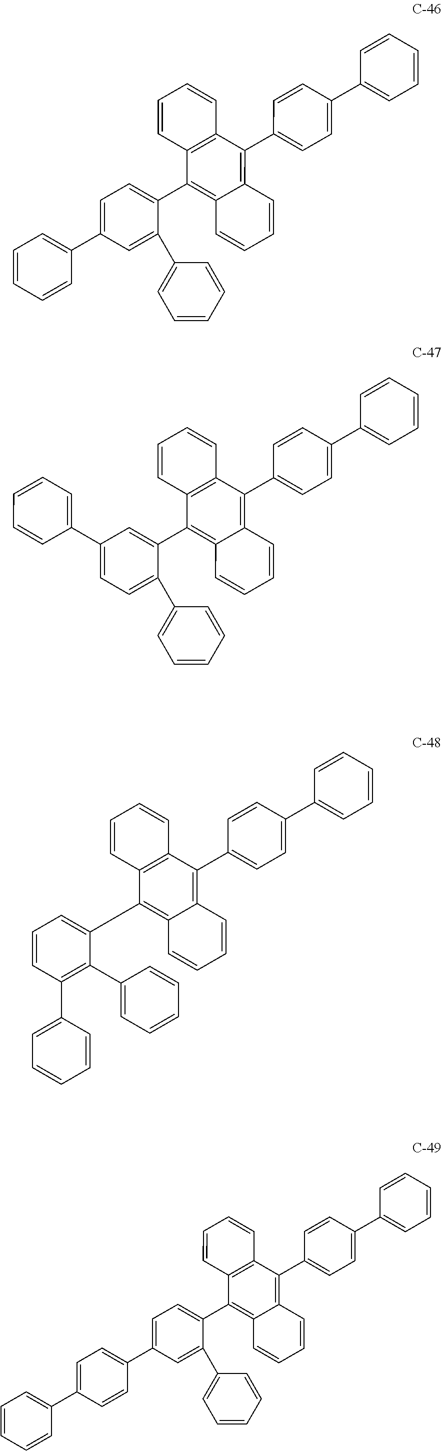 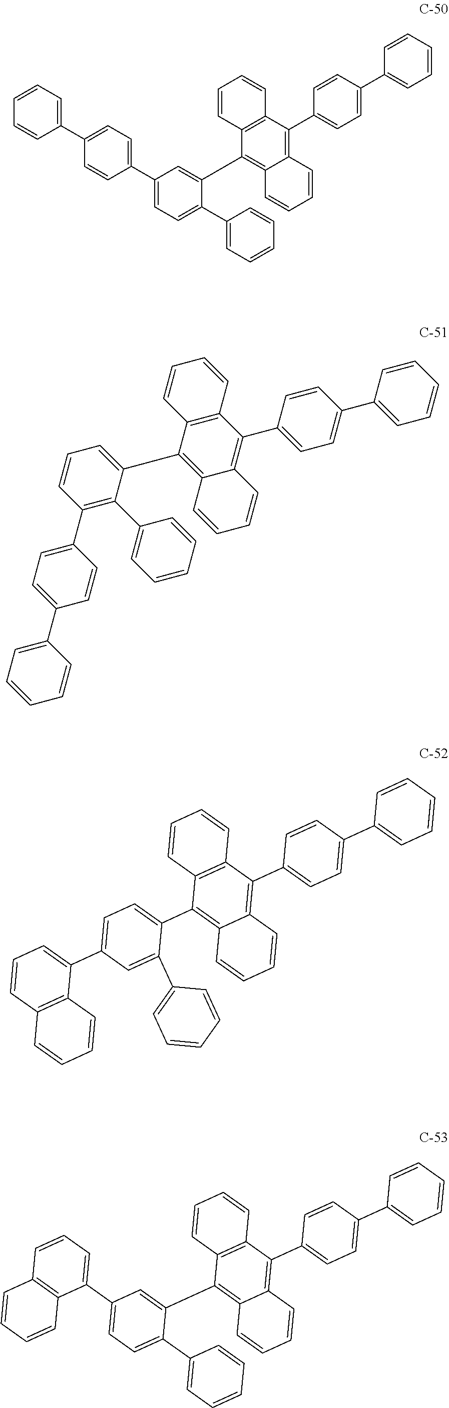 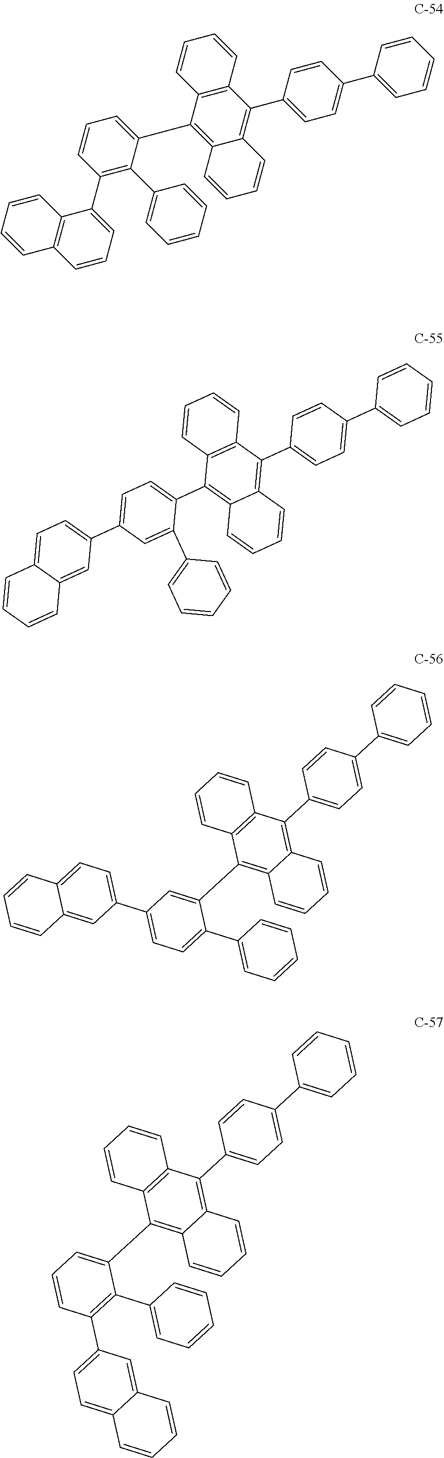 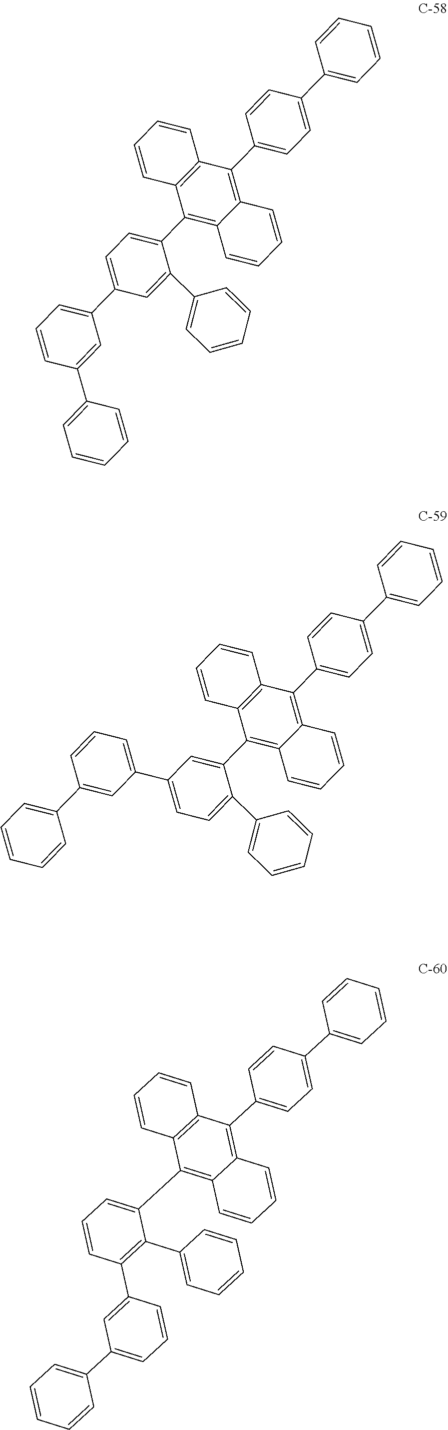 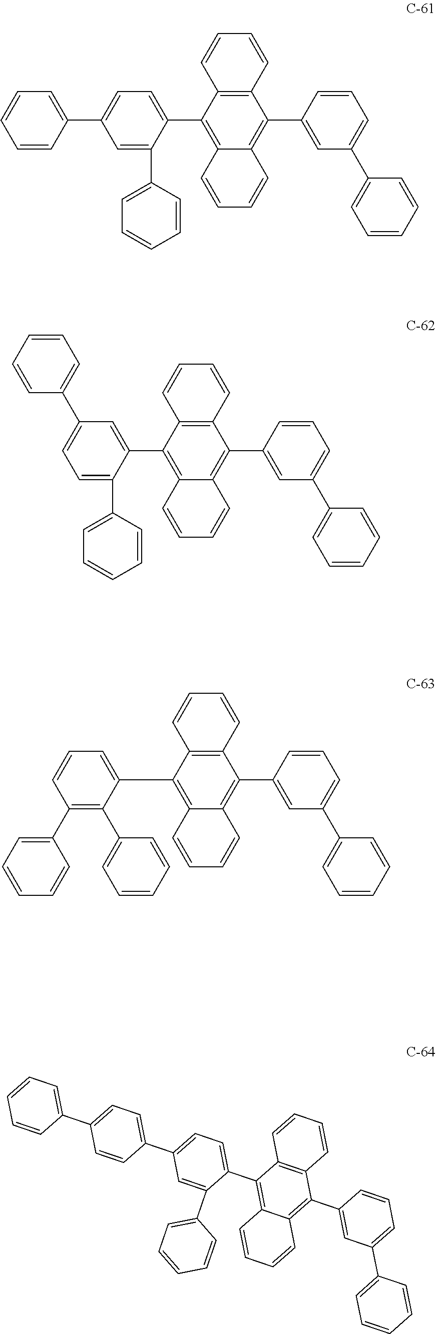 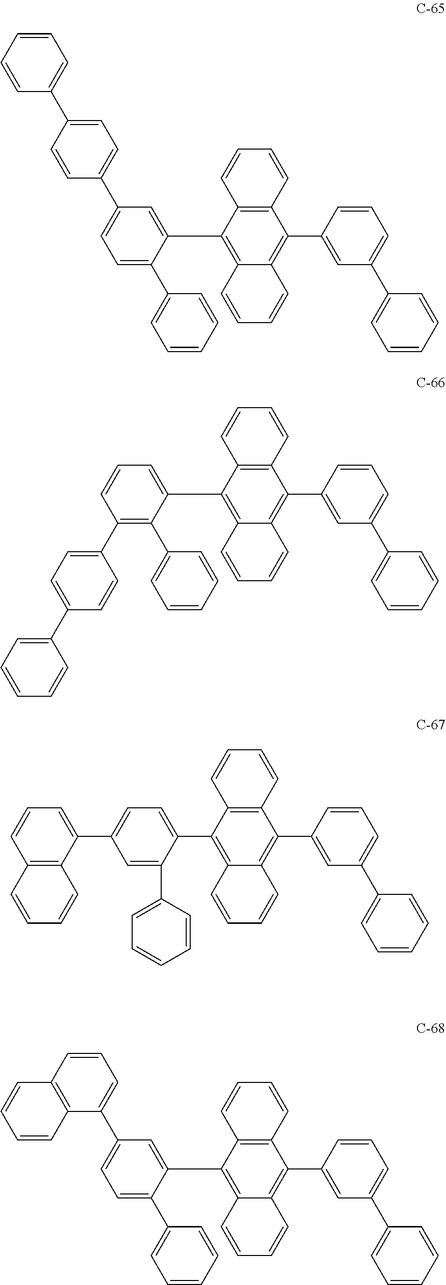 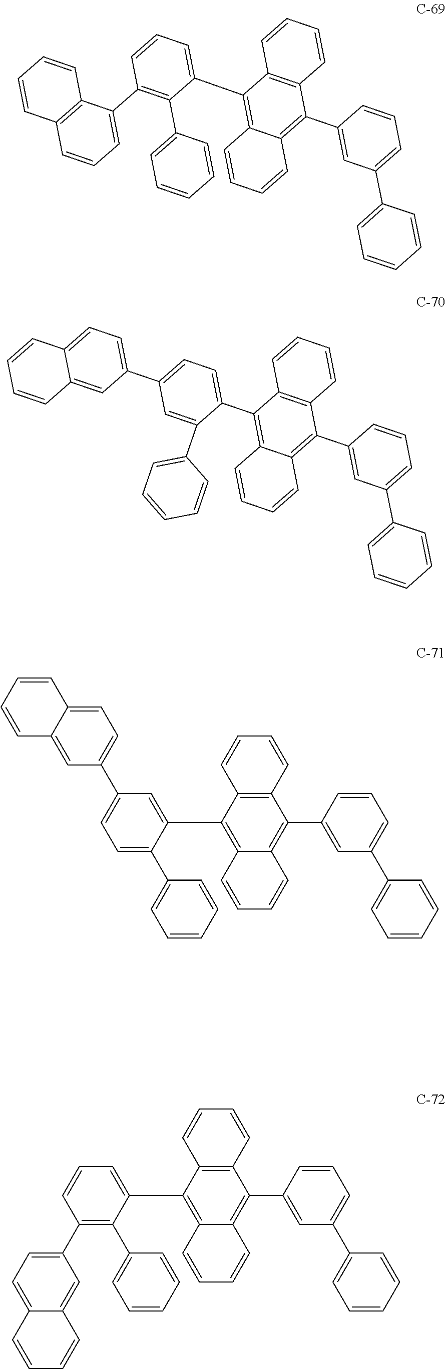 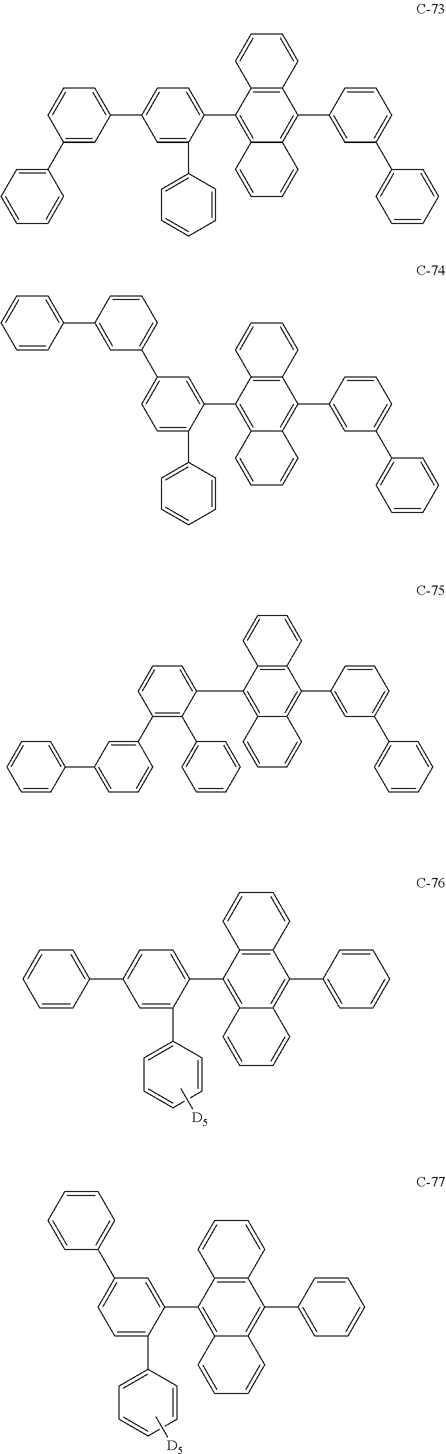 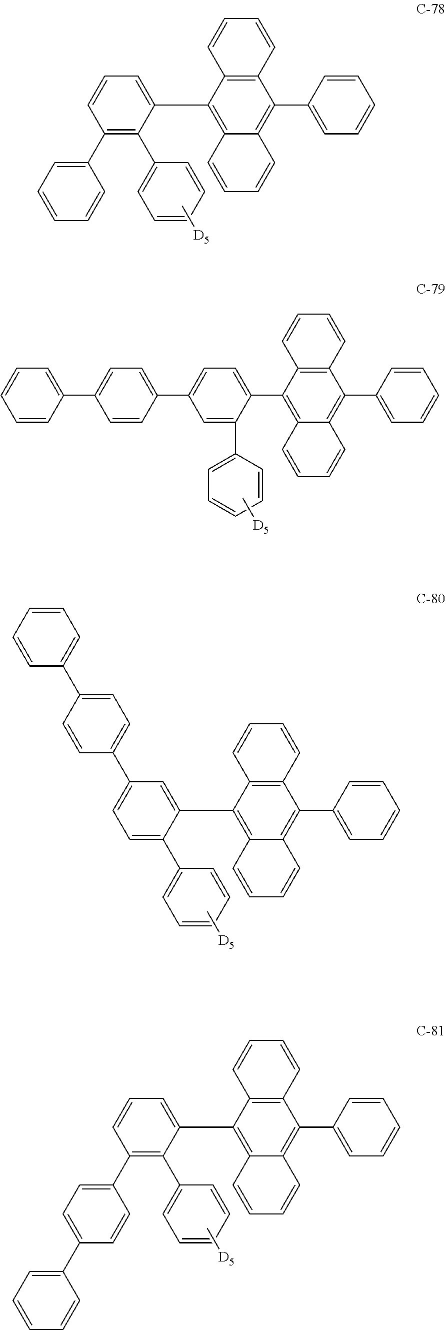 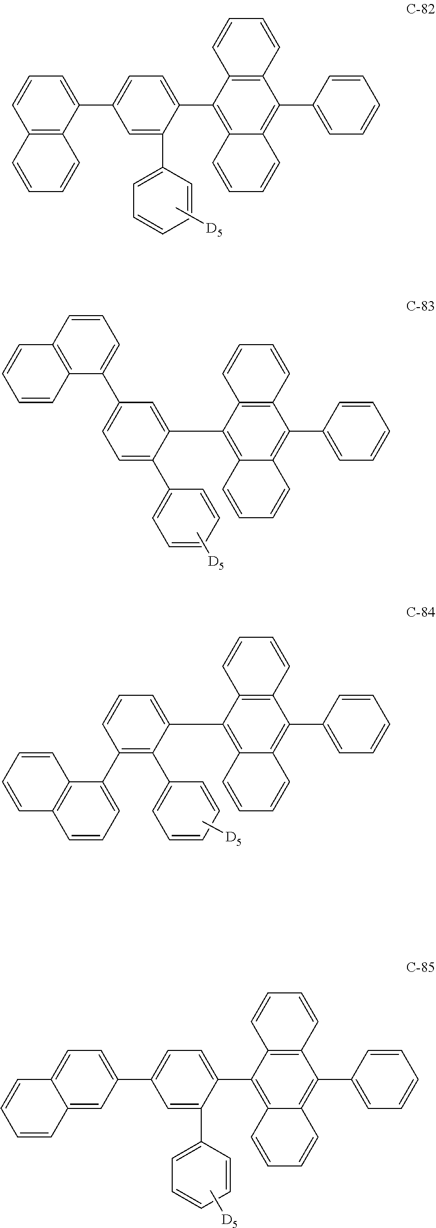 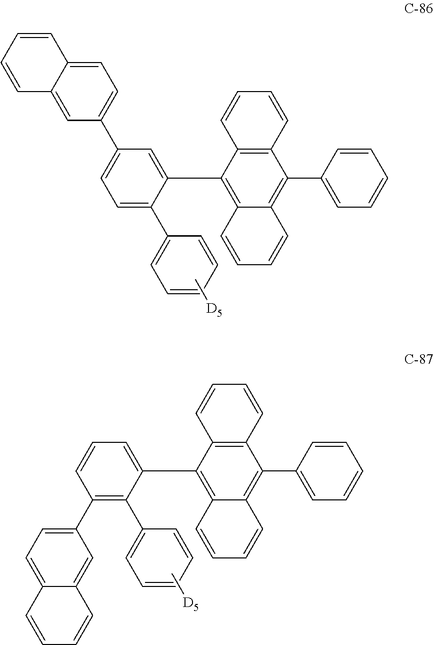 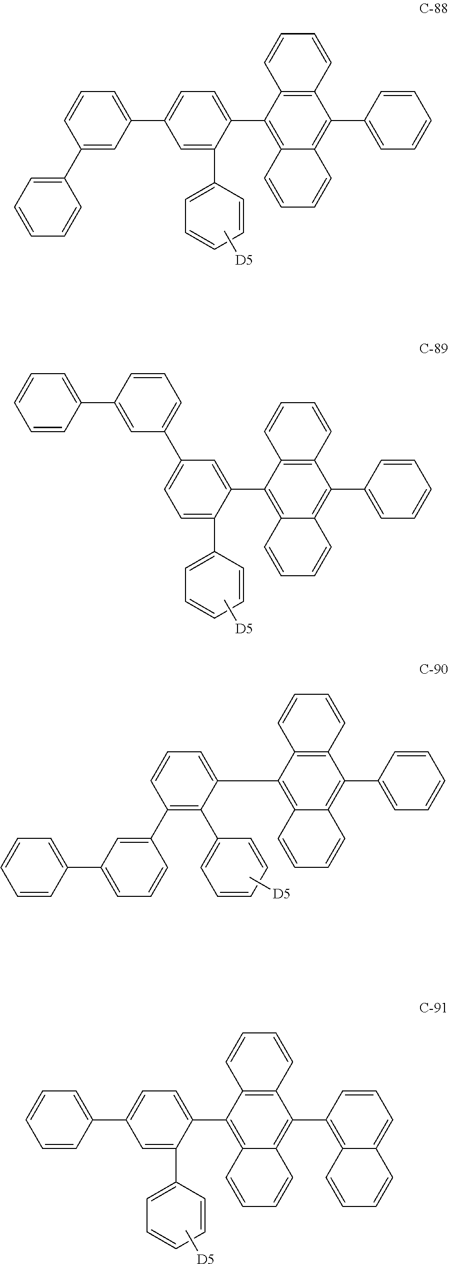 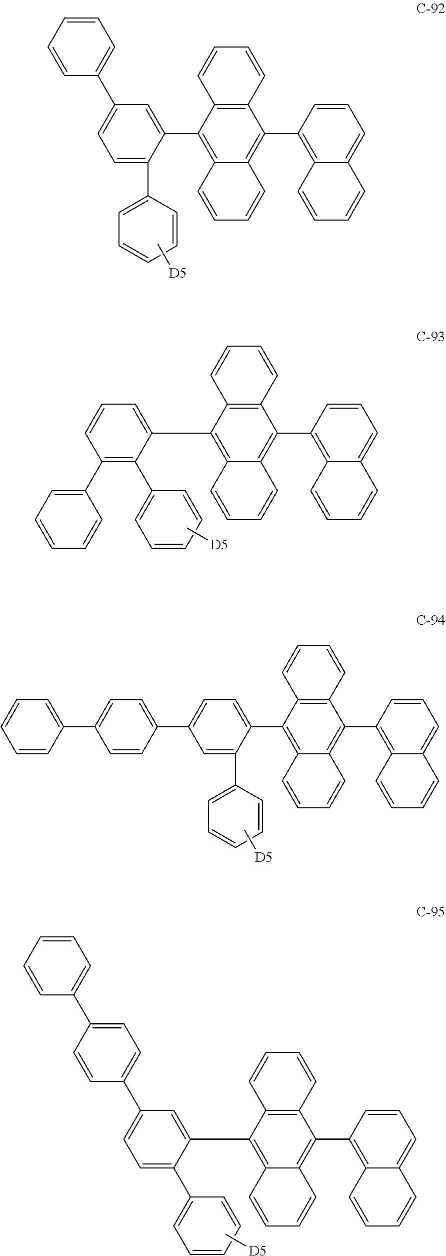 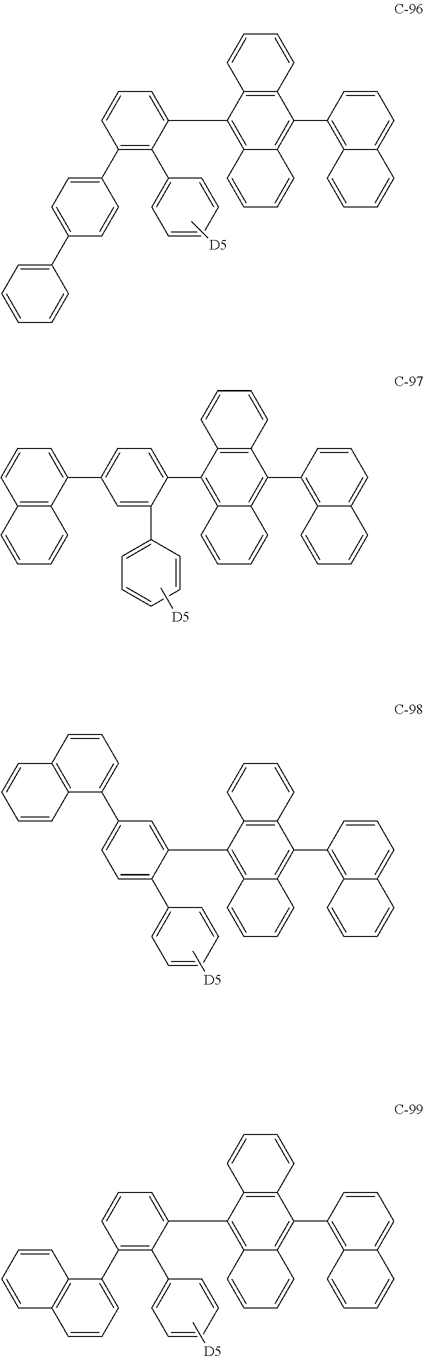 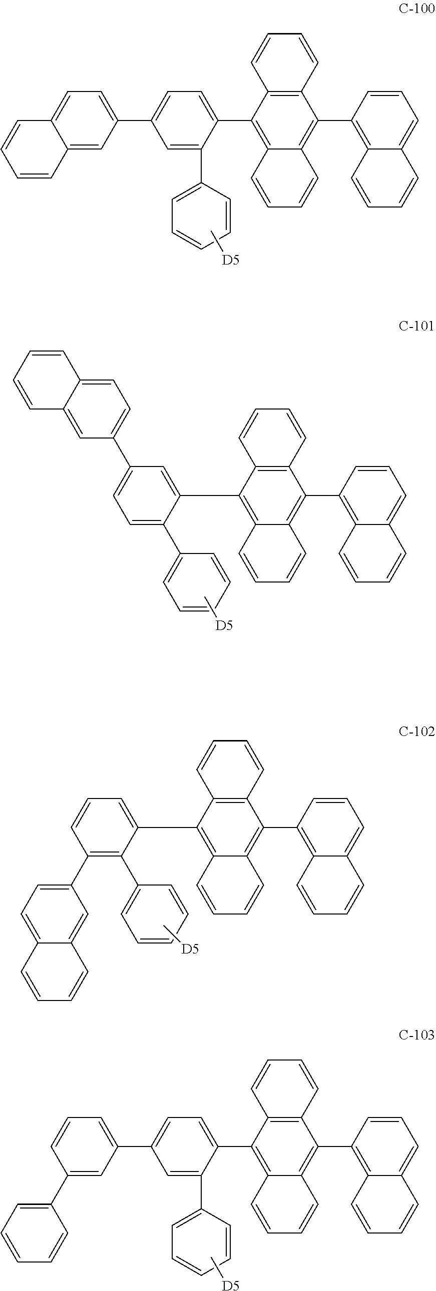 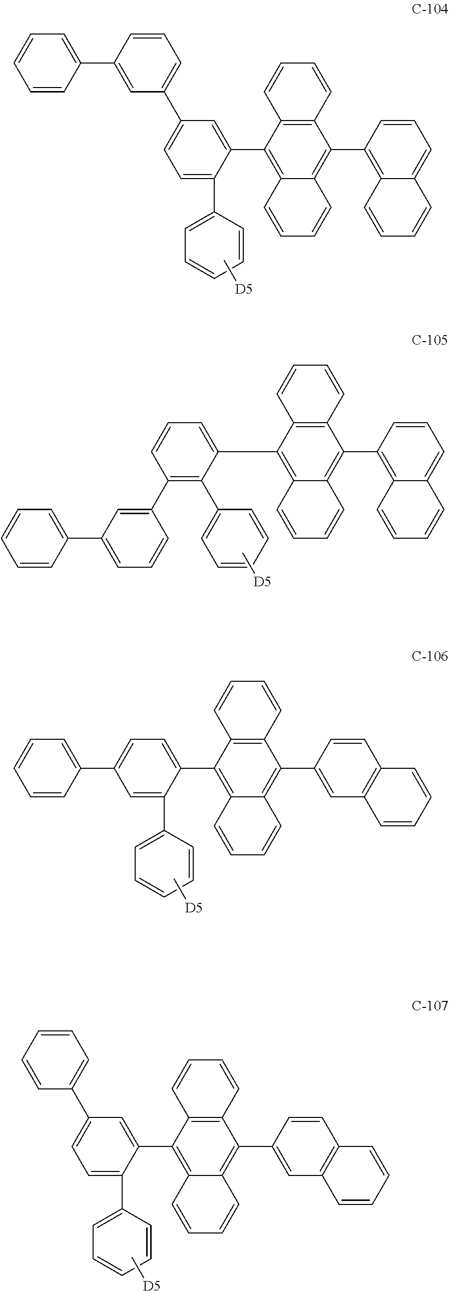 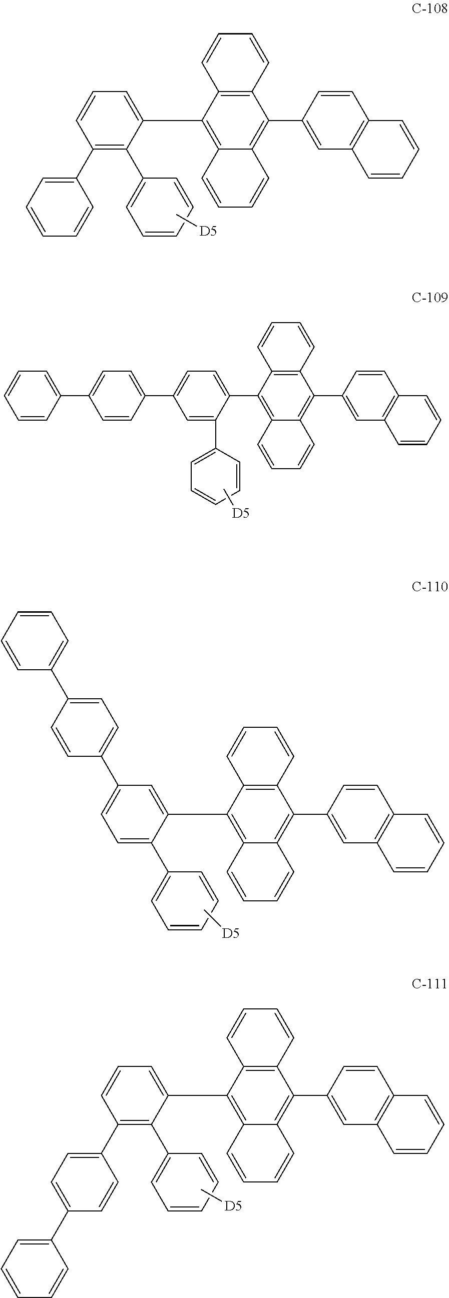 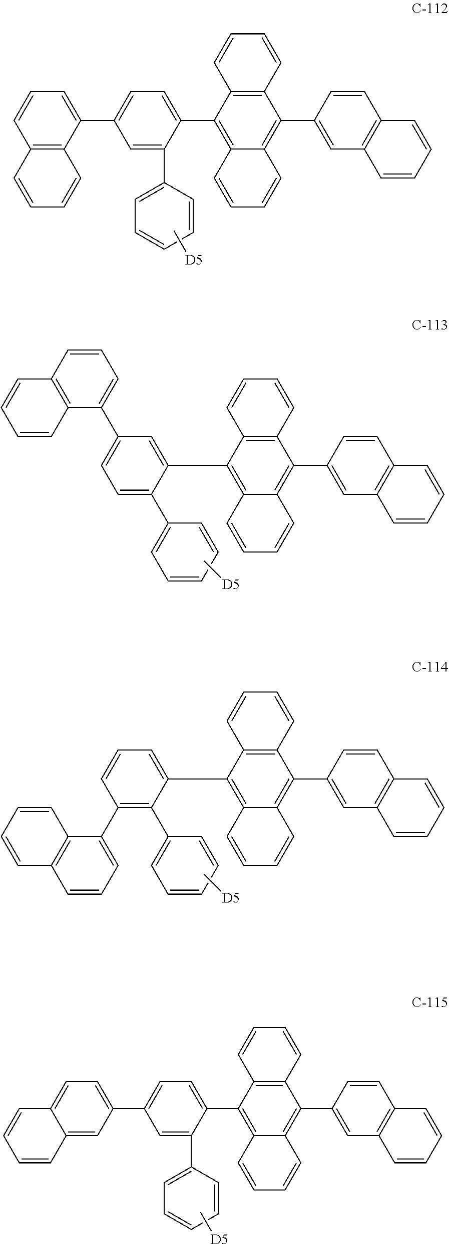 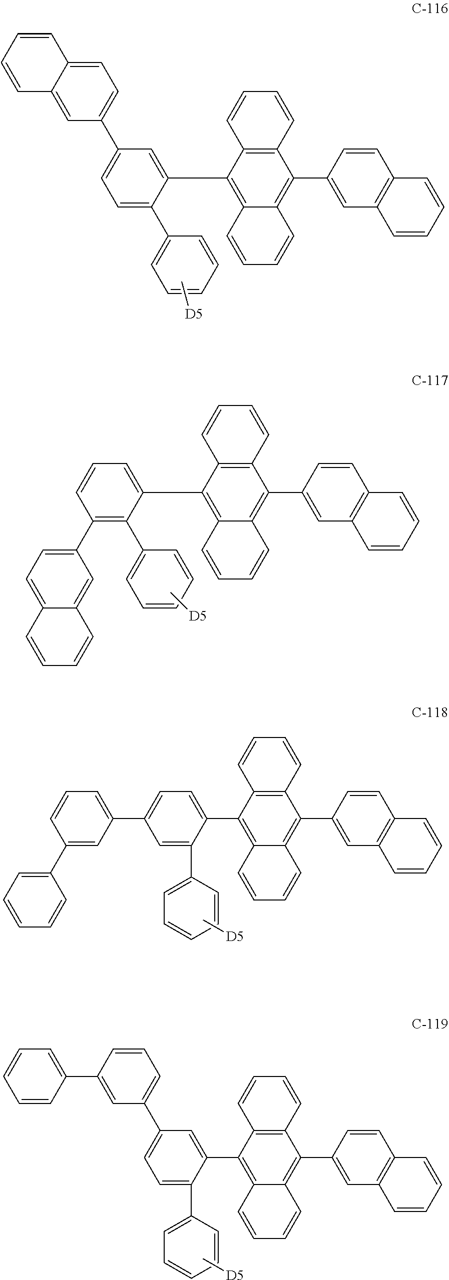 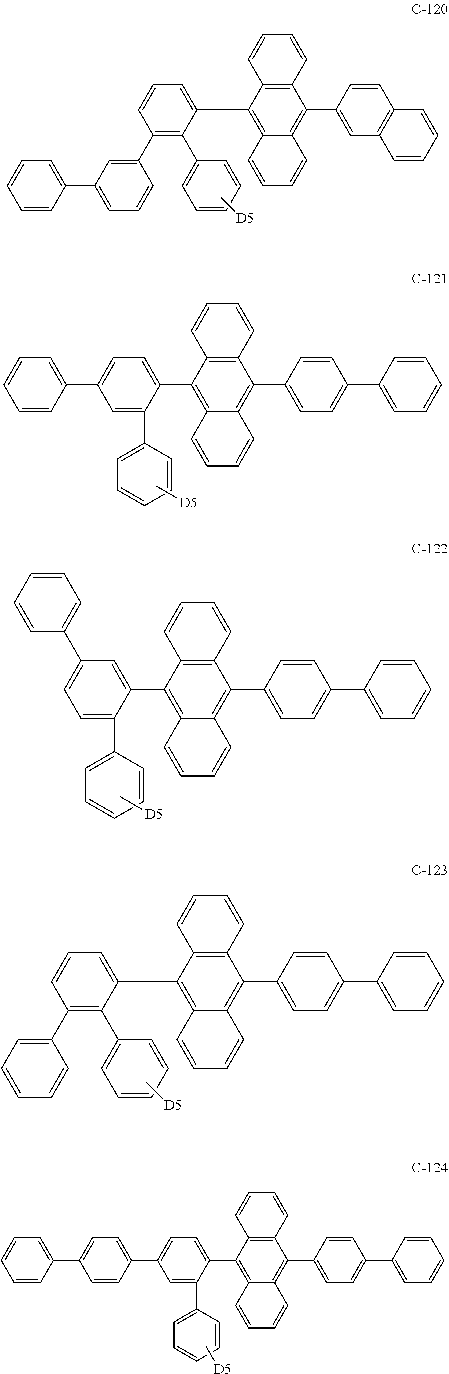 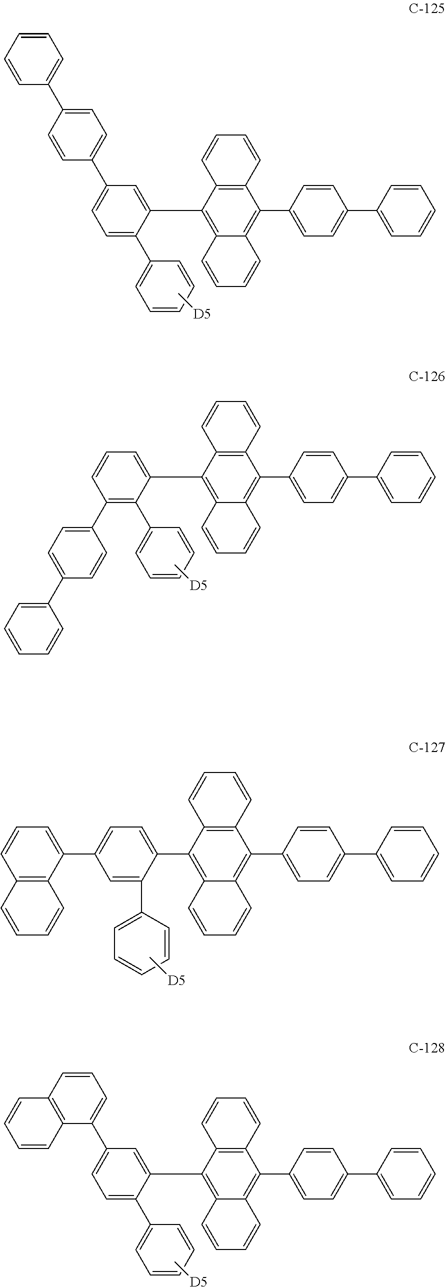 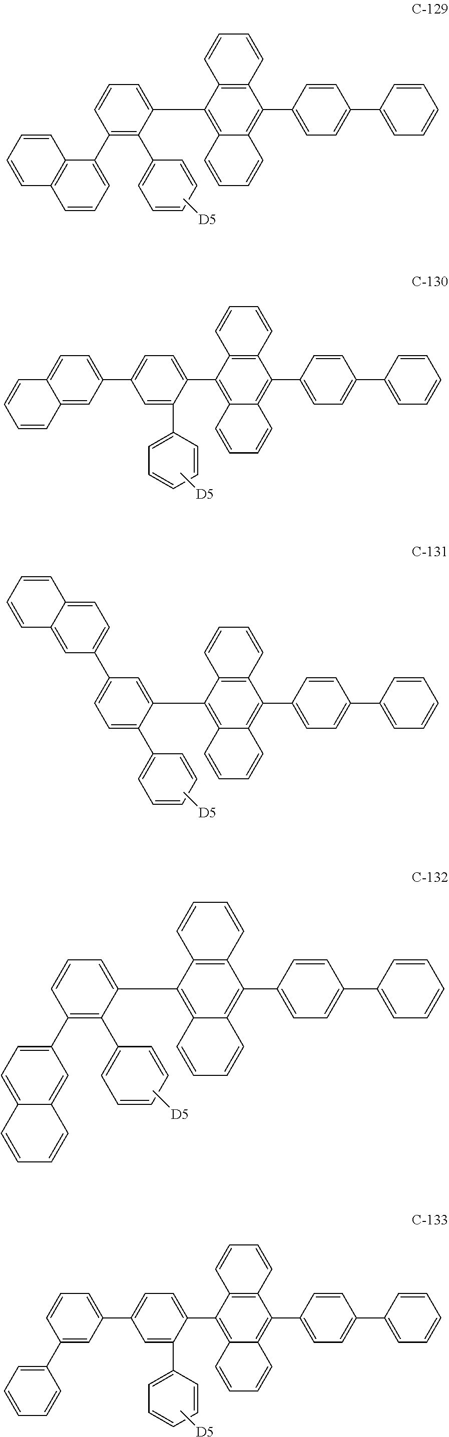 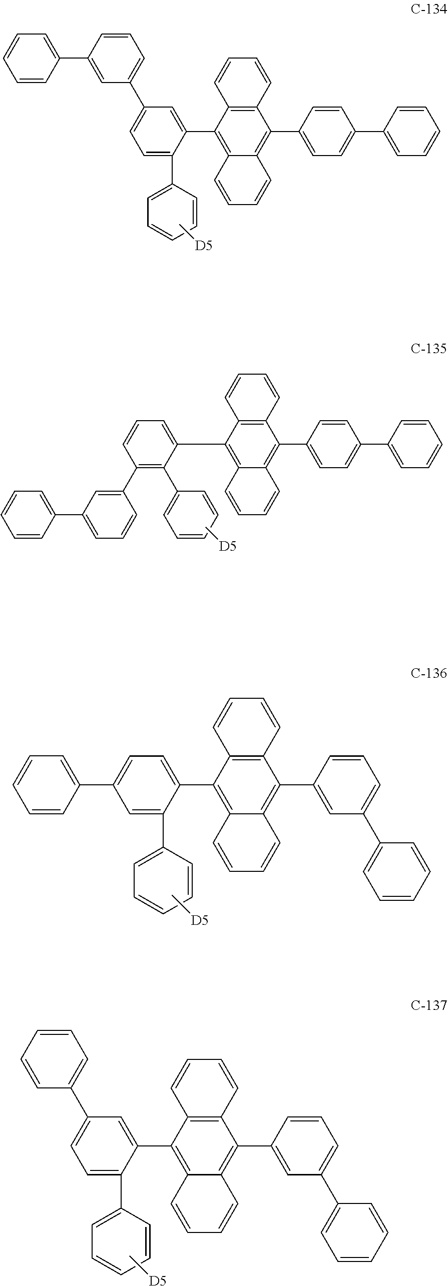 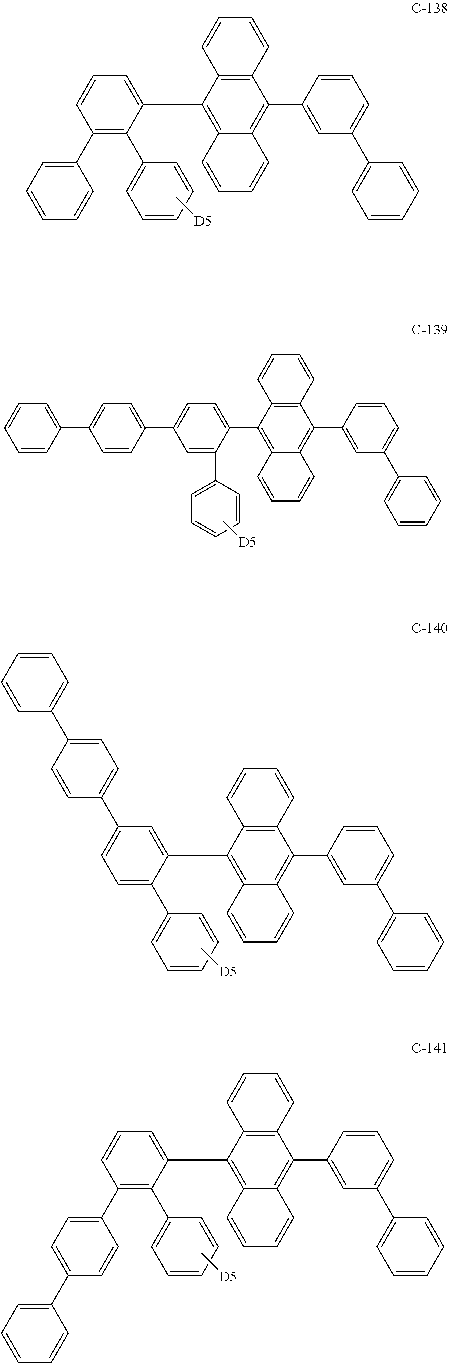 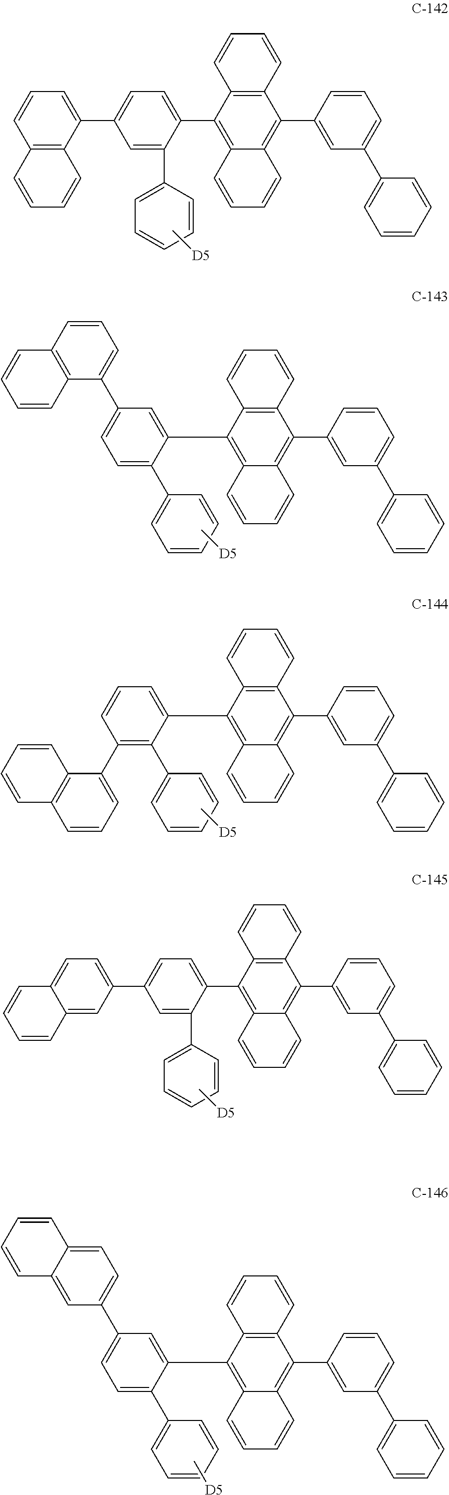 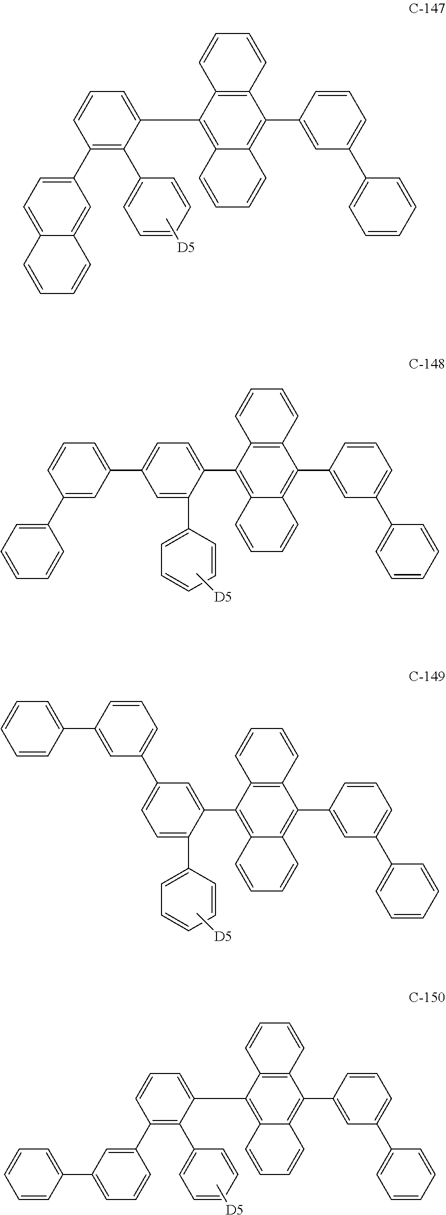
wherein, 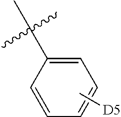
indicates 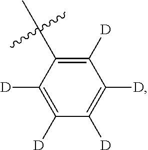
and wherein D indicates deuterium.

8. An organic light emitting diode, comprising:
a first electrode;
a second electrode facing the first electrode; and
an emissive layer disposed between the first electrode and the second electrode, wherein the emissive layer comprises an organic compound having the following structure of Chemical Formula 1: 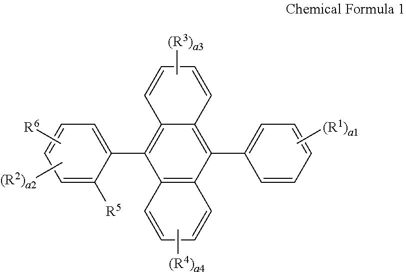
wherein, in Chemical Formula 1,
R¹ is protium, deuterium, tritium, an unsubstituted or substituted C₁-C₁₀ alkyl group or unsubstituted or substituted phenyl, where each R¹ is identical to or different from each other when a1 is 2, 3, 4 or 5, or
optionally,
two adjacent R¹ when a1 is 2, 3, 4 or 5 are further linked to form a benzene ring unsubstituted or substituted with at least one of deuterium, C₁-C₁₀ alkyl, and a group formed by connecting the two groups mentioned above;
each of R², R³ and R⁴ is independently protium, deuterium, tritium or an unsubstituted or substituted C₁-C₁₀ alkyl group, where each R² is identical to or different from each other when a2 is 2 or 3, each R³ is identical to or different from each other when a3 is 2, 3 or 4, and each R⁴ is identical to or different from each other when a4 is 2, 3 or 4;
each of R⁵ and R⁶ is independently phenyl, biphenyl or naphthyl, wherein each of the phenyl, biphenyl and the naphthyl is independently unsubstituted or substituted with at least one of deuterium, C₁-C₁₀ alkyl, and a group formed by connecting the two groups mentioned above;
a1 is 0, 1, 2, 3, 4 or 5;
a2 is 0, 1, 2 or 3; and
each of a3 and a4 is independently 0, 1, 2, 3 or 4.

9. The organic light emitting diode of claim 8, wherein the organic compound has the following structure of Chemical Formula 2 or Chemical Formula 3: 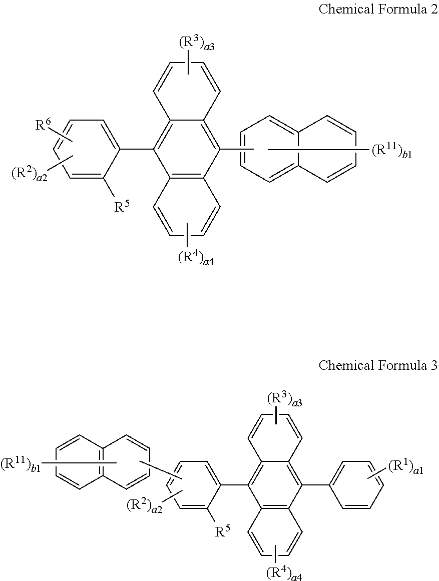
wherein, in Chemical Formulae 2 and 3,
each of R¹, R², R³, R⁴, R⁵, R⁶, a1, a2, a3 and a4 is a same as defined in Chemical Formula 1;
R¹¹ is protium, deuterium or an unsubstituted or deuterium-substituted C₁-C₁₀ alkyl group, where each R¹¹ is identical to or different from each other when b1 is 2, 3, 4, 5, 6 or 7; and
b1 is 0, 1, 2, 3, 4, 5, 6 or 7.

10. The organic light emitting diode of claim 8, wherein the organic compound has the following structure of Chemical Formula 4 or Chemical Formula 5: 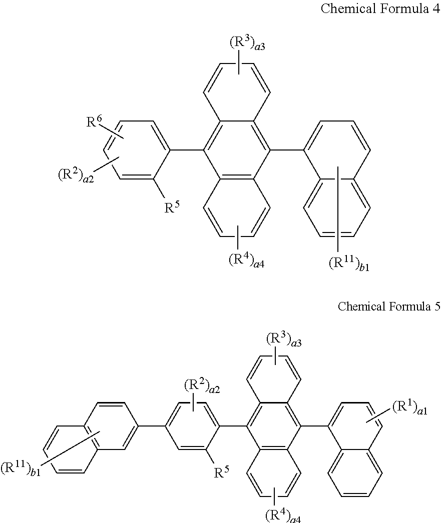
wherein, in Chemical Formulae 4 and 5,
each of R¹, R², R³, R⁴, R⁵, R⁶, a1, a2, a3 and a4 is a same as defined in Chemical Formula 1;
R¹¹ is protium, deuterium or an unsubstituted or deuterium-substituted C₁-C₁₀ alkyl group, where each R¹¹ is identical to or different from each other when b1 is 2, 3, 4, 5, 6 or 7; and
b1 is 0, 1, 2, 3, 4, 5, 6 or 7.

11. The organic light emitting diode of claim 8, wherein two adjacent R¹s are further linked to form a benzene ring, the rest three of R¹s are protium or deuterium, and R⁶ is naphthyl in Chemical Formula 1.

12. The organic light emitting diode of claim 8, wherein two adjacent R¹s are further linked to form a benzene ring, and thereby linked to the anthracene ring as 1-napthyl form, and R⁶ is 2-naphthyl.

13. The organic light emitting diode of claim 8, wherein R⁵ is phenyl, or phenyl where all hydrogens are substituted with deuterium.

14. The organic light emitting diode of claim 8, wherein the emissive layer comprises one or more emitting material layers, and wherein at least one emitting material layer comprises the organic compound.

15. The organic light emitting diode of claim 14, wherein the at least one emitting material layer comprises a host and a dopant, and wherein the host comprises the organic compound.

16. The organic light emitting diode of claim 15, wherein the dopant comprises a blue dopant.

17. The organic light emitting diode of claim 16, wherein the blue dopant comprises at least one of blue phosphorescent material, blue fluorescent material and blue delayed fluorescent material.

18. The organic light emitting diode of claim 15, wherein the dopant comprise phosphorescent material having the following structure of Chemical Formula 7: 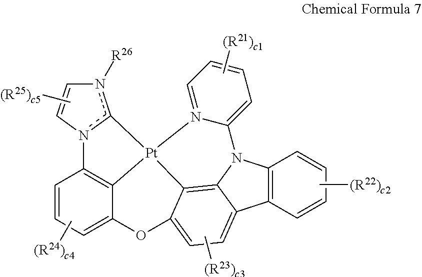
wherein, in Chemical Formula 7,
each of R²¹ to R²⁶ is independently protium, deuterium, tritium, halogen, a cyan group, an unsubstituted or substituted C₁-C₂₀ alkyl group, an unsubstituted or substituted C₂-C₂₀ alkenyl group, an unsubstituted or substituted C₂-C₂₀ alkynyl group, an unsubstituted or substituted C₁-C₂₀ alkoxy group, an amino group, an unsubstituted or substituted C₁-C₂₀ alkyl amino group, an unsubstituted or substituted C₁-C₂₀ alkyl silyl group, an unsubstituted or substituted C₃-C₃₀ cycloalkyl group, an unsubstituted or substituted C₃-C₃₀ hetero cycloalkyl group, an unsubstituted or substituted C₆-C₃₀ aryl group, an unsubstituted or substituted C₂-C₃₀ hetero aryl group, an unsubstituted or substituted C₇-C₃₀ aralkyl group, an unsubstituted or substituted C₃-C₃₀ hetero aralkyl group, an unsubstituted or substituted C₆-C₃₀ aryloxy group, an unsubstituted or substituted C₂-C₃₀ hetero aryloxy group, an unsubstituted or substituted C₆-C₃₀ aryl amino group, an unsubstituted or substituted C₂-C₃₀ hetero aryl amino group, an unsubstituted or substituted C₆-C₃₀ aryl silyl group, an unsubstituted or substituted C₂-C₃₀ hetero aryl silyl group, an unsubstituted or substituted C₁-C₂₀ alkyl germanyl group, an unsubstituted or substituted C₆-C₃₀ aryl germanyl group, an unsubstituted or substituted C₂-C₃₀ hetero aryl germanyl group, an unsubstituted or substituted C₆-C₃₀ tri-aryl methyl group or an unsubstituted or substituted C₂-C₃₀ tri-hetero aryl methyl group, where each R²¹ is identical to or different from each other when c1 is 2, 3 or 4, each R²² is identical to or different from each other when c2 is 2, 3 or 4, each R²³ is identical to or different from each other when c3 is 2, each R²⁴ is identical to or different from each other when c4 is 2 or 3, and each R²¹ is identical to different from each other when c5 is 2, or
optionally,
two adjacent R²¹, two adjacent R²², two adjacent R²³, two adjacent R²⁴ and/or two adjacent R²⁵ are further linked to form an unsubstituted or substituted benzene ring;
each of c1 and c2 is independently 0, 1, 2, 3 or 4;
each of c3 and c5 is independently 0, 1 or 2; and
c4 is 0, 1, 2 or 3.

19. The organic light emitting diode of claim 15, wherein the dopant comprises fluorescent material having the following structure of Chemical Formula 9: 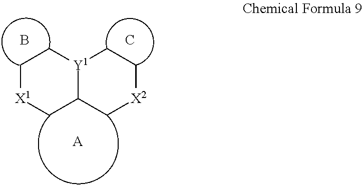
wherein, in Chemical Formula 9,
each of ring A, ring B and ring C is independently an unsubstituted or substituted C₆-C₃₀ aromatic ring or an unsubstituted or substituted C₃-C₃₀ hetero aromatic ring;
Y¹ is boron;
each of X¹ and X² is independently NR^{A}, O or S;
R^{A} is protium, deuterium, tritium, halogen, a cyan group, an unsubstituted or substituted C₁-C₂₀ alkyl group, an unsubstituted or substituted C₂-C₂₀ alkenyl group, an unsubstituted or substituted C₂-C₂₀ alkynyl group, an unsubstituted or substituted C₁-C₂₀ alkoxy group, an amino group, an unsubstituted or substituted C₁-C₂₀ alkyl amino group, an unsubstituted or substituted C₁-C₂₀ alkyl silyl group, an unsubstituted or substituted C₃-C₃₀ cycloalkyl group, an unsubstituted or substituted C₃-C₃₀ hetero cycloalkyl group, an unsubstituted or substituted C₆-C₃₀ aryl group, an unsubstituted or substituted C₂-C₃₀ hetero aryl group, an unsubstituted or substituted C₇-C₃₀ aralkyl group, an unsubstituted or substituted C₃-C₃₀ hetero aralkyl group, an unsubstituted or substituted C₆-C₃₀ aryloxy group, an unsubstituted or substituted C₂-C₃₀ hetero aryloxy group, an unsubstituted or substituted C₆-C₃₀ aryl amino group, an unsubstituted or substituted C₂-C₃₀ hetero aryl amino group, an unsubstituted or substituted tri-phenyl methyl group, an unsubstituted or substituted C₆-C₃₀ aryl silyl group, an unsubstituted or substituted C₂-C₃₀ hetero aryl silyl group, an unsubstituted or substituted C₁-C₂₀ alkyl germanyl group, an unsubstituted or substituted C₆-C₃₀ aryl germanyl group, an unsubstituted or substituted C₂-C₃₀ hetero aryl germanyl group, or -L-N—(R^{B})(R^{C}), or
optionally,
R^{A} is linked to at least one of ring A, ring B and ring C to form an unsubstituted or substituted C₃-C₃₀ alicyclic ring, an unsubstituted or substituted C₃-C₃₀ hetero alicyclic ring, an unsubstituted or substituted C₆-C₃₀ aromatic ring or an unsubstituted or substituted C₂-C₃₀ hetero aromatic ring;
L¹ is a single bond, an unsubstituted or substituted C₆-C₃₀ arylene group, an unsubstituted or substituted C₂-C₃₀ hetero arylene group, an unsubstituted or substituted divalent C₁-C₃₀ aliphatic group, or a divalent fused ring group of an unsubstituted or substituted C₃-C₃₀ alicyclic ring and an unsubstituted or substituted C₆-C₃₀ aromatic ring; and
each of R^{B} and R^{C} is independently an unsubstituted or substituted C₁-C₂₀ alkyl group, an unsubstituted or substituted C₂-C₂₀ alkenyl group, an unsubstituted or substituted C₆-C₃₀ aryl group or an unsubstituted or substituted C₂-C₃₀ hetero aryl group.

20. The organic light emitting diode of claim 15, wherein the dopant comprises a first dopant and a second dopant.

21. The organic light emitting diode of claim 20, wherein the first dopant comprises at least one of blue delayed fluorescent material and blue phosphorescent material, and the second dopant comprises blue fluorescent material.

22. The organic light emitting diode of claim 8, wherein the emissive layer comprises:
a first emitting part disposed between the first electrode and the second electrode, and comprising a first emitting material layer;
a second emitting part disposed between the first emitting part and the second electrode, and comprising a second emitting material layer; and
a first charge generation layer disposed between the first emitting part and the second emitting part, and
wherein at least one of the first emitting material layer and the second emitting material layer comprises the organic compound.

23. The organic light emitting diode of claim 22, wherein the first emitting material layer comprises the organic compound.

24. The organic light emitting diode of claim 22, wherein the second emitting material layer emits red to green color light.

25. The organic light emitting diode of claim 22, wherein the emissive layer further comprises:
a third emitting part disposed between the second emitting part and the second electrode, and comprising a third emitting material layer; and
a second charge generation layer disposed between the second emitting part and the third emitting part.

26. The organic light emitting diode of claim 25, wherein at least one of the first emitting material layer and the third emitting material layer comprises the organic compound.

27. An organic light emitting device, comprising:
a substrate; and
the organic light emitting diode of claim 8 over the substrate.

28. An organic compound having the following structure of Chemical Formula 1: 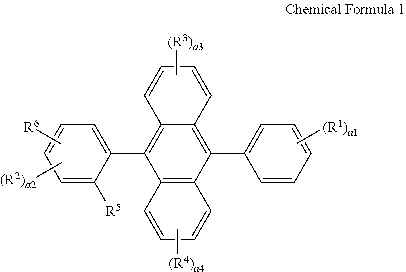
wherein, in Chemical Formula 1,
R¹ is protium, deuterium, or tritium, where each R¹ is identical to or different from each other when a1 is 2, 3, 4 or 5, or
optionally,
two adjacent R¹s are linked to form a benzene ring unsubstituted or substituted with at least one of deuterium, C₁-C₁₀ alkyl, and a group formed by connecting the two groups mentioned above, and the rest there of R¹s are protium, deuterium, or tritium;
each of R², R³ and R⁴ is independently protium, deuterium, tritium, where each R² is identical to or different from each other when a2 is 2 or 3, each R³ is identical to or different from each other when a3 is 2, 3 or 4, and each R⁴ is identical to or different from each other when a4 is 2, 3 or 4;
R⁵ is phenyl unsubstituted or substituted with at least one deuterium;
R⁶ is phenyl, biphenyl or naphthyl, wherein each of the phenyl, biphenyl and the naphthyl is independently unsubstituted or substituted with at least one of deuterium, C₁-C₁₀ alkyl, and a group formed by connecting the two groups mentioned above;
a1 is 0, 1, 2, 3, 4 or 5;
a2 is 0, 1, 2 or 3; and
each of a3 and a4 is independently 0, 1, 2, 3 or 4.

29. The organic compound of claim 28, wherein R⁵ is phenyl, or phenyl where all hydrogens are substituted with deuterium.

30. An organic light emitting diode, comprising:
a first electrode;
a second electrode facing the first electrode; and
an emissive layer disposed between the first electrode and the second electrode,
wherein the emissive layer comprises an organic compound having the following structure of Chemical Formula 1: 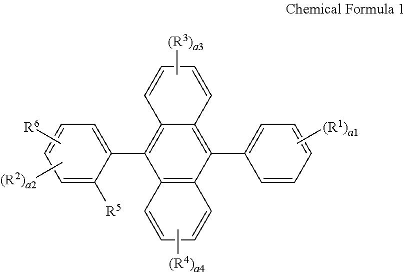
wherein, in Chemical Formula 1,
R¹ is protium, deuterium, or tritium, where each R¹ is identical to or different from each other when a1 is 2, 3, 4 or 5, or
optionally,
two adjacent R¹s are linked to form a benzene ring unsubstituted or substituted with at least one of deuterium, C₁-C₁₀ alkyl, and a group formed by connecting the two groups mentioned above, and the rest there of R¹s are protium, deuterium, or tritium;
each of R², R³ and R⁴ is independently protium, deuterium, tritium, where each R² is identical to or different from each other when a2 is 2 or 3, each R³ is identical to or different from each other when a3 is 2, 3 or 4, and each R⁴ is identical to or different from each other when a4 is 2, 3 or 4;
R⁵ is phenyl unsubstituted or substituted with at least one deuterium;
R⁶ is phenyl, biphenyl or naphthyl, wherein each of the phenyl, biphenyl and the naphthyl is independently unsubstituted or substituted with at least one of deuterium, C₁-C₁₀ alkyl, and a group formed by connecting the two groups mentioned above;
a1 is 0, 1, 2, 3, 4 or 5;
a2 is 0, 1, 2 or 3; and
each of a3 and a4 is independently 0, 1, 2, 3 or 4.

31. The organic light emitting diode of claim 30, wherein R⁵ is phenyl, or phenyl where all hydrogens are substituted with deuterium.
